SEQUENCE LISTING (164 sequences)

SEQ ID NO: 1 - protein, 14 aa, Homo sapiens
SKAVEQVKNAFNKL

SEQ ID NO: 2 - protein, 27 aa, Homo sapiens
EKGIYKAMSEFDIFINYIEAYMTMKIR

SEQ ID NO: 3 - protein, 24 aa, Homo sapiens
NLPNMLRDLRDAFSRVKTFFQMKD

SEQ ID NO: 4 - protein, 10 aa, Homo sapiens
KESLLEDFKG

SEQ ID NO: 5 - protein, 23 aa, Homo sapiens
LGCQALSEMIQFYLEEVMPQAEN

SEQ ID NO: 6 - protein, 22 aa, Homo sapiens
IKAHVNSLGENLKTLRLRLRRC

SEQ ID NO: 7 - protein, 18 aa, synthetic construct
NSPGQGTQSENSCTHFPG

SEQ ID NO: 8 - protein, 7 aa, synthetic construct
QLDNLLL

SEQ ID NO: 9 - protein, 4 aa, synthetic construct
QDPD

SEQ ID NO: 10 - protein, 160 aa, Homo sapiens
KSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 11 - protein, 161 aa, Homo sapiens
KSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 12 - protein, 42 aa, Homo sapiens
KSKAVEQVKNAFNKLQEKGIYKAMSEFDIFINYIEAYMTMKI

SEQ ID NO: 13 - protein, 43 aa, Homo sapiens
KSKAVEQVKNAFNKLQEKGIYKAMSEFDIFINYIEAYMTMKIR

SEQ ID NO: 14 - protein, 44 aa, Homo sapiens
KSKAVEQVKNAFNKLQEKGIYKAMSEFDIFINYIEAYMTMKIRN

SEQ ID NO: 15 - protein, 116 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 16 - protein, 115 aa, Homo sapiens
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 17 - protein, 114 aa, Homo sapiens
GQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 18 - protein, 113 aa, Homo sapiens
QGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 19 - protein, 112 aa, Homo sapiens
GTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 20 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 21 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 22 - protein, 178 aa, Mus sp.
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 23 - protein, 19 aa, synthetic construct
NTSPGQGTQSENSCTHFPG

SEQ ID NO: 24 - protein, 557 aa, Homo sapiens
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 25 - protein, 306 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 26 - protein, 330 aa, Mus sp.
MIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPSWIVAIILIVSVLVVFLFLLGCFVVLWLIYKKTKHTFRSGTSLPQHLKEFLGHPHHSTFLLFSFPPPEEAEVFDKLSIISEESEGSKQSPEDNCASEPPSDPGPRELESKDEAPSPPHDDPKLLTSTSEV

SEQ ID NO: 27 - protein, 171 aa, synthetic construct
KSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 28 - protein, 172 aa, synthetic construct
NKSKAVEQVKSDFNKLQDQGVYKAMNEFDIFINAIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENGGSHHHHHHHH

SEQ ID NO: 29 - protein, 111 aa, Homo sapiens
TQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 30 - protein, 110 aa, Homo sapiens
QSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 31 - protein, 109 aa, Homo sapiens
SENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 32 - protein, 108 aa, Homo sapiens
ENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 33 - protein, 107 aa, Homo sapiens
NSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 34 - protein, 106 aa, Homo sapiens
SCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 35 - protein, 105 aa, Homo sapiens
CTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 36 - protein, 159 aa, Homo sapiens
PGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 37 - protein, 158 aa, Homo sapiens
GQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 38 - protein, 157 aa, Homo sapiens
QGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 39 - protein, 156 aa, Homo sapiens
GTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 40 - protein, 155 aa, Homo sapiens
TQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 41 - protein, 154 aa, Homo sapiens
QSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 42 - protein, 153 aa, Homo sapiens
SENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 43 - protein, 152 aa, Homo sapiens
ENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 44 - protein, 151 aa, Homo sapiens
NSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 45 - protein, 150 aa, Homo sapiens
SCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 46 - protein, 320 aa, synthetic construct
KSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 47 - protein, 322 aa, synthetic construct
KSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 48 - protein, 322 aa, synthetic construct
KSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 49 - protein, 324 aa, synthetic construct
KSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 50 - protein, 320 aa, synthetic construct
KSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 51 - protein, 320 aa, synthetic construct
KSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCEN

SEQ ID NO: 52 - protein, 11 aa, synthetic construct
GGSHHHHHHHH

SEQ ID NO: 53 - protein, 160 aa, synthetic construct
KSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 54 - protein, 320 aa, synthetic construct; identical to SEQ ID NO: 50

SEQ ID NO: 55 - protein, 320 aa, synthetic construct; identical to SEQ ID NO: 51

SEQ ID NO: 56 - protein, 100 aa, synthetic construct
  VARIANT 1..100: note = This sequence may encompass 1-20 GSGGS repeating units
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 57 - protein, 80 aa, synthetic construct
  VARIANT 1..80: note = This sequence may encompass 1-20 GGGS repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 58 - protein, 100 aa, synthetic construct
  VARIANT 1..100: note = This sequence may encompass 1-20 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 59 - protein, 1200 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 801..820: note = This region may encompass 1-20 residues
  VARIANT 821..840: note = This region may encompass 1-20 residues
  VARIANT 841..860: note = This region may encompass 1-20 residues
  VARIANT 861..880: note = This region may encompass 1-20 residues
  VARIANT 881..900: note = This region may encompass 1-20 residues
  VARIANT 901..920: note = This region may encompass 1-20 residues
  VARIANT 921..940: note = This region may encompass 1-20 residues
  VARIANT 941..960: note = This region may encompass 1-20 residues
  VARIANT 961..980: note = This region may encompass 1-20 residues
  VARIANT 981..1000: note = This region may encompass 1-20 residues
  VARIANT 1001..1020: note = This region may encompass 1-20 residues
  VARIANT 1021..1040: note = This region may encompass 1-20 residues
  VARIANT 1041..1060: note = This region may encompass 1-20 residues
  VARIANT 1061..1080: note = This region may encompass 1-20 residues
  VARIANT 1081..1100: note = This region may encompass 1-20 residues
  VARIANT 1101..1120: note = This region may encompass 1-20 residues
  VARIANT 1121..1140: note = This region may encompass 1-20 residues
  VARIANT 1141..1160: note = This region may encompass 1-20 residues
  VARIANT 1161..1180: note = This region may encompass 1-20 residues
  VARIANT 1181..1200: note = This region may encompass 1-20 residues
  VARIANT 1..1200: note = This sequence may encompass 1-20 GmSoGm repeating units, wherein m = 1 to 20, o = 1 to 20
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 60 - protein, 2000 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 801..820: note = This region may encompass 1-20 residues
  VARIANT 821..840: note = This region may encompass 1-20 residues
  VARIANT 841..860: note = This region may encompass 1-20 residues
  VARIANT 861..880: note = This region may encompass 1-20 residues
  VARIANT 881..900: note = This region may encompass 1-20 residues
  VARIANT 901..920: note = This region may encompass 1-20 residues
  VARIANT 921..940: note = This region may encompass 1-20 residues
  VARIANT 941..960: note = This region may encompass 1-20 residues
  VARIANT 961..980: note = This region may encompass 1-20 residues
  VARIANT 981..1000: note = This region may encompass 1-20 residues
  VARIANT 1001..1020: note = This region may encompass 1-20 residues
  VARIANT 1021..1040: note = This region may encompass 1-20 residues
  VARIANT 1041..1060: note = This region may encompass 1-20 residues
  VARIANT 1061..1080: note = This region may encompass 1-20 residues
  VARIANT 1081..1100: note = This region may encompass 1-20 residues
  VARIANT 1101..1120: note = This region may encompass 1-20 residues
  VARIANT 1121..1140: note = This region may encompass 1-20 residues
  VARIANT 1141..1160: note = This region may encompass 1-20 residues
  VARIANT 1161..1180: note = This region may encompass 1-20 residues
  VARIANT 1181..1200: note = This region may encompass 1-20 residues
  VARIANT 1201..1220: note = This region may encompass 1-20 residues
  VARIANT 1221..1240: note = This region may encompass 1-20 residues
  VARIANT 1241..1260: note = This region may encompass 1-20 residues
  VARIANT 1261..1280: note = This region may encompass 1-20 residues
  VARIANT 1281..1300: note = This region may encompass 1-20 residues
  VARIANT 1301..1320: note = This region may encompass 1-20 residues
  VARIANT 1321..1340: note = This region may encompass 1-20 residues
  VARIANT 1341..1360: note = This region may encompass 1-20 residues
  VARIANT 1361..1380: note = This region may encompass 1-20 residues
  VARIANT 1381..1400: note = This region may encompass 1-20 residues
  VARIANT 1401..1420: note = This region may encompass 1-20 residues
  VARIANT 1421..1440: note = This region may encompass 1-20 residues
  VARIANT 1441..1460: note = This region may encompass 1-20 residues
  VARIANT 1461..1480: note = This region may encompass 1-20 residues
  VARIANT 1481..1500: note = This region may encompass 1-20 residues
  VARIANT 1501..1520: note = This region may encompass 1-20 residues
  VARIANT 1521..1540: note = This region may encompass 1-20 residues
  VARIANT 1541..1560: note = This region may encompass 1-20 residues
  VARIANT 1561..1580: note = This region may encompass 1-20 residues
  VARIANT 1581..1600: note = This region may encompass 1-20 residues
  VARIANT 1601..1620: note = This region may encompass 1-20 residues
  VARIANT 1621..1640: note = This region may encompass 1-20 residues
  VARIANT 1641..1660: note = This region may encompass 1-20 residues
  VARIANT 1661..1680: note = This region may encompass 1-20 residues
  VARIANT 1681..1700: note = This region may encompass 1-20 residues
  VARIANT 1701..1720: note = This region may encompass 1-20 residues
  VARIANT 1721..1740: note = This region may encompass 1-20 residues
  VARIANT 1741..1760: note = This region may encompass 1-20 residues
  VARIANT 1761..1780: note = This region may encompass 1-20 residues
  VARIANT 1781..1800: note = This region may encompass 1-20 residues
  VARIANT 1801..1820: note = This region may encompass 1-20 residues
  VARIANT 1821..1840: note = This region may encompass 1-20 residues
  VARIANT 1841..1860: note = This region may encompass 1-20 residues
  VARIANT 1861..1880: note = This region may encompass 1-20 residues
  VARIANT 1881..1900: note = This region may encompass 1-20 residues
  VARIANT 1901..1920: note = This region may encompass 1-20 residues
  VARIANT 1921..1940: note = This region may encompass 1-20 residues
  VARIANT 1941..1960: note = This region may encompass 1-20 residues
  VARIANT 1961..1980: note = This region may encompass 1-20 residues
  VARIANT 1981..2000: note = This region may encompass 1-20 residues
  VARIANT 1..2000: note = This sequence may encompass 1-20 GmSoGmSoGm repeating units, wherein m = 1 to 20, o = 1 to 20
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 61 - protein, 480 aa, synthetic construct
  VARIANT 5..24: note = This region may encompass 1-20 residues
  VARIANT 29..48: note = This region may encompass 1-20 residues
  VARIANT 53..72: note = This region may encompass 1-20 residues
  VARIANT 77..96: note = This region may encompass 1-20 residues
  VARIANT 101..120: note = This region may encompass 1-20 residues
  VARIANT 125..144: note = This region may encompass 1-20 residues
  VARIANT 149..168: note = This region may encompass 1-20 residues
  VARIANT 173..192: note = This region may encompass 1-20 residues
  VARIANT 197..216: note = This region may encompass 1-20 residues
  VARIANT 221..240: note = This region may encompass 1-20 residues
  VARIANT 245..264: note = This region may encompass 1-20 residues
  VARIANT 269..288: note = This region may encompass 1-20 residues
  VARIANT 293..312: note = This region may encompass 1-20 residues
  VARIANT 317..336: note = This region may encompass 1-20 residues
  VARIANT 341..360: note = This region may encompass 1-20 residues
  VARIANT 365..384: note = This region may encompass 1-20 residues
  VARIANT 389..408: note = This region may encompass 1-20 residues
  VARIANT 413..432: note = This region may encompass 1-20 residues
  VARIANT 437..456: note = This region may encompass 1-20 residues
  VARIANT 461..480: note = This region may encompass 1-20 residues
  VARIANT 1..480: note = This sequence may encompass 1-20 GSGGSm repeating units, wherein m = 1 to 20
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 62 - protein, 480 aa, synthetic construct
  VARIANT 4..23: note = This region may encompass 1-20 residues
  VARIANT 28..47: note = This region may encompass 1-20 residues
  VARIANT 52..71: note = This region may encompass 1-20 residues
  VARIANT 76..95: note = This region may encompass 1-20 residues
  VARIANT 100..119: note = This region may encompass 1-20 residues
  VARIANT 124..143: note = This region may encompass 1-20 residues
  VARIANT 148..167: note = This region may encompass 1-20 residues
  VARIANT 172..191: note = This region may encompass 1-20 residues
  VARIANT 196..215: note = This region may encompass 1-20 residues
  VARIANT 220..239: note = This region may encompass 1-20 residues
  VARIANT 244..263: note = This region may encompass 1-20 residues
  VARIANT 268..287: note = This region may encompass 1-20 residues
  VARIANT 292..311: note = This region may encompass 1-20 residues
  VARIANT 316..335: note = This region may encompass 1-20 residues
  VARIANT 340..359: note = This region may encompass 1-20 residues
  VARIANT 364..383: note = This region may encompass 1-20 residues
  VARIANT 388..407: note = This region may encompass 1-20 residues
  VARIANT 412..431: note = This region may encompass 1-20 residues
  VARIANT 436..455: note = This region may encompass 1-20 residues
  VARIANT 460..479: note = This region may encompass 1-20 residues
  VARIANT 1..480: note = This sequence may encompass 1-20 GSGSmG repeating units, wherein m = 1 to 20
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 63 - protein, 460 aa, synthetic construct
  VARIANT 4..23: note = This region may encompass 1-20 residues
  VARIANT 27..46: note = This region may encompass 1-20 residues
  VARIANT 50..69: note = This region may encompass 1-20 residues
  VARIANT 73..92: note = This region may encompass 1-20 residues
  VARIANT 96..115: note = This region may encompass 1-20 residues
  VARIANT 119..138: note = This region may encompass 1-20 residues
  VARIANT 142..161: note = This region may encompass 1-20 residues
  VARIANT 165..184: note = This region may encompass 1-20 residues
  VARIANT 188..207: note = This region may encompass 1-20 residues
  VARIANT 211..230: note = This region may encompass 1-20 residues
  VARIANT 234..253: note = This region may encompass 1-20 residues
  VARIANT 257..276: note = This region may encompass 1-20 residues
  VARIANT 280..299: note = This region may encompass 1-20 residues
  VARIANT 303..322: note = This region may encompass 1-20 residues
  VARIANT 326..345: note = This region may encompass 1-20 residues
  VARIANT 349..368: note = This region may encompass 1-20 residues
  VARIANT 372..391: note = This region may encompass 1-20 residues
  VARIANT 395..414: note = This region may encompass 1-20 residues
  VARIANT 418..437: note = This region may encompass 1-20 residues
  VARIANT 441..460: note = This region may encompass 1-20 residues
  VARIANT 1..460: note = This sequence may encompass 1-20 GGGSm repeating units, wherein m = 1 to 20
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 64 - protein, 100 aa, synthetic construct; identical to SEQ ID NO: 58
  VARIANT 1..100: note = This sequence may encompass 1-20 GGGGS repeating units

SEQ ID NO: 65 - protein, 4 aa, synthetic construct
GGSG

SEQ ID NO: 66 - protein, 5 aa, synthetic construct
GGSGG

SEQ ID NO: 67 - protein, 5 aa, synthetic construct
GSGSG

SEQ ID NO: 68 - protein, 5 aa, synthetic construct
GSGGG

SEQ ID NO: 69 - protein, 5 aa, synthetic construct
GGGSG

SEQ ID NO: 70 - protein, 5 aa, synthetic construct
GSSSG

SEQ ID NO: 71 - protein, 4 aa, synthetic construct
GGGS

SEQ ID NO: 72 - protein, 5 aa, synthetic construct
GGGGS

SEQ ID NO: 73 - protein, 10 aa, synthetic construct
GGGGSGGGGS

SEQ ID NO: 74 - protein, 15 aa, synthetic construct
GGGGSGGGGSGGGGS

SEQ ID NO: 75 - protein, 4 aa, synthetic construct
GGSG

SEQ ID NO: 76 - protein, 5 aa, synthetic construct
GGSGG

SEQ ID NO: 77 - protein, 5 aa, synthetic construct
GSGSG

SEQ ID NO: 78 - protein, 5 aa, synthetic construct
GSGGG

SEQ ID NO: 79 - protein, 5 aa, synthetic construct
GGGSG

SEQ ID NO: 80 - protein, 10 aa, synthetic construct
GGGGSGGGGS

SEQ ID NO: 81 - protein, 15 aa, synthetic construct
GGGGSGGGGSGSSSG

SEQ ID NO: 82-84 - skipped (3 entries)

SEQ ID NO: 85 - DNA, 54 bp, Homo sapiens
  source 1..54: mol_type = genomic DNA
atgcacagcagcgccctgctgtgctgcctggtgctgctgaccggcgtgagagcc

SEQ ID NO: 86 - protein, 18 aa, Homo sapiens
MHSSALLCCLVLLTGVRA

SEQ ID NO: 87 - protein, 18 aa, Mus sp.
MPGSALLCCLLLLTGMRI

SEQ ID NO: 88 - DNA, 57 bp, Mus sp.
  source 1..57: mol_type = genomic DNA
atgggatggtcctgcatcatcctgtttctggtggctaccgccaccggtgtgcacagc

SEQ ID NO: 89 - protein, 19 aa, Mus sp.
MGWSCIILFLVATATGVHS

SEQ ID NO: 90 - protein, 160 aa, Homo sapiens; identical to SEQ ID NO: 10

SEQ ID NO: 91 - protein, 161 aa, Homo sapiens; identical to SEQ ID NO: 11

SEQ ID NO: 92 - protein, 159 aa, Homo sapiens
KSKAVEQVKNAFNKLQEKGIYKAMSEFDIFINYIEAYMTMKIRNPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 93 - protein, 160 aa, Homo sapiens
MSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 94 - protein, 160 aa, Homo sapiens
KSKAVEQVKNAFNKLQEKGIYKAMSEFDIFINYIEAYMTMKIRNAVPNQRDYDPNCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 95 - protein, 159 aa, Homo sapiens
KSKAVEQVKNAFNKLQEKGIYKAMSEFDIFINYIEAYMTMKIRGKGPLTQPSEDCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 96 - protein, 160 aa, Homo sapiens
KSKAVEQVKNAFNKLQEKGIYKAMSEFDIFINYIEAYMTMKIRGSGSGSGSGSGSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 97 - protein, 160 aa, Homo sapiens; identical to SEQ ID NO: 53

SEQ ID NO: 98 - protein, 161 aa, Homo sapiens
KSKAVEQVKNAFNKLQEKGIYKAMSEFDIFINYIEAYMTMKIRNTSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 99 - DNA, 513 bp, Homo sapiens
  source 1..513: mol_type = genomic DNA
aagagtaaagctgtcgagcaggttaagaatgcttttaataaactgcaagaaaaaggtatctataaagccatgtctgagttcgatatctttattaactacattgaagcatatatgacgatgaaaatccgaaactcccccgggcagggaacccagtcagagaacagctgtacacattttcctggcaacctgcctaatatgctccgtgaccttcgggacgccttttcgagagtgaagaccttcttccaaatgaaggatcagttagacaatttactcctgaaggagtcccttttggaggacttcaaggggtacctgggatgtcaggcactcagcgagatgatacagttctacctggaagaggtaatgccccaggccgaaaaccaagatccagatattaaggcgcacgtgaactctctgggcgagaatctaaaaactctgcgcttgagactcaggcggtgccacaggtttctgccatgcgaaaatggtggatcccaccatcaccatcaccaccatcat

SEQ ID NO: 100 - DNA, 516 bp, Homo sapiens
  source 1..516: mol_type = genomic DNA
aagagcaaagctgtagagcaggttaaaaacgcattcaataagctgcaagagaagggtatttataaggccatgtccgagtttgacatcttcattaactacatcgaggcctacatgacgatgaagatcaggaacacttctcccggacagggcacacagtctgaaaattcatgcacccatttccccggaaatttgccaaacatgctgcgggacctgcgagatgcgtttagtcgggtcaaaaccttttttcagatgaaggaccagctggataatttattactcaaggagtcccttctggaggatttcaaaggctatttggggtgccaagcactgtcggagatgatacagttctacctggaagaagtgatgccacaagccgaaaaccaggaccctgatattaaagctcacgtgaatagcctgggggagaatctaaaaacacttcgtctccgcctcagaaggtgtcacagatttctcccttgtgaaaacggtggatcccaccatcaccatcaccaccatcat

SEQ ID NO: 101 - DNA, 510 bp, Homo sapiens
  source 1..510: mol_type = genomic DNA
aagagcaaggcagtcgaacaggtgaaaaatgcctttaataaactacaagaaaagggcatttataaagctatgtctgagtttgatatctttattaattacattgaggcgtatatgacgatgaagatccgcaatccaggacaaggaacccagagtgaaaactcctgtacccacttccctggtaacttacccaatatgctgcgggatctgcgtgacgctttctcaagagtaaaaacttttttccagatgaaggaccagcttgacaacctcctgctcaaggagtcccttctggaggattttaaagggtacctcgggtgtcaagccctgagcgagatgatccagttctacctggaggaagtgatgccacaggcagagaaccaggaccccgatataaaggcccacgttaactctctgggcgagaatctcaaaacattgaggttgagactgaggcgatgccatcggttcttaccttgcgaaaacggtggatcccaccatcaccatcaccaccatcat

SEQ ID NO: 102 - DNA, 480 bp, Homo sapiens
  source 1..480: mol_type = genomic DNA
atgtccaaggcagtggagcaagtcaagaacgcctttaataaattacaagagaagggcatctacaaagcgatgtccgagtttgacatcttcattaactacattgaagcttacatgaccatgaaaataaggaacagcccgggacagggcacccagagtgaaaactcttgtacgcacttccccgggaatctgcccaacatgctgagagatctgcgggacgccttctcaagagtgaagaccttcttccaaatgaaagaccagttggacaatctgctgctgaaggaaagcttattagaggatttcaaaggctatctaggatgtcaggctctgtcagaaatgatccagttctatctggaggaggtgatgccacaggccgagaatcaggacccagacattaaagcgcacgtcaattctctgggagaaaacctcaagactctccggttgcggctgagacggtgtcatagattcttgccatgtgaaaac

SEQ ID NO: 103 - DNA, 513 bp, Homo sapiens
  source 1..513: mol_type = genomic DNA
aagagtaaggcagtggaacaggtaaagaacgcctttaacaaactgcaggagaaaggcatttataaggcgatgagcgaatttgatatctttatcaactatatcgaagcctatatgacgatgaaaataaggaacgccgtcccgaaccagagggattacgaccctaattgcacccacttcccaggcaacttacctaatatgctgcgcgaccttcgagacgctttttcaagagttaaaactttcttccagatgaaagatcaactggataatctcctgttaaaagagtccctcttggaagacttcaaggggtacttgggatgccaggccctgtctgagatgattcagttttacctggaggaggtgatgccccaggcagaaaatcaagatccagacattaaggctcacgtgaatagcctgggtgagaacctgaagacactccggctcagacttcgtcggtgtcatcgcttcctaccctgtgagaatggtggatcccaccatcaccatcaccaccatcat

SEQ ID NO: 104 - DNA, 510 bp, Homo sapiens
  source 1..510: mol_type = genomic DNA
aagagcaaggctgtggagcaggtaaagaatgcctttaataagttgcaggaaaaaggaatctataaagcgatgagcgaatttgacatattcatcaattacatcgaggcttacatgaccatgaagattcggggcaaaggtccactaacacaaccgtccgaagattgcacccactttccagggaacctgcccaacatgctgcgagacctgcgggacgccttctcaagagttaaaacgtttttccagatgaaggatcaactcgataatctcctgctcaaagagagtttacttgaggattttaaaggctatctgggatgccaggcactgtctgaaatgattcagttctacctggaggaggtcatgcctcaggcagagaatcaggaccctgacattaaggcccacgtgaactcccttggggagaacttgaagactttgaggcttcgtctgagaaggtgtcatcgcttcctcccctgtgaaaacggtggatcccaccatcaccatcaccaccatcat

SEQ ID NO: 105 - DNA, 513 bp, Homo sapiens
  source 1..513: mol_type = genomic DNA
aaatctaaggcagtggagcaggtgaagaatgccttcaataaattgcaggagaaaggaatttacaaggccatgtccgagtttgatatctttatcaactatatagaagcatacatgaccatgaagatccgtgggtcaggaagtggctcaggttctgggagcggcagctgcacacactttccggggaacctcccaaacatgctcagagatctccgcgacgccttctcccgagttaaaacgtttttccaaatgaaggaccagctggacaacctgctgctgaaggaatccctgttggaggatttcaaaggttatctgggatgtcaggcgctaagtgagatgattcaattctaccttgaagaagtcatgccccaggctgagaatcaggaccccgatattaaggctcacgtaaacagcctcggcgaaaatcttaaaactctgcggttacggttaaggagatgccataggtttctgccttgtgagaatggtggatcccaccatcaccatcaccaccatcat

SEQ ID NO: 106 - DNA, 513 bp, Homo sapiens
  source 1..513: mol_type = genomic DNA
aagagtaaagctgtcgagcaggttaagaatgcttttaataaactgcaagaaaaaggtatctataaagccatgtctgagttcgatatctttattaactacattgaagcatatatgacgatgaaaatccgaaactcccccgggcagggaacccagtcagagaacagctgtacacattttcctggcaacctgcctaatatgctccgtaagcttcgggacgccttttcgagagtgaagaccttcttccaaatgaaggatcagttagacaatttactcctgaaggagtcccttttggaggacttcaaggggtacctgggatgtcaggcactcagcgagatgatacagttctacctggaagaggtaatgccccaggccgaaaaccaagatccagatattaaggcgcacgtgaactctctgggcgagaatctaaaaactctgcgcttgagactcaggcggtgccacaggtttctgccatgcgaaaatggtggatcccaccatcaccatcaccaccatcat

SEQ ID NO: 107 - DNA, 516 bp, Homo sapiens
  source 1..516: mol_type = genomic DNA
aagagcaaagctgtagagcaggttaaaaacgcattcaataagctgcaagagaagggtatttataaggccatgtccgagtttgacatcttcattaactacatcgaggcctacatgacgatgaagatcaggaacacttctcccggacagggcacacagtctgaaaattcatgcacccatttccccggaaatttgccaaacatgctgcggaagctgcgagatgcgtttagtcgggtcaaaaccttttttcagatgaaggaccagctggataatttattactcaaggagtcccttctggaggatttcaaaggctatttggggtgccaagcactgtcggagatgatacagttctacctggaagaagtgatgccacaagccgaaaaccaggaccctgatattaaagctcacgtgaatagcctgggggagaatctaaaaacacttcgtctccgcctcagaaggtgtcacagatttctcccttgtgaaaacggtggatcccaccatcaccatcaccaccatcat

SEQ ID NO: 108 - protein, 172 aa, synthetic construct
KSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 109 - protein, 170 aa, synthetic construct
KSKAVEQVKNAFNKLQEKGIYKAMSEFDIFINYIEAYMTMKIRNPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 110 - protein, 171 aa, synthetic construct
KSKAVEQVKNAFNKLQEKGIYKAMSEFDIFINYIEAYMTMKIRNAVPNQRDYDPNCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 111 - protein, 170 aa, synthetic construct
KSKAVEQVKNAFNKLQEKGIYKAMSEFDIFINYIEAYMTMKIRGKGPLTQPSEDCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 112 - protein, 171 aa, synthetic construct
KSKAVEQVKNAFNKLQEKGIYKAMSEFDIFINYIEAYMTMKIRGSGSGSGSGSGSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 113 - protein, 171 aa, synthetic construct
KSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 114 - protein, 172 aa, synthetic construct
KSKAVEQVKNAFNKLQEKGIYKAMSEFDIFINYIEAYMTMKIRNTSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 115 - DNA, 993 bp, synthetic construct
  source 1..993: mol_type = other DNA
aaaagcaaggccgtggagcaagtgaagaacgccttcaacaaactgcaagagaaagggatctataaggcgatgtccgagtttgacatatttatcaactacatcgaggcatacatgactatgaagatccgtaatagccccggccaaggcacacagtcagagaactcttgtacccatttccctgggaacctcccaaacatgctgcgtgatctgagagacgccttcagtagagtgaagactttttttcagatgaaggaccaactcgataacctgcttcttaaagaaagcctgctggaggactttaagggataccttggctgccaagccctaagcgaaatgatccaattttatttagaagaggtcatgccccaagccgagaaccaagaccccgacattaaggcccatgtgaactctctgggcgagaacctgaaaactctccgcctgagactaagaagatgccacagatttctgccctgcgagaacaaatcaaaggccgtcgagcaagtgaaaaacgcgtttaacaagctgcaagaaaagggaatatacaaggccatgtcagagttcgacatcttcataaattatatcgaagcatacatgaccatgaaaatcagaaacagccccggacaaggcacccaaagcgagaatagctgcacccacttccccggaaacctgcccaacatgctgagagacttgagagatgccttctcgagggtgaagaccttttttcaaatgaaggatcagcttgataatctgttgctgaaggagtctctgctggaggatttcaagggctacctgggctgtcaagccctgagcgaaatgattcagttttacctggaagaggtgatgccccaagctgagaatcaagaccccgacattaaagcacatgtgaacagcctgggggagaacttgaagaccttacgattgagattaagaaggtgtcacagattcctgccctgcgaaaacggcggcagccaccaccaccaccaccaccaccac

SEQ ID NO: 116 - DNA, 999 bp, synthetic construct
  source 1..999: mol_type = other DNA
aaaagcaaggccgtggagcaagtgaagaacgccttcaacaaactgcaagagaaagggatctataaggcgatgtccgagtttgacatatttatcaactacatcgaggcatacatgactatgaagatccgtaatagccccggccaaggcacacagtcagagaactcttgtacccatttccctgggaacctcccaaacatgctgcgtgatctgagagacgccttcagtagagtgaagactttttttcagatgaaggaccaactcgataacctgcttcttaaagaaagcctgctggaggactttaagggataccttggctgccaagccctaagcgaaatgatccaattttatttagaagaggtcatgccccaagccgagaaccaagaccccgacattaaggcccatgtgaactctctgggcgagaacctgaaaactctccgcctgagactaagaagatgccacagatttctgccctgcgagaacggcggtaaatcaaaggccgtcgagcaagtgaaaaacgcgtttaacaagctgcaagaaaagggaatatacaaggccatgtcagagttcgacatcttcataaattatatcgaagcatacatgaccatgaaaatcagaaacagccccggacaaggcacccaaagcgagaatagctgcacccacttccccggaaacctgcccaacatgctgagagacttgagagatgccttctcgagggtgaagaccttttttcaaatgaaggatcagcttgataatctgttgctgaaggagtctctgctggaggatttcaagggctacctgggctgtcaagccctgagcgaaatgattcagttttacctggaagaggtgatgccccaagctgagaatcaagaccccgacattaaagcacatgtgaacagcctgggggagaacttgaagaccttacgattgagattaagaaggtgtcacagattcctgccctgcgaaaacggcggcagccaccaccaccaccaccaccaccac

SEQ ID NO: 117 - DNA, 999 bp, synthetic construct
  source 1..999: mol_type = other DNA
aaaagcaaggccgtggagcaagtgaagaacgccttcaacaaactgcaagagaaaggcatttacaaagccatgtccgagtttgacatatttatcaactacattgaggcgtatatgaccatgaagatccgtaatacctctcccggacaaggcacccaaagcgaaaacagctgcacacactttcctggcaatctccccaacatgctacgtgacctgagagacgcctttagcagagtgaaaaccttctttcagatgaaggatcagctcgacaaccttcttttaaaagagagcctgctggaggatttcaaaggctatctgggctgccaagccctgtctgagatgattcagttctacctggaagaggtgatgcctcaagcagaaaatcaagaccccgatatcaaggcacacgtgaatagcctgggggaaaacttgaaaacccttagactgcgtctgagacgatgtcaccgcttcctgccctgcgagaacaaatccaaggctgtggagcaagtgaagaatgcattcaacaagcttcaagagaaaggtatctacaaggccatgagcgagttcgacatcttcataaattacatcgaggcctacatgaccatgaaaatcagaaacacaagccccggccaaggcacacagagcgaaaacagctgcacccacttccccggcaacctgcccaatatgctgagggacctaagagacgctttcagcagagttaaaactttttttcagatgaaagaccaactagataacttgctgctgaaggaaagcctgctggaagacttcaagggctacctgggatgccaagccctgtcagagatgattcaattctacctagaagaggtgatgccccaagccgaaaaccaagaccccgatataaaagcgcacgtgaacagcctgggcgagaatcttaagaccttaaggctgcgactgaggcggtgccacagattcctgccctgtgagaacggcggtagccaccaccaccaccaccaccaccac

SEQ ID NO: 118 - DNA, 1005 bp, synthetic construct
  source 1..1005: mol_type = other DNA
aaaagcaaggccgtggagcaagtgaagaacgccttcaacaaactgcaagagaaaggcatttacaaagccatgtccgagtttgacatatttatcaactacattgaggcgtatatgaccatgaagatccgtaatacctctcccggacaaggcacccaaagcgaaaacagctgcacacactttcctggcaatctccccaacatgctacgtgacctgagagacgcctttagcagagtgaaaaccttctttcagatgaaggatcagctcgacaaccttcttttaaaagagagcctgctggaggatttcaaaggctatctgggctgccaagccctgtctgagatgattcagttctacctggaagaggtgatgcctcaagcagaaaatcaagaccccgatatcaaggcacacgtgaatagcctgggggaaaacttgaaaacccttagactgcgtctgagacgatgtcaccgcttcctgccctgcgagaacggtggtaaatccaaggctgtggagcaagtgaagaatgcattcaacaagcttcaagagaaaggtatctacaaggccatgagcgagttcgacatcttcataaattacatcgaggcctacatgaccatgaaaatcagaaacacaagccccggccaaggcacacagagcgaaaacagctgcacccacttccccggcaacctgcccaatatgctgagggacctaagagacgctttcagcagagttaaaactttttttcagatgaaagaccaactagataacttgctgctgaaggaaagcctgctggaagacttcaagggctacctgggatgccaagccctgtcagagatgattcaattctacctagaagaggtgatgccccaagccgaaaaccaagaccccgatataaaagcgcacgtgaacagcctgggcgagaatcttaagaccttaaggctgcgactgaggcggtgccacagattcctgccctgtgagaacggcggtagccaccaccaccaccaccaccaccac

SEQ ID NO: 119 - DNA, 993 bp, synthetic construct
  source 1..993: mol_type = other DNA
aaaagcaaggccgtggagcaagtgaagaacgccttcaacaaactgcaagagaaagggatctataaggcgatgtccgagtttgacatatttatcaactacatcgaggcatacatgactatgaagatccgtaatagccccggccaaggcacacagtcagagaactcttgtacccatttccctgggaacctcccaaacatgctgcgtaaactgagagacgccttcagtagagtgaagactttttttcagatgaaggaccaactcgataacctgcttcttaaagaaagcctgctggaggactttaagggataccttggctgccaagccctaagcgaaatgatccaattttatttagaagaggtcatgccccaagccgagaaccaagaccccgacattaaggcccatgtgaactctctgggcgagaacctgaaaactctccgcctgagactaagaagatgccacagatttctgccctgcgagaacaaatcaaaggccgtcgagcaagtgaaaaacgcgtttaacaagctgcaagaaaagggaatatacaaggccatgtcagagttcgacatcttcataaattatatcgaagcatacatgaccatgaaaatcagaaacagccccggacaaggcacccaaagcgagaatagctgcacccacttccccggaaacctgcccaacatgctgagaaagttgagagatgccttctcgagggtgaagaccttttttcaaatgaaggatcagcttgataatctgttgctgaaggagtctctgctggaggatttcaagggctacctgggctgtcaagccctgagcgaaatgattcagttttacctggaagaggtgatgccccaagctgagaatcaagaccccgacattaaagcacatgtgaacagcctgggggagaacttgaagaccttacgattgagattaagaaggtgtcacagattcctgccctgcgaaaacggcggcagccaccaccaccaccaccaccaccac

SEQ ID NO: 120 - DNA, 993 bp, synthetic construct
  source 1..993: mol_type = other DNA
aaaagcaaggccgtggagcaagtgaagaacgccttcaacaaactgcaagagaaagggatctataaggcgatgtccgagtttgacatatttatcaactacatcgaggcatacatgactatgaagatccgtaatagccccggccaaggcacacagtcagagaactcttgtacccatttccctgggaacctcccaaacatgctgcgtgatctgagagacgccttcagtagagtgaagactttttttcagatgaaggaccaactcgataacctgcttcttaaagaaagcctgctggaggactttaagggataccttggctgccaagccctaagcgaaatgatccaattttatttagaagaggtcatgccccaagccgagaaccaagaccccgacattaaggcccatgtgaactctctgggcgagaacctgaaactcctccgcctgagactaagaagatgccacagatttctgccctgcgagaacaaatcaaaggccgtcgagcaagtgaaaaacgcgtttaacaagctgcaagaaaagggaatatacaaggccatgtcagagttcgacatcttcataaattatatcgaagcatacatgaccatgaaaatcagaaacagccccggacaaggcacccaaagcgagaatagctgcacccacttccccggaaacctgcccaacatgctgagagacttgagagatgccttctcgagggtgaagaccttttttcaaatgaaggatcagcttgataatctgttgctgaaggagtctctgctggaggatttcaagggctacctgggctgtcaagccctgagcgaaatgattcagttttacctggaagaggtgatgccccaagctgagaatcaagaccccgacattaaagcacatgtgaacagcctgggggagaacttgaagctgttacgattgagattaagaaggtgtcacagattcctgccctgcgaaaacggcggcagccaccaccaccaccaccaccaccac

SEQ ID NO: 121 - protein, 331 aa, synthetic construct
KSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 122 - protein, 333 aa, synthetic construct
KSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 123 - protein, 333 aa, synthetic construct
KSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 124 - protein, 335 aa, synthetic construct
KSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGKSKAVEQVKNAFNKLQEKGIYKAMSEFDIFINYIEAYMTMKIRNTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 125 - protein, 331 aa, synthetic construct
KSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENGGSHHHHHHHH

SEQ ID NO: 126 - protein, 331 aa, synthetic construct
KSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRNSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENGGSHHHHHHHH

SEQ ID NO: 127 - DNA, 530 bp, Mus sp.
  source 1..530: mol_type = genomic DNA
aacaagagcaaggccgtggagcaagtgaagagcgacttcaacaagctgcaagaccaaggcgtgtacaaggccatgaacgagttcgacatcttcatcaacgccatcgaggcctacatgatgatcaagatgaagagcagcagagggcagtacagcagagaggacaacaactgcacccacttccccgtggggcagagccacatgctgctggagctgagaaccgccttcagccaagtgaagaccttctttcagaccaaggatcagctggacaacatcctgctgaccgacagcctgatgcaagacttcaagggctacctgggctgccaagccctgagcgagatgattcagttctacctggtggaggtgatgccccaagccgagaagcacggccccgagatcaaggagcacctgaacagcctgggcgagaagctgaagaccctgagaatgagactgagaagatgccacagattcctgccctgcgagaacggcggatcccaccatcaccatcaccaccatcattagtgagcggccgc

SEQ ID NO: 128 - DNA, 530 bp, Mus sp.
  source 1..530: mol_type = genomic DNA
aacaagagcaaggccgtggagcaagtgaagagcgacttcaacaagctgcaagaccaaggcgtgtacaaggccatgaacgagttcgacatcttcatcaactgcatcgaggcctacatgatgatcaagatgaagagcagcagagggcagtacagcagagaggacaacaactgcacccacttccccgtggggcagagccacatgctgctggagctgagaaccgccttcagccaagtgaagaccttctttcagaccaaggatcagctggacaacatcctgctgaccgacagcctgatgcaagacttcaagggctacctgggctgccaagccctgagcgagatgattcagttctacctggtggaggtgatgccccaagccgagaagcacggccccgagatcaaggagcacctgaacagcctgggcgagaagctgaagaccctgagaatgagactgagaagatgccacagattcctgccctgcgagaacggcggcagccaccaccaccaccaccaccaccactgataagcggccgc

SEQ ID NO: 129 - DNA, 530 bp, Mus sp.
  source 1..530: mol_type = genomic DNA
aacaagagcaaggccgtggagcaagtgaagagcgacttcaacaagctgcaagaccaaggcgtgtacaaggccatgaacgagttcgacatcttcatcaactgcatcgaggcctacatgatgatcaagatgaagagcagcagagggcagtacagcagagaggacaacaactgcacccacttccccgtggggcagagccacatgctgctggagctgagaaccgccttcagccaagtgaagaccttctttcagaccaaggatcagctggacaacatcctgctgaccgacagcctgatgcaagacttcaagggctacctgggctgccaagccctgagcgagatgattcagttctacctggtggaggtgatgccccaagccgagaagcacggccccgagatcaaggagcacctgaacagcctgggcgagaagctgaagctgctgagaatgagactgagaagatgccacagattcctgccctgcgagaacggcggcagccaccaccaccaccaccaccaccactgataagcggccgc

SEQ ID NO: 130 - protein, 172 aa, synthetic construct
NKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENGGSHHHHHHHH

SEQ ID NO: 131 - protein, 172 aa, synthetic construct
NKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENGGSHHHHHHHH

SEQ ID NO: 132 - DNA, 1019 bp, synthetic construct
  source 1..1019: mol_type = other DNA
aacaaaagcaaggccgttgagcaagtgaaatcggacttcaacaaactgcaagaccaaggcgtctacaaggctatgaatgagtttgacatatttatcaactgcatcgaagcgtacatgatgataaagatgaagagcagccgcgggcagtacagcagagaagacaacaattgtacccatttccctgtcggccaatcccacatgctgctagaactgagaaccgccttcagtcaagtgaagactttctttcagaccaaggaccaactcgataacatccttcttacggatagccttatgcaagatttcaagggataccttggctgccaagccctaagcgaaatgatccaattctacctggtggaggtgatgccgcaagctgagaaacacggccctgaaattaaggagcatctgaactctctgggcgagaagctgaaaactctgagaatgagattgagaagatgtcacagatttctgccctgcgagaacggcggtggaaaaagcaaagccgtcgagcaagtgaaatcagacttcaacaagctgcaagaccaaggcgtgtacaaggcaatgaacgagttcgacatattcatcaactgtatcgaggcctacatgatgatcaagatgaagagctctagaggccaatacagtagagaggataacaactgcacccacttccccgtggggcagagccatatgctgttggagctaagaaccgctttcagccaagtgaaaactttttttcagacgaaggatcaactcgacaatatcctgctgacggacagcctgatgcaagacttcaagggctacctgggatgccaagccctgtcagagatgattcaattctacctagtggaggtaatgccccaagccgaaaagcacggacccgaaattaaagaacacctgaacagcctgggcgagaaactgaagaccttaaggatgagactgaggagatgccacagattcctgccctgtgaaaacggcggcagtcaccaccaccaccaccaccaccactgataagcggccgc

SEQ ID NO: 133 - DNA, 1019 bp, synthetic construct
  source 1..1019: mol_type = other DNA
aacaaaagcaaggccgttgagcaagtgaaatcggacttcaacaaactgcaagaccaaggcgtctacaaggctatgaatgagtttgacatatttatcaactgcatcgaagcgtacatgatgataaagatgaagagcagccgcgggcagtacagcagagaagacaacaattgtacccatttccctgtcggccaatcccacatgctgctagaactgagaaccgccttcagtcaagtgaagactttctttcagaccaaggaccaactcgataacatccttcttacggatagccttatgcaagatttcaagggataccttggctgccaagccctaagcgaaatgatccaattctacctggtggaggtgatgccgcaagctgagaaacacggccctgaaattaaggagcatctgaactctctgggcgagaagctgaaactcctgagaatgagattgagaagatgtcacagatttctgccctgcgagaacggcggtggaaaaagcaaagccgtcgagcaagtgaaatcagacttcaacaagctgcaagaccaaggcgtgtacaaggcaatgaacgagttcgacatattcatcaactgtatcgaggcctacatgatgatcaagatgaagagctctagaggccaatacagtagagaggataacaactgcacccacttccccgtggggcagagccatatgctgttggagctaagaaccgctttcagccaagtgaaaactttttttcagacgaaggatcaactcgacaatatcctgctgacggacagcctgatgcaagacttcaagggctacctgggatgccaagccctgtcagagatgattcaattctacctagtggaggtaatgccccaagccgaaaagcacggacccgaaattaaagaacacctgaacagcctgggcgagaaactgaagctgttaaggatgagactgaggagatgccacagattcctgccctgtgaaaacggcggcagtcaccaccaccaccaccaccaccactgataagcggccgc

SEQ ID NO: 134 - protein, 335 aa, synthetic construct
NKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENGGGKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENGGSHHHHHHHH

SEQ ID NO: 135 - protein, 335 aa, synthetic construct
NKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENGGGKSKAVEQVKSDFNKLQDQGVYKAMNEFDIFINCIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKLLRMRLRRCHRFLPCENGGSHHHHHHHH

SEQ ID NO: 136 - protein, 800 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 1..800: note = This sequence may encompass 1-20 GmSo repeating units, wherein m = 1 to 20, o = 1 to 20
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 137 - protein, 40 aa, synthetic construct
  VARIANT 1..40: note = This sequence may encompass 1-20 GS repeating units
GSGSGSGSGSGSGSGSGSGSGSGSGSGSGSGSGSGSGSGS

SEQ ID NO: 138 - protein, 50 aa, synthetic construct
  VARIANT 1..50: note = This sequence may encompass 1-50 residues
GGGGGGGGGGGGGGGGGGGGGGGGGGGGGGGGGGGGGGGGGGGGGGGGGG

SEQ ID NO: 139 - protein, 6 aa, synthetic construct
HHHHHH

SEQ ID NO: 140 - protein, 8 aa, synthetic construct
HHHHHHHH

SEQ ID NO: 141 - protein, 160 aa, Mus sp.
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 142 - protein, 156 aa, synthetic construct
GKSKAVEQVKSIFNKLQDQGVYKAMNEFDIFINAIEAYMMIKMKSSREDNNCTHFMVAMSHMLLELRTAFSQVKTFFQTKDQLDNILLTDSLMQDFKGYLGCQALSEMIQFYLVEVLPQAEKHGPEIKEHLNSLGEKLKTLRMRLRRCHRFLPCEN

SEQ ID NO: 143 - protein, 156 aa, synthetic construct
GKSKAVEQVKSIFNKLQDQGVYKAMNEFDIFINAIEAYMMIKMKSSREDNNCTHAPVAFWHMLLELRTAFSQVKTFFQTKDQLDNILLTDSLMQDFKGYLGCQALSEMIQFYLVEVLPQAEKHGPEIKEHLNSLGEILKTLRMRLRRCHRFLPCEN

SEQ ID NO: 144 - protein, 156 aa, synthetic construct
GKSKAVEQVKSIFNKLQDQGVYKAMNEFDIFINAIEAYMMIKMKSSREDNNCTHAPVGMSHMLLELRTAFSQVKTFFQTKDQLDNILLTDSLMQDFKGYLGCQALSEMIQFYLVEVLPQAEKHGPEIKEHLISLGEILKTLRMRLRRCHRFLPCEN

SEQ ID NO: 145 - protein, 156 aa, synthetic construct
GKSKAVEQVKSIFNKLQDQGVYKAMNEFDIVINAIEAYMMIKMKSSREDNNCTHAPVMFSHMLLELRTAFSQVKTFFQTKDQLDNILLTDSLMQDFKGYLGCQALSEMIQFYLVEVLPQAEKHGPEIKEHLNSLGEILKTLRMRLRRCHRFLPCEN

SEQ ID NO: 146 - protein, 156 aa, synthetic construct
GKSKAVEQVKSIFNKLQDQGVYKAMNEFDIFINAIEAYMMIKMKSSREDNNCTHFNVGLSHMLLELRTAFSQVKTFFQTKDQLDNILLTDSLMQDFKGYLGCQALSEMIQFYLVEVLPQAEKHGPEIKEHLNSLGEILKTLRMRLRRCHRFLPCEN

SEQ ID NO: 147 - protein, 156 aa, synthetic construct
GKSKAVEQVKSIFNKLQDQGVYKAMNEFDIFINAIEAYMMIKMKSSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCEN

SEQ ID NO: 148 - protein, 156 aa, synthetic construct
GKSKAVEQVKSIFNKLQDQGVYKAMNEFDIFINAIEAYMMIKMKSSREDNNCTHAPVGQSHMLLELRTAFSQVKTFFQTKDQLDNILLTDSLMQDFKGYLGCQALSEMIQFYLVEVLPQAEKHGPEIKEHLNSLGEILKTLRMRLRRCHRFLPCEN

SEQ ID NO: 149 - protein, 156 aa, synthetic construct
GKSKAVEQVKSIFNKLQDQGVYKAMNEFDIFINAIEAYMMIKMKSSREDNNCTHAPVGQSHMLLELRTAFSQVKTFFQTKDQLDNILLTDSLMQDFKGYLGCQALSEMIQFYLVEVLPQAEKHGPEIKEHLNSLGEKLKTLRMRLRRCHRFLPCEN

SEQ ID NO: 150 - protein, 156 aa, synthetic construct
GKSKAVEQVKSDFNKLQDQGVYKANNEFMIFITAIEAYMMIKMKSSREDNNCTHFWVWMSHMLLELRTAASQVKTFFQTKDQLDNILLTDSLMQDFKGYLGCQALSEMIQFYLVEVLPQAEKHGPEIKEHLISLGELLKTLRMRLRRCHRFLPCEN

SEQ ID NO: 151 - protein, 156 aa, synthetic construct
GKSKAVEQVKSDFNKLQDQGVYKAMVEIDIFINAIEAYMMIKMKSSREDNNCTHFWVIMLHMLLELRTAMSQVKTFFQTKDQLDNILLTDSLMQDFKGYLGCQALSEMIQFYLVEVLPQAEKHGPEIKEHLISLGELLKTLRMRLRRCHRFLPCEN

SEQ ID NO: 152 - protein, 156 aa, synthetic construct
GKSKAVEQVKSDFNKLQDQGVYKAMNEFDIFINAIEAYMMIKMKSSREDNNCTHFWVAMSHMLLELRTAASQVKTFFQTKDQLDNILLTDSLMQDFKGYLGCQALSEMIQFYLVEVLPQAEKHGPEIKEHLISLGEILKTLRMRLRRCHRFLPCEN

SEQ ID NO: 153 - protein, 156 aa, synthetic construct
GKSKAVEQVKSDFNKLQDQGVYKAMVEFDIFINAIEAYMMIKMKSSREDNNCTHFMVALSHMLLELRTAFSQVKTFFQTKDQLDNILLTDSLMQDFKGYLGCQALSEMIQFYLVEVLPQAEKHGPEIKEHLISLGEILKTLRMRLRRCHRFLPCEN

SEQ ID NO: 154 - protein, 156 aa, synthetic construct
GKSKAVEQVKSDFNKLQDQGVYKAMNEFDIFINAIEAYMMIKMKSSREDNNCTHFNVVMSHMLLELRTAFSQVKTFFQTKDQLDNILLTDSLMQDFKGYLGCQALSEMIQFYLVEVLPQAEKHGPEIKEHLNSLGEKLKTLRMRLRRCHRFLPCEN

SEQ ID NO: 155 - protein, 156 aa, synthetic construct
GKSKAVEQVKSDFNKLQDQGVYKAMNEFDIFINAIEAYMMIKMKSSREDNNCTHAPVGQSHMLLELRTAFSQVKTFFQTKDQLDNILLTDSLMQDFKGYLGCQALSEMIQFYLVEVLPQAEKHGPEIKEHLNSLGEILKTLRMRLRRCHRFLPCEN

SEQ ID NO: 156 - protein, 156 aa, synthetic construct
GKSKAVEQVKSDFNKLQDQGVYKAMNEFDIFINAIEAYMMIKMKSSREDNNCTHAPVGQSHMLLELRTAFSQVKTFFQTKDQLDNILLTDSLMQDFKGYLGCQALSEMIQFYLVEVLPQAEKHGPEIKEHLNSLGEKLKTLRMRLRRCHRFLPCEN

SEQ ID NO: 157 - protein, 158 aa, synthetic construct
GKSKCVEQVKSDFNKLQDQGVYKAMNEFDIFINAIEAYMMIKMKSQYSREDNNCTHFPVGQSHMLLELRTAFSQVKTFFQTKDQLDNILLTDSCMQDFKGYLGCQALSEMIQFYLVEVMPQAEKHGPEIKEHLNSLGEKLKTLRMRLRRCHRFLPCEN

SEQ ID NO: 158 - protein, 158 aa, synthetic construct
GKSKCVEQVKSDFNKLQDQGVYKAMNEFDIFINAIEAYMMIKMKSQYSREDNNCTHFPVGQSHMLLELRTAFSQVKTFFQTKDQLDNILLTDSLMQCFKGYLGCQALSEMIQFYLVEVMPQAEKHGPEIKEHLNSLGEKLKTLRMRLRRCHRFLPCEN

SEQ ID NO: 159 - protein, 158 aa, synthetic construct
GKCKAVEQVKSDFNKLQDQGVYKAMNEFDIFINAIEAYMMIKMKSQYSREDNNCTHFPVGQSHMLLELRTAFSQVKTFFQTKDQLDNILLTDSLMQCFKGYLGCQALSEMIQFYLVEVMPQAEKHGPEIKEHLNSLGEKLKTLRMRLRRCHRFLPCEN

SEQ ID NO: 160 - protein, 158 aa, synthetic construct
GKSKACEQVKSDFNKLQDQGVYKAMNEFDIFINAIEAYMMIKMKSQYSREDNNCTHFPVGQSHMLLELRTAFSQVKTFFQTKDQLDNILLTDSLMQDFKGYLGCQALSCMIQFYLVEVMPQAEKHGPEIKEHLNSLGEKLKTLRMRLRRCHRFLPCEN

SEQ ID NO: 161 - protein, 155 aa, synthetic construct
GKSKAVEQVKSDFNKLQDQGVYKAMNEFDIFINAIEAYMMIKMKSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCEN

SEQ ID NO: 162 - protein, 156 aa, synthetic construct
GKSKAVEQVKSDFNKLQDQGVYKAMNEFDIFINAIEAYMMIKMKSSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCEN

SEQ ID NO: 163 - protein, 168 aa, synthetic construct
AHHHHHHHHGSQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 164 - protein, 171 aa, synthetic construct
NKSKAVEQVKSDFNKLQDQGVYKAMNEFDIFINAIEAYMMIKMKSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENGGSHHHHHHH

(19) United States
(12) Patent Application Publication
(10) Pub. No.: US 2026/0365709 A1
(43) Pub. Date: Nov. 19, 2026

(54) IL10 INVERTED MONOMERS

(71) Applicant: Synthekine, Inc., Menlo Park, CA (US)

(72) Inventors: Andrew Morin, Menlo Park, CA (US); Tatjana Petojevic, Menlo Park, CA (US); Mahalakshmi Ramadass, Fremont, CA (US); Bhargavi Jayaraman, Menlo Park, CA (US)

(21) Appl. No.: 19/488,561

(22) PCT Filed: May 31, 2024
(86) PCT No.: PCT/US2024/032147

§ 371 Date: Nov. 26, 2025

Related U.S. Application Data
(60) Provisional application No. 63/505,651, filed on Jun. 1, 2023.

(51) Int. Cl. C07K 14/54; A61K 38/00; A61K 47/60; A61P 29/00; A61P 37/02

(52) U.S. Cl. CPC C07K 14/5428; A61K 47/60; A61P 29/00; A61P 37/02; A61K 38/00

(57) ABSTRACT

The present disclosure relates to engineered polypeptides comprising components of IL10 molecules that modulate signal transduction associated with IL10 and methods of use thereof. In some aspects, the polypeptides of the disclosure exhibit cell type biased activity. In some aspects the polypeptides of the disclosure substantially retain the anti-inflammatory properties of IL10 but have significantly diminished pro-inflammatory properties of IL-10. The disclosure provides methods of use of the engineered polypeptides in the treatment of disease.

### CROSS-REFERENCE TO RELATED APPLICATION

The present application is a National Phase Application of PCT/US2024/032147, filed May 31, 2024, which claims priority to U.S. Provisional Patent Application No. 63/505,651, filed on Jun. 1, 2023, the disclosure of which is incorporated herein by reference in its entirety for all purposes.

### SEQUENCE LISTING

The contents of the electronic sequence listing (106249-008610PC-1445591_SL.xml; size 289,448 bytes; and date of creation Mar. 11, 2026) is herein incorporated by reference.

### BACKGROUND

Cytokine and growth-factor ligands typically signal through multimerization of cell surface receptor subunits. In some instances, cytokines act as multi-specific (e.g., bispecific or tri-specific) ligands which facilitate the association of the extracellular domains of receptor subunits and resultingly bring the intracellular domains of the receptor subunits into proximity facilitating intracellular signaling. The nature of the cytokine ligand and its interaction with the extracellular domains of receptor subunits determines which and how receptor subunits associate to form a ligand receptor complex and the intracellular signaling characteristic of such complex.

The intracellular domains of some cytokine receptor subunits possess Janus kinase (“JAK”) binding domains. JAK binding domains are typically located in the box1/box region of the intracellular domain of the cytokine receptor subunit near the interior surface of the cell membrane. Intracellular Janus kinases associate with and phosphorylate the JAK binding domains. Four Janus kinases have been identified in mammalian cells: JAK1, JAK2, JAK3 and TYK2. Ihle, et al. (1995) Nature 377(6550):591-4, 1995; O'Shea and Plenge (2012) Immunity 36(4):542-50. When the intracellular domains of cytokine receptor subunits containing JAK binding domains are brought into proximity, the Janus kinases facilitate transphosphorylation of the JAK binding domains. Phosphorylation of the JAK induces conformational changes in the JAK providing the ability of the JAK to further phosphorylate other intracellular proteins. The resulting phosphorylation cascade results in activation of multiple intracellular factors which transduce the intracellular signals associated with the receptor activation by the cytokine ligand. In some instances, the intracellular proteins which are phosphorylated by the JAKs are members of the signal transducer and activator of transcription (“STAT”) protein family. Seven members of the mammalian STAT family have been identified to date: STAT1, STAT2, STAT3, STAT4, STAT5a STAT5b, and STAT6. Delgoffe, et al., (2011) Curr Opin Immunol. 23(5):632-8; Levy and Darnell (2002) Nat Rev Mol Cell Biol. 3(9):651-62 and Murray, (2007) J Immunol. 178(5):2623-9. The selective interplay of activated JAK and STAT proteins, collectively referred to as the JAK/STAT pathway, are involved in the variety of intracellular responses observed in response to cytokine binding. Such intracellular responses initiated by the binding of a cytokine to its receptor are frequently referred to as downstream signaling.

The cytokine human interleukin-10 (hIL10), also known as human cytokine synthesis inhibitory factor (CSIF), is classified as a type (class)-2 cytokine, a group of cytokines that includes IL19, IL20, IL22, IL24 (Mda-7), and IL26, interferons (IFN-α, -β, -γ, -δ, —ε, -κ, -Ω, and -τ) and interferon-like molecules (such as limitin, IL28A, IL28B, and IL29). hIL10 is a non-covalent homodimer comprised of two hIL10 monomer polypeptides. Each hIL10 monomer polypeptide is a 160 amino acid polypeptide with two intramolecular disulfide bonds. Each hIL10 monomer is expressed as a proprotein comprised of 178 amino acids, the first 18 amino acids of which comprise a signal peptide. Although hIL10 is predominantly expressed by macrophages, expression has also been detected in activated T cells, B cells, mast cells, NK cells, dendritic cells, eosinophils, neutrophils and monocytes.

Human IL10 exerts its effect on cells through its interaction with the hIL10 receptor (hIL10R). hIL10R is a type II cytokine receptor comprising the hIL10Rα and hIL10Rβ subunits, which are also referred to as hIL10R1 and hIL10R2, respectively. The hIL10Rα receptor subunit is a “private” or “proprietary” subunit exclusive to the hIL10 receptor. In contrast, the hIL10Rβ subunit is shared with other cytokine receptors including IL22, IL26, IL28, and the interferon lambda L1 (IFNλ1) receptor complexes. Activation of the hIL10R is characterized by the binding of each hIL10 monomer to one hIL10Rα and one hIL10Rβ subunit of hIL10R. Each monomer of the dimeric hIL10 cytokine associates with one hIL10Rα and one hIL10Rβ subunit resulting in a hexameric ligand/receptor hIL10R complex comprised of two hIL10 monomers, two hIL10Rα subunits and two hIL10Rβ subunits

The hIL10Rα receptor subunit is a transmembrane protein expressed as a 578 amino acid proprotein comprising a N-terminal 21 amino acid signal sequence. The amino acid sequence of the mature canonical hIL10Ra receptor subunit is a 557 amino acid polypeptide of the sequence:

(SEQ ID NO: 24)
HGTELPSPPSVWFEAEFFHHILHWTPIPNQSESTCYEVALLRYGIESWNSI
SNCSQTLSYDLTAVTLDLYHSNGYRARVRAVDGSRHSNWTVTNTRFSVDEV
TLTVGSVNLEIHNGFILGKIQLPRPKMAPANDTYESIFSHFREYEIAIRKV
PGNFTFTHKKVKHENFSLLTSGEVGEFCVQVKPSVASRSNKGMWSKEECIS
LTRQYFTVTNVIIFFAFVLLLSGALAYCLALQLYVRRRKKLPSVLLFKKPS
PFIFISQRPSPETQDTIHPLDEEAFLKVSPELKNLDLHGSTDSGFGSTKPS
LQTEEPQFLLPDPHPQADRTLGNREPPVLGDSCSSGSSNSTDSGICLQEPS
LSPSTGPTWEQQVGSNSRGQDDSGIDLVQNSEGRAGDTQGGSALGHHSPPE
PEVPGEEDPAAVAFQGYLRQTRCAEEKATKTGCLEEESPLTDGLGPKFGRC
LVDEAGLHPPALAKGYLKQDPLEMTLASSGAPTGQWNQPTEEWSLLALSSC
SDLGISDWSFAHDLAPLGCVAAPGGLLGSFNSDLVTLPLISSLQSSE

(UniProt Reference No. Q13651). Residues 22-235 of SEQ ID NO:24 (amino acids 1-214 of the mature hIL10Rβ protein) correspond to the extracellular domain (ECD), residues 236-256 of SEQ ID NO:24 (amino acids 215-235 of the mature hIL10Rβ protein) correspond to the transmembrane domain™ and residues 257-578 of SEQ ID NO:24 (amino acids 236-557) of the mature hIL10Rβ protein) correspond to the intracellular domain (ICD).

The human IL10Rβ (hIL10Rβ) receptor subunit is a transmembrane protein expressed as a 325 amino acid pro-protein comprising a 19 amino acid N-terminal signal. The amino acid sequence of the mature canonical hIL10Rb receptor subunit is a 306 amino acid polypeptide of the sequence:

(SEQ ID NO:25)
MVPPPENVRMNSVNFKNILQWESPAFAKGNLTFTAQYLSYRIFQDKCMNTT
LTECDFSSLSKYGDHTLRVRAEFADEHSDWVNITFCPVDDTIIGPPGMQVE
VLADSLHMRFLAPKIENEYETWTMKNVYNSWTYNVQYWKNGTDEKFQITPQ
YDFEVLRNLEPWTTYCVQVRGFLPDRNKAGEWSEPVCEQTTHDETVPSWMV
AVILMASVFMVCLALLGCFALLWCVYKKTKYAFSPRNSLPQHLKEFLGHPH
HNTLLFFSFPLSDENDVFDKLSVIAEDSESGKQNPGDSCSLGTPPGQGPQS

(UniProt Reference No. Q08334). Amino acids 20-220 (amino acids 1-201 of the mature hIL10Rβ protein) correspond to the extracellular domain, amino acids 221-242 (amino acids 202-223 of the mature hIL10Rβ protein) correspond to the 22 amino acid transmembrane domain, and amino acids 243-325 (amino acids 224-306 of the mature hIL10Rβ protein) correspond to the intracellular domain.

The murine IL10Rβ (mIL10Rβ) receptor subunit is expressed as a 349 amino acid pro-protein comprising a 19 amino acid N-terminal signal sequence. The amino acid sequence of the mature canonical hIL10Rb receptor subunit is a 330 amino acid polypeptide of the sequence:

(SEQ ID NO: 26)
MIPPPEKVRMNSVNFKNILQWEVPAFPKTNLTFTAQYESYRSFQDHCKRTA
STQCDFSHLSKYGDYTVRVRAELADEHSEWVNVTFCPVEDTIIGPPEMQIE
SLAESLHLRFSAPQIENEPETWTLKNIYDSWAYRVQYWKNGTNEKFQVVSP
YDSEVLRNLEPWTTYCIQVQGFLLDQNRTGEWSEPICERTGNDEITPSWIV
AIILIVSVLVVFLFLLGCFVVLWLIYKKTKHTFRSGTSLPQHLKEFLGHPH
HSTFLLFSFPPPEEAEVFDKLSIISEESEGSKQSPEDNCASEPPSDPGPRE
LESKDEAPSPPHDDPKLLTSTSEV

mIL10Rβ (mIL10R2) is referenced at UniProtKB database as entry Q61190. Amino acids 20-220 (amino acids 1-201 of the mature protein) correspond to the extracellular domain, amino acids 221-241 (amino acids 202-222 of the mature protein) correspond to the 21 amino acid transmembrane domain, and amino acids 242-349 (amino acids 223-330 of the mature protein) correspond to the intracellular domain.

The interaction of IL10 with its receptor and receptor subunits have been studied and described in the scientific literature. Pletnev, et al. provide information relating to the structures of the soluble receptor chain of IL10Rβ and the ternary complex of IL10/sIL10Rα/sIL10Rβ and residues involved in ligand-receptor and receptor-receptor interactions. Pletnev, et al. (2005) BMC Structural Biology 5:10. Although the interaction between hIL10 and the hIL10Rα receptor subunit is a specific high-affinity interaction, the association of hIL10 with hIL10Rβ is a comparatively low affinity interaction. Reports suggest that the interaction of hIL10 with hIL10Rα induces a conformational change in hIL10 and/or hIL10Rα which facilitates the binding of the [hIL10:hIL10Rα] complex to hIL10Rβ. The formation of the ternary [hIL10:hIL10Rα: hIL10Rβ] complex is suggested as the limiting factor in initiating hIL10 signaling.

The interaction of IL-10 with the IL10R effects the activation of JAK1 (associated with hIL10Rα) and Tyk2 (associated with hIL10Rβ) and induces the activation of STAT1, STAT3, and, in some cells, STAT5. STAT3 is recruited directly to the hIL-10/hIL-10R complex via either of two tyrosine residues in the hIL10Rα cytoplasmic domain that become phosphorylated in response to hIL-10 and are required for hIL-10 signaling. Homodimerization of STAT3 results in its release from the receptor and translocation of the phosphorylated STAT homodimer into the nucleus, where it binds to STAT3-binding elements in the promoters of numerous genes, including the promoter of IL10, which is positively regulated by STAT3. The hIL10 receptor intracellular domain possesses sequences that are linked to its anti-inflammatory activity that are not shared by other STAT3 activating cytokine receptors. Riley, et al. (1999) Journal of Biological Chemistry 274(23):15967-16664.

The expression of the hIL10Rα and IL10Rb receptor subunits vary with respect to cell type and the activation state of the cell. The activation state of cells that express hIL10Rα may result in substantial variability in the levels of expression. The expression hIL10Rα in hematopoietic cells, which constitutively express low levels of hIL10Rα, is frequently substantially upregulated by various stimuli. In contrast to hIL10Rα, which is expressed primarily on hematopoietic cells, the IL10Rb receptor subunit is expressed ubiquitously. While certain cell types express hIL10Rβ at different levels, the level of hIL10Rβ expression in a given cell type is typically less affected by the activation state of the cell than hIL10Rα.

hIL10 is associated with a wide variety of functions and exhibits both immunosuppressive and immunostimulatory activities through its interaction with T cells, B cells, macrophages, and antigen presenting cells (APCs). The immunosuppressive activity of hIL10 is well documented. hIL10 is associated with suppressing the expression of IL1α, IL1β, IL6, IL8, TNFα, GM-CSF and G-CSF in activated monocytes and activated macrophages, as well as suppression of proinflammatory cytokine interferon-gamma (INFγ) production by NK cells. However, hIL10 also demonstrates an immunostimulatory effect by stimulation of the production of the proinflammatory cytokine IFN-γ by CD8+ T cells. The immunostimulatory and immunosuppressive properties of hIL10 have proved to be a challenge in the clinical application of hIL10 in the treatment of human disease.

Saxton, et al. describe the interaction of IL10 with the IL10Rb subunit and amino acid residues that are involved in the binding of IL10 to IL10Rβ and that modification of such residues can potentially provide IL10 variants that retain the immunosuppressive function of IL10 on myeloid cells but have diminished pro-inflammatory activity on CD8+ T cells. Saxton, et al. (2021) Science 371 (6535), eabc8433.

### SUMMARY OF THE DISCLOSURE

The present disclosure provides compositions that are useful for modulating signal transduction mediated by interleukin-10 (IL10). In particular, the disclosure provides inverted monomers and dimers thereof that are partial agonists of STAT3-mediated signaling (“STAT3 signaling”). In some embodiments, the inverted monomers and dimers thereof described herein activate STAT3 signaling in some cell types, and result in decreased STAT3 signaling in other cell types. In some embodiments, the inverted monomers and dimers thereof described herein activate STAT3 signaling in myeloid cells, and have decreased STAT3 signaling in lymphocytes. In some embodiments, the inverted monomers and dimers thereof described herein demonstrated a preferential activation of STAT3 signaling in myeloid cells relative to lymphocytes as compared to wt hIL10.

In one aspect a polypeptide is provided comprising an amino acid sequence of the formula 1:

[A]-L1x-[B]-L2-[C]-L3-[D]-L4y-[E]-L5-[F] (1)

wherein:
x and y are independently selected from 0 (absent) or 1 (present);
[A] is a polypeptide having 0, 1 or 2 amino acid substitutions relative to the amino acid sequence SKAVEQVKNAFNKL (SEQ ID NO:1);
x=0 (no linker, L1 is absent), or
x=1 and L1 comprises a polypeptide linker of from 1 to 5 amino acids;
[B] is a polypeptide having 0, 1 or 2 amino acid substitutions relative to the amino acid sequence “EKGIYKAMSEFDIFINYIEAYMTMKIR” (SEQ ID NO:2);
L2 comprises a linker of from 10 to 25 amino acids;
[C] is a polypeptide having 0, 1 or 2 amino acid substitutions relative to the amino acid sequence “NLPNMLRDLRDAFSRVKTFFQMKD” (SEQ ID NO:3);
L3 comprises a linker of from 4 to 11 amino acids;
[D] is a polypeptide having 0, 1 or 2 amino acid substitutions relative to the amino acid sequence “KESLLEDFKG” (SEQ ID NO:4);
y=0 (L4 is absent), or
y=1 and L4 comprises a polypeptide linker of from 1 to 5 amino acids;
[E] is a polypeptide having 0, 1 or 2 amino acid substitutions relative to the amino acid sequence “LGCQALSEMIQFYLEEVMPQAEN” (SEQ ID NO:5);
L5 comprises a linker of from 1 to 7 amino acids; and
[F] is a polypeptide having 0, 1 or 2 amino acid substitutions relative to the amino acid sequence “IKAHVNSLGENLKTLRLRLRRC” (SEQ ID NO:6).

In some embodiments, L1, L2, L3, L4 and/or L5 comprise a GS-linker. In some embodiments, L1 is the amino acid glutamine (Q). In some embodiments, L2 is a polypeptide having the amino acid sequence NSPGQGTQSENSCTHFPG (SEQ ID NO:7) or NTSPGQGTQSENSCTHFPG (SEQ ID NO:23). In some embodiments, L3 is a polypeptide having the amino acid sequence QLDNLLL (SEQ ID NO:8). In some embodiments, L4 is the amino acid glycine (“G”) or comprises the amino acid sequence GY. In some embodiments, L5 is a polypeptide having the amino acid sequence QDPD (SEQ ID NO:9).

In some embodiments, the polypeptide comprises an amino acid sequence having at least 95% sequence identity to SEQ ID NO:10 or SEQ ID NO:11.

In some embodiments, the polypeptide comprises the amino acid sequence

(SEQ ID NO:10)
KSKAVEQVKNAFNKLQEKGIYKAMSEFDIFINYIEAYMTMKIRNSPGQGTQ
SENSCTHFPGNLPNMLRDLRDAFSRVKTFFQMKDQLDNLLLKESLLEDFKG
YLGCQALSEMIQFYLEEVMPQAENQDPDIKAHVNSLGENLKTLRLRLRRCH
RFLPCEN,

In some embodiments, the polypeptide comprises the amino acid sequence
KSKAVEQVKNAFNKLQEKGIYKAMSEFDIFINYIEAYMTMKIRNTSPGQGTQS ENSCTHFPGNLPNMLRDLRDAFSRVKTFFQMKDQLDNLLLKESLLEDFKGYL GCQALSEMIQFYLEEVMPQAENQDPDIKAHVNSLGENLKTLRLRLRRCHRFL PCEN (SEQ ID NO:11), or an amino acid sequence having at least 95% sequence identity to SEQ ID NO:10 or SEQ ID NO: 11.

In some embodiments, the polypeptide comprises an amino acid sequence having at least 90%, 91%, 92%, 93%, 94%, 95%, 96%, 97%, 98%, 99% or 100% sequence identity hIL10 inverted monomer selected from the group consisting of SEQ ID NO: 10, SEQ ID NO:11, SEQ ID NO:90, SEQ ID NO:91, SEQ ID NO:92, SEQ ID NO:93, SEQ ID NO:94, SEQ ID NO:95, SEQ ID NO:96, SEQ ID NO:97, and SEQ ID NO:98

In some embodiments, a polypeptide is provided comprising formula (2):

[Monomer1]-linkerₓ-[Monomer2] (2)

wherein Monomer 1 and Monomer 2 are polypeptides of formula 1 and Monomer 1 and Monomer 2 are the same or different, and x=0 (linker absent) or 1 (linker present).

In some embodiments, the polypeptide of formula (2) comprises in the following amino to carboxyl order:
(a) Monomer 1 comprising:
   (i) a first polypeptide comprising an amino acid sequence having at least 90%, 91%, 92%, 93%, 94%, 95%, 96%, 97%, 98%, 99% or 100% sequence identity to a polypeptide selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature wild-type human IL-10 (SEQ ID NO:20);
   (ii) a first linkerₓ, wherein x=0 (linker absent) or 1 (linker present); and
   (iii) a second polypeptide comprising an amino acid having at least 90%, 91%, 92%, 93%, 94%, 95%, 96%, 97%, 98%, 99% or 100% sequence identity to a polypeptide sequence selected from the group consisting of amino acid residues 1-160 of human IL-10 (SEQ ID NO:20); amino acid residues 2-160 of human IL-10 (SEQ ID NO:36); amino acid residues 3-160 of human IL-10 (SEQ ID NO:37); amino acid residues 4-160 of human IL-10 (SEQ ID NO:38); amino acid residues 5-160 of human IL-10 (SEQ ID NO:39); amino acid residues 6-160 of human IL-10 (SEQ ID NO:40); amino acid residues 7-160 of human IL-10 (SEQ ID NO:41); amino acid residues 8-160 of human IL-10 (SEQ ID NO:42); amino acid residues 9-160 of human IL-10 (SEQ ID NO:43); amino acid residues 10-160 of human IL-10 (SEQ ID NO:44); and amino acid residues 11-160 of human IL-10 (SEQ ID NO:45) numbered in accordance with mature wild-type hIL10 (SEQ ID NO:20);
(b) a second linkery, wherein y=0 (linker absent) or 1 (linker present); and
(c) Monomer 2 comprising a polypeptide having at least 90%, 91%, 92%, 93%, 94%, 95%, 96%, 97%, 98%, 99% or 100% sequence identity to a polypeptide sequence selected from the group consisting of amino acid residues 1-116 of human IL-10 (SEQ ID NO:15); amino acid residues 2-116 of human IL-10 (SEQ ID NO:16); amino acid residues 3-116 of human IL-10 (SEQ ID NO:17), amino acid residues 4-116 of human IL-10 (SEQ ID NO:18), amino acid residues 5-116 of human IL-10 (SEQ ID NO:19), amino acid residues 6-116 of human IL-10 (SEQ ID NO:29), amino acid residues 7-116 of human IL-10 (SEQ ID NO:30), amino acid residues 8-116 of human IL-10 (SEQ ID NO:31), amino acid residues 9-116 of human IL-10 (SEQ ID NO:32), amino acid residues 10-116 of human IL-10 (SEQ ID NO:33); and amino acid residues 11-116 of human IL-10 (SEQ ID NO:34), numbered in accordance with mature human IL-10 (SEQ ID NO:20).

In some embodiments, the polypeptide of formula (2) comprises in the following amino to carboxyl order:
(a) Monomer 1 comprising:
   (i) a first polypeptide comprising an amino acid sequence selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature human IL-10 (SEQ ID NO:20),
   (ii) a first linkerₓ;
   (iii) a second polypeptide comprising an amino acid sequence selected from the group consisting of amino acid residues 1-116 of human IL-10 (SEQ ID NO:15);
(b) a second linkery;
(c) Monomer 2 comprising:
   (i) a first polypeptide comprising an amino acid sequence selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature human IL-10 (SEQ ID NO:20),
   (ii) a third linker_{z}; and
   (iii) a second polypeptide comprising an amino acid sequence selected from the group consisting of amino acid residues 1-116 of human IL-10 (SEQ ID NO:15).

In some embodiments, the polypeptide comprises an amino acid sequence having at least 90%, 91%, 92%, 93%, 94%, 95%, 96%, 97%, 98%, 99% or 100% sequence identity hIL10 inverted monomer selected from the group consisting of SEQ ID NOS:46-51.

In some embodiments, the polypeptide exhibits cell type biased activity relative to the wild type IL10 species from which the inverted IL10 monomer was derived. In some embodiments, the polypeptide (a) exhibits a significant level of at least one anti-inflammatory property of wild type IL10 and (b) exhibits a significantly reduced level of at least one pro-inflammatory property of wild type IL10. In some embodiments, the at least one anti-inflammatory property is selected from the group consisting of suppression of expression or secretion of IL1β, TNFα or IL6 in a myeloid cell. In some embodiments, the at least one pro-inflammatory property is selected from the group consisting of suppression of expression or secretion of IFNγ, granzyme A or granzyme B in a T cell. In some embodiments, the at least one anti-inflammatory property is selected from the group consisting of suppression of expression or secretion of IL1 β, TNFα or IL6 in a myeloid cell and the at least one pro-inflammatory property is selected from the group consisting of suppression of expression or secretion of IFNγ, granzyme A or granzyme B in a T cell.

In some embodiments, the cell type biased activity is the production of phospho-STAT3. In some embodiments, wherein the pSTAT3 Emax of the polypeptide is greater than 20%, greater than 30%, greater than 40%, greater than 50%, greater than 60%, or greater than 70% of the pSTAT3 Emax of wild-type hIL10 in myeloid cells.

In some embodiments, the pSTAT3 Emax in a lymphocyte less than 70%, less than 60%, less than 50%, less than 40%, or less than 30%, of the pSTAT3 Emax of a wild-type hIL10 in the lymphocyte.

In some embodiments, (a) the pSTAT3 Emax of the polypeptide is greater than 20%, greater than 30%, greater than 40%, greater than 50%, greater than 60%, or greater than 70% of the pSTAT3 Emax of wild-type hIL10 in myeloid cells and (b) the pSTAT3 Emax in a lymphocyte less than 70%, less than 60%, less than 50%, less than 40%, or less than 30%, of the pSTAT3 Emax of a wild-type hIL10 in the lymphocyte.

In some embodiments, a polypeptide is provided comprising formula (2):

[Monomer1]-linkerₓ-[Monomer2] (2)

wherein Monomer 1 and Monomer 2 each, independently, comprise a polypeptide selected from a polypeptide of claim 1, and x=0 (linker absent) or 1 (linker present).

In some embodiments, Monomer 1 and Monomer 2 each, independently, comprise a polypeptide comprising an amino acid sequence having at least 90%, 91%, 92%, 93%, 94%, 95%, 96%, 97%, 98%, 99% or 100% sequence identity to an amino acid sequence selected from the group consisting of SEQ ID NO:10, SEQ ID NO:11, SEQ ID NO: 90, SEQ ID NO:91, SEQ ID NO:92, SEQ ID NO:93, SEQ ID NO:94, SEQ ID NO:95, SEQ ID NO:96, SEQ ID NO:97, and SEQ ID NO:98.

In some embodiments, Monomer 1 and Monomer 2 each comprise a polypeptide comprising the amino acid sequence of SEQ ID NO:10.

In some embodiments, Monomer 1 and Monomer 2 each comprise a polypeptide comprising the amino acid sequence of SEQ ID NO:11.

In some embodiments, x is 1 (linker present) and the linker comprises a GS-linker.

In some embodiments, x is 0 (linker absent).

In some embodiments, the polypeptide of formula (2) comprises in the following amino to carboxyl order:
(a) Monomer 1 comprising:
   (i) a first polypeptide comprising an amino acid sequence having at least 90%, 91%, 92%, 93%, 94%, 95%, 96%, 97%, 98%, 99% or 100% sequence identity to a polypeptide selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature wild-type human IL-10 (SEQ ID NO:20);
   (ii) a first linkerₓ, wherein x=0 (linker absent) or 1 (linker present); and
   (iii) a second polypeptide comprising an amino acid having at least 90%, 91%, 92%, 93%, 94%, 95%, 96%, 97%, 98%, 99% or 100% sequence identity to a polypeptide sequence selected from the group consisting of amino acid residues 1-160 of human IL-10 (SEQ ID NO:20); amino acid residues 2-160 of human IL-10 (SEQ ID NO:36); amino acid residues 3-160 of human IL-10 (SEQ ID NO:37); amino acid residues 4-160 of human IL-10 (SEQ ID NO:38); amino acid residues 5-160 of human IL-10 (SEQ ID NO:39); amino acid residues 6-160 of human IL-10 (SEQ ID NO:40); amino acid residues 7-160 of human IL-10 (SEQ ID NO:41); amino acid residues 8-160 of human IL-10 (SEQ ID NO:42); amino acid residues 9-160 of human IL-10 (SEQ ID NO:43); amino acid residues 10-160 of human IL-10 (SEQ ID NO:44); and amino acid residues 11-160 of human IL-10 (SEQ ID NO:45) numbered in accordance with mature wild-type hIL10 (SEQ ID NO:20);
(b) a second linkery, wherein y=0 (linker absent) or 1 (linker present); and
(c) Monomer 2 comprising a polypeptide having at least 90%, 91%, 92%, 93%, 94%, 95%, 96%, 97%, 98%, 99% or 100% sequence identity to a polypeptide sequence selected from the group consisting of amino acid residues 1-116 of human IL-10 (SEQ ID NO:15); amino acid residues 2-116 of human IL-10 (SEQ ID NO:16); amino acid residues 3-116 of human IL-10 (SEQ ID NO:17), amino acid residues 4-116 of human IL-10 (SEQ ID NO:18), amino acid residues 5-116 of human IL-10 (SEQ ID NO:19), amino acid residues 6-116 of human IL-10 (SEQ ID NO:29), amino acid residues 7-116 of human IL-10 (SEQ ID NO:30), amino acid residues 8-116 of human IL-10 (SEQ ID NO:31), amino acid residues 9-116 of human IL-10 (SEQ ID NO:32), amino acid residues 10-116 of human IL-10 (SEQ ID NO:33); and amino acid residues 11-116 of human IL-10 (SEQ ID NO:34), numbered in accordance with mature human IL-10 (SEQ ID NO:20).

In some embodiments, the polypeptide of formula (2) comprises in the following amino to carboxyl order:
(a) Monomer 1 comprising:
   (i) a first polypeptide comprising an amino acid sequence selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature human IL-10 (SEQ ID NO:20),
   (ii) a first linkerₓ, wherein x=0 (linker absent) or 1 (linker present);
   (iii) a second polypeptide comprising an amino acid sequence selected from the group consisting of amino acid residues 1-116 of human IL-10 (SEQ ID NO:15);
(b) a second linkery, wherein y=0 (linker absent) or 1 (linker present);
(c) Monomer 2 comprising:
   (i) a first polypeptide comprising an amino acid sequence selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature human IL-10 (SEQ ID NO:20),
   (ii) a third linker_{z}, wherein z=0 (linker absent) or 1 (linker present); and
   (iii) a second polypeptide comprising an amino acid sequence selected from the group consisting of amino acid residues 1-116 of human IL-10 (SEQ ID NO:15).

In some embodiments, the polypeptide comprises an amino acid sequence having at least 90%, 91%, 92%, 93%, 94%, 95%, 96%, 97%, 98%, 99% or 100% sequence identity hIL10 inverted monomer selected from the group consisting of SEQ ID NOS:46-51.

In some embodiments, the polypeptide is PEGylated. In some embodiments, the PEG molecule is linear or branched and has a molecular weight of from about 10 kD to about 80 kD. In some embodiments, the PEG molecule is a 40 kD branched PEG molecule comprising two 20 kD arms. In some embodiments, the PEG molecule is covalently attached to the N-terminus of the polypeptide.

In some embodiments, the polypeptide exhibits cell type biased activity relative to the wild type IL10 species from which the inverted IL10 monomer was derived. In some embodiments, the polypeptode: (a) exhibits a significant level of at least one anti-inflammatory property of wild type IL10 and (b) exhibits a significantly reduced level of at least one pro-inflammatory property of wild type IL10. In some embodiments, the at least one anti-inflammatory property is selected from the group consisting of suppression of expression or secretion of IL1b, TNFa or IL6 in a myeloid cell In some embodiments, the at least one pro-inflammatory property is selected from the group consisting of suppression of expression or secretion of IFNγ, granzyme A or granzyme B in a T cell. In some embodiments, the at least one anti-inflammatory property is selected from the group consisting of suppression of expression or secretion of IL1b, TNFa or IL6 in a myeloid cell and the at least one pro-inflammatory property is selected from the group consisting of suppression of expression or secretion of IFNγ, granzyme A or granzyme B in a T cell.

In some embodiments, the cell type biased activity is the production of phospho-STAT3. In some embodiments, the pSTAT3 Emax of the polypeptide is greater than 20%, greater than 30%, greater than 40%, greater than 50%, greater than 60%, or greater than 70% of the pSTAT3 Emax of wild-type hIL10 in myeloid cells. In some embodiments, the pSTAT3 Emax in a lymphocyte less than 70%, less than 60%, less than 50%, less than 40%, or less than 30%, of the pSTAT3 Emax of a wild-type hIL10 in the lymphocyte. In some embodiments, (a) the pSTAT3 Emax of the polypeptide is greater than 20%, greater than 30%, greater than 40%, greater than 50%, greater than 60%, or greater than 70% of the pSTAT3 Emax of wild-type hIL10 in myeloid cells and (b) the pSTAT3 Emax in a lymphocyte less than 70%, less than 60%, less than 50%, less than 40%, or less than 30%, of the pSTAT3 Emax of a wild-type hIL10 in the lymphocyte.

Also provided is a nucleic acid sequence encoding a polypeptide as described above or elsewhere herein.

Also provided is a recombinant vector comprising the nucleic acid sequence as described above or elsewhere herein operably linked to one or more expression control sequences.

Also provided is a recombinant cell transformed with the recombinant vector as described above or elsewhere herein.

Also provided is a method of making a polypeptide as described above or elsewhere herein, the method comprising the steps of (a) culturing the host cell as described above or elsewhere herein under conditions suitable for the expression of the polypeptide and (b) recovering the polypeptide from the host cell culture. In some embodiments, the host cell is a mammalian host cell. In some embodiments, the host cell is a bacterial cell.

Also provided is a composition comprising a polypeptide as described above or elsewhere herein, a nucleic acid sequence as described above or elsewhere herein, or a recombinant vector as described above or elsewhere herein and one or more pharmaceutically acceptable salts, excipients, and/or diluents.

Also provided is a method of treating a mammalian subject suffering from a disease, disorder or condition, the method comprising administering to said subject a therapeutically effective amount a polypeptide as described above or elsewhere herein, or a composition as described above or elsewhere herein. In some embodiments, the disease, disorder or condition is an autoimmune disease, disorder or condition. In some embodiments, the autoimmune disease, disorder or condition is selected from the group consisting of ulcerative colitis, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, and SEA Syndrome (Seronegativity, Enthesopathy, Arthropathy Syndrome). In some embodiments, disease disorder or condition is a cancer associated with chronic inflammation. In some embodiments, the treating prevents the progression of the disease, disorder or condition. In some embodiments, the treating ameliorates one or more symptoms of the disease, disorder or condition.

Also provided is a method of preventing a disease, disorder or condition in a mammalian subject at risk for developing the disease, disorder or condition, the method comprising administering to said subject a prophylactically effective amount of the composition as described above or elsewhere herein prior to the onset of symptoms of the disease, disorder or condition as described above or elsewhere herein the disease disorder or condition is a cancer associated with chronic inflammation.

### BRIEF DESCRIPTION OF THE DRAWINGS

FIG. 1 shows thermal stability data relating to an IL10 inverted monomer of the present disclosure as more fully described in Examples. The data indicates that the hIL10 inverted monomer possesses enhanced thermal stability relative to hIL10.

FIG. 2A shows results of a flow cytometric assay evaluating the level of intracellular STAT3 (y-axis) in human monocytes exposed various concentrations (x-axis) of wild type human IL10 and an inverted hIL10 monomer as more fully described in Examples.

FIG. 2B shows results of a flow cytometric assay evaluating the level of intracellular STAT3 (y-axis) in human naïve CD8 T cells exposed various concentrations (x-axis) of wild type human IL10 and an inverted hIL10 monomer as more fully described in Examples.

FIG. 3 shows the results of a human monocyte activity assay to detect the secretion of IL-10 (y-axis) in human monocytes exposed to various in concentrations (x-axis) of wild-type hIL10 (h_SM0043_AA) and an inverted monomer of the disclosure (h_DR1061_AA) as more fully described in the Examples.

FIGS. 4A-4C show the secreted levels (y-axes) of IFNγ (FIG. 4A), Granzyme A (FIG. 4B) and Granzyme B (FIG. 4C) in activated human CD8+ T cell in response to various concentrations (x-axes)( ) of wild-type hIL10 (h_SM0043_AA) and an inverted monomer of the disclosure (h_DR1061_AA) as more fully described in the Examples.

FIG. 5A shows results of a flow cytometric assay evaluating the level of intracellular STAT3 (y-axis) in mouse myeloid cells exposed various concentrations (x-axis) of wild type murine IL10 (m_DR756_AA) and an inverted mIL10 monomer (m_WC161_AA) as more fully described in Examples. FIG. 5B shows results of a flow cytometric assay evaluating the level of intracellular STAT3 (y-axis) in mouse CD8 T cells exposed various concentrations (x-axis) of wild type murine IL10 (m_DR756_AA) and an inverted mIL10 monomer (m_WC161_AA) as more fully described in Examples.

FIG. 6A shows the results of an experiment to evaluate IL6 secretion (y-axis) in mouse splenocytes in response to varying concentrations (x-axis) of wild type murine IL10 (m_DR756_AA) and an inverted mIL10 monomer (m_WC161_AA) as more fully described in Examples.

FIG. 6B shows the results of an experiment to evaluate TNFα secretion (y-axis) in mouse splenocytes in response to varying concentrations (x-axis) of wild type murine IL10 (m_DR756_AA) and an inverted mIL10 monomer (m_WC161_AA) as more fully described in Examples a mouse splenocyte TNF-alpha assay.

FIG. 7 shows the results of an experiment to evaluate granzyme B (y-axis) produced in activated mouse CD8+ T cells in response to varying concentrations (x-axis) of wild type murine IL10 (m_DR756_AA) and an inverted mIL10 monomer (m_WC161_AA) as more fully described in Examples.

FIG. 8 shows the results of an experiment to survival of activated CD8+ mouse T cells (y-axis) produced in activated mouse CD8+ T cells in response to varying concentrations (x-axis) of wild type murine IL10 (m_DR756_AA) and an inverted mIL10 monomer (m_WC161_AA) as more fully described in Examples.

FIG. 9 shows the results of a pharmacokinetic study to evaluate the serum stability (y-axis) over time (x-axis) of various dosage levels of PEGylated mouse inverted IL10 monomer (WC161) relative to the pegylated wild-type mouse IL10 control (DR756) as more fully described in Examples.

FIG. 10 shows the results of an experiment to evaluate the level of cell surface expression of CD64 (y-axis) on human monocytes treated with varying (0.1 pM-100 nM) concentrations (x-axis) of IL10 proteins for 48 hours at 37 °C as more fully described in Examples.

FIG. 11 shows an alignment of a human (DR1060_aa/1-171; SEQ ID NO:27) and mouse (WC161_aa/1-172; SEQ ID NO:28) inverted monomer of the disclosure.

FIG. 12A shows results of a flow cytometric assay evaluating the level of intracellular STAT3 (y-axis) in human monocytes exposed various concentrations (x-axis) of wild type human IL10 (h_DR757_AA) and inverted hIL10 monomers (h_DR1060_AA and h_DR1061_AA) as more fully described in Examples.

FIG. 12B shows results of a flow cytometric assay evaluating the level of intracellular STAT3 (y-axis) in human naïve CD8 T cells exposed various concentrations (x-axis) of wild type human IL10 (h_DR757_AA) and inverted hIL10 monomers (h_DR1060_AA and h_DR1061_AA) as more fully described in Examples.

FIG. 13 shows the data relating to bodyweight of mice (y-axis) over time (x-axis) in a DSS model of ulcerative colitis as more fully described in the Examples. The upper panel reflects simple bodyweight measurements while the lower panel provides the weights of the animals relative to their initial weight upon starting the study.

FIG. 14 shows the data relating to bodyweight of mice (y-axis) treated with test agents (x-axis) in a DSS model of ulcerative colitis assessed upon termination of the study (D15) as more fully described in the Examples.

FIG. 15 shows the data relating to colon length of mice (y-axis) treated with test agents (x-axis) in a DSS model of ulcerative colitis assessed upon termination of the study (D15) as more fully described in the Examples.

FIG. 16 shows the data relating to hematocrit levels from mice (y-axis) treated with test agents (x-axis) in a DSS model of ulcerative colitis assessed on study day 4 (D4) as more fully described in the Examples.

FIG. 17 shows the data relating to the percent of peritoneal CD163+ macrophages (y-axis) from mice treated with test agents (x-axis) in a DSS model of ulcertative colitis as more fully described in the Examples.

FIG. 18 shows the data relating to the percent of peritoneal CD64 macrophages (y-axis) from mice treated with test agents (x-axis) in a DSS model of ulcerative colitis as more fully described in the Examples.

FIG. 19 shows the data relating to serum cytokine levels from mice (y-axis), over time (x-axis), with respect to the various treatments in a DSS model of ulcerative colitis as more fully described in the Examples.

FIG. 20 shows the data relating to serum cytokine levels from mice (y-axis), over time (x-axis), with respect to the various treatments in a DSS model of ulcerative colitis as more fully described in the Examples.

FIG. 21 shows the data relating to the percent epithelial damage (y-axis) from mice treated with test agents (x-axis) measured on D15 of the DSS model of ulcerative colitis as more fully described in the Examples.

FIG. 22, left panel, shows the data relating to the levels of CD11b cells in the intestinal mucosa (y-axis) from mice treated with test agents (x-axis) measured on D15 of the DSS model of ulcerative colitis as more fully described in the Examples. FIG. 22, right panel, shows the data relating to the levels of Th17 cells in the intestinal mucosa (y-axis) from mice treated with test agents (x-axis) measured on D15 of the DSS model of ulcerative colitis as more fully described in the Examples.

FIG. 23 shows results of a pharmacokinetic study performed in mice to evaluate the serum concentrations of test agents (y-axis) over time (x-axis) in response to administration of various levels the test agents as more fully described in the Examples.

FIG. 24 shows the data relating to the levels of STAT3 induction (y-axis) in CD4T cells (upper panel), CD8 T cells (middle panel) and B cells (lower panel) in response to administration of various levels the test agents as more fully described in the Examples.

### DETAILED DESCRIPTION

To facilitate the understanding of present disclosure, certain terms and phrases are defined below as well as throughout the specification. The definitions provided herein are non-limiting and should be read in view of the knowledge of one of skill in the art.

Before the present methods and compositions are described, it is to be understood that this invention is not limited to a particular method or composition described. It is also to be understood that the terminology used herein is for the purpose of describing embodiments only and is not intended to be limiting.

Where a range of values is provided, it is understood that each intervening value, to the tenth of a unit, between the upper and lower limits of the stated range is also specifically disclosed unless the context clearly dictates otherwise. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, technical and scientific terms used herein are construed as commonly understood by one of ordinary skill in the art to which this invention belongs. Although any methods and materials similar or equivalent to those described herein may be used in the practice or testing of the present invention, some potential and preferred methods and materials are now described. All publications, patents, published patent applications, GenBank accession numbers and UniProt reference numbers mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (°C), and pressure is at or near atmospheric. Standard abbreviations are used, including the following: bp=base pair(s); kb=kilobase(s); pl=picoliter(s); s or see=second(s); min=minute(s); h or hr=hour(s); AA or aa=amino acid(s); kb=kilobase(s); nt=nucleotide(s); pg=picogram; ng=nanogram; μ=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m. =intramuscular(ly); i.p. =intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes.

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### Nomenclature of Amino Acid Substitutions and Deletions

The present disclosure provides variant polypeptides comprising amino acid substitutions relative to the wild-type or parent polypeptide. The following nomenclature is used herein to refer to substitutions, deletions or insertions. Residues may be designated herein by the one-letter or three-letter amino acid code of the naturally occurring amino acid found in the wild-type molecule. In the present disclosure, the numbering of amino acid residues of human IL10 polypeptide inverted monomers is made in reference to the number of the residue as provided in SEQ ID NO:10. In reference to the hIL10 muteins, substitutions are designated herein by the one letter amino acid code followed by the wt hIL10 (SEQ ID NO:10) amino acid position followed by the one letter amino acid code which is substituted. For example, an hIL10 mutein having the modification “D25K” refers to a substitution of the aspartic acid (D) residue at position 25 of the (SEQ ID NO:10) with a lysine (K) residue at this position. A deletion of an amino acid reside is referred to as “des” or the symbol “A” followed by the amino acid residue and its position.

Immunoglobulin, Upper Hinge and Fe Residue Numbering: There are a variety of numbering conventions that are employed with respect to the numbering of amino acid residues of immunoglobulins including Kabat numbering, Chothia numbering, EU numbering and IMGT numbering conventions. In the context of the present disclosure, the numbering of amino acid residues of immunoglobulin molecules including domains thereof including the upper hinge and Fc domain (comprising the lower hinge, CH2 and CH3 domains) is made in accordance with EU Numbering conventions. Translation of EU numbering conventions used herein to Kabat numbering, Chothia numbering, or IMGT numbering conventions is readily understood by those of skill in the art. Dondelinger, et al. (2018) Understanding the Significance and Implications of Antibody Numbering and Antigen-Binding Surface Residue Definition Frontiers in Immunology Volume 9 Article #:2278.

### DEFINITIONS

Unless otherwise indicated, the following terms are intended to have the meaning set forth below. Other terms are defined elsewhere throughout the specification.

About: The term “about” refers to a value that is plus or minus 10% of a numerical value described herein, such as plus or minus 0%, 1%, 2%, 3%, 4%, 5%, 6%, 7%, 8%, 9%, or 10% of numerical value described herein. The term “about” also applies to all numerical ranges described herein. All values described herein are understood to be modified by the term “about” whether or not the term “about” is explicitly recited in reference to a given value.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand. The term activate or activated may also be used in reference to a cell state in response to the exposure of the cell to an activating agent.

Activity: As used herein, the term “activity” is used with respect to a molecule to describe a property of the molecule with respect to a test system (e.g., an assay) or biological or chemical property (e.g., the degree of binding of the molecule to another molecule), the effect of an agent on a cell (e.g., activation of a cell) or of a physical property of a material or cell (e.g., modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT3 phosphorylation]/[mg protein], [proliferation]/[mg protein], plaque forming units (pfu), etc. As used herein, the term proliferative activity refers to the activity of an agent that promotes cell proliferation and/or replication.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g., an inverted monomer, a nucleic acid encoding the inverted monomer, a vector (e.g. an expression vector) comprising a nucleic acid encoding the inverted monomer, or an engineered cell expressing an inverted monomer) or a pharmaceutical formulation comprising one or more of the foregoing agents. Administration of an agent may be achieved through any of a variety of art recognized methods including but not limited to the topical administration, oral administration, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), inhalation (e.g., respiratory inhalers including dry-powder inhalers), intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant (K_{D}), a ratio of the dissociation rate constant between the molecule and its target (K_{off}) and the association rate constant between the molecule and its target (Kₒₙ).

Agonist: As used herein, the term “agonist” refers a first agent that specifically binds a second agent (“target”) and interacts with the target to cause or promote an increase in the activation of the target. In some instances, agonists are activators of receptor proteins that modulate cell activity, enhance activation, sensitize cells to activation by a second agent, or up-regulate the expression of one or more genes, proteins, ligands, receptors, biological pathways, that may result in modulation of cellular activity including but not limited to cell activation and/or proliferation or the cell cycle. In some embodiments, an agonist is an agent that binds to a receptor and alters the receptor state resulting in a biological response that mimics the effect of the endogenous ligand of the receptor. In some embodiments, an agonist is a modified form of a cognate ligand that binds to its cognate receptor and alters the state of the cognate receptor in a biological response that mimics the biological effect of the interaction of the naturally occurring cognate ligand with its cognate receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction). A “partial agonist” is a type of agonist that can produce a maximal response less than the endogenous agonist for the target receptor, and thus has a maximal activity of less than 100% of the native ligand for the target. In some embodiments, a superagonist exhibits less than 100% but greater than 10%, alternatively greater than 20%, alternatively greater than 30%, alternatively greater than 40%, alternatively greater than 50%, alternatively greater than 60%, alternatively greater than 70%, alternatively greater than 80% or alternatively greater than 90% of the response in an evaluable quantitative or qualitative parameter of the endogenous agonist for the target receptor when evaluated at similar concentrations in a comparable assay. A “superagonist” is a type of agonist that can produce a maximal response greater than the endogenous agonist for the target receptor, and thus has a maximal activity of more than 100% of the native ligand for the target. In some embodiments, a superagonist exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the endogenous agonist for the target receptor when evaluated at similar concentrations in a comparable assay. It should be noted that the biological effects associated with the partial agonist, full agonist or superagonist may differ not only in degree but also may differ in kind from those biological effects of endogenous agonist for the target receptor.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway including an immune checkpoint pathway, or cell.

Biological Sample: As used herein, the term “biological sample” or “sample” refers to a sample obtained (or derived) from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, tumor tissue, including immunoglobulin enriched or cell-type specific enriched fractions derived from one or more of such tissues.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 35%, alternatively by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Conservative Amino Acid Substitution: As used herein, the term “conservative amino acid substitution” refers to an amino acid replacement that changes a given amino acid to a different amino acid with similar biochemical properties (e.g., charge, hydrophobicity, and size). Amino acids in each of the following groups are typically considered as conservative amino acids of each other: (1) hydrophobic amino acids: alanine, isoleucine, leucine, tryptophan, phenylalanine, valine, proline, and glycine; (2) polar amino acids: glutamine, asparagine, histidine, serine, threonine, tyrosine, methionine, and cysteine; (3) basic amino acids: lysine and arginine; and (4) acidic amino acids: aspartic acid and glutamic acid.

Corresponding To: As used herein, the terms “correspondence” or “corresponding to” in the context of an amino acid or nucleotide in a polypeptide or polynucleotide, respectively, refers to the equivalent position (e.g. amino acid or nucleotide) of a reference polypeptide or polynucleotide sequence when the reference sequence is aligned with a second sequences to maximize the percentage of sequence identity. For example, an “amino acid position corresponding to amino acid position [X]” of a specified IL10 polypeptide refers to equivalent positions, based on alignment, in other IL10 polypeptides, including structural homologues and variants or IL10 derived from different species (e.g. human IL10 and mouse IL10). The corresponding position can be based on a reference sequence, wild-type sequence or parental sequence, for example, with respect to hIL10 muteins a reference sequence may be the amino acid sequence of wild type hIL10 (SEQ ID NO:10).

Derived From: As used herein, the term “derived from” in the context of polypeptide or polynucleotide variants or muteins is meant to indicate that the polypeptide or polynucleotide variant or mutein has a sequence that is based on that of a reference polypeptide (e.g., the amino acid sequence of wild type hIL10 (SEQ ID NO:10)) or polynucleotide sequence (e.g. the cDNA encoding wt hIL10). The term “derived from” is not to be understood as limiting with respect to the source or method in which the polypeptide or polynucleotide variants or mutein is made.

Effective Concentration (EC): As used herein, the terms “effective concentration” or its abbreviation “EC” are used interchangeably to refer to the concentration of an agent in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of a test agent. When the abbreviation EC is provided with a subscript (e.g., EC₄₀, EC₅₀, etc.) the subscript refers to the percentage of the Emax of the biological response observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results in the maximal (100%) response of a measurable parameter in response to such agent. Similarly, the term EC₅₀ (which is commonly used in the field of pharmacodynamics) refers to the concentration of an agent sufficient to result in the half-maximal (about 50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect.

Enriched: As used herein in the term “enriched” refers to a sample that is non-naturally manipulated so that a species (e.g., a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., a recombinantly modified bacterial or mammalian cell).

Extracellular Domain: As used herein the term “extracellular domain” or its abbreviation “ECD” refers to the portion(s) of a cell surface protein (e.g., a cell surface receptor) which is (are) external to of the plasma membrane of a cell. The cell surface protein may be transmembrane protein, a cell surface or membrane associated protein. The cell surface protein may be a multi-pass transmembrane protein having multiple non-contiguous extracellular domains.

Fusion Protein: The term “fusion protein” refers to a polypeptide that comprises amino acid sequences derived from different proteins or synthetic sequences providing differing functions. Examples of fusion proteins include but are not limited to polypeptides comprising a protein and at least one additional functional domain including but not limited to immunogenic domains (e.g. diphtheria or tetanus toxin), polypeptide domains to facilitate expression (e.g. signal peptides) sequences to facilitate isolation and/or purification (e.g. chelating peptides), polypeptide targeting domains (e.g. single domain antibodies), a protein having a distinct additional (e.g. complementary, especially complementary therapeutic) function, polypeptide domains that promote extended duration of action in vivo (e.g. albumin). The domains of a fusion protein may further be joined by polypeptide linkers. The functional domains of the fusion protein may be provided in any N-terminal to C-terminal order. Fusion proteins are typically produced by translation of a single recombinant DNA sequence such that the protein and the additional functional domain of the fusion protein are covalently joined by peptide bonds.

Identity: The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as BLAST 2.0 algorithms, which are described in Altschul et al. (1990) J Mol. Biol. 215: 403-410 and Altschul, et al. (1977) Nucleic Acids Res. 25: 3389-3402. Software for performing BLAST analyses is publicly available through the National Center for Biotechnology Information (NCBI) web site. The algorithm involves first identifying high scoring sequence pairs (HSPs) by identifying short words of length W of the query sequence, which either match or satisfy some positive-valued threshold score “T” when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (the reward score for a pair of matching residues; always >0) and “N” (the penalty score for mismatching residues; always <0). For amino acid sequences, a scoring matrix is used to calculate the cumulative score. Extension of the word hits in each direction are halted when: (a) the cumulative alignment score falls off by the quantity X from its maximum achieved value; the cumulative score goes to zero or below, due to the accumulation of one or more negative-scoring residue alignments; or (b) the end of either sequence is reached. The BLAST algorithm parameters “W”, “T”, and “X” determine the sensitivity and speed of the alignment. The BLASTN program (for nucleotide sequences) functions similarly but uses as defaults a word size (“W”) of 28, an expectation (“E”) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, (1989) PNAS(USA) 89:10915-10919).

In An Amount Sufficient Amount to Effect a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system is an in vivo system which involves the measurement of a change in the level a parameter of a cell, tissue, or organism reflective of a biological function before and after the application of the test agent to the cell, tissue, or organism. In some embodiments, the indicator is reflective of biological function or state of development of a cell evaluated in an assay in response to the administration of a quantity of the test agent. In some embodiments, the test system involves the measurement of a change in the level an indicator of a cell, tissue, or organism reflective of a biological condition before and after the application of one or more test agents to the cell, tissue, or organism. The term “in an amount sufficient to effect a response” may be sufficient to be a therapeutically effective amount but may also be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise.

Inhibitor: As used herein the term “inhibitor” refers to a molecule that decreases, blocks, prevents, delays activation of, inactivates, desensitizes, antagonizes or down-regulates, e.g., a gene, protein, ligand, receptor, or cell. An inhibitor can also be defined as a molecule that reduces, blocks, or inactivates a constitutive activity of a cell or organism.

Intracellular Domain: As used herein the term “intracellular domain” or its abbreviation “ICD” refers to the portion(s) of a cell surface protein (e.g., a cell surface receptor) which is (are) inside of the plasma membrane of a cell. The ICD may include the entire cytoplasmic portion of a transmembrane protein or membrane associated protein, or intracellular protein. The cell surface protein may be a multi-pass transmembrane protein having multiple non-contiguous intracellular domains.

Isolated: As used herein the term “isolated” is used in reference to a molecule of interest that, if naturally occurring, is in an environment different from that in which it can naturally occurs. “Isolated” is meant to include molecule that are within samples that are substantially enriched for the molecule of interest and/or in which the molecule of interest is partially or substantially purified. Where the molecule is not naturally occurring, “isolated” indicates that the molecule has been separated from an environment in which it was synthesized.

Ligand: As used herein, the term “ligand” refers to a molecule that specifically binds a receptor and causes a change in the receptor so as to effect a change in the activity of the receptor or cause a measurable response in cell that expresses the receptor. In one embodiment, the term “ligand” refers to a molecule or complex thereof that can act as an agonist or antagonist of a receptor. As used herein, the term “ligand” encompasses natural and synthetic ligands. “Ligand” also encompasses small molecules, peptide mimetics of cytokines and antibodies. The complex of a ligand and receptor is termed a “ligand-receptor complex.” A ligand may comprise one domain of a polyprotein or fusion protein (e.g., either domain of an antibody/ligand fusion protein).

Linker: As used herein the terms “linker” refers to a molecule that is used to join a first and second heterologous molecules. In some embodiments, a linker may be a chemical linker. In some embodiments the linker is “polypeptide linker” are to describe a polypeptide employed to join functional subunits of a polypeptide composed of multiple functional domains or subunits (e.g. a fusion protein).

Modified: As used herein, the term “modified” refers to a molecule, such as a polypeptide, whose structure has been changed relative to an unmodified parental molecule. A modified polypeptide typically retains one or more activities or functions of the unmodified parental molecule. For example, an inverted monomer of the disclosure can activate IL10 signaling in a cell expressing the IL10 receptor as part of a homodimer, but can have improved properties relative to the unmodified polypeptide. The term modified includes amino acid substitutions that are not present in a parental or wild-type IL10 polypeptides or inverted configurations of the wild-type monomer polypeptide, and includes variants and muteins of an IL10 inverted monomer polypeptide.

Modulate: As used herein, the terms “modulate”, “modulation” and the like refer to the ability of a test agent to cause a response, either positive or negative or directly or indirectly, in a system, including a biological system, or biochemical pathway. The term modulator includes both agonists (including partial agonists, full agonists and superagonists), inhibitors and antagonists.

Nucleic Acid: As used herein, the terms “nucleic acid”, “nucleic acid molecule”, “polynucleotide” and the like are used interchangeably herein to refer to a polymeric form of nucleotides (including deoxyribonucleotides or ribonucleotides) of any length. Non-limiting examples of polynucleotides include linear and circular nucleic acids, messenger RNA (mRNA), small nuclear RNA (snRNA), short interfering RNA (siRNA), guide RNA (gRNA) complementary DNA (cDNA), recombinant polynucleotides, recombinant viral or non-viral vectors, recombinant viral or non-viral expression vectors, hybridization probes, PCR primers, and the like.

One or More Amino Acid Substitutions: As used herein, the term “one or more amino acid substitutions” refers to a single amino acid substitution, or one, two, three, four, five or more amino acid substitutions relative to a reference sequence. In some embodiments, the reference sequence is wild-type hIL10 monomer or an inverted hIL10 of the disclosure.

Operably Linked: The term “operably linked” is used herein to refer to the relationship between a first and second component molecules, typically polypeptides or nucleic acids, which are arranged in a construct such that at least one of the functions of the component molecules is retained in the construct although the operable linkage may result in a construct exhibiting modulated activity, either positively or negatively, of the individual components of the construct. For example, the operable linkage of a polyethylene glycol (PEG) molecule to a wild-type protein may result in a construct where the biological activity of the protein is diminished relative to the to the wild-type molecule, however the two are nevertheless considered operably linked. When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of one or more of the nucleic acid sequences, and expression of one or more proteins in the cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally, in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Parent Polypeptide: As used herein, the terms “parent polypeptide” or “parent protein” are used interchangeably to designate the source of a second polypeptide (e.g., a derivative, mutein or variant) which is modified with respect to a first “parent” polypeptide. In some instances, the parent polypeptide is a wild-type or naturally occurring form of a protein. In some instance, the parent polypeptide may be a modified form a naturally occurring protein that is further modified. The term parent polypeptide can also be used interchangeably with “reference polypeptide.”

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist may act as a competitive inhibitor of the full agonist by competing with the full agonist for the receptor binding resulting in net decrease in receptor activation relative to the contact of the receptor with the full agonist in the absence of the partial agonist. Partial agonists can be used to activate receptors to give a desired submaximal response in a subject in the absence or depleted levels of the endogenous ligand are present in the subject. Partial agonists can be used to reduce the overstimulation of receptors when excess amounts of the endogenous ligand are present. The maximum response (Emax) produced by a partial agonist is called its intrinsic activity and may be expressed on a percentage scale where a full agonist produces a 100% response. An partial agonist may have greater than 10% but less than 100%, alternatively greater than 20% but less than 100%, alternatively greater than 30% but less than 100%, alternatively greater than 40% but less than 100%, alternatively greater than 50% but less than 100%, alternatively greater than 60% but less than 100%, alternatively greater than 70% but less than 100%, alternatively greater than 80% but less than 100%, or alternatively greater than 90% but less than 100%, of the activity of the reference full agonist polypeptide ligand when evaluated at similar concentrations in a given assay system.

Percent Identity: As used herein, the term “percent (%) sequence identity” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

Polypeptide: As used herein the terms “polypeptide,” “peptide,” and “protein”, used interchangeably herein, refer to a polymeric form of amino acids of any length. A polypeptide may include genetically coded and non-genetically coded amino acids, chemically or biochemically modified or derivatized amino acids, and polypeptides having modified polypeptide backbones. The term polypeptide includes fusion proteins. The term polypeptide also includes fusion proteins of a first polypeptide and a second heterologous polypeptide carrier protein (e.g., human serum albumin (HSA)).

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof. A course of action to prevent a disease, disorder or condition in a subject is typically applied in the context of a subject who is predisposed to developing a disease, disorder or condition due to genetic, experiential or environmental factors of developing a particular disease, disorder or condition.

Receptor: As used herein, the term “receptor” refers to a polypeptide having a domain that specifically binds a ligand that binding of the ligand results in a change to at least one biological property of the polypeptide. In some embodiments, the receptor is a cell membrane associated protein that comprises and extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain™. The binding of a cognate ligand to the receptor results in a change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex that, when assembled, results in intracellular signaling. For example, the ligand may bind a cell surface receptor that is not associated with any intracellular signaling alone but upon ligand binding facilitates the formation of a heteromultimeric (including heterodimeric, heterotrimeric, etc.) or homomultimeric (including homodimeric, homotrimeric, homotetrameric, etc.) complex that results in a measurable biological effect in the cell such as activation of an intracellular signaling cascade (e.g., the Jak/STAT pathway). In some embodiments, a receptor is a membrane spanning single chain polypeptide comprising ECD, TM and ICD domains wherein the ECD, TM and ICD domains are derived from the same or differing naturally occurring receptor variants or synthetic functional equivalents thereof.

Recombinant: As used herein, the term “recombinant” is used as an adjective to refer to the method by which a polypeptide, nucleic acid, or cell was modified using recombinant DNA technology. A “recombinant protein” is a protein produced using recombinant DNA technology and is frequently abbreviated with a lower case “r” preceding the protein name to denote the method by which the protein was produced (e.g., recombinantly produced human growth hormone is commonly abbreviated “rhGH”).

Similarly a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation) where the change is correlated with the activation, stimulation, or treatment, with or contact with exogenous agents or internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. A “response” may be evaluated in vitro such as through the use of assay systems, flow cytometric assays, surface plasmon resonance, enzymatic activity, mass spectroscopy, amino acid or protein sequencing technologies. A “response” may be evaluated in vivo quantitatively by evaluation of objective physiological parameters such as body temperature, bodyweight, tumor volume, blood pressure, results of X-ray or other imaging technology or qualitatively through changes in reported subjective feelings of well-being, depression, agitation, or pain. In some embodiments, the level of activation of T cells in response to the administration of a test agent may be determined by flow cytometric methods as described as determined by the level of one or more STATs (e.g., STAT1, STAT3, or STAT5) in accordance with methods well known in the art.

Significantly Reduced Binding: As used herein, the term “exhibits significantly reduced binding” is used with respect a variant of a first molecule (e.g., a ligand or antibody) which exhibits a significant reduction in the affinity for a second molecule (e.g., receptor or antigen) relative the parent form of the first molecule. With respect to antibody variants, an antibody variant “exhibits significantly reduced binding” if the affinity of the variant antibody for an antigen if the variant binds to the native form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent antibody from which the variant was derived. With respect to variant ligands or ligand muteins, a variant ligand or mutein “exhibits significantly reduced binding” if the affinity of the variant ligand or mutein binds to a receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent ligand from which the variant ligand was derived. Similarly, with respect to variant receptors, a variant receptor “exhibits significantly reduced binding” if the affinity of the variant receptors to a ligand binds with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent receptor from which the variant receptor was derived.

Specifically Binds: As used herein the term “specifically binds” refers to the degree of affinity for which a first molecule exhibits with respect to a second molecule. In the context of binding pairs (e.g., ligand/receptor, antibody/antigen) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. Specific binding may be assessed using techniques known in the art including but not limited to competition ELISA assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays)) and surface plasmon resonance assays (see, e.g., Drescher et al., (2009) Methods Mol Biol 493:323-343 with commercially available instrumentation such as the Biacore 8K, Biacore 8K+, Biacore S200, Biacore T200 (Cytiva, 100 Results Way, Marlborough MA 01752).

Subject: The terms “recipient”, “individual”, “subject”, and “patient”, are used interchangeably herein and refer to any mammalian subject for whom diagnosis, treatment, or therapy is desired. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially Pure: As used herein, the term “substantially pure” indicates that a component of a composition makes up greater than about 50%, alternatively greater than about 60%, alternatively greater than about 70%, alternatively greater than about 80%, alternatively greater than about 90%, alternatively greater than about 95% of the total content of the composition. A protein that is “substantially pure” comprises greater than about 50%, alternatively greater than about 60%, alternatively greater than about 70%, alternatively greater than about 80%, alternatively greater than about 90%, alternatively greater than about 95% of the total content of the composition.

Suffering From: As used herein, the term “suffering from” refers to a determination made by a physician with respect to a subject based on the available objective or subjective information accepted in the field for the identification of the presence of a disease, disorder or condition in a subject including but not limited to X-ray, CT-scans, conventional laboratory diagnostic tests (e.g., blood count, etc.), genomic data, protein expression data, immunohistochemistry, that the subject requires or will benefit from treatment.

T-cell: As used herein the term “T-cell” or “T cell” is used in its conventional sense to refer to a lymphocytes that differentiate in the thymus. In some embodiments the term T cell includes, without limitation, naïve CD8⁺ T cells, cytotoxic CD8⁺ T cells, naïve CD4⁺ T cells, helper T cells, e.g., T_{H}1, T_{H}2, T_{H}9, T_{H}11, T_{H}22, T_{FH}; regulatory T cells, e.g., T_{R}1, Tregs, inducible Tregs; memory T cells, e.g., central memory T cells, effector memory T cells, NKT cells, tumor infiltrating lymphocytes (TILs) and engineered variants of such T-cells including but not limited to CAR-T cells, recombinantly modified TILs and TCR-engineered cells.

Terminus/Terminal: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” refers to the position of a first amino acid residue relative to a second amino acid residue in a contiguous polypeptide sequence, the first amino acid being closer to the N-terminus of the polypeptide. “Immediately C-terminal” refers to the position of a first amino acid residue relative to a second amino acid residue in a contiguous polypeptide sequence, the first amino acid being closer to the C-terminus of the polypeptide.

Therapeutically Effective Amount: As used herein to the phrase “therapeutically effective amount” refers to the quantity of an agent when administered to a subject, either alone or as part of a pharmaceutical composition or treatment regimen, in a single dose or as part of a series of doses, provides a positive effect on any quantitative or qualitative symptom, aspect, or characteristic of a disease, disorder or condition. A therapeutically effective amount can be ascertained by measuring relevant physiological effects, and it may be adjusted in connection with a dosing regimen and in response to diagnostic analysis of the subject's condition. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in, increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. In one embodiment, a therapeutically effective amount is an amount of an agent when used alone or in combination with another agent provides a improvement in any quantitative or qualitative symptom, aspect, or characteristic of a disease, disorder or condition and does not result in non-reversible serious adverse events in the course of administration of the agent to the mammalian subject.

Treat: The terms “treat”, “treating”, treatment” and the like refer to a course of action (such as contacting the subject with pharmaceutical composition comprising an inverted monomer and/or dimer thereof alone or in combination with a supplementary agent) that is initiated with respect to a subject in response to a diagnosis that the subject is suffering from a disease, disorder or condition, or a symptom thereof, the course of action being initiated so as to eliminate, reduce, suppress, mitigate, or ameliorate, either temporarily or permanently, at least one of: (a) the underlying causes of such disease, disorder, or condition afflicting a subject; and/or (b) at least one of the symptoms associated with such disease, disorder, or condition. In some embodiments, treating includes a course of action taken with respect to a subject suffering from a disease where the course of action results in the inhibition (e.g., arrests the development of the disease, disorder or condition or ameliorates one or more symptoms associated therewith) of the disease in the subject. In some embodiments, the term “treat” is used to refer to a course of action that slows the the progression of a disease, disorder or condition from an existing state to a more deleterious state.

Variant: The terms “variant”, “protein variant” or “variant protein” or “variant polypeptide” are used interchangeably herein to refer to a polypeptide that differs from a parent polypeptide by virtue of at least one amino acid modification, substitution, or deletion. The parent polypeptide may be a naturally occurring or wild-type (WT) polypeptide or may be a modified version (e.g., an inverted version) of a WT polypeptide. The term variant polypeptide may refer to the polypeptide itself, a composition comprising the polypeptide, or the nucleic acid sequence that encodes it. In some embodiments, the variant polypeptide comprises from about one to about ten, alternatively about one to about eight, alternatively about one to about seven, alternatively about one to about five, alternatively about one to about four, alternatively from about one to about three alternatively from one to two amino acid modifications, substitutions, or deletions, or alternatively a single amino acid amino acid modification, substitution, or deletion compared to the parent polypeptide. A variant may be at least about 99% identical, alternatively at least about 98% identical, alternatively at least about 97% identical, alternatively at least about 95% identical, or alternatively at least about 90% identical to the parent polypeptide from which the variant is derived. The term “variant” also includes a nucleic acid molecule that encodes a protein or polypeptide having an altered or modified amino acid sequence compared to a parent polypeptide.

Wild Type: By “wild type” or “WT” or “native” herein is meant an amino acid sequence or a nucleotide sequence that is found in nature, including allelic variations. A wild-type protein, polypeptide, antibody, immunoglobulin, IgG, etc. has an amino acid sequence or a nucleotide sequence that has not been modified by the hand of man.

It will be understood that individual embodiments, which are separately described herein for clarity and brevity, can be combined without limitation. Thus, the present disclosure includes one or more, or all, combinations of the embodiments described herein as if each and every combination was individually and explicitly disclosed. This also applies to any and all sub-combinations of the embodiments disclosed herein, such that the present disclosure includes one or more, or all, sub-combinations of the embodiments described herein as if each and every sub-combination was individually and explicitly disclosed.

### Inverted Monomers

The present disclosure provides inverted monomers of IL10 including inverted monomers of hIL10. In some embodiments, the IL10 inverted monomer is derived from the sequence of a mature IL10 (human or mouse) monomer. The canonical 160 amino acid sequence of the mature (“wild-type”) human IL10 monomer (UniProt Reference No. P22301) without the signal sequence (corresponding to amino acids 19-178 of the pre-protein) has the amino acid sequence:

(SEQ ID NO: 20)
SPGQGTQSENSCTHFPGNLPNMLRDLRDAFSRVKTFFQMKDQLDNLLLKES
LLEDFKGYLGCQALSEMIQFYLEEVMPQAENQDPDIKAHVNSLGENLKTLR
LRLRRCHRFLPCENKSKAVEQVKNAFNKLQEKGIYKAMSEFDIFINYIEAY
MTMKIRN

The canonical amino acid sequence of the mature murine IL10 protein (UniProt Reference No. P18893) monomer without the signal sequence (corresponding to amino acids 19-178 of the pre-protein) is:

(SEQ ID NO: 141)
SRGQYSREDN NCTHFPVGQS HMLLELRTAF SQVKTFFQTK
DQLDNILLTD SLMQDFKGYL GCQALSEMIQ FYLVEVMPQA
EKHGPEIKEH LNSLGEKLKT LRMRLRRCHR FLPCENKSKA
VEQVKSDFNK LQDQGVYKAM NEFDIFINCI EAYMMIKMKS.

Based on the crystal structure, wild-type IL10 comprises six (6) alpha helical domains. See PDB DOI: 10.2210/pdb2ILK/pdb. The amino acid sequences of the helices and their positions relative to SEQ ID NO:11 are shown in the Table 1 below:

TABLE 1
Alpha helices of hIL 10

| | | wt hIL 10 amino | |
|---|---|---|---|
| | | acid residue | |
| Helix | Reference Sequence | numbers: | SEQ ID NO: |
| 1 | NLPNMLRDLRDAFSRVKTFFQMKD | 18-41 | 3 |
| 2 | KESLLEDFKG | 49-58 | 4 |
| 3 | LGCQALSEMIQFYLEEVMPQAEN | 60-82 | 5 |
| 4 | IKAHVNSLGENLKTLRLRLRRC | 87-108 | 6 |
| 5 | SKAVEQVKNAFNKL | 118-131 | 1 |

The terms “inverted monomer of IL10” and “IL10 inverted monomer” are used interchangeably to refer to a IL10 variant comprising partial sequences derived from wild-type IL10 that are rearranged relative to native N-terminal to C-terminal order of such partial sequences present in naturally occurring IL10 monomers. For example, in some embodiments, one or more helices of IL10 are rearranged in their relative positions to the sequential arrangement of such helices in the wild type IL10 sequence. In some embodiments of the inverted monomers of the present disclosure, a polypeptide sequence comprising helix 5 and helix 6 is provided N-terminal to a polypeptide sequence comprising helices 1-4. In some embodiments, the inverted IL10 monomer comprises a polypeptide comprising helices 5 and 6 located N-terminal to a polypeptide comprising helices 1, 2, 3, and 4 in amino to carboxyl order (i.e., helices 5, 6, 1, 2, 3, and 4 in amino to carboxyl order).

In some embodiments, the individual helices are linked by one or more independent amino acid or polypeptide linkers. In some embodiments, a linker may comprise a polypeptide derived from the wildtype IL10 sequence. In some embodiments, the linker comprises a heterologous or synthetic amino acid or polypeptide sequence. In some embodiments, the inverted monomer may further comprise amino acid sequences N-terminal of the first helix (e.g., helix 5) and C-terminal of the last helix (e.g., helix 4). The additional N-terminal and C-terminal amino acid sequences can comprise native or wild-type IL10 sequences that are normally located N-terminal or C-terminal of the particular helix.

In some embodiments, the inverted monomer polypeptide comprises an amino acid sequence of the formula 1:

[A]-L1x-[B]-L2-[C]-L3-[D]-L4y-[E]-L5-[F] (1)

wherein:
x and y are independently selected from 0 (absent) or 1 (present), and L1 to L5 refer to linkers 1 to 5, respectively.

In some embodiments, [A] corresponds to helix 5 of wildtype IL10, corresponding to amino acid residues 118-131 of hIL10 (numbered according to SEQ ID NO:11). In some embodiments, [A] comprises the amino acid sequence SKAVEQVKNAFNKL (SEQ ID NO:1). In some embodiments, [A] comprises a variant of SEQ ID NO:1 having 1 or 2 amino acid substitutions relative to SEQ ID NO:1). In some embodiments, the variant sequence substantially retains the helical structure of [A] compared to the wildtype helix.

In some embodiments, x=0 (no linker, meaning L1 is absent) or x=1 (L1 is present) and L1 comprises a polypeptide linker of any suitable length, for example from about 1 to 40, 1 to 30, 1 to 25, 1 to 20, 1 to 15, 1 to 10, or 1 to 5 amino acids. In some embodiments, L1 comprises 1, 2, 3, 4, or 5 amino acids. In some embodiments, L1 comprises a GS linker as described herein. In some embodiments, L1 is the amino acid glutamine (Q).

In some embodiments, [B] corresponds to helix 6 of wildtype IL10, corresponding to amino acid residues 133-159 of hIL10 (numbered according to SEQ ID NO:11). In some embodiments, [B] comprises the amino acid sequence EKGIYKAMSEFDIFINYIEAYMTMKIR (SEQ ID NO:2). In some embodiments, [B] comprises a variant of SEQ ID NO:2 having 1 or 2 amino acid substitutions relative to SEQ ID NO:2. In some embodiments, the variant sequence substantially retains the helical structure of [B] compared to the wildtype helix.

In some embodiments, L2 comprises a linker of any suitable length, e.g., from about 1 to 40, 1 to 30, 1 to 25, 1 to 20, 5 to 100, 5 to 90, 5 to 80, 5 to 70, 5 to 60, 5 to 50, 5 to 40, 5 to 30, 5 to 25, 5 to 20, 10 to 100, 10 to 90, 10 to 80, 10 to 70, 10 to 60, 10 to 50, 10 to 40, 10 to 30, 10 to 25, or 10 to 20 amino acids. In some embodiments, L2 comprises 10, 11, 12, 13, 14, 15, 16, 17, 18, 19, 20, 21, 22, 23, 24 or 25 amino acids. In some embodiments, L2 comprises a GS linker described herein. In some embodiments, L2 is a polypeptide comprising the amino acid sequence NSPGQGTQSENSCTHFPG (SEQ ID NO:7). In some embodiments, L2 is a polypeptide comprising the amino acid sequence

(SEQ ID NO: 23)
NTSPGQGTQSENSCTHFPG.

In some embodiments, [C] corresponds to helix 1 of wildtype IL10, corresponding to amino acid residues 18-41 of hIL10 (numbered according to SEQ ID NO:11). In some embodiments, [C] comprises the amino acid sequence NLPNMLRDLRDAFSRVKTFFQMKD (SEQ ID NO:3). In some embodiments, [C] comprises a variant of SEQ ID NO:3 having 1 or 2 amino acid substitutions relative to SEQ ID NO:3. In some embodiments, the variant sequence substantially retains the helical structure of [C] compared to the wildtype helix.

In some embodiments, L3 comprises a linker of any suitable length, e.g., from about 1 to 40, 1 to 30, 1 to 25, 1 to 20, 1 to 10, 2 to 50, 2 to 40, 2 to 30, 2 to 20, 2 to 10, 3 to 50, 3 to 40, 3 to 30, 3 to 20, 3 to 10, 4 to 50, 4 to 40, 4 to 30, 4 to 20, 4 to 15, 4 to 14, 4 to 13, 4 to 12, 4 to 11, 4 to 10, 5 to 50, 5 to 40, 5 to 30, 5 to 25, 5 to 20, 5 to 15, or 5 to 10, amino acids. In some embodiments, L3 comprises 4, 5, 6, 7, 8, 9, 10, or 11 amino acids. In some embodiments, L3 comprises a GS linker described herein. In some embodiments, L3 is a polypeptide comprising the amino acid sequence QLDNLLL (SEQ ID NO:8).

In some embodiments, [D] corresponds to helix 2 of wildtype IL10, corresponding to amino acid residues 49-58 of hIL10 (numbered according to SEQ ID NO:11). In some embodiments, [D] comprises the amino acid sequence KESLLEDFKG (SEQ ID NO:4). In some embodiments, [D] comprises a variant of SEQ ID NO:4 having 1 or 2 amino acid substitutions relative to SEQ ID NO:4. In some embodiments, the variant sequence substantially retains the helical structure of [D] compared to the wildtype helix.

In some embodiments, y=0 (L4 is absent), or y=1 and L4 comprises a polypeptide linker. The L4 linker can be of any suitable length, e.g., from about 1 to 40, 1 to 30, 1 to 25, 1 to 20, 1 to 15, 1 to 10, or about 1 to 5 amino acids. In some embodiments, L4 comprises 1, 2, 3, 4, or 5 amino acids. In some embodiments, L4 comprises a GS linker described herein. In some embodiments, L4 is the amino acid glycine (G). In some embodiments, L4 comprises the amino acid sequence “GY”.

In some embodiments, [E] corresponds to helix 3 of wildtype IL10, corresponding to amino acid residues 60-82 of hIL10 (numbered according to SEQ ID NO:11). In some embodiments, [E] comprises the amino acid sequence LGCQALSEMIQFYLEEVMPQAEN (SEQ ID NO:5). In some embodiments, [E] comprises a variant of SEQ ID NO:5 having 1 or 2 amino acid substitutions relative to SEQ ID NO:5. In some embodiments, the variant sequence substantially retains the helical structure of [E] compared to the wildtype helix.

In some embodiments, L5 comprises a linker. Linker L5 can be of any suitable length, e.g., from about 1 to 40, 1 to 30, 1 to 25, 1 to 20, 1 to 15, 1 to 10, 1 to 9, 1 to 8, 1 to 7, 1 to 6, or about 1 to 5 amino acids. In some embodiments, L5 comprises 1, 2, 3, 4, 5, 6 or 7 amino acids. In some embodiments, L5 comprises a GS linker described herein. In some embodiments, L5 is a polypeptide comprising the amino acid sequence QDPD (SEQ ID NO:9).

In some embodiments, [F] corresponds to helix 3 of wildtype IL10, corresponding to amino acid residues 87-108 of hIL10 (numbered according to SEQ ID NO:11). In some embodiments, [F] comprises the amino acid sequence IKAHVNSLGENLKTLRLRLRRC (SEQ ID NO:6). In some embodiments, [F] comprises a variant of SEQ ID NO:6 having 1 or 2 amino acid substitutions relative to SEQ ID NO:6. In some embodiments, the variant sequence substantially retains the helical structure of [F] compared to the wildtype helix.

In some embodiments, the domains of Formula 1 are shown in Table 2.

TABLE 2
Reference Sequences of Domains [A], [B], [C],
[D], [E], and [F] of Formula 1:

| Domain | Reference Sequence | SEQ ID NO |
|---|---|---|
| [A] | SKAVEQVKNAFNKL | 1 |
| [B] | EKGIYKAMSEFDIFINYIEAYMTMKIR | 2 |
| [C] | NLPNMLRDLRDAFSRVKTFFQMKD | 3 |
| [D] | KESLLEDFKG | 4 |
| [E] | LGCQALSEMIQFYLEEVMPQAEN | 5 |
| [F] | IKAHVNSLGENLKTLRLRLRRC | 6 |

Each of the linkers described above can be independently selected as wild-type, i.e., the amino acid sequence of the wild-type hIL10 sequence linking the two helices in question. Alternatively, one or more linkers can be variants of the wild-type linker sequence, i.e., having 1, 2, or more amino acid substitutions, deletions or insertions compared to the wild-type linker sequence in question. In some embodiments, L1 is the amino acid glutamine (Q). In some embodiments, L2 is a polypeptide comprising the amino acid sequence NSPGQGTQSENSCTHFPG (SEQ ID NO:7). In some embodiments, L2 is a polypeptide comprising the amino acid sequence NTSPGQGTQSENSCTHFPG (SEQ ID NO:23). In some embodiments, L3 is a polypeptide comprising the amino acid sequence QLDNLLL (SEQ ID NO:8). In some embodiments, L4 is the amino acid glycine (“G”). In some embodiments, L4 comprises the amino acid sequence “GY”. In some embodiments, L5 is a polypeptide comprising the amino acid sequence QDPD (SEQ ID NO:9).

In some embodiments, one or more (e.g., all) of L1, L2, L3, L4 and/or L5 comprise a linker of a different sequence than the wild-type linker sequence. For example, in some embodiments, each of L1, L2, L3, L4 and/or L5 comprise a GS-linker. Linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of linkers useful in the practice of the present disclosure include but are not limited to glycine polymers (G)n where is an integer from 1-50. Examples of linkers useful in the practice of the present disclosure include but are not limited to glycine-alanine polymers, alanine-serine polymers, and glycine-serine polymers also referred to herein as “GS-linkers.” Glycine and glycine-serine polymers are relatively unstructured, and therefore may serve as a flexible tether between domains or subunits of a polypeptide. In some embodiments, a GS-linker is a polymer selected of the formulae: (GmSo)n (SEQ ID NO: 136), (GS)n (SEQ ID NO: 137), (GSGGS)n (SEQ ID NO:56), (GGGS)n (SEQ ID NO:57), (GGGGS)n (SEQ ID NO:58), (GmSoGm)n (SEQ ID NO:59), (GmSoGmSoGm)n (SEQ ID NO:60), (GSGGSm)n (SEQ ID NO:61), (GSGSmG)n (SEQ ID NO:62) and (GGGSm)n (SEQ ID NO:63), and (GGGGS)n (SEQ ID NO:64), and combinations thereof, where m, n, and o are each independently selected from an integer from 1 to 20, e.g., 1-18, 2-16, 3-14, 4-12, 5-10, 1, 2, 3, 4, 5, 6, 7, 8, 9, or 10. Examples of GS-linkers include, but are not limited to GGSG (SEQ ID NO:65), GGSGG (SEQ ID NO:66), GSGSG (SEQ ID NO:67), GSGGG (SEQ ID NO:68), GGGSG (SEQ ID NO:69), and GSSSG (SEQ ID NO:70), GGGS (SEQ ID NO:71), GGGGS (SEQ ID NO:72), GGGGSGGGGS (SEQ ID NO:73), GGGGSGGGGSGGGGS (SEQ ID NO:74), GGSG (SEQ ID NO:75), GGSGG (SEQ ID NO:76), GSGSG (SEQ ID NO:77), GSGGG (SEQ ID NO:78), GGGSG (SEQ ID NO:79), GGGGSGGGGS (SEQ ID NO:80), GGGGSGGGGSGSSSG (SEQ ID NO:81) and multimers thereof.

In some embodiments, the inverted monomer polypeptide comprises or consists of an amino acid sequence derived from human IL10. In some embodiments, the inverted monomer polypeptide comprises or consists of SEQ ID NO:10 below or an amino acid sequence having at least 90%, 91%, 92%, 93%, 94%, 95%, 96%, 97%, 98%, 99% or 100% sequence identity to SEQ ID NO:10:

(SEQ ID NO: 10)
KSKAVEQVKNAFNKLQEKGIYKAMSEFDIFINYIEAYMTMKIRNSPGQGT
QSENSCTHFPGNLPNMLRDLRDAFSRVKTFFQMKDQLDNLLLKESLLEDF
KGYLGCQALSEMIQFYLEEVMPQAENQDPDIKAHVNSLGENLKTLRLRLR
RCHRFLPCEN.

In some embodiments, the inverted monomer polypeptide comprises or consists of SEQ ID NO:10 or an amino acid sequence having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to SEQ ID NO: 10.

In some embodiments, the inverted monomer polypeptide comprises or consists of an amino acid sequence derived from human IL10. In some embodiments, the inverted monomer polypeptide comprises or consists of SEQ ID NO:10 below or an amino acid sequence having at least 90%, 91%, 92%, 93%, 94%, 95%, 96%, 97%, 98%, 99% or 100% sequence identity to SEQ ID NO: 11:

(SEQ ID NO: 11)
KSKAVEQVKNAFNKLQEKGIYKAMSEFDIFINYIEAYMTMKIRNTSPGQG
TQSENSCTHFPGNLPNMLRDLRDAFSRVKTFFQMKDQLDNLLLKESLLED
FKGYLGCQALSEMIQFYLEEVMPQAENQDPDIKAHVNSLGENLKTLRLRL
RRCHRFLPCEN.

In some embodiments, the inverted monomer polypeptide comprises or consists of SEQ ID NO:10 below or an amino acid sequence having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to SEQ ID NO:11:

In some embodiments, the disclosure provides a polypeptide comprising, in the following amino to carboxyl order:
(a) a first polypeptide comprising an amino acid sequence selected from the group consisting of:

(i) amino acid residues 117-158 of human IL-10
(KSKAVEQVKNAFNKLQEKGIYKAMSEFDIFINYIEAYMTMKI; SEQ
ID NO: 12);
(ii) amino acid residues 117-159 of human IL-10
(KSKAVEQVKNAFNKLQEKGIYKAMSEFDIFINYIEAYMTMKIR;
SEQ ID NO: 13);
and
(iii) amino acid residues 117-160 of human IL-10
(KSKAVEQVKNAFNKLQEKGIYKAMSEFDIFINYIEAYMTMKIRN;
SEQ ID NO: 14), numbered in accordance with
mature human IL-10 (SEQ ID NO: 20),

(b) linkerₓ, wherein x=0 (linker absent) or 1 (linker present), and
(c) a second polypeptide comprising an amino acid sequence selected from the group consisting of

(i) amino acid residues 1-116 of human IL-10
(SPGQGTQSENSCTHFPGNLPNMLRDLRDAFSRVKTFFQMKDQLDNLLLK
ESLLEDFKGYLGCQALSEMIQFYLEEVMPQAENQDPDIKAHVNSLGENLK
TLRLRLRRCHRFLPCEN; SEQ ID NO: 15);
(ii) amino acid residues 2-116 of human IL-10
(PGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKT
LRLRLRRCHRFLPCEN; SEQ ID NO: 16);
(iii) amino acid residues 3-116 of human IL-10
(GQGTQSENSCTHFPGNLPNMLRDLRDAFSRVKTFFQMKDQLDNLLLKES
LLEDFKGYLGCQALSEMIQFYLEEVMPQAENQDPDIKAHVNSLGENLKTL
RLRLRRCHRFLPCEN; SEQ ID NO: 17);
(iv) amino acid residues 4-116 of human IL-10
(QGTQSENSCTHFPGNLPNMLRDLRDAFSRVKTFFQMKDQLDNLLLKESL
LEDFKGYLGCQALSEMIQFYLEEVMPQAENQDPDIKAHVNSLGENLKTLR
LRLRRCHRFLPCEN; SEQ ID NO: 18);
(v) amino acid residues 5-116 of human IL-10
(GTQSENSCTHFPGNLPNMLRDLRDAFSRVKTFFQMKDQLDNLLLKESLL
EDFKGYLGCQALSEMIQFYLEEVMPQAENQDPDIKAHVNSLGENLKTLRL
RLRRCHRFLPCEN; SEQ ID NO: 19) numbered in
accordance with mature human IL-10 (SEQ ID NO:
20).
(vi) amino acid residues 6-116 of human IL-10
(TQSENSCTHFPGNLPNMLRDLRDAFSRVKTFFQMKDQLDNLLLKESLLE
DFKGYLGCQALSEMIQFYLEEVMPQAENQDPDIKAHVNSLGENLKTLRLR
LRRCHRFLPCEN; SEQ ID NO: 29);
(vii) amino acid residues 7-116 of human IL-10
(QSENSCTHFPGNLPNMLRDLRDAFSRVKTFFQMKDQLDNLLLKESLLED
FKGYLGCQALSEMIQFYLEEVMPQAENQDPDIKAHVNSLGENLKTLRLRL
RRCHRFLPCEN; SEQ ID NO: 30);
(viii) amino acid residues 8-116 of human IL-10
(SENSCTHFPGNLPNMLRDLRDAFSRVKTFFQMKDQLDNLLLKESLLEDF
KGYLGCQALSEMIQFYLEEVMPQAENQDPDIKAHVNSLGENLKTLRLRLR
RCHRFLPCEN; SEQ ID NO: 31);
(ix) amino acid residues 9-116 of human IL-10
(ENSCTHFPGNLPNMLRDLRDAFSRVKTFFQMKDQLDNLLLKESLLEDFK
GYLGCQALSEMIQFYLEEVMPQAENQDPDIKAHVNSLGENLKTLRLRLRR
CHRFLPCEN; SEQ ID NO: 32);
(x) amino acid residues 10-116 of human IL-10
(NSCTHFPGNLPNMLRDLRDAFSRVKTFFQMKDQLDNLLLKESLLEDFKG
YLGCQALSEMIQFYLEEVMPQAENQDPDIKAHVNSLGENLKTLRLRLRRC
HRFLPCEN; SEQ ID NO: 33);
(xi) amino acid residues 11-116 of human IL-10
(SCTHFPGNLPNMLRDLRDAFSRVKTFFQMKDQLDNLLLKESLLEDFKGY
LGCQALSEMIQFYLEEVMPQAENQDPDIKAHVNSLGENLKTLRLRLRRCH
RFLPCEN; SEQ ID NO: 34);
and
(xii) amino acid residues 12-116 of human IL-10
(CTHFPGNLPNMLRDLRDAFSRVKTFFQMKDQLDNLLLKESLLEDFKGYL
GCQALSEMIQFYLEEVMPQAENQDPDIKAHVNSLGENLKTLRLRLRRCHR
FLPCEN; SEQ ID NO: 35).

In some embodiments, the fusion polypeptide comprises, in the following amino to carboxyl order:
(i) a first polypeptide comprising amino acid residues 117-158 of human IL-10 (SEQ ID NO:12), or an amino acid sequence having at least 90%, 91%, 92%, 93%, 94%, 95%, 96%, 97%, 98%, 99% or 100% sequence identity SEQ ID NO:12; and
(ii) a second polypeptide comprising amino acid residues 5-116 of human IL-10 (SEQ ID NO: 19), or an amino acid sequence having at least 90%, 91%, 92%, 93%, 94%, 95%, 96%, 97%, 98%, 99% or 100% sequence identity to SEQ ID NO: 19.

In some embodiments, the fusion polypeptide comprises, in the following amino to carboxyl order:
(i) a first polypeptide comprising amino acid residues 117-158 of human IL-10 (SEQ ID NO:12), or an amino acid sequence having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity SEQ ID NO:12; and
(ii) a second polypeptide comprising amino acid residues 5-116 of human IL-10 (SEQ ID NO:19), or an amino acid sequence having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to SEQ ID NO:19.

In some embodiments, the first polypeptide comprises 1, 2, or 3 amino acid substitutions relative to SEQ ID NO:12. In some embodiments, the second polypeptide comprises 1, 2, or 3 amino acid substitutions relative to SEQ ID NO:19.

In some embodiments, the first and second polypeptides are covalently linked by peptide bond (i.e., without an additional linker sequence). In some embodiments, the C-terminus of the first polypeptide is covalently linked to the N-terminus of the second polypeptide by a peptide bond (i.e., without an additional linker sequence). In some embodiments, the first and second polypeptides are covalently linked by a linker described herein.

In some embodiments, the inverted monomer comprises an amino acid sequence in Table 6 or Table 10, or an amino acid sequence having at least 90%, 91%, 92%, 93%, 94%, 95%, 96%, 97%, 98%, 99% or 100% sequence identity to an amino acid sequence in Table 6, Table 8, Table 10 or Table 16.

In some embodiments, the inverted monomer comprises an amino acid sequence in Table 6, Table 10, or Table 16 or an amino acid sequence having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to an amino acid sequence in Table 6, Table 8, Table 10, or Table 16.

In some embodiments, the inverted monomer comprises an amino acid sequence having at least 90%, 91%, 92%, 93%, 94%, 95%, 96%, 97%, 98%, 99% or 100% sequence identity to an hIL10 inverted monomer selected from the group consisting of SEQ ID NO: 10, SEQ ID NO:11, SEQ ID NO:91, SEQ ID NO:92, SEQ ID NO:93, SEQ ID NO:94, SEQ ID NO:95, SEQ ID NO:96, SEQ ID NO:97, and SEQ ID NO:98.

In some embodiments, the inverted monomer comprises an amino acid sequence having at least 90%, alternatively at least 91%, alternatively at least 92%, alternatively at least 93%, alternatively at least 94% alternatively at least 95% alternatively at least 96%, alternatively at least 97% alternatively at least 98%, alternatively at least 9900 or 100% sequence identity to an hIL10 inverted monomer selected from the group consisting of SEQ ID NO: 10, SEQ ID NO: 11, SEQ ID NO:91, SEQ ID NO: 92, SEQ ID NO: 93, SEQ ID NO:94, SEQ ID NO:95, SEQ ID NO:96, SEQ ID NO:97, and SEQ ID NO:98.

TABLE 16
Human Inverted Monomer Amino Acid Sequences

| SEQ ID | |
|---|---|
| NO | Amino Acid Sequence |
| 10 | KSKAVEQVKNAFNKLQEKGIYKAMSEFDIFINYIEAYMTMKIRNSPGQGTQSENSCTHFPGNLP |
| | NMLRDLRDAFSRVKTFFQMKDQLDNLLLKESLLEDFKGYLGCQALSEMIQFYLEEVMPQAEN |
| | QDPDIKAHVNSLGENLKTLRLRLRRCHRFLPCEN |
| 11 | KSKAVEQVKNAFNKLQEKGIYKAMSEFDIFINYIEAYMTMKIRNTSPGQGTQSENSCTHFPGNL |
| | PNMLRDLRDAFSRVKTFFQMKDQLDNLLLKESLLEDFKGYLGCQALSEMIQFYLEEVMPQAE |
| | NQDPDIKAHVNSLGENLKTLRLRLRRCHRFLPCEN |
| 92 | KSKAVEQVKNAFNKLQEKGIYKAMSEFDIFINYIEAYMTMKIRNPGQGTQSENSCTHFPGNLP |
| | NMLRDLRDAFSRVKTFFQMKDQLDNLLLKESLLEDFKGYLGCQALSEMIQFYLEEVMPQAEN |
| | QDPDIKAHVNSLGENLKTLRLRLRRCHRFLPCEN |
| 93 | MSKAVEQVKNAFNKLQEKGIYKAMSEFDIFINYIEAYMTMKIRNSPGQGTQSENSCTHFPGNL |
| | PNMLRDLRDAFSRVKTFFQMKDQLDNLLLKESLLEDFKGYLGCQALSEMIQFYLEEVMPQAE |
| | NQDPDIKAHVNSLGENLKTLRLRLRRCHRFLPCEN |
| 94 | KSKAVEQVKNAFNKLQEKGIYKAMSEFDIFINYIEAYMTMKIRNAVPNQRDYDPNCTHFPGNL |
| | PNMLRDLRDAFSRVKTFFQMKDQLDNLLLKESLLEDFKGYLGCQALSEMIQFYLEEVMPQAE |
| | NQDPDIKAHVNSLGENLKTLRLRLRRCHRFLPCEN |
| 95 | KSKAVEQVKNAFNKLQEKGIYKAMSEFDIFINYIEAYMTMKIRGKGPLTQPSEDCTHFPGNLPN |
| | MLRDLRDAFSRVKTFFQMKDQLDNLLLKESLLEDFKGYLGCQALSEMIQFYLEEVMPQAENQ |
| | DPDIKAHVNSLGENLKTLRLRLRRCHRFLPCEN |
| 96 | KSKAVEQVKNAFNKLQEKGIYKAMSEFDIFINYIEAYMTMKIRGSGSGSGSGSGSCTHFPGNLP |
| | NMLRDLRDAFSRVKTFFQMKDQLDNLLLKESLLEDFKGYLGCQALSEMIQFYLEEVMPQAEN |
| | QDPDIKAHVNSLGENLKTLRLRLRRCHRFLPCEN |
| 97 | KSKAVEQVKNAFNKLQEKGIYKAMSEFDIFINYIEAYMTMKIRNSPGQGTQSENSCTHFPGNLP |
| | NMLRKLRDAFSRVKTFFQMKDQLDNLLLKESLLEDFKGYLGCQALSEMIQFYLEEVMPQAEN |
| | QDPDIKAHVNSLGENLKTLRLRLRRCHRFLPCEN |
| 98 | KSKAVEQVKNAFNKLQEKGIYKAMSEFDIFINYIEAYMTMKIRNTSPGQGTQSENSCTHFPGNL |
| | PNMLRKLRDAFSRVKTFFQMKDQLDNLLLKESLLEDFKGYLGCQALSEMIQFYLEEVMPQAE |
| | NQDPDIKAHVNSLGENLKTLRLRLRRCHRFLPCEN |
| 90 | KSKAVEQVKNAFNKLQEKGIYKAMSEFDIFINYIEAYMTMKIRNSPGQGTQSENSCTHFPGNLP |
| | NMLRDLRDAFSRVKTFFQMKDQLDNLLLKESLLEDFKGYLGCQALSEMIQFYLEEVMPQAEN |
| | QDPDIKAHVNSLGENLKLLRLRLRRCHRFLPCEN |
| 91 | KSKAVEQVKNAFNKLQEKGIYKAMSEFDIFINYIEAYMTMKIRNTSPGQGTQSENSCTHFPGNL |
| | PNMLRDLRDAFSRVKTFFQMKDQLDNLLLKESLLEDFKGYLGCQALSEMIQFYLEEVMPQAE |
| | NQDPDIKAHVNSLGENLKLLRLRLRRCHRFLPCEN |

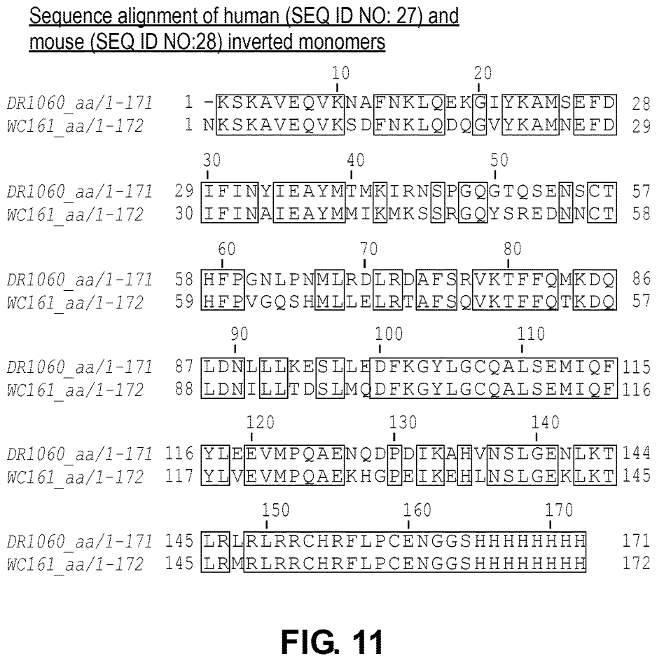

To facilitate evaluation of the activity of the inverted monomers of the present disclosure in murine in vivo models of human disease, the present disclosure further provides murine inverted IL10 monomers as surrogates of the human inverted IL10 monomers of the present disclosure. The preparation of the murine surrogate inverted monomers may be accomplished by substitution of the corresponding human polypeptide substitution(s) into the mouse polypeptide in accordance with the following alignment of the mouse and human sequences (see FIG. 11).

In FIG. 11, DR1060_aa/1-171 comprises the amino
acid sequence:
(SEQ ID NO: 27)
KSKAVEQVKNAFNKLQEKGIYKAMSEFDIFINYIEAYMTMKIRNSPGQGT
QSENSCTHFPGNLPNMLRDLRDAFSRVKTFFQMKDQLDNLLLKESLLEDF
KGYLGCQALSEMIQFYLEEVMPQAENQDPDIKAHVNSLGENLKTLRLRLR
RCHRFLPCENGGSHHHHHHHH
and
WC161_aa/1-172 comprises the amino acid sequence:
(SEQ ID NO: 28)
NKSKAVEQVKSDFNKLQDQGVYKAMNEFDIFINAIEAYMMIKMKSSRGQY
SREDNNCTHFPVGQSHMLLELRTAFSQVKTFFQTKDQLDNILLTDSLMQD
FKGYLGCQALSEMIQFYLVEVMPQAEKHGPEIKEHLNSLGEKLKTLRMRL
RRCHRFLPCENGGSHHHHHHHH.

### Inverted Monomer Dimers

In some embodiments, the disclosure provides polypeptides of formula (1) that are linked into a dimeric format. In some embodiments, the polypeptides of formula (1) have the structure of formula (2):

[Monomer1]-linkerₓ-[Monomer2] (2)

wherein Monomer 1 and Monomer 2 are polypeptides of formula 1. In some embodiments, Monomer 1 and Monomer 2 are the same (i.e., comprise the same amino acid sequence). In some embodiments, Monomer 1 and Monomer 2 are or different (i.e., comprise different amino acid sequences). In some embodiments, x=0 (linker absent) or 1 (linker present).

In some embodiments, the inverted monomer dimer of formula (2) comprises in the following amino to carboxyl order:
(a) Monomer 1 comprising:
   (i) a first polypeptide comprising an amino acid sequence having at least 90%, 91%, 92%, 93%, 94%, 95%, 96%, 97%, 98%, 99% or 100% sequence identity to a polypeptide selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature wild-type human IL-10 (SEQ ID NO:20);
   (ii) a first linkerₓ, wherein x=0 (linker absent) or 1 (linker present); and
   (iii) a second polypeptide comprising an amino acid having at least 90%, 91%, 92%, 93%, 94%, 95%, 96%, 97%, 98%, 99% or 100% sequence identity to a polypeptide sequence selected from the group consisting of amino acid residues 1-160 of human IL-10 (SEQ ID NO:20); amino acid residues 2-160 of human IL-10 (SEQ ID NO:36); amino acid residues 3-160 of human IL-10 (SEQ ID NO:37); amino acid residues 4-160 of human IL-10 (SEQ ID NO:38); amino acid residues 5-160 of human IL-10 (SEQ ID NO:39); amino acid residues 6-160 of human IL-10 (SEQ ID NO:40); amino acid residues 7-160 of human IL-10 (SEQ ID NO:41); amino acid residues 8-160 of human IL-10 (SEQ ID NO:42); amino acid residues 9-160 of human IL-10 (SEQ ID NO:43); amino acid residues 10-160 of human IL-10 (SEQ ID NO:44); and amino acid residues 11-160 of human IL-10 (SEQ ID NO:45) numbered in accordance with mature wild-type hIL10 (SEQ ID NO:20);
(b) a second linkery, wherein y=0 (linker absent) or 1 (linker present); and
(c) Monomer 2 comprising a polypeptide having at least 90%, 91%, 92%, 93%, 94%, 95%, 96%, 97%, 98%, 99% or 100% sequence identity to a polypeptide sequence selected from the group consisting of amino acid residues 1-116 of human IL-10 (SEQ ID NO:15); amino acid residues 2-116 of human IL-10 (SEQ ID NO:16); amino acid residues 3-116 of human IL-10 (SEQ ID NO:17), amino acid residues 4-116 of human IL-10 (SEQ ID NO:18), amino acid residues 5-116 of human IL-10 (SEQ ID NO:19), amino acid residues 6-116 of human IL-10 (SEQ ID NO:29), amino acid residues 7-116 of human IL-10 (SEQ ID NO:30), amino acid residues 8-116 of human IL-10 (SEQ ID NO:31), amino acid residues 9-116 of human IL-10 (SEQ ID NO:32), amino acid residues 10-116 of human IL-10 (SEQ ID NO:33); and amino acid residues 11-116 of human IL-10 (SEQ ID NO:34), numbered in accordance with mature human IL-10 (SEQ ID NO: 20).

In some embodiments, the inverted monomer dimer of formula (2) comprises in the following amino to carboxyl order:
(b) Monomer 1 comprising:
   (j) a first polypeptide comprising an amino acid sequence having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature wild-type human IL-10 (SEQ ID NO:20);
   (ii) a first linkerₓ, wherein x=0 (linker absent) or 1 (linker present); and
   (iii) a second polypeptide comprising an amino acid having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide sequence selected from the group consisting of amino acid residues 1-160 of human IL-10 (SEQ ID NO:20); amino acid residues 2-160 of human IL-10 (SEQ ID NO:36); amino acid residues 3-160 of human IL-10 (SEQ ID NO:37); amino acid residues 4-160 of human IL-10 (SEQ ID NO:38); amino acid residues 5-160 of human IL-10 (SEQ ID NO:39); amino acid residues 6-160 of human IL-10 (SEQ ID NO:40); amino acid residues 7-160 of human IL-10 (SEQ ID NO:41); amino acid residues 8-160 of human IL-10 (SEQ ID NO:42); amino acid residues 9-160 of human IL-10 (SEQ ID NO:43); amino acid residues 10-160 of human IL-10 (SEQ ID NO:44); and amino acid residues 11-160 of human IL-10 (SEQ ID NO:45) numbered in accordance with mature wild-type hIL10 (SEQ ID NO: 20);
(b) a second linker_{y}, wherein y=0 (linker absent) or 1 (linker present); and
(c) Monomer 2 comprising a polypeptide having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide sequence selected from the group consisting of amino acid residues 1-116 of human IL-10 (SEQ ID NO:15); amino acid residues 2-116 of human IL-10 (SEQ ID NO:16); amino acid residues 3-116 of human IL-10 (SEQ ID NO:17), amino acid residues 4-116 of human IL-10 (SEQ ID NO:18), amino acid residues 5-116 of human IL-10 (SEQ ID NO:19), amino acid residues 6-116 of human IL-10 (SEQ ID NO:29), amino acid residues 7-116 of human IL-10 (SEQ ID NO:30), amino acid residues 8-116 of human IL-10 (SEQ ID NO:31), amino acid residues 9-116 of human IL-10 (SEQ ID NO:32), amino acid residues 10-116 of human IL-10 (SEQ ID NO:33); and amino acid residues 11-116 of human IL-10 (SEQ ID NO:34), numbered in accordance with mature human IL-10 (SEQ ID NO:20).

In some embodiments of the inverted monomer of formula (2), the first linkerₓ is independent and distinct of the second linkery. In some embodiments inverted monomer of formula (2), x=0 (linker absent) or 1 (linker present). In some embodiments, y=0 (linker absent) or 1 (linker present). In some embodiments, if the first linkerₓ and second linkery are present, they can be the same or different. In some embodiments, a linker is not present between the first and second polypeptides of Monomer 1 and/or between Monomer 1 and Monomer 2 of formula (2). In some embodiments, a first linker (ii) (e.g., linkerₓ) is present between the first and second polypeptides of Monomer 1 and/or a second linker (b) (e.g., linker_{y}) is present between Monomer 1 and Monomer 2 of formula (2). In some embodiments, the first and second linkers comprise an amino acid sequence of 1, 2, 3, 4, or 5 amino acids.

Exemplary embodiments of the polypeptides of formula (2) are provided in Table 13 below.

TABLE 13
Exemplary Dimeric Inverted Monomers

| SEQ ID | | |
|---|---|---|
| NO | Name | Dimer Amino Acid Sequence |
| 46 | TP0020 | KSKAVEQVKNAFNKLQEKGIYKAMSEFDIFINYIEAYMT |
| | | MKIRNSPGQGTQSENSCTHFPGNLPNMLRDLRDAFSRVK |
| | | TFFQMKDQLDNLLLKESLLEDFKGYLGCQALSEMIQFYL |
| | | EEVMPQAENQDPDIKAHVNSLGENLKTLRLRLRRCHRFL |
| | | PCENKSKAVEQVKNAFNKLQEKGIYKAMSEFDIFINYIE |
| | | AYMTMKIRNSPGQGTQSENSCTHFPGNLPNMLRDLRDA |
| | | FSRVKTFFQMKDQLDNLLLKESLLEDFKGYLGCQALSE |
| | | MIQFYLEEVMPQAENQDPDIKAHVNSLGENLKTLRLRLR |
| | | RCHRFLPCEN (SEQ ID NO: 46) |
| 47 | TP0021 | KSKAVEQVKNAFNKLQEKGIYKAMSEFDIFINYIEAYMT |
| | | MKIRNSPGQGTQSENSCTHFPGNLPNMLRDLRDAFSRVK |
| | | TFFQMKDQLDNLLLKESLLEDFKGYLGCQALSEMIQFYL |
| | | EEVMPQAENQDPDIKAHVNSLGENLKTLRLRLRRCHRFL |
| | | PCENGGKSKAVEQVKNAFNKLQEKGIYKAMSEFDIFINY |
| | | IEAYMTMKIRNSPGQGTQSENSCTHFPGNLPNMLRDLRD |
| | | AFSRVKTFFQMKDQLDNLLLKESLLEDFKGYLGCQALSE |
| | | MIQFYLEEVMPQAENQDPDIKAHVNSLGENLKTLRLRLR |
| | | RCHRFLPCEN (SEQ ID NO: 47) |
| 48 | TP0022 | KSKAVEQVKNAFNKLQEKGIYKAMSEFDIFINYIEAYMT |
| | | MKIRNTSPGQGTQSENSCTHFPGNLPNMLRDLRDAFSRV |
| | | KTFFQMKDQLDNLLLKESLLEDFKGYLGCQALSEMIQFY |
| | | LEEVMPQAENQDPDIKAHVNSLGENLKTLRLRLRRCHRF |
| | | LPCENKSKAVEQVKNAFNKLQEKGIYKAMSEFDIFINYIE |
| | | AYMTMKIRNTSPGQGTQSENSCTHFPGNLPNMLRDLRD |
| | | AFSRVKTFFQMKDQLDNLLLKESLLEDFKGYLGCQALSE |
| | | MIQFYLEEVMPQAENQDPDIKAHVNSLGENLKTLRLRLR |
| | | RCHRFLPCEN (SEQ ID NO: 48) |
| 49 | TP0023 | KSKAVEQVKNAFNKLQEKGIYKAMSEFDIFINYIEAYMT |
| | | MKIRNTSPGQGTQSENSCTHFPGNLPNMLRDLRDAFSRV |
| | | KTFFQMKDQLDNLLLKESLLEDFKGYLGCQALSEMIQFY |
| | | LEEVMPQAENQDPDIKAHVNSLGENLKTLRLRLRRCHRF |
| | | LPCENGGKSKAVEQVKNAFNKLQEKGIYKAMSEFDIFIN |
| | | YIEAYMTMKIRNTSPGQGTQSENSCTHFPGNLPNMLRDL |
| | | RDAFSRVKTFFQMKDQLDNLLLKESLLEDFKGYLGCQA |
| | | LSEMIQFYLEEVMPQAENQDPDIKAHVNSLGENLKTLRL |
| | | RLRRCHRFLPCEN (SEQ ID NO: 49) |
| 50 | TP0024 | KSKAVEQVKNAFNKLQEKGIYKAMSEFDIFINYIEAYMT |
| | | MKIRNSPGQGTQSENSCTHFPGNLPNMLRKLRDAFSRVK |
| | | TFFQMKDQLDNLLLKESLLEDFKGYLGCQALSEMIQFYL |
| | | EEVMPQAENQDPDIKAHVNSLGENLKTLRLRLRRCHRFL |
| | | PCENKSKAVEQVKNAFNKLQEKGIYKAMSEFDIFINYIE |
| | | AYMTMKIRNSPGQGTQSENSCTHFPGNLPNMLRKLRDA |
| | | FSRVKTFFQMKDQLDNLLLKESLLEDFKGYLGCQALSE |
| | | MIQFYLEEVMPQAENQDPDIKAHVNSLGENLKTLRLRLR |
| | | RCHRFLPCEN (SEQ ID NO: 50) |
| 51 | TP0045 | KSKAVEQVKNAFNKLQEKGIYKAMSEFDIFINYIEAYMT |
| | | MKIRNSPGQGTQSENSCTHFPGNLPNMLRDLRDAFSRVK |
| | | TFFQMKDQLDNLLLKESLLEDFKGYLGCQALSEMIQFYL |
| | | EEVMPQAENQDPDIKAHVNSLGENLKLLRLRLRRCHRFL |
| | | PCENKSKAVEQVKNAFNKLQEKGIYKAMSEFDIFINYIE |
| | | AYMTMKIRNSPGQGTQSENSCTHFPGNLPNMLRDLRDA |
| | | FSRVKTFFQMKDQLDNLLLKESLLEDFKGYLGCQALSE |
| | | MIQFYLEEVMPQAENQDPDIKAHVNSLGENLKLLRLRLR |
| | | RCHRFLPCEN (SEQ ID NO: 51) |

In some embodiments, the inverted hIL10 monomer comprises an amino acid sequence having at least 90%, 91%, 92%, 93%, 94%, 95%, 96%, 97%, 98%, 99% or 100% sequence identity to an hIL10 inverted monomer selected from the group consisting of SEQ ID NO:46, SEQ ID NO:47, SEQ ID NO:48, SEQ ID NO:49, SEQ ID NO:50, and SEQ ID NO:51.

In some embodiments, the inverted hIL10 monomer comprises an amino acid sequence having least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to an hIL10 inverted monomer selected from the group consisting of SEQ ID NO:46, SEQ ID NO:47, SEQ ID NO:48, SEQ ID NO:49, SEQ ID NO:50, and SEQ ID NO: 51.

In some embodiments, the inverted monomer dimer of formula (2) comprises in the following amino to carboxyl order:
(a) Monomer 1 comprising:
   (i) a first polypeptide comprising an amino acid sequence selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature human IL-10 (SEQ ID NO:20),
   (ii) a first linkerₓ;
   (iii) a second polypeptide comprising an amino acid sequence selected from the group consisting of amino acid residues 1-116 of human IL-10 (SEQ ID NO:15);
(b) a second linkery;
(c) Monomer 2 comprising:
   (i) a first polypeptide comprising an amino acid sequence selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature human IL-10 (SEQ ID NO:20),
   (ii) a third linker_{z}; and
   (iii) A second polypeptide comprising an amino acid sequence selected from the group consisting of amino acid residues 1-116 of human IL-10 (SEQ ID NO:15).

In some embodiments, the first, second and third linkers are independent and distinct of each other. In some embodiments, x, y and/or z=0 (linker absent) or 1 (linker present). In some embodiments, if the first, second and/or third linkers (e.g., linkerₓ, linkery and linker_{z}) are present, they can independently be the same or different. For example, in some embodiments, the first, second and/or third linkers are the same (e.g., comprise the same amino acid sequence). In some embodiments, the first, second and/or third linkers are different (e.g., each linker comprises a different amino acid sequence, or one linker comprises a different amino acid sequence than the other two linkers). In some embodiments, a linker is not present between the first and second polypeptides of Monomer 1, between Monomer 1 and Monomer 2, or between the first and second polypeptides of Monomer 2 of formula (2).

In some embodiments, a first linker (e.g., linkerₓ) is present between the first and second polypeptides of Monomer 1. In some embodiments, a second linker (e.g., linker_{y}) is present between Monomer 1 and Monomer 2 of formula (2). In some embodiments, a third linker (e.g., linker_{z}) is present between the first and second polypeptides of Monomer 2. In some embodiments, the first, second and/or third linkers comprise an amino acid sequence of 1, 2, 3, 4, or 5 amino acids.

In any of the embodiments of the disclosure, the inverted monomer or inverted monomer dimer polypeptide can further optionally comprise a polypeptide sequence at the carboxy terminal (—COOH) end that is useful for purification of the polypeptide. In some embodiments, the sequence at the carboxy terminal (—COOH) end is GGS-8xHis (GGSHMHHHHHH (SEQ ID NO:52)).

In some embodiments, the inverted monomer dimer comprises an amino acid sequence in Table 8, Table 12, Table 13, or Table 16 or an amino acid sequence having at least 90%, 91%, 92%, 93%, 94%, 95%, 96%, 97%, 98%, or 99% sequence identity to an amino acid sequence in Table 8, Table 12, Table 13 or Table 16.

In some embodiments, the inverted monomer dimer comprises an amino acid sequence in Table 8, Table 12, Table 13, or Table 16 or an amino acid sequence having least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to an amino acid sequence in Table 8, Table 12, Table 13 or Table 16.

### Inverted Monomer Variants

In addition to the reference sequences of domains [A], [B], [C], [D], [E], and [F](comprising amino acid sequences derived from the wild type hIL10 sequence), the present disclosure provides polypeptides having 1 or 2 amino acid substitutions relative to the reference sequences of one or more (e.g., 1, 2, 3, 4, 5, or all 6) domains [A], [B], [C], [D], [E], and [F]. The reference sequences of domains [A], [B], [C], [D], [E], and [F] are primarily composed of alpha helical domains. As used herein, the term “helix” is as described in the RCSB Protein Data Bank, entry 2ILK, version 1.2. (See the internet at www.rcsb.org/sequence/2ILK). Consequently, in some embodiments of the disclosure, the amino acid substitutions to be included in one or more of domains [A], [B], [C], [D], [E], and [F] of the polypeptide of formula 1 are with amino acid substitutions that favor the formation (and/or do not contribute to destabilization) of alpha helices. In some embodiments, the amino acid substitution(s) at a given position(s) in the one or more of domains [A], [B], [C], [D], [E], and [F] of the polypeptide of formula 1 result(s) in a minimal free energy difference (Δ(ΔG)) relative to the amino acid(s) in the same position(s) of the reference sequence. In some embodiments, the amino acid(s) to be substituted in one or more of domains [A], [B], [C], [D], [E], and [F] are selected from the group consisting of methionine (M), alanine (A), leucine (L), glutamic acid (E), and lysine (K). In some embodiments, the polypeptide of formula 1 comprises 1 or 2 amino acid substitutions relative to the reference sequence of one or more of domains [A], [B], [C], [D], [E], and [F] with the proviso that the amino acid substitution(s) is not one or more amino acids selected from the group consisting of glycine (G) and proline (P).

In some embodiments, domain [C] comprises a D to K substitution at position 8 of SEQ ID NO:3. In some embodiments, domain [F] comprises a T to L substitution at position 14 of SEQ ID NO:6.

In some embodiments, the present disclosure provides variant inverted monomer or dimers thereof comprising one or more amino acid substitutions at positions corresponding to a residue of mature human or mouse IL10. In some embodiments, the inverted monomer or dimer thereof comprises one or more amino acid substitutions at positions corresponding to residues D25 or T100 of the mature human or mouse IL10 sequence. In some embodiments, the variant inverted monomer comprises the amino acid substitution D25K numbered in accordance with the mature human or mouse IL10 sequence. In some embodiments, the inverted monomer comprises the amino acid sequence:

(SEQ ID NO: 53)
KSKAVEQVKNAFNKLQEKGIYKAMSEFDIFINYIEAYMTMKIRNSPGQ
GTQSENSCTHFPGNLPNMLRKLRDAFSRVKTFFQMKDQLDNLLLKES
LLEDFKGYLGCQALSEMIQFYLEEVMPQAENQDPDIKAHVNSLGENL
KTLRLRLRRCHRFLPCEN,

or a sequence having at least 95% sequence identity to SEQ ID NO:53).

In some embodiments, the amino acid substitution at the residue corresponding to T100 is T100L.

In some embodiments, the variant inverted monomer comprises the amino acid substitution T100L numbered in accordance with the mature human or mouse IL10 sequence. In some embodiments, the inverted monomer comprises the amino acid sequence:

(SEQ ID NO: 90)
KSKAVEQVKNAFNKLQEKGIYKAMSEFDIFINYIEAYMTMKIRNSPG
QGTQSENSCTHFPGNLPNMLRDLRDAFSRVKTFFQMKDQLDNLLLK
ESLLEDFKGYLGCQALSEMIQFYLEEVMPQAENQDPDIKAHVNSLGE
NLKLLRLRLRRCHRFLPCEN

or a sequence having at least 95% sequence identity to SEQ ID NO: 91.

In some embodiments, one inverted monomer of a dimerized inverted monomer comprises an amino acid substitution(s) either or both of positions D25 or T100 (numbered in accordance with the mature human or mouse IL10 sequence). In some embodiments, each inverted monomer of a dimerized inverted monomer comprises an amino acid substitution one or both of positions D25 or T100 (numbered in accordance with the mature human or mouse IL10 sequence). In some embodiments, one inverted monomer of a dimerized inverted monomer comprises the amino acid substitution D25K (numbered in accordance with the mature human or mouse IL10 sequence). In some embodiments, one inverted monomer of a dimerized inverted monomer comprises the amino acid substitution T100L (numbered in accordance with the mature human or mouse IL10 sequence). In some embodiments, each inverted monomer of a dimerized inverted monomer comprises an amino acid substitution one or both of positions D25 or T100 (numbered in accordance with the mature human or mouse IL10 sequence). In some embodiments, the variant inverted monomer of the comprises the amino acid substitution D25K numbered in accordance with the mature human or mouse IL10 sequence. In some embodiments, the inverted monomer dimer comprises the amino acid sequence:

(SEQ ID NO: 54)
KSKAVEQVKNAFNKLQEKGIYKAMSEFDIFINYIEAYMTMKIRNSPGQ
GTQSENSCTHFPGNLPNMLRKLRDAFSRVKTFFQMKDQLDNLLLKES
LLEDFKGYLGCQALSEMIQFYLEEVMPQAENQDPDIKAHVNSLGENL
KTLRLRLRRCHRFLPCENKSKAVEQVKNAFNKLQEKGIYKAMSEFDIF
INYIEAYMTMKIRNSPGQGTQSENSCTHFPGNLPNMLRKLRDAFSRVK
TFFQMKDQLDNLLLKESLLEDFKGYLGCQALSEMIQFYLEEVMPQAE
NQDPDIKAHVNSLGENLKTLRLRLRRCHRFLPCEN,

or a sequence having at least 95% sequence identity to SEQ ID NO:54).

In some embodiments, the amino acid substitution at the residue corresponding to T100 of the inverted monomer dimer is T100L. In some embodiments, the inverted monomer dimer comprises the amino acid sequence:

(SEQ ID NO: 55)
KSKAVEQVKNAFNKLQEKGIYKAMSEFDIFINYIEAYMTMKIRNSPGQ
GTQSENSCTHFPGNLPNMLRDLRDAFSRVKTFFQMKDQLDNLLLKES
LLEDFKGYLGCQALSEMIQFYLEEVMPQAENQDPDIKAHVNSLGENL
KLLRLRLRRCHRFLPCENKSKAVEQVKNAFNKLQEKGIYKAMSEFDIF
INYIEAYMTMKIRNSPGQGTQSENSCTHFPGNLPNMLRDLRDAFSRVK
TFFQMKDQLDNLLLKESLLEDFKGYLGCQALSEMIQFYLEEVMPQAE
NQDPDIKAHVNSLGENLKLLRLRLRRCHRFLPCEN,

or a sequence having at least 95% sequence identity to SEQ ID NO:55).

### Biased Activity

The inflammatory response is a series of biological events in a mammal initiated in response to an infectious and/or injurious stimulus that mitigates the potential for systemic infection. Typically, the mammalian inflammatory response mediated by myeloid cells, particularly macrophages which are activated by foreign stimuli, for example components of bacterial cell walls such as the lipopolysaccharide of gram-negative bacteria (“LPS”). The activated myeloid cells act as the harbingers of infection and/or injury by secreting various pro-inflammatory signaling molecules including but not limited to interleukin-6 (IL6), interleukin-1 (IL1, especially IL1β) and tumor necrosis factor alpha (TNFα) that initiate and/or mediate the multiple biological processes associated with the inflammatory response.

Although the inflammatory response is essential for protecting the mammalian subject against infection, excessive and/or chronic activation of immune cells, particularly myeloid cells, is associated with tissue damage, organ malfunction, and autoimmune disease. A wide variety of human diseases are associated with excessive and/or chronic inflammation including but not limited to inflammatory bowel disease (IBD), rheumatoid arthritis (RA), alzheimer's disease, asthma, type 1 and type 2 diabetes, and cancer). The subject produces certain molecules, e.g., IL10, that act (in additional to other activities) to dampen the inflammatory response to prevent the deleterious effects associated with excess inflammation. Genetic loss of IL10 in both mice and humans is associated with severe inflammatory bowel disease (IBD). Expression and secretion of IL10 is correlated with suppression of the inflammatory response in immune cells including but not limited to inhibition of expression and/or secretion of proinflammatory cytokines and antigen presentation by activated myeloid cells. Despite a central role in the suppression of the inflammatory response, IL10 has also associated with pro-inflammatory activity particularly in activated CD8+ T cells. The contacting of activated CD8+ T cells with IL10 is observed to result in enhanced secretion of the pro-inflammatory cytokine interferon-gamma (IFNγ) as well the release of cytolytic factors such as granzyme A and granzyme B. These competing pro- and anti-inflammatory effects present a challenge to the therapeutic use of IL10 in the treatment of inflammatory disease in mammalian subjects.

In some embodiments, the inverted IL10 monomer is a biased IL10 partial agonist comprising a polypeptide of formula (1) or formula (2) that exhibits cell type biased activity relative to the wild type IL10 species from which the inverted IL10 monomer was derived. As used herein the term “biased” when used in the in the context of an IL10 inverted monomer (and/or dimers thereof) is used to indicated that the biased IL10 inverted monomer (or dimers thereof) exhibits a greater fraction of the level of wild type IL10 activity in a first cell type than a the level of wild type IL10 activity in second cell type relative to the wild type IL10 species from which the inverted IL10 monomer was derived. In one embodiment, the first cell type is a cell of myeloid origin, including a myeloid cell. In some embodiments, the myeloid cell is selected from a myelocyte, granulocyte, (e.g. neutrophil, eosinophil, or basophil), mast cell, or monocyte. In some embodiments, the monocyte is a macrophage or dendritic cell. In some embodiments, the macrophage is a Kupffer cell. In one embodiment, the first cell type is an activated myeloid cell. In one embodiment, the first cell type is a LPS activated human myeloid cell. In some embodiments, the second cell type is an T cell.

The inverted IL10 monomers and/or dimers thereof of the present disclosure can inhibit pro-inflammatory responses and/or STAT3-mediated signaling in a cell-type dependent manner, such that inflammatory macrophage activation is inhibited without substantially promoting the production of inflammatory cytokines such as interferon-γ by T cells. In some embodiments, an inverted monomer and/or dimer thereof of the present disclosure retains the immunosuppressive functions of wild-type hIL10, such as inhibiting the production of inflammatory cytokines, while decreasing the immunostimulatory functions of wild-type hIL10, such as the production of IFN-gamma by CD8+ T cells. For example, in some embodiments the inverted monomers and/or dimers thereof of the present disclosure retain activity comparable to wild-type hIL10 to suppress myeloid cell activation (e.g., as evaluated by increased STAT3-mediated signaling in myeloid cells), but possess substantially reduced activation (e.g., as evaluated by decreased production of IFN-gamma) in PBMCs, T cells, B cells and NK cells.

In some embodiments, the inverted monomers and/or dimers thereof of the present disclosure are hIL10 partial agonists.

### Pro-Inflammatory and Anti-Inflammatory Activity

In some embodiments, the inverted IL10 monomer comprising a polypeptide of formula (1) or formula (2) is a biased IL10 partial agonist that: (a) exhibits a significant level of at least one anti-inflammatory property of wild type IL10 and (b) exhibits a significantly reduced level of at least one pro-inflammatory property of wild type IL10. In some embodiments, “a significant level of at least one anti-inflammatory property” means that the Emax of the biased IL10 inverted monomer with respect to such anti-inflammatory property is greater than 10%, alternatively greater than 20%, alternatively greater than 30%, alternatively greater than 40%, alternatively greater than 50%, alternatively greater than 60%, alternatively greater than 70%, alternatively greater than 80%, alternatively greater than 90% of the Emax level of such anti-inflammatory property exhibited by wild-type IL10 as determined in a test system. Examples of anti-inflammatory properties that may be measured in a test system include but are not limited to: (a) the suppression of expression and/or secretion of hILβ by activated human myeloid cells, (b) the suppression of expression and/or secretion of hIL6 by activated human myeloid cells, and (c) the suppression of expression and/or secretion of hTNFα by activated human myeloid cells. In some embodiments, activated human myeloid cells are obtained by isolating human monocytes from the buffy coat of a centrifuged anticoagulated human blood sample, and activating the isolated monocytes by contacting the isolated monocytes with lipopolysaccharide [LPS] in accordance with procedures well known in the art. The levels of hILβ, hIL6, and hTNFα expressed and/or secreted by the activated monocytes may be determined by immunoassay or flow-cytometry methods in accordance with procedures well known in the art. One protocol for the evaluation of the suppression of expression and/or secretion of hILβ, hIL6, and hTNF by LPS activated human monocytes is provided in Examples herein.

In some embodiments, “a significantly reduced level of at least one pro-inflammatory property” means that the Emax of the biased IL10 inverted monomer with respect to such pro-inflammatory property that is less than 90%, alternatively less than 80%, alternatively less than 70%, alternatively less than 60%, alternatively less than 50%, alternatively less than 40%, alternatively less than 30%, alternatively less than 20%, alternatively less than 10% of the Emax of that pro-inflammatory property of wild-type IL10 as determined in a test system. Examples of pro-inflammatory properties include but are not limited to: (a) the suppression of expression and/or secretion of IFNγ by activated human CD8+ T cells, (b) the suppression of expression and/or secretion of granzyme A by activated human CD8+ T cells, and (c) the suppression of expression and/or secretion of granzyme A by activated human CD8+ T cells. In some embodiments, activated human T cells are obtained by isolating CD8+ T cells human whole blood and activated by contacting the isolated CD8+ cells with anti-CD3 and anti-CD28 antibodies in accordance with procedures well known in the art. The levels of IFNγ, granzyme A, and granzyme B expressed and/or secreted by the isolated CD8+ T cells may be determined by immunoassay or flow-cytometry methods in accordance with procedures well known in the art. One protocol for the evaluation of the expression and/or secretion of IFNγ, granzyme A, and granzyme B expressed and/or secreted by CD3/CD28 activated CD8+ T cells is provided in the Examples below.

In some embodiments, the inverted IL10 monomer comprises a polypeptide of formula (1) or formula (2) is a biased hIL10 partial agonist that exhibits a significant level of at least one anti-inflammatory property of wild type hIL10 and exhibits a significantly reduced level of at least one pro-inflammatory property of wild type IL10, wherein: (a) the significant level of at least one anti-inflammatory property of wild type hIL10 is an Emax of at least one anti-inflammatory property greater than 30% of the Emax of such anti-inflammatory property exhibited by wild-type hIL10 wherein the at least one anti-inflammatory property is selected from the group consisting of (i) the suppression of expression and/or secretion of hILb in LPS activated human monocytes, (ii) the suppression of expression and/or secretion of hIL6 in LPS activated human monocytes, or (iii) the suppression of expression and/or secretion of hTNFa in LPS activated human monocytes; and (b) the significantly reduced level of at least one pro-inflammatory property of wild type hIL10 is an Emax of at least one anti-inflammatory property less than 30% of the Emax of such anti-inflammatory property exhibited by wild-type hIL10 wherein the at least one pro-inflammatory property is selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

In some embodiments, the inverted IL10 monomer of the present disclosure comprising a polypeptide of formula (1) or formula (2) is a biased hIL10 partial agonist that exhibits a significant level of at least one anti-inflammatory property of wild type hIL10 and exhibits a significantly reduced level of at least one pro-inflammatory property of wild type IL10, wherein: the significant level of at least one anti-inflammatory property of wild type hIL10 is an Emax of at least one anti-inflammatory property greater than 30% of the Emax of such anti-inflammatory property exhibited by wild-type hILL10 wherein the at least one anti-inflammatory property is selected from the group consisting of (i) the suppression of expression and/or secretion of hILb in LPS activated human monocytes, (ii) the suppression of expression and/or secretion of hIL6 in LPS activated human monocytes, or (iii) the suppression of expression and/or secretion of hTNFa in LPS activated human monocytes; and (b) the significantly reduced level of at least one pro-inflammatory property of wild type hIL10 is an Emax of at least one anti-inflammatory property less than 30% of the Emax of such anti-inflammatory property exhibited by wild-type hIL10 wherein the at least one pro-inflammatory property is selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

In some embodiments, the inverted IL10 monomer of the present disclosure comprising a polypeptide of formula (1) or formula (2) is a biased inverted hIL10 monomer wherein: (a) the Emax of the inverted hIL10 monomer greater than 30% of the Emax of wild type hIL10 in an assay of anti-inflammatory activity selected from the group consisting of (i) the suppression of expression and/or secretion of hILβ in LPS activated human monocytes, (ii) the suppression of expression and/or secretion of hIL6 in LPS activated human monocytes, or (iii) the suppression of expression and/or secretion of hTNFα in LPS activated human monocytes; and (b) the Emax of the inverted hIL10 monomer less than 10% of the Emax of wild type hIL10 in an assay of pro-inflammatory activity selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

In some embodiments, the inverted IL10 monomer of the present disclosure comprising a polypeptide of formula (1) or formula (2) is a biased inverted hIL10 monomer wherein: (a) the Emax of the inverted hIL10 monomer greater than 50% of the Emax of wild type hIL10 in an assay of anti-inflammatory activity selected from the group consisting of (i) the suppression of expression and/or secretion of hILβ in LPS activated human monocytes, (ii) the suppression of expression and/or secretion of hIL6 in LPS activated human monocytes, or (iii) the suppression of expression and/or secretion of hTNFα in LPS activated human monocytes; and (b) the Emax of the inverted hIL10 monomer less than 20% of the Emax of wild type hIL10 in an assay of pro-inflammatory activity selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

In some embodiments, the inverted IL10 monomer of the present disclosure comprising a polypeptide of formula (1) or formula (2) is a biased inverted hIL10 monomer wherein: (a) the Emax of the inverted hIL10 monomer greater than 50% of the Emax of wild type hIL10 in an assay of anti-inflammatory activity selected from the group consisting of (i) the suppression of expression and/or secretion of hILβ in LPS activated human monocytes, (ii) the suppression of expression and/or secretion of hIL6 in LPS activated human monocytes, or (iii) the suppression of expression and/or secretion of hTNFα in LPS activated human monocytes; and (b) the Emax of the inverted hIL10 monomer less than 10% of the Emax of wild type hIL10 in an assay of pro-inflammatory activity selected from the group consisting of (i) the suppression of expression and/or secretion of IFNγ by activated human CD8+ T cells, (i) the suppression of expression and/or secretion of granzyme A by activated human CD8+ T cells, and (iii) the suppression of expression and/or secretion of granzyme A by activated human CD8+ T cells.

### STAT3

As previously noted, the interaction of IL10 with the IL10 receptor results in the intracellular signaling characterized by the enhanced intracellular production of phosphorylated STAT3 (phospho-STAT3). Consequently, one measure of IL10 activity may be evaluated using a cell expressing the IL10 receptor (comprised of IL10Ra and IL10Rb) is the intracellular production of phospho-STAT3.

In one embodiment, polypeptide of formula (1) or formula (2) is a biased hIL10 partial agonist, the first cell type is an activated human myeloid cell and the second cell type is an activated human T cell wherein the level of IL10 activity is measured by intracellular production of phospho-STAT3. In one embodiment, the polypeptide of formula (1) or formula (2) retains is a biased hIL10 partial agonist that retains a greater fraction of hIL10 activity on activated human monocytes than activated human CD8+ T cells wherein the level of IL10 activity is measured by intracellular production of phospho-STAT3. In some embodiments, the level of IL10 activity in the first cell type and second cell type is measured by the production of phospho-STAT3 in a first cell type in response to contacting the first cell type and second cell type with an inverted IL10 monomer comprising a polypeptide of formula (1) or formula (2).

In some embodiments, the relative activation of STAT3 signaling of inverted monomers and/or dimers thereof described herein in a first cell type versus a second cell type is different from the relative activation of STAT3 signaling of a wild type human or murine IL10 in the first cell type versus the second cell type. In some embodiments, the level of intracellular phospho-STAT3 induced in a human myeloid cell in response to contacting the myeloid cell with an effective amount of a human IL10 inverted monomer (or dimers thereof) is at least 10 fold, alternatively at least 100 fold, alternatively at least 1000 fold, greater than the level of intracellular phospho-STAT3 induced in a human lymphocyte cell in response to contacting the human lymphocyte cell with the same amount of the human IL10 inverted monomer (or dimers thereof). In one embodiment, the ratio of the level of STAT3 signaling induced in a myeloid cell in response to contacting a myeloid cell with a human IL10 inverted monomer relative to the level of STAT3 signaling induced in a lymphocyte cell in response to contacting a lymphocyte with the human IL10 inverted monomer is different than (greater than or lesser than) the ratio of the level of STAT3 signaling induced in the myeloid cell in response to contacting the myeloid cell with wild type hIL10 relative to the level of STAT3 signaling induced in the lymphocyte in response to contacting the lymphocyte with wild type hIL10. In some embodiments, the ratio of the activity (as determined by the level of intracellular phospho-STAT3) of a human IL10 inverted monomer (and/or dimers thereof) in human myeloid cells relative to human lymphocytes is greater than the ratio of activity of wild type human IL10 in human myeloid cells relative to human lymphocytes. In some embodiments, the myeloid cell is a neutrophil, eosinophil, mast cell, basophil or monocyte. In some embodiments, the monocyte is a macrophage or a dendritic cell. In some embodiments, the macrophage is a Kupffer cell. In some embodiments, the lymphocyte is a CD8+ T cell, a CD4+ T cell, a B cell or an NK cell.

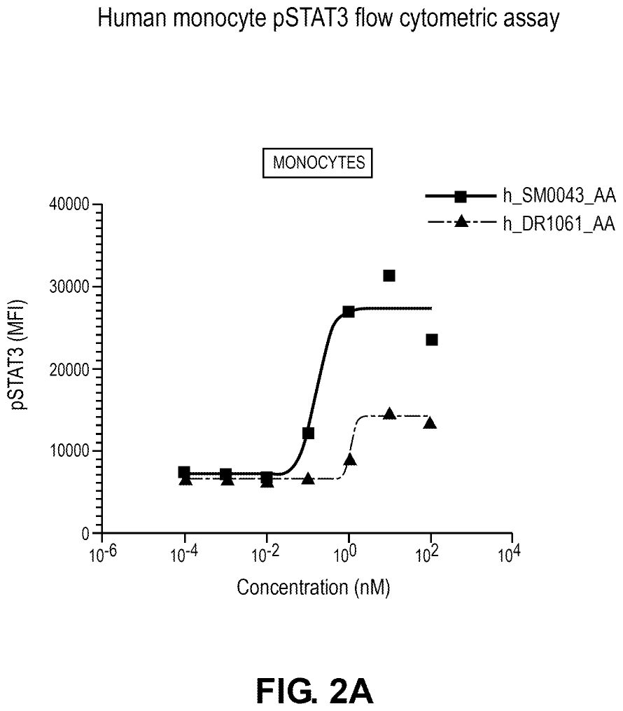

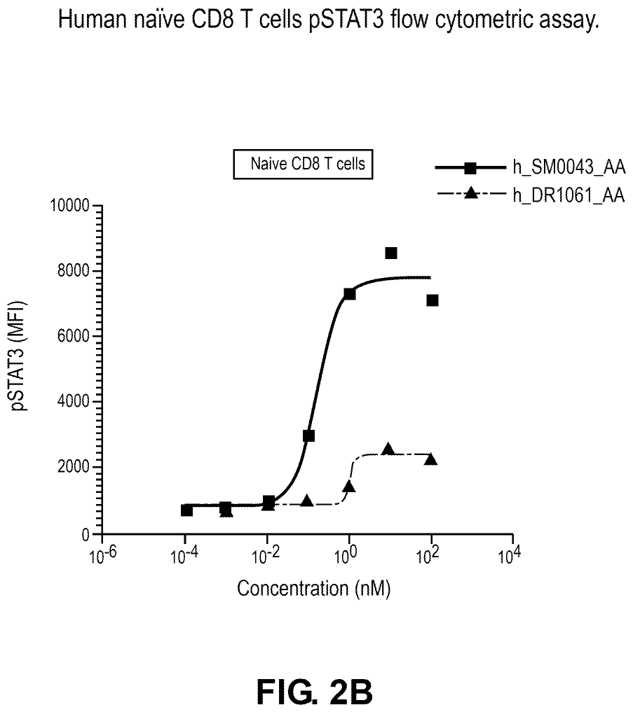

In some embodiments, the inverted monomers and/or dimers thereof of the present disclosure have a pSTAT3 Eₘₐₓ of greater than 20%, greater than 30%, greater than 40%, greater than 50%, greater than 60%, or greater than 70% of the pSTAT3 Eₘₐₓ of wild-type hIL10 in myeloid cells (See, for example FIG. 2A). In some embodiments, an inverted monomer and/or dimer thereof of the present disclosure exhibits decreased STAT3-mediated signaling in lymphocytes such as T cells, B cells or NK cells compared to wild-type hIL10 (See, for example FIG. 2B). In some embodiments, an inverted monomer and/or dimer thereof of the present disclosure has a pSTAT3 Eₘₐₓ in a lymphocyte less than 70%, less than 60%, less than 50%, less than 40%, or less than 30%, of the pSTAT3 Eₘₐₓ of a wild-type hIL10 in lymphocytes. In some embodiments, the inverted monomers and/or dimers thereof result in a pSTAT3 Eₘₐₓ in a lymphocyte less than 70% (e.g., less than 70%, less than 60%, less than 50%, less than 40%, or less than 30%) but greater than 20% of the pSTAT3 Eₘₐₓ of a wild-type or parental IL10 polypeptide in the lymphocyte. In some embodiments, the lymphocyte is selected from a CD8+ T cell, a CD4+ T cell, a B cell or an NK cell.

### Linkers

In some embodiments, the disclosure provides polypeptide linkers that can be used to covalently join functional subunits of a polypeptide composed of multiple functional domains or subunits. Linkers are typically of sufficient length to permit some movement between the functional domains or subunits of the polypeptide they join. In some embodiments, a linker is present between one or more different helices or domains [A], [B], [C], [D], [E], and [F] of formula (1). In some embodiments, a linker is present between the two monomers of formula (2).

Linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of linkers useful in the practice of the present disclosure include but are not limited to glycine polymers (G)n where is an integer from 1-50 (SEQ ID NO: 138). Examples of linkers useful in the practice of the present disclosure include but are not limited to glycine-alanine polymers, alanine-serine polymers, and glycine-serine polymers also referred to herein as “GS-linkers.” Glycine and glycine-serine polymers are relatively unstructured, and therefore may serve as a flexible tether between domains or subunits of a polypeptide. In some embodiments, a GS-linker is a polymer selected of the formulae: (GmSo)n (SEQ ID NO: 136), (GS)n (SEQ ID NO: 137), (GSGGS)n (SEQ ID NO:56), (GGGS)n (SEQ ID NO:57), (GGGGS)n (SEQ ID NO:58), (GmSoGm)n (SEQ ID NO:59), (GmSoGmSoGm)n (SEQ ID NO:60), (GSGGSm)n (SEQ ID NO:61), (GSGSmG)n (SEQ ID NO:62) and (GGGSm)n (SEQ ID NO:63), and (GGGGS)n (SEQ ID NO:64), and combinations thereof, where m, n, and o are each independently selected from an integer from 1 to 20, e.g., 1-18, 2-16, 3-14, 4-12, 5-10, 1, 2, 3, 4, 5, 6, 7, 8, 9, or 10. Examples of GS-linkers include, but are not limited to GGSG (SEQ ID NO:65), GGSGG (SEQ ID NO:66), GSGSG (SEQ ID NO:67), GSGGG (SEQ ID NO:68), GGGSG (SEQ ID NO:69), and GSSSG (SEQ ID NO:70), GGGS (SEQ ID NO:71), GGGGS (SEQ ID NO:72), GGGGSGGGGS (SEQ ID NO:73), GGGGSGGGGSGGGGS (SEQ ID NO:74), GGSG (SEQ ID NO:75), GGSGG (SEQ ID NO:76), GSGSG (SEQ ID NO:77), GSGGG (SEQ ID NO:78), GGGSG (SEQ ID NO:79), GGGGSGGGGS (SEQ ID NO:80), GGGGSGGGGSGSSSG (SEQ ID NO:81) and multimers thereof.

### Modifications to Provide Additional Functions

In some embodiments, the inverted monomer and/or dimer thereof may comprise a functional domain of a chimeric polypeptide. Inverted monomer fusion proteins of the present disclosure may be readily produced by recombinant DNA methodology by techniques known in the art by constructing a recombinant vector comprising a nucleic acid sequence comprising a nucleic acid sequence encoding the inverted monomer in frame with a nucleic acid sequence encoding the fusion partner either at the N-terminus or C-terminus of the inverted monomers, the sequence optionally further comprising a nucleic acid sequence in frame encoding a linker or spacer polypeptide.

In other embodiments, the inverted monomer and/or dimer thereof can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding molecule further comprises a C-terminal c-myc epitope tag.

In some embodiments, the inverted monomer and/or dimer thereof is conjugated to a molecule (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the inverted monomer sequence and the sequence of the targeting domain of the fusion protein.

In other embodiments, a chimeric polypeptide including an inverted monomer and an antibody or antigen-binding portion thereof can be generated. The antibody or antigen-binding component of the chimeric protein can serve as a targeting moiety. For example, it can be used to localize the chimeric protein to a particular subset of cells or target molecule. In some embodiments, the targeting domain is an antibody. As used herein, the term “antibody” means any form of antibody (also known as an immunoglobulin (Ig)) that exhibits the desired biological activity of binding to an antigen epitope, as described herein. The term “antibody” specifically covers, but is not limited to, polyclonal antibodies, monoclonal antibodies (including full length monoclonal antibodies comprising two light chains and two heavy chains), multispecific antibodies (e.g., bispecific antibodies that bind to two or more antigens or antigen epitopes on a single antigen), fully human antibodies (huAb), humanized antibodies (hzAb), chimeric antibodies, single chain variable fragment antibodies (scFv), single-domain antibodies (sdAb), variable heavy (VH) domain antibodies, diabodies (dAb), and antigen-binding fragments of heavy chain only antibodies (VHH), comprising the amino acid sequences of the variable region, as described herein. As used herein, the term “antibody” refers collectively to: (a) glycosylated and non-glycosylated immunoglobulins (including but not limited to mammalian immunoglobulin classes IgG1, IgG2, IgG3, and IgG4) that specifically bind to a target molecule, such as an antigen, and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that compete with the immunoglobulin from which it was derived for binding to the target molecule. The term antibody is not restricted to immunoglobulins derived from any particular mammalian species and includes murine, human, equine, camelids, and uman antibodies. The term antibody includes “heavy chain antibodies,” “VHHs” and “nanobodies” as typically obtained from immunization of camelids (including camels, llamas, and alpacas, such as described by e.g., Hamers-Casterman et al. 1993. Nature. 363:446-448, as described in greater detail below in the definition of “VHH.” The term “antibody” encompasses antibodies isolatable from natural sources or from animals following immunization with an antigen, as well as engineered antibodies including monoclonal antibodies, bispecific antibodies, tri-specific, chimeric antibodies, humanized antibodies, human antibodies, CDR-grafted, veneered, or deimmunized (e.g., to remove B and/or T-cell epitopes) antibodies. In some embodiments the targeting domain specifically binds to a cell surface marker of pro-inflammatory cell such as activated immune cell. In some embodiments, the targeting domain is an antibody that selectively binds to a cell surface marker including but not limited to the IL1R1 receptor, IL-1 receptor accessory protein, the IL6 receptor subunit (IL6R), HLA-DR, HLA-DR α-chain, HLA-DR β-chain the TNFR1, TNFR2, CD4, CD8, F4/80, CCR2, CD169, CX3CR1, CD206, CD163 or, Lyvel. Methods of generating cytokine-antibody chimeric polypeptides are described, for example, in U.S. Pat. No. 6,617,135.

Association with Carrier Molecules to Increase Duration of Action

The inverted monomers and/or dimers thereof described herein can be modified to provide for an extended lifetime in vivo and/or extended duration of action in a subject. In some embodiments, the inverted monomer and/or dimer thereof is conjugated to a carrier molecule to provide desired pharmacological properties such as an extended half-life. In some embodiments, the inverted monomer and/or dimer thereof is covalently linked to the Fc domain of an IgG, albumin, water soluble polymers, or other molecules to extend its half-life, e.g., glycosylation, acylation and the like as known in the art. In some embodiments, the inverted monomer and/or dimer thereof is modified to provide an extended duration of action in a mammalian subject has a half-life in a mammalian subject of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the inverted monomer and/or dimer thereof to provide an extended duration of action in a mammalian subject include (but are not limited to);
conjugation of the inverted monomer to one or more protein carrier molecules,
conjugation of the inverted monomer to protein carrier molecules, optionally in the form of a fusion protein with additional polypeptide sequences (e.g., inverted monomer-Fc fusions) and
conjugation to polymers, (e.g., water soluble polymers to provide a PEGylated IL10 inverted monomer polypeptide).

It should be noted that the more than one type of modification that provides for an extended duration of action in a mammalian subject may be employed with respect to a given inverted monomer and/or dimer thereof. For example, a hIL10 inverted monomer and/or dimer thereof of the present disclosure may comprise both amino acid substitutions that provide for an extended duration of action as well as conjugation to a carrier molecule such as a polyethylene glycol (PEG) molecule.

### Protein Carrier Molecules:

Examples of protein carrier molecules which may be covalently attached to the inverted monomer and/or dimer thereof to provide an extended duration of action in vivo include, but are not limited to albumins, antibodies and antibody fragments such and Fc domains of IgG molecules.

### Fc Fusions:

In some embodiments, an inverted monomer of the disclosure is conjugated to an Fc domain. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule. In a typical presentation, each monomer of the dimeric Fc can carry a heterologous polypeptide, the heterologous polypeptides being the same or different.

As indicated, the linkage of an inverted monomer to the Fc subunit may incorporate a linker molecule between the inverted monomer and Fc subunit. In some embodiments, the inverted monomer is expressed as a fusion protein with the Fc domain incorporating an amino acid sequence of a hinge region of an IgG antibody. The Fc domains engineered in accordance with the foregoing may be derived from IgG1, IgG2, IgG3 and IgG4 mammalian IgG species. In some embodiments, the Fc domains may be derived from human IgG1, IgG2, IgG3 and IgG4 IgG species. In some embodiments, the hinge region is the hinge region of an IgG1. In one particular embodiment, the inverted monomer is linked to an Fc domain using a human IgG1 hinge domain.

In some embodiments, the linker is a chemical linker. Examples of chemical linkers include aryl acetylene, ethylene glycol oligomers containing 2-10 monomer units, diamines, diacids, amino acids, or combinations thereof. In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). Glycine and glycine-serine polymers are relatively unstructured, and therefore may serve as a neutral tether between components. Examples of glycine polymers include (G)n, glycine-alanine polymers, alanine-serine polymers, glycine-serine polymers (for example, (GmSo)n (SEQ ID NO: 136), (GSGGS)n (SEQ ID NO:56), (GmSoGm)n (SEQ ID NO:59), (GmSoGmSoGm)n (SEQ ID NO:60), (GSGGSm)n (SEQ ID NO:61), (GSGSmG)n (SEQ ID NO:62) and (GGGSm)n (SEQ ID NO:63), and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers.

In some embodiments the amino acid sequence of the Fc domain is modified to reduce effector function. In some embodiments, the Fc domain may be modified to substantially reduce binding to Fc receptors (FcyR and FcR) which reduces or abolishes antibody directed cytotoxicity (ADCC) effector function. Modification of Fc domains to reduce effector function are well known in the art. See, e.g., Wang, et al. (2018) IgG Fc engineering to modulate antibody effector functions, Protein Cell 9(1):63-73. For example, mutation of the lysine residue at position 235 (EU numbering) from leucine (L) to glutamic acid (E) is known to reduce effector function by reducing FcgR and C1q binding. Alegre, et al. (1992) J. Immunology 148:3461-3468. Additionally, substitution of the two leucine (L) residues at positions 234 and 235 (EU numbering) in the IgG1 hinge region with alanine (A), i.e., L234A and L235A, results in decreased complement dependent cytotoxicity (CDC) and antibody dependent cellular cytotoxicity (ADCC). Hezereh et al., (2001) J. Virol 75(24):12161-68. Furthermore, mutation of the proline at position 329 (EU numbering) to alanine (P329A) or glycine, (P329G) mitigates effector function and may be combined with the L234A and L235A substitutions. In some embodiments, the Fc domains may comprise the amino acid substitutions L234A/L235A/P329A (EU numbering) referred to as the “LALAPA” substitutions or L234A/L235A/P329G (EU numbering) referred to as the “LALAPG” substitutions. In some embodiments, the Fe domains may comprise the amino acid substitutions E233P/L234V/L235A/AG237 (referred to in the scientific literature as the PVAdelG mutation).

In some embodiments, the Fc domain is derived from hIgG4. It has been shown that glycosylation at position 297 (EU numbering) contributes to effector function in IgG4. Edelman, et al (1969) PNAS (USA) 63:78-85 Examples of modifications at N297 to eliminate glycosylation sites and effector functions in the Fc domain of hIgG4include the amino acid substitutions selected from N297Q and N297G (EU numbering).

In some embodiments the amino acid sequence of the Fc domain is modified to incorporate amino acid substitutions which extend the duration of action of the molecule and prevent clearance. In some embodiments, such modifications to the Fc domain include the amino acid substitutions M428L and N434S (EU numbering) referred to as the “LS” modification. The LS modification may optionally be combined with amino acid substitutions to reduce effector function.

In some embodiments the amino acid sequence of the Fc domain may be further modified to eliminate N-linked or O-linked glycosylation sites. Aglycosylated variants of Fc domains, particularly of the IgG1 subclass are known to be poor mediators of effector function. Jefferies et al. 1998, Immol. Rev., vol. 163, 50-76).

In some embodiments, an inverted monomer or dimer thereof of the disclosure may be further modified to extend its duration of action in vivo. In some embodiments, conjugation of the PEG moiety may be accomplished via a sulfhydryl (—SH) group of a cysteine residue. In some embodiments, PEGylation of the inverted monomer/Fc fusion polypeptides is provided at the naturally occurring cysteine residues at position 220 (C220, EU Numbering) of the upper hinge region of the Fc domain.

### Albumin Carrier Molecules

In some embodiments, an inverted monomer and/or dimer thereof is conjugated to an albumin molecule (e.g., human serum albumin) which is known in the art to facilitate extended exposure in vivo. In some embodiments, the inverted monomer and/or dimer thereof is conjugated to albumin via chemical linkage or expressed as a fusion protein with an albumin molecule (referred to herein as a “inverted monomer-albumin fusion”). The term “albumin” as used in the context inverted monomer-albumin fusions includes albumins such as human serum albumin (HSA), cyno serum albumin, and bovine serum albumin (BSA). In some embodiments, the HSA comprises a C34S or K573P amino acid substitution relative to the wild-type HSA sequence. According to the present disclosure, albumin can be conjugated to an inverted monomer at the carboxyl terminus, the amino terminus, both the carboxyl and amino termini, and internally (see, e.g., U.S. Pat. Nos. 5,876,969, 7,056,701). In the -HSA fusions of the inverted il10 monomer contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising an inverted monomer fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. As an alternative to chemical linkage between the inverted monomer and the albumin molecule, the inverted monomer—albumin complex may be provided as a fusion protein comprising an albumin polypeptide sequence and an inverted monomer recombinantly expressed in a host cell as a single polypeptide chain, optionally comprising a linker molecule between the albumin and inverted monomer. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein isolated from the host cell culture by techniques well known in the art.

### Polymeric Carriers

In some embodiments, extended in vivo duration of action of the inverted monomer and/or dimer thereof may be achieved by conjugation to one or more polymeric carrier molecules such as XTEN polymers or water soluble polymers.

### XTEN Conjugates

The inverted monomer and/or dimer thereof may further comprise an XTEN polymer. The XTEN polymer conjugated (either chemically or as a fusion protein) to an inverted monomer and/or dimer thereof can provide extended duration akin to PEGylation and may be produced as a recombinant fusion protein in E. coli. XTEN polymers suitable for use in conjunction with the inverted monomer and/or dimer thereof of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI:10.1371/journal.pone.0157193 Jun. 13, 2016. The XTEN polymer fusion protein may incorporate a protease sensitive cleavage site between the XTEN polypeptide and the inverted monomer and/or dimer thereof such as an MMP-2 cleavage site.

### Water Soluble Polymers

In some embodiments, the inverted monomer and/or dimer thereof can be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol,), polysaccharides), poly-alpha-hydroxy acid), polyvinyl alcohol (PVA), polyphosphazene, polyoxazolines (POZ), poly(N-acryloylmorpholine), or a combination thereof.

In some embodiments, the inverted monomer and/or dimer thereof can be conjugated to one or more polyethylene glycol molecules or “PEGylated.” Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature, and have the general formula

R(O—CH₂—CH₂)ₙO—R,

where R is hydrogen or a protective group such as an alkyl or an alkanol group, and where n is an integer from 1 to 1000. When R is a protective group, it generally has from 1 to 8 carbons.

The PEG can be linear or branched. Branched PEG derivatives, “star-PEGs” and multi-armed PEGs are contemplated by the present disclosure.

In some embodiments, selective PEGylation of the inverted monomer and/or dimer thereof, for example, by the incorporation of non-natural amino acids having side chains to facilitate selective PEG conjugation, may be employed. Specific PEGylation sites can be chosen such that PEGylation of the binding molecule does not affect its binding to the target receptors.

In certain embodiments, the increase in half-life is greater than any decrease in biological activity. PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

In some embodiments, the present disclosure provides a “monoPEGylated” inverted monomer dimer (i.e., an inverted monomer dimer wherein only one inverted monomer of the inverted monomer dimer is PEGylated) and a “diPEGylated” inverted monomer dimer (i.e., an inverted monomer dimer wherein both inverted monomers of the dimer are PEGylated).

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding molecules is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10kDA linear PEG molecules (e.g., Sunbright® GL2-200AL3, NOF), a 20 kDa 2-arm branched PEG-NHS ester the 20 kDA PEG-NHS ester comprising two 10kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20kDA linear PEG molecules (e.g., Sunbright® GL2-400AL3), a 40 kDa 2-arm branched PEG-NHS ester the 40 kDA PEG-NHS ester comprising two 20kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can used to join the inverted monomer and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding molecule is acetylated at the N-terminus by enzymatic reaction with N-terminal acetyltransferase and, for example, acetyl CoA. Alternatively, or in addition to N-terminal acetylation, the binding molecule can be acetylated at one or more lysine residues, e.g., by enzymatic reaction with a lysine acetyltransferase. See, for example Choudhary et al. (2009) Science 325 (5942):834-840.

In some embodiments, the present disclosure provides an inverted monomer that is PEGylated, wherein the PEG is conjugated to the inverted monomer and the PEG is a linear or branched PEG molecule having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons.

In one embodiment of the disclosure, the PEG is a 40 kD branched PEG comprising two 20 kD arms.

In some embodiments, the present disclosure provides a PEGylated inverted monomer, wherein the PEGylated inverted monomer is a molecule of the formula 2 or 3:

PEG-Lₘ-[INVERTED MONOMER] [2]

[INVERTED MONOMER]-Lₘ-PEG [3]

wherein:
(a) PEG is a linear or branched polyethylene glycol molecule having a molecular weight of from about 10 kD to about 80 kD;
(v) INVERTED MONOMER is an inverted monomer of formula 1;
(c) L is a polypeptide or chemical linker;
(d) and m=0 (absent) or 1 (present);

In some embodiments, the PEGylated inverted monomer of formula 2 or 3 comprises a 40 kDa PEG linear or branched PEG. In some embodiments, the 40 kDa PEG is a branched 40 kDa PEG comprising two 20 kDa arms. In some embodiments, PEGylated inverted monomer of formula 2 or 3 comprises a 40 kDa branched PEG of the structure:

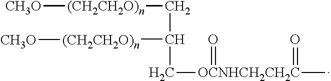

In some embodiments, the inverted monomer of the present disclosure is a PEGylated inverted monomer of formula 2 wherein: (a) the INVERTED MONOMER is polypeptide selected from the group consisting of SEQ ID NO;10 and SEQ ID NO:11; (b) m=1; and PEG is a 40 kDa branched PEG of the structure:

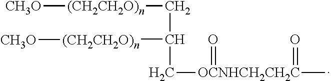

In some embodiments, the inverted monomer of the present disclosure is a PEGylated inverted monomer of formula 3 wherein: (a) the INVERTED MONOMER is polypeptide selected from the group consisting of SEQ ID NO;10 and SEQ ID NO:11; (b) m=1; and PEG is a a 40 kDa branched PEG of the structure:

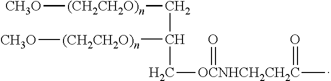

### Fatty Acid Carriers

In some embodiments an inverted monomer and/or dimer thereof having an extended duration of action in a mammalian subject and useful in the practice of the present disclosure is achieved by covalent attachment of the inverted monomer and/or dimer thereof to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine—SH groups such as DHHC proteins catalyze S-palmitoylation. Palmitoleylation of serine and threonine residues is typically achieved enzymatically using PORCN enzymes. In some embodiments, the inverted monomer and/or dimer thereof is acetylated at the N-terminus by enzymatic reaction with N-terminal acetyltransferase and, for example, acetyl CoA. Alternatively, or in addition to N-terminal acetylation, the inverted monomer and/or dimer thereof can be acetylated at one or more lysine residues, e.g., by enzymatic reaction with a lysine acetyltransferase. See, for example Choudhary et al. (2009) Science 325 (5942):834L2 ortho840.

### Recombinant Production

In some embodiments, the inverted monomers and/or dimers thereof of the present disclosure are produced by recombinant DNA technology. In the typical practice of recombinant production of polypeptides, a nucleic acid sequence encoding the desired polypeptide is incorporated into an expression vector suitable for the host cell in which expression will be accomplished, the nucleic acid sequence being operably linked to one or more expression control sequences encoded by the vector and functional in the target host cell. The recombinant protein may be recovered through disruption of the host cell or from the cell medium if a secretion leader sequence (signal peptide) is incorporated into the polypeptide.

In certain embodiments, the inverted monomers and/or dimers thereof of the present disclosure contain amino acid substitutions which provide enhanced recombinant expression relative to the expression of wild-type hIL10 or a mutein not containing such substitution; pharmaceutical compositions comprising an inverted monomer and/or dimer thereof, recombinant nucleic acid molecules comprising a nucleic acid sequence encoding an inverted monomer and/or dimer thereof; recombinant cells engineered to express the inverted monomer and/or dimer thereof, and kits comprising the inverted monomer and/or dimer thereof, nucleic acids encoding the inverted monomer and/or dimer thereof or recombinant cells expressing the inverted monomer and/or dimer thereof.

Nucleic Acid Sequences Encoding Inverted Monomers and/or Dimers Thereof:

In some embodiments, the inverted monomer and/or dimer thereof is produced by recombinant methods using a nucleic acid sequence encoding the inverted monomer and/or dimer thereof (or fusion protein comprising the inverted monomer and/or dimer thereof). The nucleic acid sequence encoding the desired the inverted monomer and/or dimer thereof can be synthesized by chemical means using an oligonucleotide synthesizer.

In some embodiments, the inverted monomer and/or dimer thereof is produced by recombinant methods using a nucleic acid sequence encoding the inverted monomer and/or dimer thereof (or a fusion protein comprising the inverted monomer and/or dimer thereof). The nucleic acid sequence encoding the desired inverted monomer and/or dimer thereof can be synthesized by chemical means using an oligonucleotide synthesizer.

The nucleic acid molecules are not limited to sequences that encode polypeptides; some or all of the non-coding sequences that lie upstream or downstream from a coding sequence can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the inverted monomer and/or dimer thereof (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the inverted monomer and/or dimer thereof may be obtained from various commercial sources that provide custom made nucleic acid sequences. Amino acid sequence variants of the inverted monomer and/or dimer thereof of the present disclosure can be prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding an inverted monomer and/or dimer thereof of the disclosure and expressing those sequences in a suitably transformed host include, but are not limited to, using a PCR-assisted mutagenesis technique. Mutations that consist of deletions or additions of amino acid residues to an inverted monomer and/or dimer thereof can also be made with standard recombinant techniques. In the event of a deletion or addition, the nucleic acid molecule encoding an inverted monomer and/or dimer thereof is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

An inverted monomer and/or dimer thereof of the present disclosure may be produced recombinantly not only directly, but also as a fusion polypeptide with a heterologous polypeptide, e.g., a signal sequence or other polypeptide having a specific cleavage site at the N-terminus or C-terminus of the inverted monomer. In some embodiments, the nucleic acid molecule further comprises a nucleic acid sequence encoding a signal peptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the inverted monomer and/or dimer thereof from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the inverted monomer and/or dimer thereof into the culture medium as described in Singh, U.S. Pat. No. 7,198,919 B1 issued Apr. 3, 2007. In some embodiments, the signal peptide comprises an endogenous or wild-type IL10 signal peptide. In some embodiments, the signal peptide comprises the amino acid sequence of the human IL10 polypeptide: MHSSALLCCLVLLTGVRA (SEQ ID NO:86). In some embodiments, the signal peptide comprises the amino acid sequence of murine IL10 polypeptide: MPGSALLCCLLLLTGMRI (SEQ ID NO:87).

In the event the inverted monomer and/or dimer thereof is to be expressed as a chimera (e.g., a fusion protein comprising an inverted monomer and/or dimer thereof and a heterologous polypeptide sequence), the chimeric protein can be encoded by a hybrid nucleic acid molecule comprising a first sequence that encodes all or part of the inverted monomer and/or dimer thereof and a second sequence that encodes all or part of the heterologous polypeptide. For example, subject inverted monomer and/or dimers thereof described herein may be fused to a hexa-/octa-histidine tag (“HHHHHH” and “HHHHHHHH” disclosed as SEQ ID NOS 139-140, respectively) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the inverted monomer and/or dimers thereof. For example, the N-terminus may be linked to a targeting domain and the C-terminus linked to a hexa-histidine (His6) tag (SEQ ID NO: 139) purification handle.

The complete amino acid sequence of the polypeptide (or fusion/chimera) to be expressed can be used to construct a back-translated gene. A DNA oligomer containing a nucleotide sequence encoding an inverted monomer and/or dimer thereof can be synthesized. For example, several small oligonucleotides coding for portions of the desired polypeptide can be synthesized and then ligated. The individual oligonucleotides typically contain 5′ or 3′ overhangs for complementary assembly.

### Codon Optimization:

In some embodiments, the nucleic acid sequence encoding the inverted monomer and/or dimer thereof may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

### Expression Vectors:

Once assembled (by synthesis, site-directed mutagenesis or another method), the nucleic acid sequence encoding an inverted monomer and/or dimer thereof can be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

In some embodiments, such as where the first and second inverted monomers of the dimer are different such as in the context of heterodimeric inverted monomers, the vector comprises a first nucleic acid sequence encoding the first inverted monomer and as second nucleic acid sequence encoding the second inverted monomer where the first and second nucleic acid sequences are operably linked to an expression control element (e.g. a promoter) and the first and second nucleic acid sequences are separated by a sequence which facilitates co-expression (e.g. an IRES or T2A sequence). Alternatively, the vector comprises a first nucleic acid sequence encoding the first inverted monomer and a second nucleic acid sequence encoding the second inverted monomer where the first and second nucleic acid sequences are each operably linked to an expression control sequence, the expression control sequences being the same or different.

To facilitate efficient expression of the recombinant polypeptide, the nucleic acid sequence encoding the polypeptide sequence to be expressed can be operably linked to transcriptional and translational regulatory control sequences that are functional in the chosen expression host.

### Selectable Marker:

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences:

Expression vectors for an inverted monomer and/or dimer thereof of the present disclosure contain a regulatory sequence that is recognized by the host organism and is operably linked to a nucleic acid sequence encoding the inverted monomer and/or dimer thereof. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject inverted monomer and/or dimer thereof, particularly as regards potential secondary structures.

### Promoters:

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers:

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus.

Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells:

The present disclosure further provides prokaryotic or eukaryotic cells that contain and express one or more nucleic acid molecules that encoding an inverted monomer and/or dimer thereof. A cell of the present disclosure is a transfected cell, i.e., a cell into which a nucleic acid molecule, for example a nucleic acid molecule encoding an inverted monomer and/or dimer thereof, has been introduced by means of recombinant DNA techniques.

In some embodiments, such as where the first and second inverted monomers of a dimer thereof are different such as in the context of heterodimeric inverted monomers, the recombinantly modified cell comprises a vector, the vector comprising a first nucleic acid sequence encoding a first inverted monomer and a second nucleic acid sequence encoding the second inverted monomer where the first and second nucleic acid sequences are operably linked to a single expression control sequence and the first and second nucleic acid sequences are separated by a sequence which facilitates co-expression. In other embodiments, the recombinantly modified cell comprises a vector, the vector comprising a first nucleic acid sequence encoding a first inverted monomer and a second nucleic acid sequence encoding the second inverted monomer where the first and second nucleic acid sequences are each operably linked to an expression control sequence. In other embodiments, where the first and second inverted monomers of the dimer are different such as in the context of heterodimeric inverted monomers, the recombinantly modified cell may comprise two vectors, first, the vector comprising a first nucleic acid sequence encoding a first inverted monomer operably linked to an expression control sequence and a second vector comprising a nucleic acid sequence encoding the second inverted monomer. In some embodiments, the recombinantly modified cell is a prokaryotic cell, such as a bacterial cell. In some embodiments, the recombinantly modified cell is a eukaryotic cell, such as a mammalian cell.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant inverted monomer can also be made in eukaryotes, such as yeast or human cells. Suitable eukaryotic host cells include insect cells (examples of baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerevisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The inverted monomer and/or dimers thereof may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, an inverted monomer obtained will be glycosylated or unglycosylated depending on the host organism used to produce the polypeptide. If bacteria are chosen as the host then the inverted monomer produced will be unglycosylated. Eukaryotic cells, on the other hand, will typically result in glycosylation of the inverted monomer.

In some embodiments, it is possible that an amino acid sequence (particularly a CDR sequence) of an sdAb to be incorporated into an inverted monomer and/or dimer thereof may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the N-linked glycosylation motif is disrupted by the incorporation of conservative amino acid substitution of the Asn (N) residue of the N-linked glycosylation motif.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection:

The expression constructs of the can be introduced into host cells to thereby produce an inverted monomer and/or dimer thereof disclosed herein. The expression vector comprising a nucleic acid sequence encoding the inverted monomer and/or dimer thereof can be introduced into the prokaryotic or eukaryotic host cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins:

Recombinantly produced inverted monomer and/or dimer thereof can be recovered from the culture medium as a secreted polypeptide if a secretion leader sequence is employed. Alternatively, the inverted monomers and/or dimers thereof can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g., affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinant inverted monomer and/or dimer thereof expressed by the transformed host can be purified according to any suitable method. Recombinant inverted monomers and/or dimers thereof can be isolated from inclusion bodies generated in E. coli, or from conditioned medium from either mammalian or yeast cultures producing a given polypeptide using cation exchange, gel filtration, and or reverse phase liquid chromatography. The substantially purified forms of the recombinant inverted monomers and/or dimers thereof can be purified from the expression system using routine biochemical procedures, and can be used, e.g., as therapeutic agents, as described herein.

In some embodiments, where the inverted monomer and/or dimer thereof is expressed with a purification tag as discussed above, this purification handle may be used for isolation of the inverted monomer and/or dimer thereof from the cell lysate or cell medium. Where the purification tag is a chelating peptide, methods for the isolation of such molecules using immobilized metal affinity chromatography are well known in the art. See, e.g., Smith, et al. U.S. Pat. No. 4,569,794.

The biological activity of the inverted monomer and/or dimer thereof recovered can be assayed for activity by any suitable method known in the art and may be evaluated as substantially purified forms or as part of the cell lysate or cell medium when secretion leader sequences are employed for expression.

### Pharmaceutical Formulations

In some embodiments, the subject inverted monomer and/or dimer thereof (and/or nucleic acids encoding the inverted monomer and/or dimer thereof or recombinant cells incorporating a nucleic acid sequence and modified to express the inverted monomer and/or dimer thereof) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the inverted monomer and/or dimer thereof is to be administered to the subject in need of treatment

### Carriers:

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor EL™ (BASF, Parsippany, N.J.) or phosphate buffered saline (PBS).

### Buffers:

The term “buffers” includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

### Dispersions:

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Preservatives:

The pharmaceutical formulations for parenteral administration to a subject should be sterile and should be fluid to facilitate easy syringability. It should be stable under the conditions of manufacture and storage and are preserved against the contamination.

Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

### Tonicity Agents:

In many cases, it will be preferable to include isotonic agents, for example, sugars, polyalcohols such as mannitol, sorbitol, sodium chloride in the composition.

### Routes of Administration:

In some embodiments the therapeutic methods of the present disclosure involve the administration of a pharmaceutical formulation comprising an inverted monomer and/or dimer thereof (and/or nucleic acids encoding the inverted monomer and/or dimer thereof or recombinantly modified host cells expressing the inverted monomer and/or dimer thereof) to a subject in need of treatment. The pharmaceutical formulation comprising an inverted monomer and/or dimer thereof of the present disclosure may be administered to a subject in need of treatment or prophyaxis by a variety of routes of administration, including parenteral administration, oral, topical, or inhalation routes.

### Parenteral Administration:

In some embodiments, the methods of the present disclosure involve the parenteral administration of a pharmaceutical formulation comprising an inverted monomer and/or dimer thereof (and/or nucleic acids encoding the inverted monomer and/or dimer thereof or recombinantly modified host cells expressing the inverted monomer and/or dimer thereof) to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe.

### Oral Administration:

In some embodiments, the methods of the present disclosure involve the oral administration of a pharmaceutical formulation comprising an inverted monomer and/or dimer thereof (and/or nucleic acids encoding the inverted monomer and/or dimer thereof or recombinantly modified host cells expressing the inverted monomer and/or dimer thereof) to a subject in need of treatment. Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

### Inhalation Formulations:

In some embodiments, the methods of the present disclosure involve the inhaled administration of a pharmaceutical formulation comprising a inverted monomer and/or dimer thereof (and/or nucleic acids encoding the inverted monomer and/or dimer thereof or recombinantly modified host cells expressing the inverted monomer and/or dimer thereof) to a subject in need of treatment. In the event of administration by inhalation, subject inverted monomers and/or dimers thereof, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

### Mucosal and Transdermal Formulations:

In some embodiments, the methods of the present disclosure involve the mucosal or transdermal administration of a pharmaceutical formulation comprising an inverted monomer and/or dimer thereof (and/or nucleic acids encoding the inverted monomer and/or dimer thereof or recombinantly modified host cells expressing the inverted monomer and/or dimer thereof) to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

### Extended Release and Depot Formulations:

In some embodiments of the method of the present disclosure, the inverted monomer and/or dimer thereof is administered to a subject in need of treatment in a formulation to provide extended release of the inverted monomer and/or dimer thereof agent. Examples of extended release formulations of the injectable compositions can be brought about by including in the composition an agent which delays absorption, for example, aluminum monostearate and gelatin. In one embodiment, the subject inverted monomer and/or dimer thereof or nucleic acids are prepared with carriers that will protect the inverted monomer and/or dimer thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Administration of Nucleic Acids Encoding the Inverted Monomer and/or Dimer Thereof

In some embodiments of the method of the present disclosure, delivery of the inverted monomer and/or dimer thereof to a subject in need of treatment is achieved by the administration of a nucleic acid encoding the inverted monomer and/or dimer thereof. Methods for the administration nucleic acid encoding the inverted monomer and/or dimer thereof to a subject is achieved by transfection or infection using methods known in the art, including but not limited to the methods described in McCaffrey et al. (Nature (2002) 418:6893), Xia et al. (Nature Biotechnol. (2002) 20:1006-1010), or Putnam (Am. J. Health Syst. Pharm. (1996) 53: 151-160 erratum at Am. J. Health Syst. Pharm. (1996) 53:325). In some embodiments, the inverted monomer and/or dimer thereof is administered to a subject by the administration of a pharmaceutically acceptable formulation of recombinant expression vector comprising a nucleic acid sequence encoding the inverted monomer and/or dimer thereof operably linked to one or more expression control sequences operable in a mammalian subject. In some embodiments, the expression control sequence may be selected that is operable in a limited range of cell types (or single cell type) to facilitate the selective expression of the inverted monomer and/or dimer thereof in a particular target cell type. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways in a human cell. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in the target cell type.

In some embodiments, particularly for treatment of diseases of the intestinal tract or bacterial infections in a subject, the nucleic acid encoding the inverted monomer and/or dimer thereof may be delivered to the subject by the administration of a recombinantly modified bacteriophage vector encoding the inverted monomer and/or dimer thereof. As used herein, the terms “procaryotic virus,” “bacteriophage” and “phage” are used interchangeably hereinto describe any of a variety of bacterial viruses that infect and replicate within a bacterium. Bacteriophage selectively infect procaryotic cells, restricting the expression of the inverted monomer and/or dimer thereof to procaryotic cells in the subject while avoiding expression in mammalian cells. A wide variety of bacteriophages capable of selection a broad range of bacterial cells have been identified and characterized extensively in the scientific literature. In some embodiments, the phage is modified to remove adjacent motifs (PAM). Elimination of Cas9 sequences from the phage genome reduces ability of the Cas9 endonuclease of the target procaryotic cell to neutralize the invading phage encoding the inverted monomer and/or dimer thereof.

Administration of Recombinantly Modified Cells Expressing the Inverted Monomer or Dimer thereof.

In some embodiments of the methods of the present disclosure, delivery of the inverted monomer and/or dimer thereof to a subject in need of treatment is achieved by the administration of recombinant host cells modified to express the inverted monomer and/or dimer thereof. Recombinant host cells may be administered in the therapeutic and prophylactic applications described herein. In some embodiments, the recombinant host cells are mammalian cells, e.g., human cells. In some embodiments, the recombinant host cells are procaryotic cells, e.g., bacterial cells associated with the intestinal flora such as E. coli or Lactobacillus lacti.

In some embodiments, the nucleic acid sequence encoding the inverted monomer and/or dimer thereof (or vectors comprising same) may be maintained extrachromosomally in the recombinantly modified host cell for administration. In other embodiments, the nucleic acid sequence encoding the inverted monomer and/or dimer thereof may be incorporated into the genome of the host cell to be administered using at least one endonuclease to facilitate incorporate insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37(16):5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23(3):967-973, a TALEN-nuclease, a Cas9 endonuclease from CRISPR system as or a modified restriction endonuclease to extended sequence specificity (Eisenschmidt, et al. 2005; 33(22): 7039-7047).

In some embodiments, particularly for administration of inverted monomers and/or dimers thereof to the intestinal tract, the inverted monomer and/or dimer thereof may be delivered to the subject by a recombinantly modified procaryotic cell (e.g., Lactobacillus lacti). The use of engineered procaryotic cells for the delivery of recombinant proteins to the intestinal tract are known in the art. See, e.g., Lin, et al. (2017) Microb Cell Fact 16:148. In some embodiments, the engineered bacterial cell expressing the inverted monomer and/or dimer thereof may be administered orally, typically in aqueous suspension, or rectally (e.g., enema).

### Methods of Use

The present disclosure further provides methods of treating a subject suffering from a disease disorder or condition by the administration of a therapeutically effective amount of: (a) a inverted IL10 monomer and/or dimer thereof, (b) a pharmaceutically acceptable formulation containing as an active ingredient an inverted IL10 monomer and/or dimer thereof; (c) a recombinant non-viral or eucaryotic viral or bacteriophage vectors comprising a nucleic acid sequence encoding an inverted monomer and/or dimer thereof operably linked to one or more expression control sequences, (d) a recombinant procaryotic or mammalian cells genomically modified to comprise a nucleic acid sequence encoding the inverted monomer and/or dimer thereof operably linked to one or more expression control sequences, or (e) a recombinant procaryotic or mammalian cell comprising a nucleic acid encoding an inverted monomer and/or dimer thereof operably linked to one or more expression control sequences.

### Dosing:

The biased IL10 inverted monomers (and dimers thereof) of the present disclosure exhibit a broadened therapeutic window as compared to wild type IL10. The “therapeutic window” refers to the range of dosing of the biased IL10 inverted monomer (and/or dimers thereof) that provides a concentration of the biased IL10 inverted monomer (or dimer thereof) in the subject sufficient to provide substantial level of an IL10 activity (e.g., STAT3 signaling) in monocytes but that is less than the dose that results in a concentration in the subject sufficient to induce a substantial level of the IL10 activity signaling in T cells. Consequently, in some embodiments the biased IL10 inverted monomers are dosed to a subject at a level that results in a concentration in the subject sufficient to provide substantial level STAT3 signaling in monocytes but that is less than the dose that results in a concentration in the subject sufficient to induce a substantial level of STAT3 signaling in T cells. In some embodiments, the present disclosure provides a method of treating a subject suffering from an inflammatory or autoimmune disease, disorder or condition characterized by the administration of a therapeutically effective amount of an inverted IL10 monomer and/or dimer thereof (or a pharmaceutically acceptable formulation containing as an active ingredient an inverted IL10 monomer and/or dimer thereof) wherein the therapeutically effective amount is that amount that results in a concentration in the subject sufficient to provide substantial level STAT3 signaling in monocytes but that is less than the dose that results in a concentration in the subject sufficient to induce a substantial level of STAT3 signaling in T cells.

In one embodiment, the present disclosure provides a method of treating a subject suffering from an inflammatory or autoimmune disease, disorder, or condition by the administration of a therapeutically effective amount of an inverted IL10 monomer and/or dimer thereof (or a pharmaceutically acceptable formulation containing as an active ingredient an inverted IL10 monomer and/or dimer thereof) at a dose level that monomer that maximizes the ratio of the Emax of an IL10 activity on monocytes relative to the Emax of such IL10 activity on T cells. In some embodiments, the present disclosure provides a methods of treating a subject suffering from a disease disorder or condition by the administration of a therapeutically effective amount of an inverted IL10 monomer and/or dimer thereof (or a pharmaceutically acceptable formulation containing as an active ingredient an inverted IL10 monomer and/or dimer thereof) wherein the therapeutically effective amount is a dose to provide a concentration that is: (a) greater than the STAT3 EC₂₀ on activated human monocytes, alternatively greater than the STAT3 EC₃₀ on activated human monocytes, alternatively greater than the STAT3 EC₄₀ on activated human monocytes, alternatively greater than the STAT3 EC₅₀ on activated human monocytes, alternatively greater than the STAT3 EC₆₀ on activated human monocytes, STAT3 EC₇₀ on activated human monocytes, alternatively greater than the STAT3 EC₅₀ on activated human monocytes, alternatively greater than the STAT3 EC₉₀ on activated human monocytes, alternatively greater than the Eₘₐₓ (EC₁₀₀) on activated human monocytes; and (b) less than the STAT3 EC₄₀ on activated human T cells, alternatively less than the STAT3 EC₃₀ on activated human T cells, alternatively less than the STAT3 EC₂₀ on activated human T cells, alternatively less than the STAT3 EC₁₀ on activated human T cells, alternatively less than the STAT3 EC₅ on activated human T cells. In some embodiments, the present disclosure provides a methods of treating a subject suffering from a disease disorder or condition by the administration of a therapeutically effective amount of an inverted IL10 monomer and/or dimer thereof (or a pharmaceutically acceptable formulation containing as an active ingredient an inverted IL10 monomer and/or dimer thereof) wherein the therapeutically effective amount is a dose to provide a concentration that is: greater than the STAT3 EC₂₀ on activated human monocytes but less than the STAT3 EC₃₀ on activated human T cells; alternatively greater than the STAT3 EC₅₀ on activated human monocytes but less than the STAT3 EC₂₀ on activated human T cells; alternatively greater than the STAT3 EC₇₀ on activated human monocytes but less than the STAT3 EC₂₀ on activated human T cells; alternatively greater than the STAT3 EC₅₀ on activated human monocytes but less than the STAT3 EC₂₀ on activated human T cells; alternatively greater than the STAT3 Eₘₐₓ on activated human monocytes but less than the STAT3 EC₃₀ on activated human T cells, or alternatively greater than the STAT3 EC₅₀ on activated human monocytes but less than the STAT3 EC₁₀ on activated human T cells.

### Inflammatory and Autoimmune Disorders

Disorders amenable to treatment with an inverted monomer and/or dimer thereof (including pharmaceutically acceptable formulations comprising an inverted monomer and/or dimer thereof and/or the nucleic acid molecules that encode them including recombinant viruses encoding such an inverted monomer and/or dimer thereof) of the present disclosure include inflammatory or autoimmune diseases including but not limited to, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome(Seronegativity, Enthesopathy, Arthropathy Syndrome).

Other examples of proliferative and/or differentiative disorders amenable to treatment with an inverted monomer and/or dimer thereof of the disclosure (including pharmaceutically acceptable formulations comprising inverted monomers and/or dimers thereof and/or the nucleic acid molecules that encode them including recombinant viruses encoding such inverted monomers and/or dimers thereof) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of inflammatory or autoimmune skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

The compositions of the present disclosure (including pharmaceutically acceptable formulations comprising inverted monomer and/or dimer thereof and/or the nucleic acid molecules that encode them including recombinant viruses encoding such inverted monomers and/or dimers thereof) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

### Ulcerative Colitis

In some embodiments, the present disclosure provides a method of treating a mammalian subject suffering from ulcerative colitis, the method comprising the step of administering to the subject a hIL10 inverted monomer or hIL10 inverted monomer dimer (or pharmaceutical formulation comprising a hIL10 inverted monomer or hIL10 inverted monomer dimer) of the present disclosure, the administering providing an improvement in one or more symptoms of the ulcerative colitis. In some embodiments, the method comprises administering to the subject an inverted monomer of formula 1 or formula 2 or pharmaceutical formulation comprising an inverted monomer of formula 1 or formula 2, the administering providing an improvement in one or more symptoms of the ulcerative colitis. In one embodiment, the present disclosure provides a method of treating a mammalian subject suffering from ulcerative colitis the method comprising the step of administering to the subject a hIL10 inverted monomer or hIL10 inverted monomer dimer having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS: 10, 11, 27, 90, 91, 92, 93, 94, 95, 96, 97, 98, 99, 53, 108, 109, 110, 111, 112, 113, 114, 46, 47, 48, 49, 50, 51, 52, 53, 54, 121, 122, 123, 124, 125, and 126 wherein the administering providing an improvement in one or more symptoms of the ulcerative colitis.

### IFNγ Induced Anemia:

In some embodiments, the present disclosure provides a method of treating and/or preventing IFNγ induced anemia in a subject. the method comprising the step of administering to the subject a therapeutically effective amount of a fused IL10 dimer of the present disclosure wherein the administering results in an amelioration of one or more symptoms of the IFNγ induced anemia and/or a reduction in serum IFNγ levels in the subject. In some embodiments, the method comprises administering to the subject a therapeutically effective amount of an inverted monomer of formula 1 wherein the administering results in an amelioration of one or more symptoms of the IFNγ induced anemia and/or a reduction in serum IFNγ levels in the subject. IFNγ induced anemia is a frequent complication in patients with chronic inflammatory diseases. Chronic inflammation may arise from a variety of conditions such as infectious disease, AIDS, malignancies and/or autoimmune diseases such as inflammatory bowel disease, rheumatoic arthritis. Activated T cells secrete IFNγ and interleukin 2 (IL-2). IFNγ has been observed to increase the cytotoxic effects of TNFα. The compositions of the present disclosure were evaluated in a murine model of IFNγ induced anemia (Example 32). In one embodiment, the present disclosure provides a method of treating and/or preventing IFNγ induced anemia in a subject the method comprising the step of administering to the subject a hIL10 inverted monomer or hIL10 inverted monomer dimer having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS: 10, 11, 27, 90, 91, 92, 93, 94, 95, 96, 97, 98, 99, 53, 108, 109, 110, 111, 112, 113, 114, 46, 47, 48, 49, 50, 51, 52, 53, 54, 121, 122, 123, 124, 125, and 126, wherein the administering providing an improvement in one or more symptoms of the IFNγ induced anemia.

### Macrophage Activation Syndrome

In some embodiments, the present disclosure provides a method of treating and/or preventing macrophage activation syndrome (MAS) in a subject. the method comprising the step of administering to the subject a therapeutically effective amount of an inverted IL10 monomer of formula 1 or formula 2 of the present disclosure. Macrophage activation syndrome (MAS) is a potentially life-threatening complication of systemic inflammatory disorders, including but not limited to systemic juvenile idiopathic arthritis (sJIA), Kawasaki disease, systemic lupus erythematosus (SLE) and infections, particularly Epstein-Barr Virus (EBV) infection, malignancy, and primary immunodeficiencies. Elevated CD163 is associated with MAS. In some embodiments, the present disclosure provides a method of treating and/or preventing macrophage activation syndrome (MAS) in a subject. the method comprising the step of administering to the subject a therapeutically effective amount of a fused IL10 dimer of the present disclosure, wherein the administering results in an amelioration of one or more symptoms of the macrophage activation syndrome. In one embodiment, the present disclosure provides a method of treating and/or preventing macrophage activation syndrome in a subject the method comprising the step of administering to the subject a hIL10 inverted monomer or hIL10 inverted monomer dimer having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% or 100% sequence identity to a polypeptide selected from the group consisting of SEQ ID NOS: 10, 11, 27, 90, 91, 92, 93, 94, 95, 96, 97, 98, 99, 53, 108, 109, 110, 111, 112, 113, 114, 46, 47, 48, 49, 50, 51, 52, 53, 54, 121, 122, 123, 124, 125, and 126, wherein the administering providing an improvement in one or more symptoms of the macrophage activation syndrome.

As previously discussed, chronic inflammation is associated with the development of a wide variety of cancers. See. E.g. Coussens and Werb (2002) Nature 420: 860-867. Consequently, compositions of the inverted of the present disclosure may be used in the treatment and/or prevention of cancers arising from chronic inflammation. In one embodiment, the cancer associated with chronic inflammation is selected from the group consisting of colon cancer, colorectal cancer, pancreatic cancer and liver cancer. In one embodiment, the present disclosure provides a method of preventing the development of cancer associated with chronic inflammation in a human subject by the administration to the subject of a prophylactically effective amount of a composition comprising, vector encoding or recombinant cell expressing an inverted hIL10 monomer (or dimer thereof) of the present disclosure.

### Methods for Modulating IL10-Mediated Signaling

In another aspect, the disclosure provides methods for modulating IL10-mediated signaling in a subject. In some embodiments, the method comprises administering to the subject an effective amount of a pharmaceutical composition to the subject, where the pharmaceutical composition comprises an inverted monomer and/or dimer thereof described herein, a nucleic acid molecule encoding an inverted monomer and/or dimer thereof described herein, or a recombinantly modified cell comprising a nucleic acid molecule encoding an inverted monomer and/or dimer thereof described herein. In some embodiments, the pharmaceutical composition comprises a pharmaceutically acceptable carrier.

In some embodiments, the method for modulating IL10-mediated signaling in a subject comprises determining STAT3-mediated signaling in one or more cells obtained from the subject. In some embodiments, the STAT3-mediated signaling is determined by an assay selected from the group consisting of a gene expression assay, a phospho-flow signaling assay, and an enzyme-linked immunosorbent assay (ELISA). In some embodiments, the STAT3-mediated signaling in the subject is reduced by about 20% to about 100% compared to a reference level. In some embodiments, the administered composition results in a reduced capacity to induce expression of a pro-inflammatory gene selected from IFN-γ, granzyme B, granzyme A, perforin, TNF-α, GM-CSF, and MIP1α in the subject.

### Kits

Also provided are kits comprising the inverted monomers and/or dimers thereof of the disclosure. In some embodiments, the kit comprises one or more components for modulating IL10-mediated signaling in a subject, or treating a health condition in a subject in need thereof, wherein the components are selected from an inverted monomer and/or dimer thereof described herein, a nucleic acid molecule encoding an inverted monomer and/or dimer thereof described herein, a recombinantly modified cell comprising a nucleic acid molecule encoding a inverted monomer and/or dimer thereof described herein, or a pharmaceutical composition comprising one of more the components. In some embodiments, the pharmaceutical composition of the kit comprises a pharmaceutically acceptable carrier.

### EXAMPLES

The following Examples are provided to illustrate, but not to limit the claimed invention.

### Example 1: Methods

This example describes methods for making and testing the biophysical characteristics of the invented monomer and dimers of the disclosure.

Protein expression and Purification

Protein-coding DNA sequences were inserted into pEXSyn 2.0 between EcoRI and NotI restriction sites as C-terminal His-tag fusions with either the native signal peptide for human molecules (DNA seq:
ATGCACAGCAGCGCCCTGCTGTGCTGCCTGGTGCTGCTGACCGGCGTGAGAGCC (SEQ ID NO:85); amino acid seq: MHSSALLCCLVLLTGVRA (SEQ ID NO:86)) or the mouse Ig heavy chain signal peptide (DNA seq:
ATGGGATGGTCCTGCATCATCCTGTTTCTGGTGGCTACCGCCACCGGTGTGCACA GC (SEQ ID NO:88); amino acid seq: MGWSCIILFLVATATGVHS (SEQ ID NO:89)) for mouse molecules. The nucleic acid and amino acid sequences of the human and mouse inverted monomers and dimers are provided in Tables 5-12.

Proteins were expressed in expi293 cells by transient transfection. The polypeptides containing the C-terminal His-tag were purified from harvested cell culture supernatants using Ni-affinity chromatography. Culture supernatants were supplemented with 5 mM Imidazole and were purified on gravity columns containing Ni-Excel (Cytiva) resin and equilibrated in PBS containing 5 mM Imidazole. Washes and elutions were carried out in PBS supplemented with 5 and 250 mM Imidazole, respectively. Proteins were dialyzed into PBS before storage and testing. Protein purity and quality was evaluated using SD-PAGE, LCMS and analytical SEC.

### Protein PEGylation

Purified proteins were concentrated to 2-3 mg/ml in a 10k Amicon (Millipore) in 100 mM Sodium Phosphate buffer pH 6.3, 100 mM NaCl. Protein was PEGylated at the N-terminus via aldehyde chemistry using 20 kDa linear PEG (Sunbright® ME-200AL, NOF) for native IL10 and 40 kDa branched PEG (Sunbright® GL2-400AL3) for the inverted monomer. The PEGylation reaction was carried out at room temperature with end-to-end rotation for 24-48 hours with addition of 5-fold molar excess of PEG over protein and 10 mM sodium cyanoborohydride (NaCNBH3, Sigma). PEGylation progress was evaluated by analytical SEC. The PEGylation reaction mixture was diluted 10-fold and purified over a cation exchange column (SP HP, Cytiva) with step-elution using 20 mM Sodium Phosphate pH 6.3, 0.7M NaCl to separate the protein from free PEG and NaCNBH3. The eluted protein contains a mix of unPEGylated, mono and multi-PEGylated protein species and was further purified by size exclusion chromatography with a Superose 6 increase column (Cytiva) equilibrated in Phosphate-buffered Saline (PBS). Elution peaks containing the PEGylated protein were pooled and concentrated to 0.5-2 mg/ml and sterile-filtered before use.

### Surface Plasmon Resonance (Biacore) Binding

All experiments were conducted in 10 mM Hepes, 150 mM NaCl, 0.05% (v/v) Polysorbate 20 (PS20) and 3 mM EDTA (HBS-EP+buffer) on a Biacore T200 instrument equipped with Anti-human capture (AHC)-CM5 chip (Cytiva).

Human or mouse IL10RuaFc (human: R&D systems #9044-RI; mouse: Sino biologicals #51298-MO2H) were captured on an AHC surface. Since IL10 analytes can be bivalent, ligand capture density was set to 12-25 RU to restrict Rₘₐₓ to <8.5 RU (determined in a separate experiment) to measure binding kinetics under non-avid conditions. IL10 analytes were injected at 0.74, 2.22, 6.66, 20, 60, 180 nM in high performance mode (90 second association, 360 second dissocation) followed by surface regeneration with 3M MgCl₂ (40 seconds, 30 μL/min). Buffer-subtracted sensograms were processed with Biacore T200 Evaluation Software and globally fit with a 1:1 Langmuir binding model to extract kinetics and affinity constants (ka, kd, KD). Rₘₐₓ ranged from 4 to 8.5 RU, indicating surface density supporting true binding kinetics measurements in the absence of avidity.

Binding to human or mouse IL10RP is not reported since the affinity of IL10 for IL10Rβ is expected to be weak (μM to mM) and not detectable under these conditions.

Affinity of the inverted monomer to IL10Ra showed a 3-6 fold reduction in K_{D} compared to IL10 WT. See Tables 3 and 4 below.

TABLE 3
Human molecules

| | | | | Affinity | Rmax | Load | Calc. Rmax | |
|---|---|---|---|---|---|---|---|---|
| Ligand | Analyte | k_{ON} (1/Ms) | k_{OFF} (1/s) | (nM) | (RU) | (RU) | (RU) | Activity |
| hIL10RaFc | hIL10 WT | 2.42E+06 | 1.51E−04 | 0.06 | 7.6 | 22.5 | 12.5 | 61% |
| (R&D 9044- | h_DR1060_AA | 1.62E+06 | 6.17E−04 | 0.38 | 6.8 | 23.7 | 6.8 | 100% |
| RI) | (hIL10 WT | | | | | | | |
| | inverted | | | | | | | |
| | monomer) | | | | | | | |

TABLE 4
Mouse molecules

| | | | | Affinity | Rmax | Load | Calc. Rmax | |
|---|---|---|---|---|---|---|---|---|
| Ligand | Analyte | k_{ON} (1/Ms) | k_{OFF} (1/s) | (nM) | (RU) | (RU) | (RU) | Activity |
| mIL10RaFc(Sino | mIL10 WT | 1.44E+07 | 1.16E−02 | 0.81 | 5.4 | 12.8 | 7.1 | 76% |
| 51298-M02H) | WC161_AA (mIL10 | 2.95E+06 | 1.93E−02 | 6.54 | 4 | 13.4 | 3.8 | 104% |
| | WT inverted | | | | | | | |
| | monomer) | | | | | | | |

### Thermal Stability Measurements

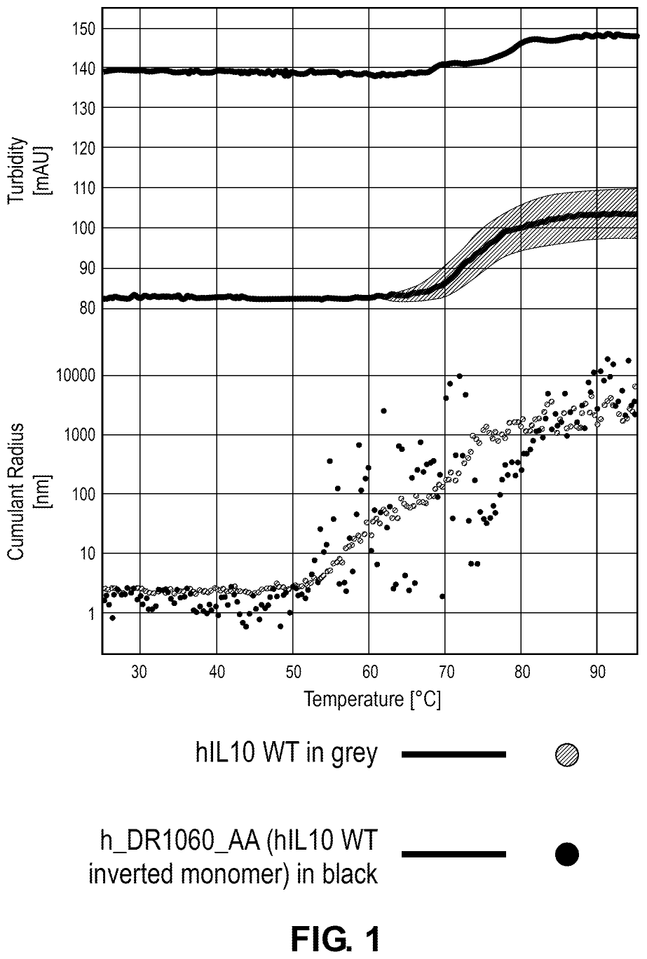

Proteins were formulated at −1 mg/mL in PBS and 0.1 im-filtered to remove aggregates. Each protein was loaded in 4 capillaries and dynamic light scattering (DLS) and turbidity were measured over a temperature ramp from 25 to 95 °C at 1 °C/min on a Prometheus Panta instrument. Unfolding data via differential scanning fluorimetry was not analyzed due to lack of tryptophans in IL10 molecules, which resulted in poor signal. The data indicate that the engineered molecules display equivalent thermal stability to the IL10 WT with aggregation onset (DLS) at −52 °C. The data are shown in FIG. 1.

### Example 2. Evaluation of STAT3 Activity

This example describes methods and results of testing the activity of the polypeptides of the disclosure.

pSTAT3 Flow Cytometric Assay:

PBMCs were purified from healthy non-smoking donor blood collected in leukoreduction system (LRS) chambers using the human Miltenyi MACSprep™ PBMC isolation kit. The purified PBMCs (500,000 cells per well) were stimulated with the IL10 proteins h_DR1061_AA (SEQ ID NO:108) and h_SM0043_AA at concentrations ranging from 0.1 pM-100 nM for 20 min at 37 °C. h_SM0043_AA is a wild type human IL10 sequence expressed in E. coli having the amino acid sequence:

(SEQ ID NO: 166)
MSPGQGTQSENSCTHFPGNLPNMLRDLRDAFSRVKTFFQMKDQLDNLLLK
ESLLEDFKGYLGCQALSEMIQFYLEEVMPQAENQDPDIKAHVNSLGENLK
TLRLRLRRCHRFLPCENKSKAVEQVKNAFNKLQEKGIYKAMSEFDIFINY
IEAYMTMKIRN

The cells were fixed with BD Phosflow™ Fix Buffer I (BD Biosciences, Catalog No. 557870) for 15 mins at 37 °C. The cells were then washed and permeabilized with chilled BD Phosflow™ Perm Buffer III (Catalog No. 558050) overnight at −20 °C. The cells were washed to remove the permeabilization buffer and blocked with Human Trustain Fcx (Biolegend, Catalog No. 422301) and mouse serum for 5 minutes at room temperature. The cells were then treated with the antibody cocktail (listed in Table 14 below) for 1 hour at room temperature.

TABLE 14
Antibody cocktail.

| | | Phos- | | | |
|---|---|---|---|---|---|
| Fluoro- | | phorylation | | | |
| chrome | Antibody | Site | Clone | Catalog# | Vendor |
| BV480 | CD4 | | L200 | 566148 | BD Biosciences |
| BV605 | CD8a | | RPA-T8 | 301040 | Biolegend |
| BV786 | CD56 | | 5.1H11 | 362550 | Biolegend |
| AF488 | pSTAT3 | Tyr705 | D3A7 | 4323S | CST |
| PE-Cy5 | CD3 | | UCHT1 | 555334 | BD Biosciences |
| PerCP-Cy | CD14 | | M5E2 | 301824 | Biolegend |
| 5.5 | | | | | |
| APC | CD20 | | 1412 | 340516 | Biolegend |

Following antibody staining, the cells were washed, fixed and ran on the Cytek® Aurora Spectral flow cytometer. The data was analyzed using the FlowJo software (www.flowjo.com). The various cell lineages were gated using their lineage markers and the geometric mean fluorescence intensity of pSTAT3 expression was calculated on FlowJo. The data is shown in FIGS. 2A and 2B. The data presented shows that the inverted monomeric IL10 molecule (h_DR1060_AA) exhibits decreased pSTAT3 signalling in monocytes and CD8 T cells as compared to the wild type hIL10 molecule (h_SM0043_AA).

### Example 3. Monocyte IL10 Activity Assay

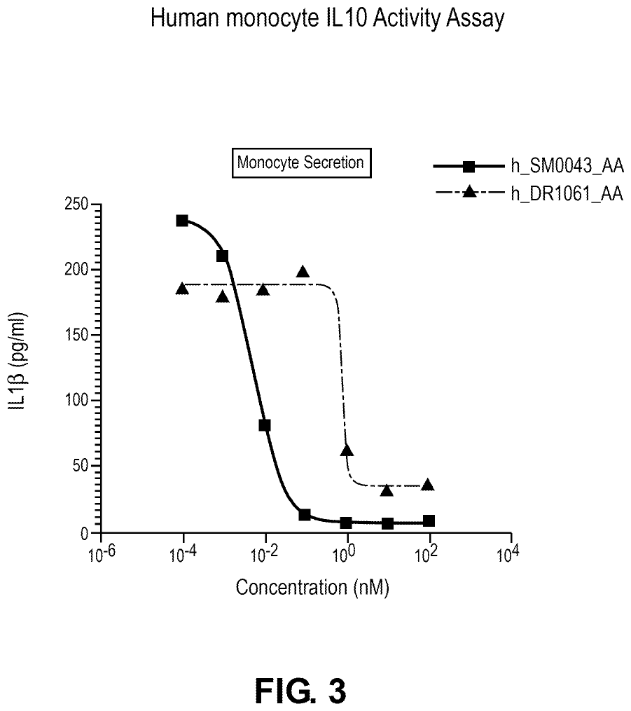

Human PBMCs were isolated from LRS chambers obtained from non-smoking human donors by Ficoll® gradient purification. Human monocytes were purified from the PBMCs by positive selection using Human CD14 MicroBeads (Miltenyi Biotech, Catalog No. 130-050-201) following manufacturer's protocol. Monocytes were treated with 1 ng/ml of LPS (Millipore Sigma, Catalog No. L2630) in the presence of varying concentrations of the IL10 proteins (0.05 pM-200 nM) for 48 hours at 37 °C. Post-treatment, cells were centrifuged at 400g for 5 min and supernatants were collected. Supernatants were assessed for pro-inflammatory cytokines using the MSD kit (Meso Scale Discovery, Catalog No. K151A9H-4) in substantial accordance with the manufacturer's protocol. The data was graphed using Prism software (www.prismsoftware.com). The data is shown in FIG. 3.

The data demonstrate that the inverted monomeric IL10 molecule (h_DR1061_AA) inhibits LPS-induced IL-1β secretion by monocytes, albeit with a 150-fold lower potency compared to the native dimeric IL-10 molecule (h_SM0043_AA).

### Example 4. CD8 T cell blasts IL10 Activity Assay

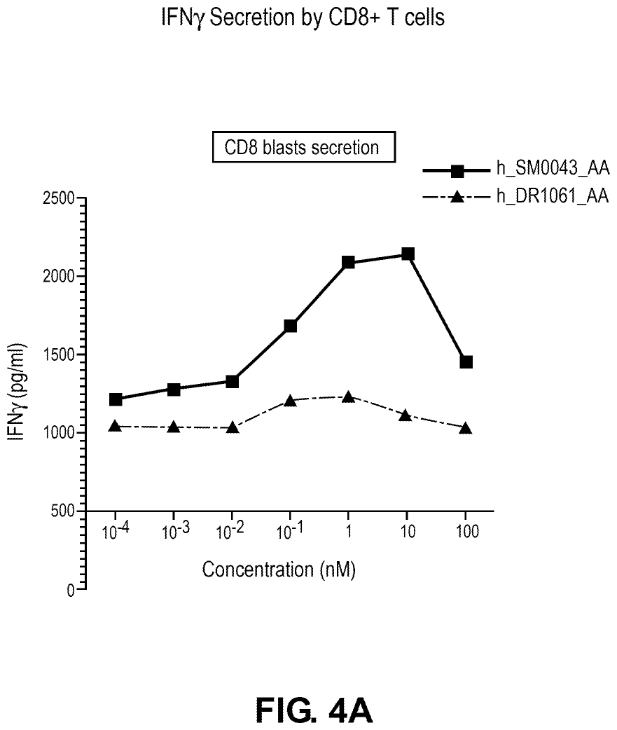

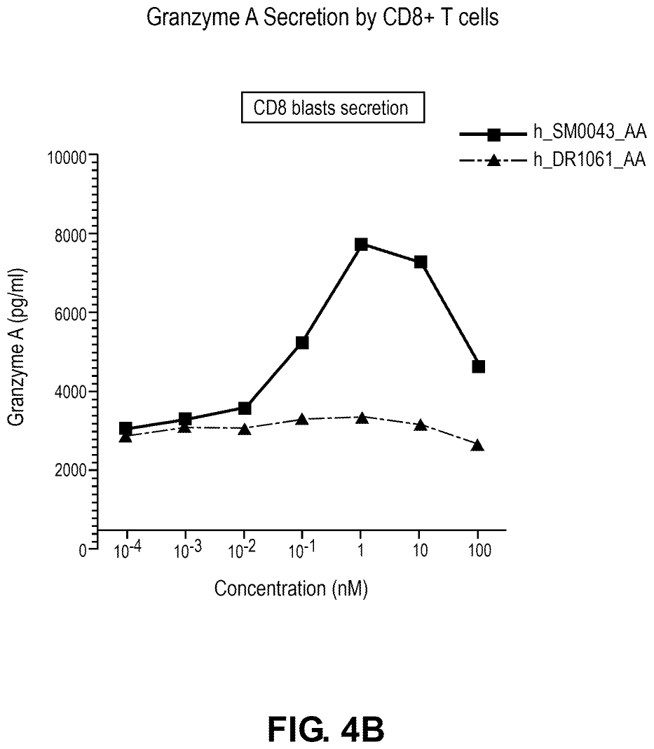

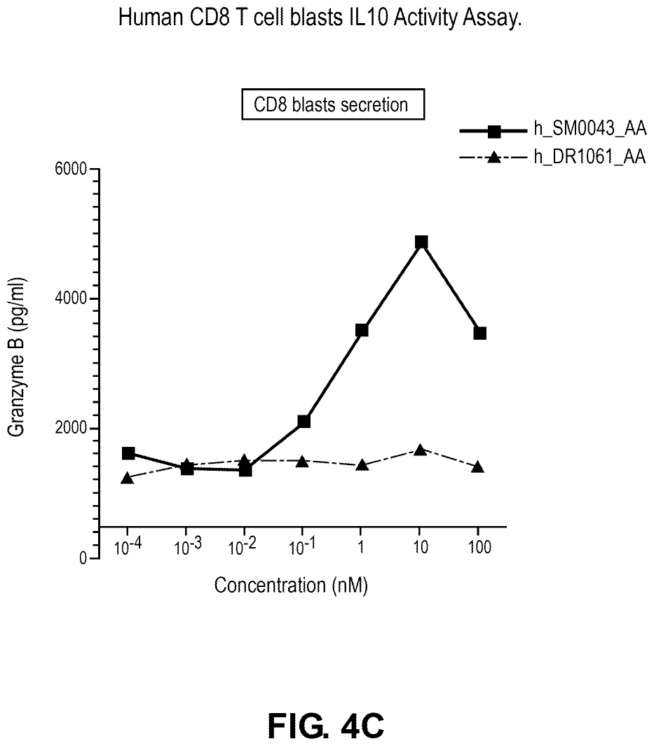

Human PBMCs were isolated from LRS chambers obtained from non-smoking human donors by Ficoll gradient purification. Human CD8 T cells were purified from the PBMCs by negative selection using the CD8+ T Cell Isolation Kit (Miltenyi Biotech-130-096-495) in substantial accordance with the manufacturer's protocol. Isolated CD8 T cells were activated using the human T Cell Activation/Expansion Kit (Miltenyi Biotech, Catalog No. 130-091-441) in the presence of 100 pM human IL2 for 72 hours at 37 °C. The activated CD8 T cells were washed and the beads were removed magnetically following which the cells were treated with varying concentrations of the IL10 proteins (0.05 pM-200 nM) for 72 hours at 37 °C. Post-treatment, cells were centrifuged at 400g for 5 min and the supernatants were collected. Supernatants were assessed for the cytokines IFNγ, Granzyme A and Granzyme B using the MSD kit (Meso Scale Discovery, Catalog No. K151AEL-4) in substantial accordance with the manufacturer's protocol. The data was graphed using Prism software and is shown in FIGS. 4A-4C.

The data demonstrates that compared to native hIL10 dimers (h-SM0043_AA), the inverted monomeric IL10 molecule (h_DR1061_AA) does not induce IFN-γ, Granzyme A or Granzyme B secretion by activated CD8 T cells.

### Example 5. Mouse pSTAT3 Flow Cytometric Assay

Spleens were removed from naïve C57BL/6 mice. Splenocytes were isolated by crushing the spleens with the plunger end of a syringe in complete RPMI medium (Gibco RPMI 1640+10% FBS+1% penicillin-streptomycin) and straining through a 70 pM strainer. The cells were centrifuged at 400g for 5 min and the supernatant was discarded. The cell pellet was resuspended in 10 ml ammonium-chloride-potassium (ACK) lysis buffer (ThermoFisher) and allowed to sit at 37 °C for 2 min. The cells were washed and resuspended in complete RPMI. The purified splenocytes (500,000 cells per well) were stimulated with IL10 proteins at concentrations ranging from 0.1 pM -100 nM for 20 min at 37 °C. The cells were fixed with BD Phosflow™ Fix Buffer I (BD Biosciences, Catalog No. 557870) for 15 mins at 37 °C. The cells were then washed and permeabilized with chilled BD Phosflow™ Perm Buffer III (BD Biosciences, Catalog No. 558050) overnight at −20 °C. The cells were washed to remove the permeabilization buffer and blocked with Mouse Trustain Fcx (Biolegend, Catalog No. 101320) for 5 minutes at room temperature. The cells were then treated with the antibody cocktail ((listed in the Table 15 below) for 1 hour at room temperature.

TABLE 15
Antibody cocktail.

| | | Phos- | | | |
|---|---|---|---|---|---|
| Fluoro- | | phorylation | | | |
| chrome | Antibody | Site | Clone | Catalog# | Vendor |
| BV480 | CD4 | | RM4-5 | 565634 | BD Biosciences |
| BV711 | CD8a | | 53-6.7 | 563046 | BD Biosciences |
| BV750 | CD11b | | M1/70 | 746910 | BD Biosciences |
| BV786 | CD3 | | 17A2 | 564010 | BD Biosciences |
| AF488 | pSTAT3 | Tyr705 | D3A7 | 4323S | CST |

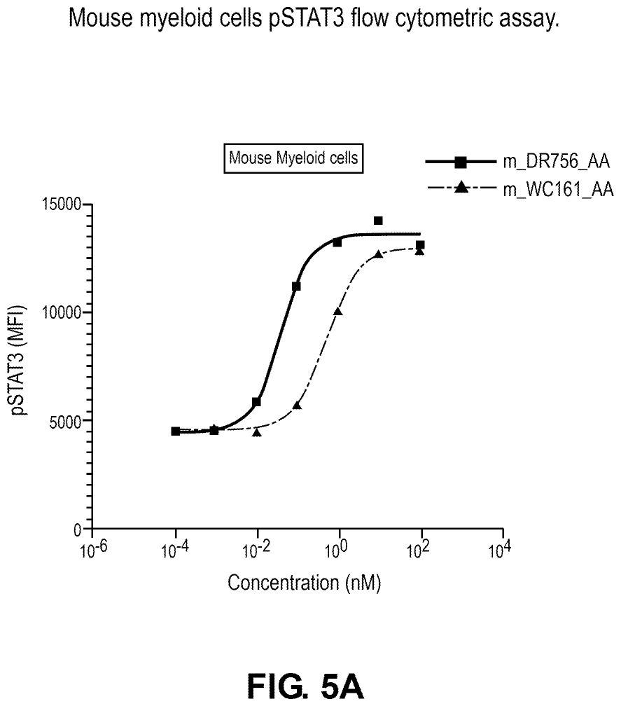

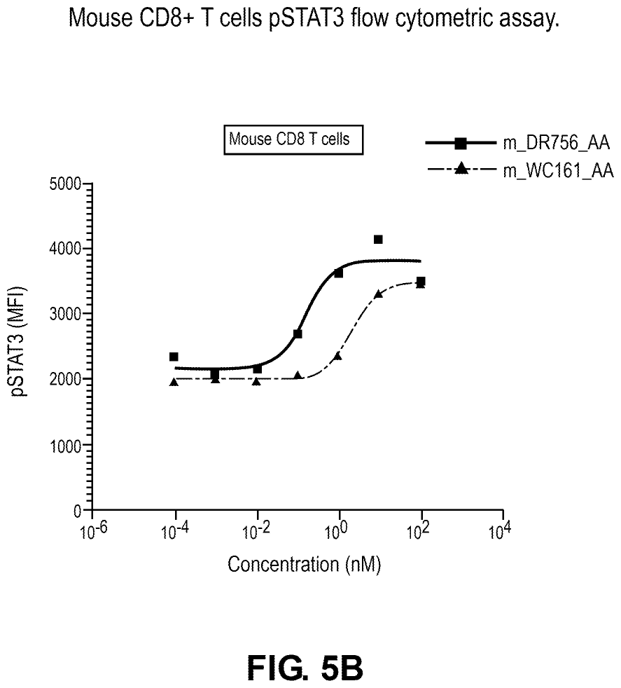

Following antibody staining, the cells were washed, fixed and ran on the Cytek® Aurora Spectral flow cytometer. The data was analyzed using the FlowJo software. The various cell lineages were gated using their lineage markers and the geometric mean fluorescence intensity of pSTAT3 expression was calculated on FlowJo. The data for mouse pSTAT3 flow cytometric assay are shown in FIGS. 5A and 5B.

The data shows that compared to the native dimeric mIL-10 (m_DR768_AA), the mouse inverted monomeric IL-10 molecule (m_WC161_AA) has a lower EC₅₀ for pSTAT3 induction on mouse myeloid cells and CD8 T cells.

### Example 6. Mouse Splenocyte LPS Assay

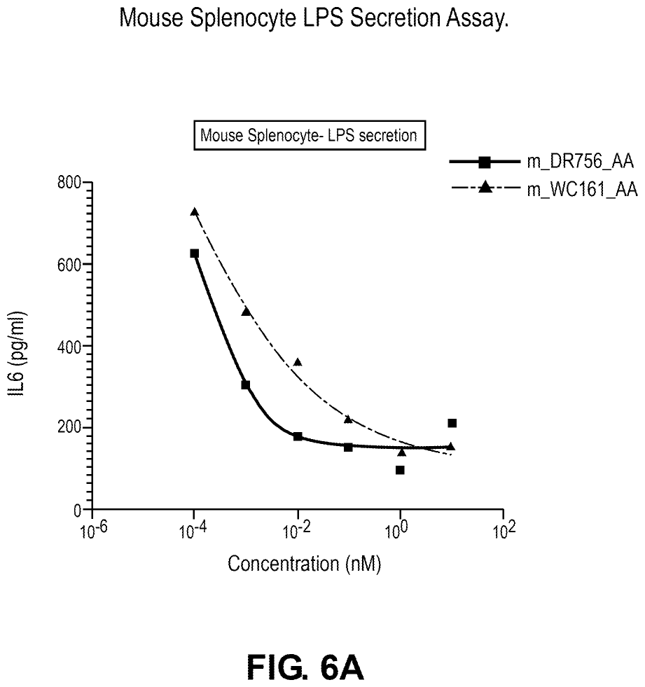

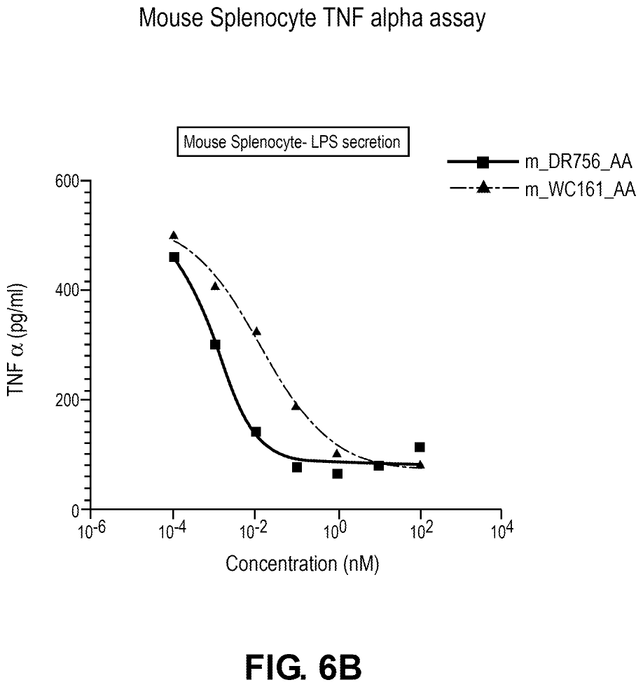

Spleens were removed from naïve C57BL/6 mice. Splenocytes were isolated by crushing the spleens with the plunger end of a syringe in complete RPMI medium (Gibco RPMI 1640 medium+10% FBS+1% penicillin-streptomycin) and straining through a 70 pM strainer. The cells were centrifuged at 400g for 5 min and the supernatant was discarded. The cell pellet was resuspended in 10 ml ACK lysis buffer and allowed to sit at 37 °C for 2 min. The cells were washed and resuspended in complete RPMI. The purified splenocytes (500,000 cells per well) were treated with 1 ng/ml of LPS (Millipore Sigma, Catalog No. L2630) in the presence of varying concentrations of the IL10 proteins (0.1 pM-100 nM) for 48 hours at 37 °C. Post-treatment, cells were centrifuged at 400g for 5 min and supernatants were collected. Supernatants were assessed for pro-inflammatory cytokines IL6 and TNF-alpha using the MSD kit (Meso Scale Discovery, Catalog No. K152A0H-2) in substantial accordance with the manufacturer's protocol. The data as shown in FIGS. 6A and 6B was graphed using Prism software. The data shows that the inverted monomeric mouse IL10 molecule (m_WC161_AA) inhibits IL-6 and TNF-α secretion by mouse splenocytes, albeit with a lower potency compared to the compared to the native dimeric IL-10 molecule (m_DR768_AA).

### Example 7. Mouse CD8 T Cell Blast Assay

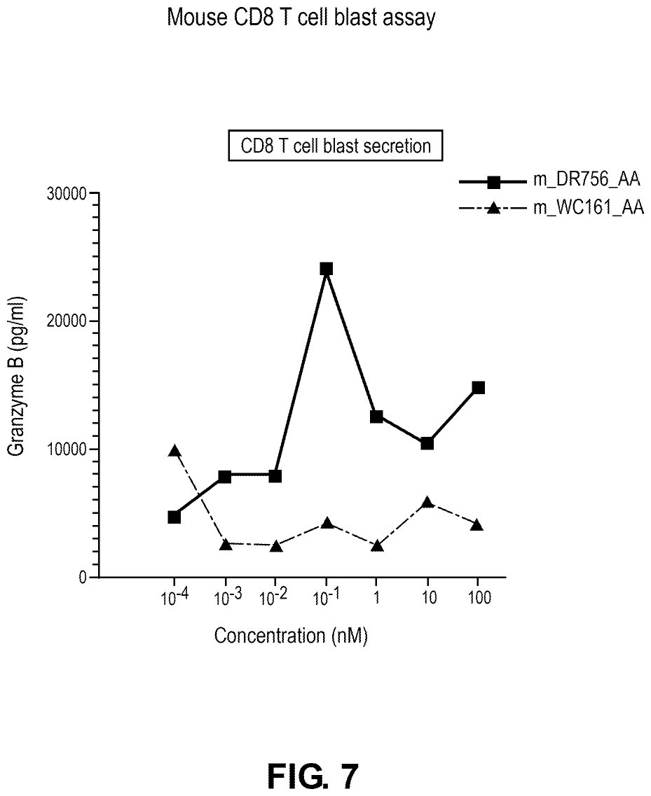

Spleens were removed from naïve C57BL/6 mice. Splenocytes were isolated by crushing the spleens with the plunger end of a syringe in complete RPMI medium (Gibco RPMI 1640+10% FBS+1% penicillin-streptomycin) and straining through a 70 μM strainer. The cells were centrifuged at 400g for 5 min and the supernatant was discarded. The cell pellet was resuspended in 10 ml ACK lysis buffer and allowed to sit at 37 °C for 2 min. The cells were washed and resuspended in complete RPMI. Mouse CD8 T cells were purified from the PBMCs by negative selection using a kit (Miltenyi Biotech, Catalog No. 130-104-075) following manufacturer's protocol. Isolated CD8 T cells were activate using the Mouse T cell Activation and Expansion Kit (Miltenyi Biotech-130-093-627) in the presence of 100 pM mouse IL2 for 72 hours at 37 °C. The activated CD8 T cells were washed, the beads were removed magnetically following which the cells were treated with varying concentrations of the IL10 proteins (0.1 pM-100 nM) in the presence of 100 pM mouse IL2 for 72 hours at 37 °C. Post-treatment, cells were centrifuged at 400g for 5 min and supernatants were collected. Supernatants were assessed for cytokines (Granzyme B) using the MSD kit (Meso Scale Discovery, Catalog No. K1523XR-2) in substantial accordance with the manufacturer's protocol. The data was graphed using Prism software. The data for mouse CD8 T cell blast assay are shown in FIG. 7.

The data shows that the mouse monomeric IL10 molecule (m_WC161_AA) does not induce Granzyme B secretion by activated CD8+ T cells compared to the native dimeric mIL-10 molecule (m_DR756_AA).

### Example 8. Mouse CD8 T Cell Blast Survival Assay

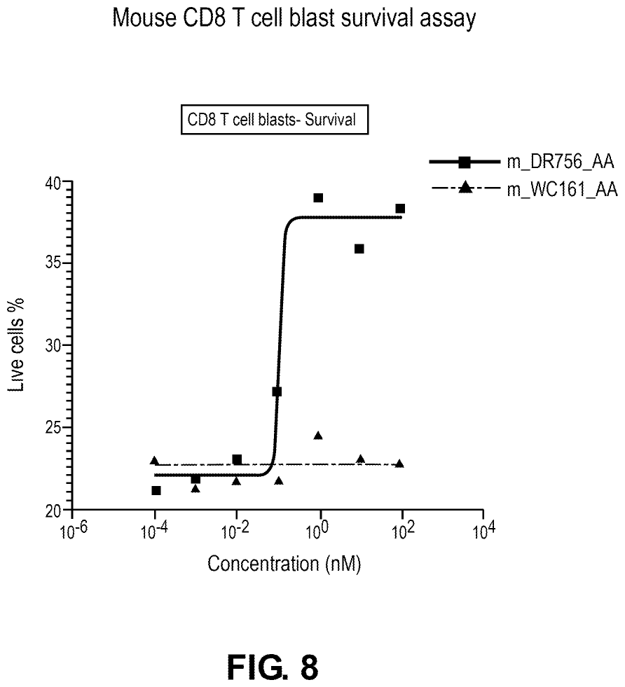

Spleens were removed from naïve C57BL/6 mice. Splenocytes were isolated by crushing the spleens with the plunger end of a syringe in complete RPMI medium (RPMI+10% FBS+1% Pennicillin-Streptomycin) and straining through a 70 pM strainer. The cells were centrifuged at 400g for 5 min and the supernatant was discarded. The cell pellet was resuspended in 10 ml ACK lysis buffer and allowed to sit at 37 °C for 2 min. The cells were washed and resuspended in complete RPMI. Mouse CD8 T cells were purified from the PBMCs by negative selection using a kit (Miltenyi Biotech, Catalog No. 130-104-075) in substantial accordance with the manufacturer's protocol. 200,000 CD8 T cells were added to each well of a 96-well plate pre-coated with 2.5 μg/ml of anti-mouse CD3E antibody (Biolegend 100302). The cells were then treated with IL10 proteins ranging in concentration from 0.1 pM to 100 nM in the presence of 5 μg/ml of anti-mouse CD28 antibody (Biolegend 102102) for 72 hours at 37 °C. The cells were then centrifuged at 400 g for 5 min and the supernatant was discarded. Cells were stained for Annexin V and Propidium Iodide using the Dead Cell Apoptosis Kits with Annexin V for Flow Cytometry (Invitrogen, Catalog No. V13241) in substantial accordance with the manufacturer's protocol. The data was analyzed using the FlowJo software. Following staining, the cells were run on the Cytek Aurora Spectral flow cytometer. The data was analyzed using the FlowJo software. The data as shown in FIG. 8 was graphed using Prism software.

The data demonstrates that the mouse inverted monomeric IL10 molecule (m_WC161_AA) does not enhance survival of activated mouse CD8+ T cells in contrast to the native dimeric mIL-10 molecule (m_DR756_AA).

### Example 9. Serum Concentration of Proteins

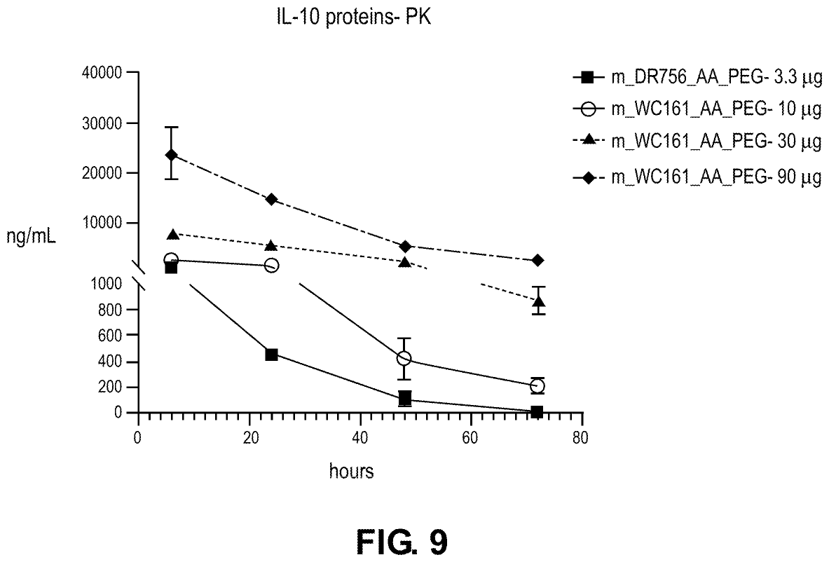

IL-10 proteins produced substantially as described in Example 1 were subcutaneously injected into 10 week old C57BL/6J female mice (RRID:IMSR_JAX:000664) at indicated doses. Blood was collected via submandibular bleeds and serum was prepared using serum microtubes (Sarstedt, Product number 41.1378.005). Serum IL-10 concentration was measured using the MSD kit (Meso Scale Discovery, K15069M). The concentrations were interpolated using four parameter (4PL) model in GraphPad Prism 9.3.1 software and are shown in FIG. 9.

The data shows that pegylated mouse monomeric IL10 molecules (m_WC161_AAPEG) have increased serum concentrations compared to the native dimeric IL-10 molecule (m_DR756_AA_PEG) over a 72 hour time period following a single dose injection.

### Example 10. Monocyte Cell Surface CD64 Staining

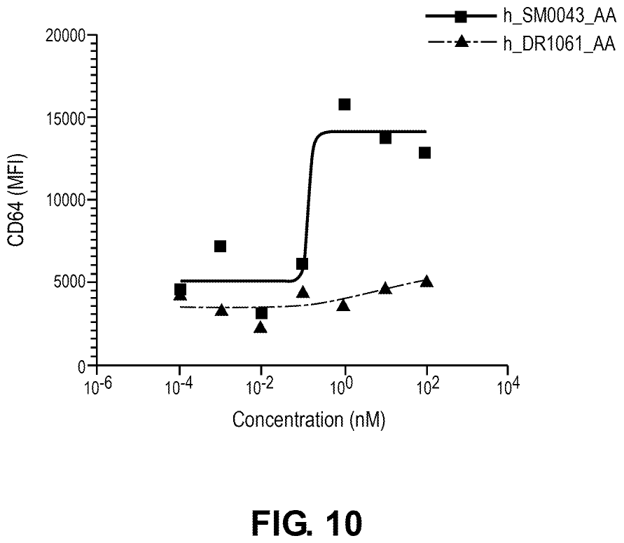

Human PBMCs were isolated from LRS chambers obtained from non-smoking human donors by Ficoll gradient purification. Human Monocytes were purified from the PBMCs by positive selection using Human CD14 Microbeads (Miltenyi Biotech, Catalog No. 130-050-201) following manufacturer's protocol. Monocytes were treated with varying concentrations of the fused IL10 polypeptides (0.1 pM -100 nM) for 48 hours at 37 °C. Post-treatment, cells were spun down at 400 g for 5 min. The cells were then washed and blocked with Human Trustain Fcx (Biolegend, Catalog No. 422301) and mouse serum for 15 minutes at 4 °C. The cells were then treated with CD64 antibody (Biolegend, Catalog No. 305006) for 30 min at 4 °C. Following antibody staining, the cells were washed, fixed and ran on the Cytek Aurora Spectral flow cytometer. The data was analyzed using the FlowJo software. The data shown in FIG. 10 was graphed using Prism software.

The data shows the inverted monomer (h_DR1061_AA) induces much lower CD64 expression on the surface of monocytes as compared to the native dimeric IL-10 molecule (h_SM0043_AA).

### Example 11: Evaluation of Inverted Monomers in pSTAT3 Flow Cytometric Assay

PBMCs were purified from healthy non-smoking donor blood collected in leukoreduction system chambers (LRS) using the human Miltenyi MACSprep PBMC isolation kit (Miltenyi Biotec, San Diego CA) in substantial accordance with the manufacturer's instructions. The purified PBMCs (500,000 cells per well) were stimulated with IL10 proteins at concentrations ranging from 0.1 pM-100 nM for 20 min at 37 °C. The cells were fixed with BD fix buffer I (BD Biosciences, catalog #557870) for 15 mins at 37 °C. The cells were then washed and permeabilized with chilled BD perm buffer III (BD Biosciences, catalog #558050) overnight at −20 °C. The cells were washed to remove the permeabilization buffer and blocked with Human Trustain Fcx (Biolegend, catalog #422301) and mouse serum for 5 minutes at room temperature. The cells were then treated with the antibody cocktail provided in Table 16 below for 1 hour at room temperature.

TABLE 16
Antibody Cocktail Components

| Fluoro- | Antibody | Phosphorylation | | | |
|---|---|---|---|---|---|
| chrome | Target | Site | Clone | Catalog# | Source |
| BV480 | CD4 | | L200 | 566148 | BD |
| | | | | | Biosciences |
| BV605 | CD8a | | RPA-T8 | 301040 | Biolegend |
| BV786 | CD56 | | 5.1H11 | 362550 | Biolegend |
| AF488 | pSTAT3 | Tyr705 | D3A7 | 4323S | CST |
| PE-Cy5 | CD3 | | UCHT1 | 555334 | BD |
| | | | | | Biosciences |
| PerCP-Cy | CD14 | | M5E2 | 301824 | Biolegend |
| 5.5 | | | | | |
| APC | CD20 | | 1412 | 340516 | Biolegend |

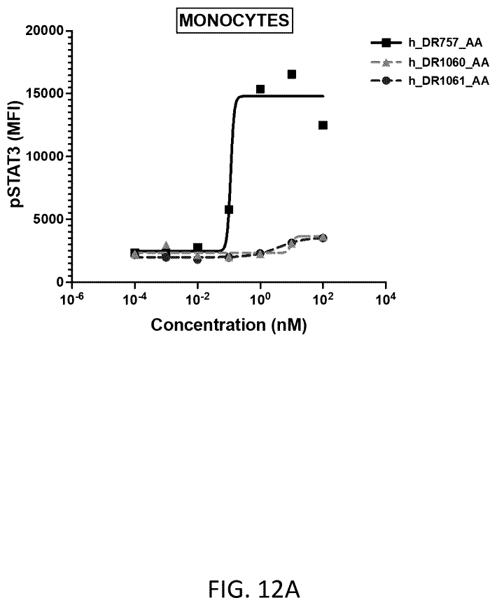

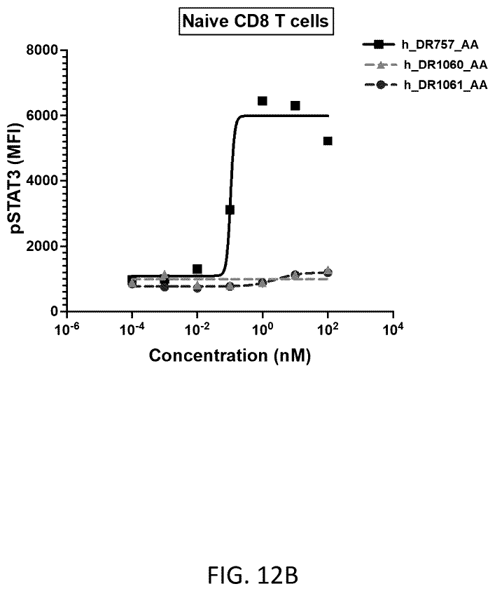

Following antibody staining, the cells were washed, fixed and ran on the Cytek Aurora Spectral flow cytometer (Cytek Biosciences). The data was analyzed using the FlowJo software (BD Biosciences, www.flowjo.com). The various cell lineages were gated using their lineage markers and the geometric mean fluorescence intensity of pSTAT3 expression was calculated using FlowJo. The data from this experiment is provided in FIGS. 12A and 12B of the attached drawings. As illustrated by the data provided in FIGS. 12A and 12B illustrates that the monomeric IL10 molecules (h_DR1060_AA and h_DR1061_AA show decreased pSTAT3 signal in monocytes and CD8 T cells as compared to the native dimeric IL10 molecule (h_DR757_AA).

### Example 12. In Vivo Evaluation in DSS Model of Ulcerative Colitis

The IL10 variants of the present disclosure were evaluated for therapeutic efficacy in a murine DSS model of ulcerative colitis. The murine DSS-induced colitis model is widely used and possess many similarities with human ulcerative colitis. Chassaing, B., et al. (2014) Current Protocols in Immunology 104:15.25.1-15.25.14.

The canonical sequence of mature (absent the endogenous 18 amino acid signal peptide) wild type murine IL10 molecule (UniProt Reference P18893) is a 160 amino acid polypeptide having the amino acid sequence:

(SEQ ID NO: 165)
SRGQYSREDNNCTHFPVGQSHMLLELRTAFSQVKTFFQTKDQLDNILLTD
SLMQDFKGYLGCQALSEMIQFYLVEVMPQAEKHGPEIKEHLNSLGEKLKT
LRMRLRRCHRFLPCENKSKAVEQVKSDENKLQDOGVYKAMNEFDIFINCI
EAYMMIKMK

When referencing amino acid substitutions or deletions in the murine IL10 variant molecules described herein are made in reference SEQ ID NO: 141.

The following Table 17 describes the pegylated murine IL10 (mIL10) test articles were used in the DSS study.

TABLE 17
DSS Study Murine Test Articles

| | Parent Polypeptide | PEGylation Location |
|---|---|---|
| Name | (SEQ ID) | and Description |
| m_DR756_AA_PEG | m_DR756_AA | N-terminus, 20 kDa |
| | | Linear PEG |
| m_WC161_AA_PEG | m_WC161_AA | N-terminus, 40 kDa |
| | | (branched 2 × 20) PEG |

The amino acid sequences of the parent polypeptides of the m_DR756_AA_PEG m_WC161_AA_PEG test articles are provided in Table 18 below:

TABLE 18
Parent Polypeptides of PEGylated DSS Study Articles

| SEQ | | | |
|---|---|---|---|
| ID NO | Name | Description | Amino Acid Sequence |
| 163 | m_DR756_AA | Alanine;8xHis chelating | AHHHHHHHHGSQYSREDNNCTHFPVGQ |
| | | peptide; Gly-Ser linker; | SHMLLELRTAFSQVKTFFQTKDQLDNI |
| | | ΔS1/ΔR2/ΔG3 mIL 10 | LLTDSLMQDFKGYLGCQALSEMIQFYL |
| | | | VEVMPQAEKHGPEIKEHLNSLGEKLKT |
| | | | LRMRLRRCHRFLPCENKSKAVEQVKSD |
| | | | FNKLQDQGVYKAMNEFDIFINCIEAYM |
| | | | MIKMKS |
| 164 | m_WC161_AA | AAs 116-160 wt mIL10; | NKSKAVEQVKSDFNKLQDQGVYKAMNE |
| | | AAs 1-115 wt mIL10; | FDIFINAIEAYMMIKMKSSRGQYSRED |
| | | GGS linker; | NNCTHFPVGQSHMLLELRTAFSQVKTF |
| | | 8xHis chelating peptide | FQTKDQLDNILLTDSLMQDFKGYLGCQ |
| | | | ALSEMIQFYLVEVMPQAEKHGPEIKEH |
| | | | LNSLGEKLKTLRMRLRRCHRFLPCENG |
| | | | GSHHHHHHHH |

Briefly, the DSS study was conducted as follows. Female C57Bl/6 mice (Jackson Laboratories) were administered 2.5% DSS, ad libitum, in drinking water on study days 0-6. Mice given normal water served as a negative disease control. Mice were treated with either PBS, m_DR756_AA_PEG (3 or 10 micrograms administered subcutaneously every other day) or m_WC161_AA_PEG (10 or 30 micrograms administered subcutaneously every other day) in accordance with the following Table 19.

TABLE 19
DSS Study Treatment Groups

| | | Treatment/ | Dose | Schedule |
|---|---|---|---|---|
| Group | DSS | Test Article | [ug] | (Study Days) |
| A | Water | n/a | — | — |
| B | 2.5% | PBS | — | D0, D2, D4, D6, D8 |
| C | 2.5% | m_DR756_AA_PEG | 3 | D0, D2, D4, D6, D8 |
| D | 2.5% | m_DR756_AA_PEG | 10 | D0, D2, D4, D6, D8 |
| E | 2.5% | m_WC161_AA_PEG | 10 | D0, D2, D4, D6, D8 |
| F | 2.5% | m_WC161_AA_PEG | 30 | D0, D2, D4, D6, D8 |

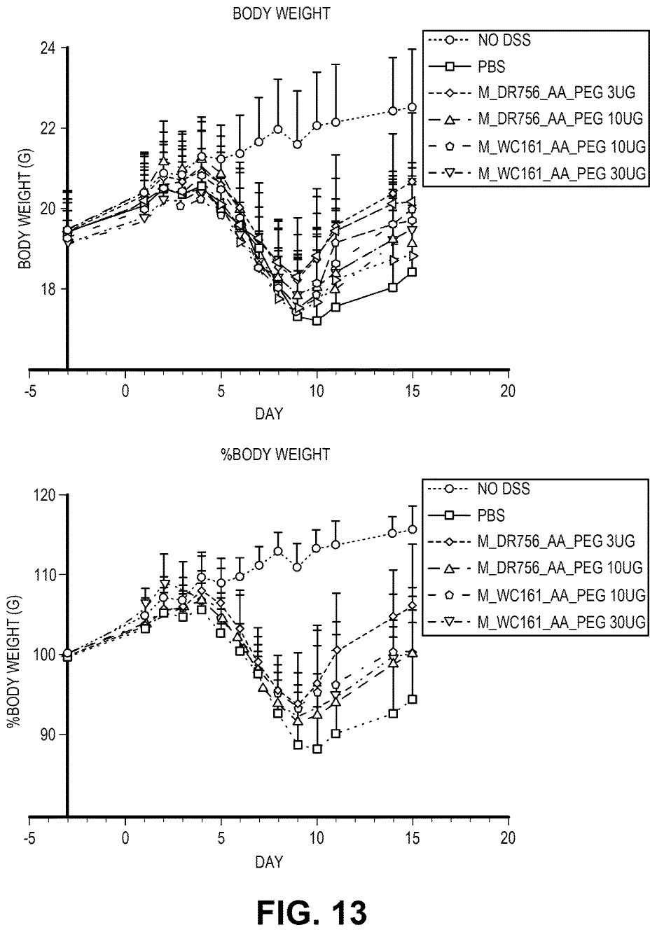

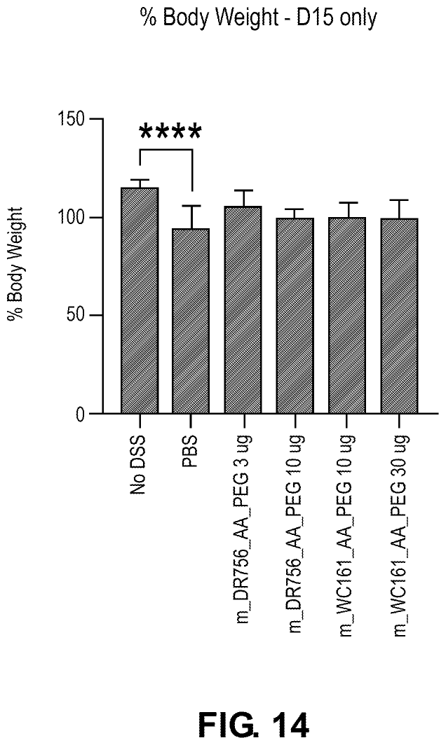

The bodyweights of the mice were evaluated throughout the study. Mice were weighed three days prior to initiation of DSS (Study Day -3) and after DSS administration and test article treatment. The data relating to bodyweight for each treatment group is provided in FIG. 13 of the attached drawings. Both average body weight and average % body weight +/− SEM are shown. Percent body weight was calculated as =(Body weight on Study Day X—Baseline body weight on Study Day -3)/(Baseline body weight on Study Day -3). Terminal bodyweight of the mice was evaluated on Study Day 15 and the average % body weight +/− SEM on day 15 is shown in FIG. 14 of the attached drawings. Significance was determined by One-Way ANOVA with Holm-Šidik's multiple comparisons test. ****p<0.0001

On Study Day 15, terminal blood was harvested via cardiac puncture and collected into serum collection tubes. In-life and terminal serum was isolated by centrifuging clotted blood for 5 minutes at 10,000 RPM. Serum was collected after centrifugation and placed on ice temporarily before freezing for later analysis. Serum cytokines were assessed using Meso Scale Discovery multiplex kits (Meso Scale Discovery) in substantial accordance with the manufacturer's instructions.

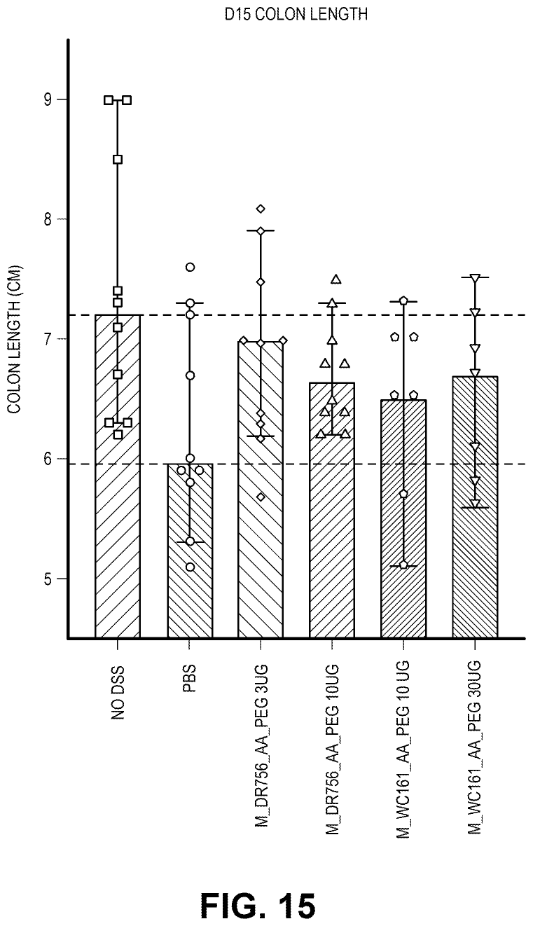

On Study Day 15, mice were terminated by CO₂ asphyxiation. Colons were harvested, cleared of feces, and measured for length. The data relating to colon length in response to the various treatments is provided in FIG. 15 and reflects average colon length +/− SEM. As shown by the data presented in FIG. 15, m_DR756_AA_PEG, m_DR2677_AA_PEG, and m_DR2678_AA_PEG show evidence of protection against DSS-associated outcomes and pathology.

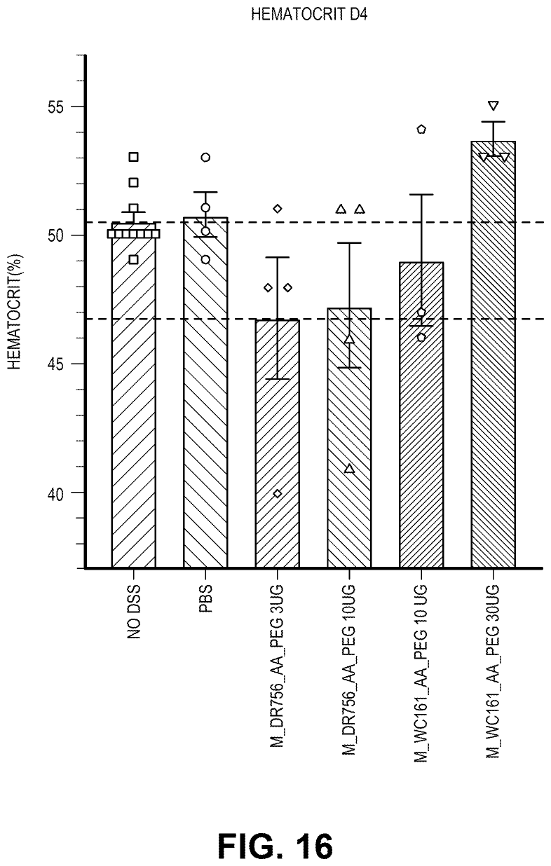

Hematocrit levels of were evaluated on Study Day 4 (D4). Blood samples collected via submandibular blood collection on Study Day 4 were placed into serum collection tubes and quantified using a Hemata Stat II hematocrit microcentrifuge (EKF Diagnostics, Inc.) in substantial accordance with the manufacturer's instructions. The data relating to hematocrit levels is provided in FIG. 16 of the attached drawings and illustrates average hematocrit +/− SEM. As illustrated, a putative reduction in HCT may be observed on Day 4 with m_DR756_AA_PEG.

### Example 13. Evaluation of Peritoneal Exudate Cell Scavenger CD164 and CD64 Expression Over Time

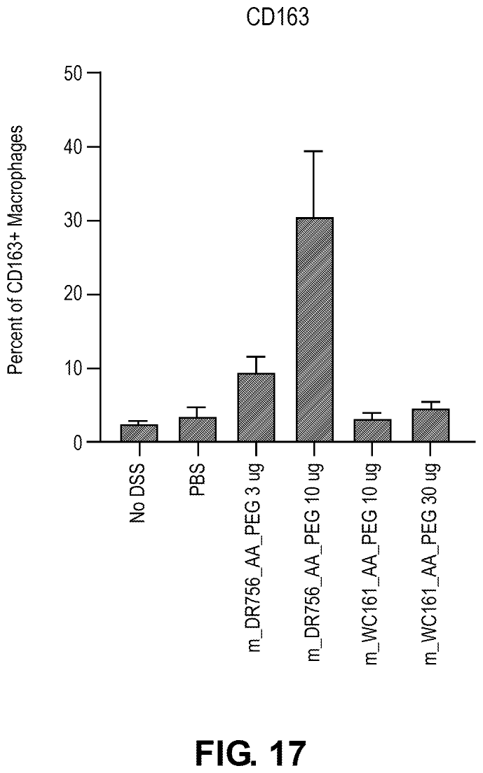

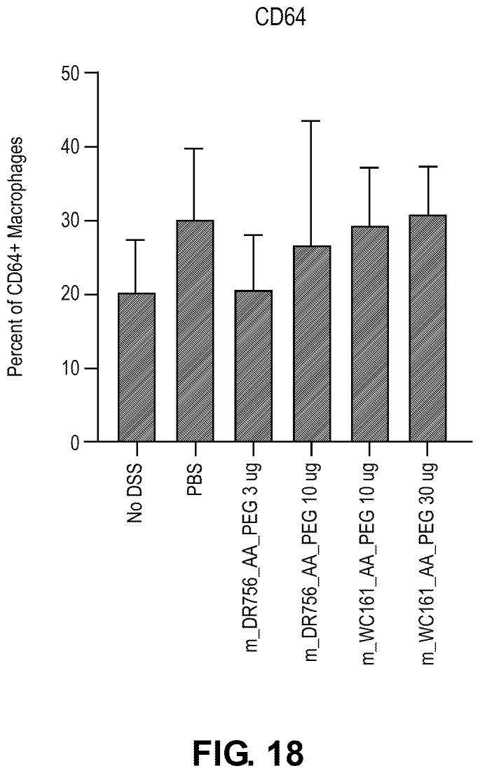

For peritoneal exudate cell harvesting, peritoneal lavages were performed on euthanized mice and the exudate was collected (peritoneal exudate cells, PEC). Peritoneal lavage fluid was centrifuged (1500 rpm, 5 mins), supernatant removed, and cell pellets were resuspended in 1 ml PBS and kept on ice. Blood was collected into EDTA containing tubes, transferred to 5 ml tubes, and red blood cells were lysed using ACK lysing buffer (ThernoFisher #A1059201) then centrifuged (1500 rpm, 5 mins). Supernatants were removed and cell pellets were resuspended in 1 ml PBS. Peritoneal exudate cells were collected over time and CD163 or CD64 was detected by flow cytometry. Individual and average median fluorescence intensity +/− SEM are also shown. APC=antigen presenting cell. As illustrated in FIG. 17, treatment with the PEGylated wild type murine IL10 surrogate molecule (m_DR756_AA_PEG) exhibits CD163 upregulation on macrophages. CD64 expression was also evaluated and the results are presented in FIG. 18.

### Example 14. Evaluation of Peritoneal Exudate Cell Scavenger CD164 and CD64 Expression Over Time

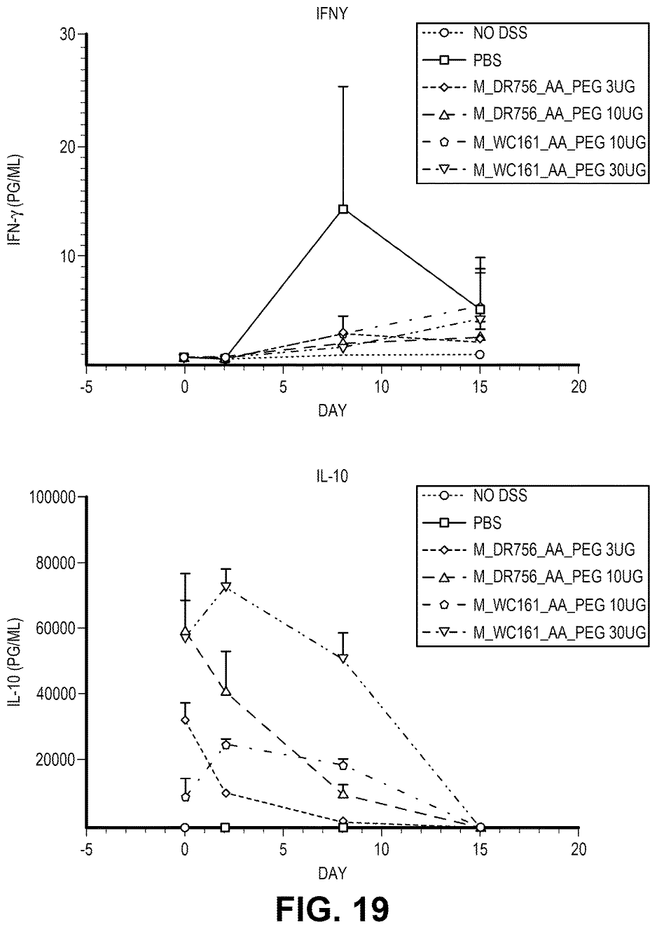

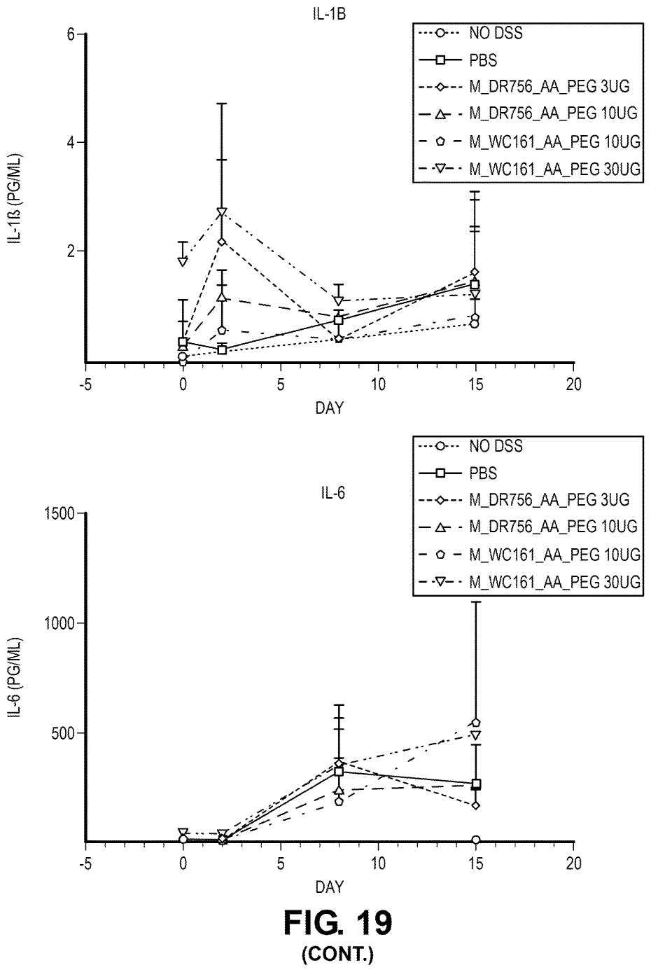

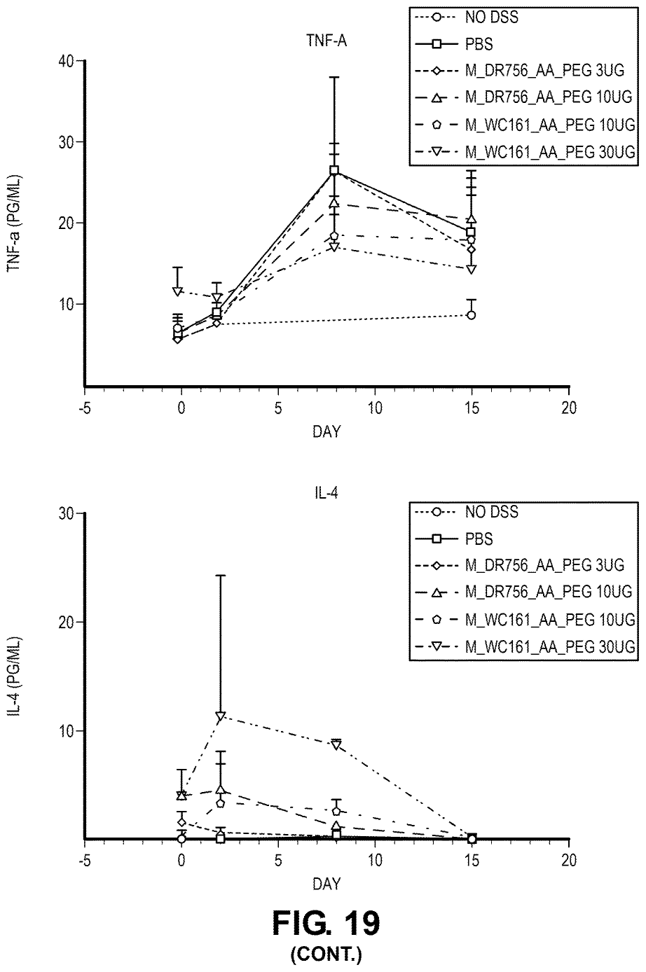

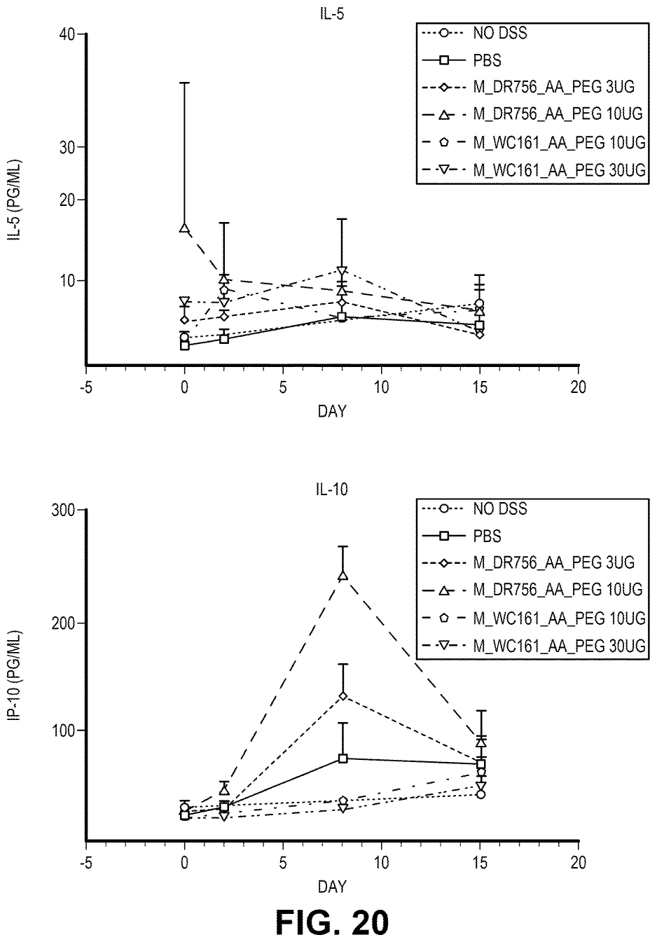

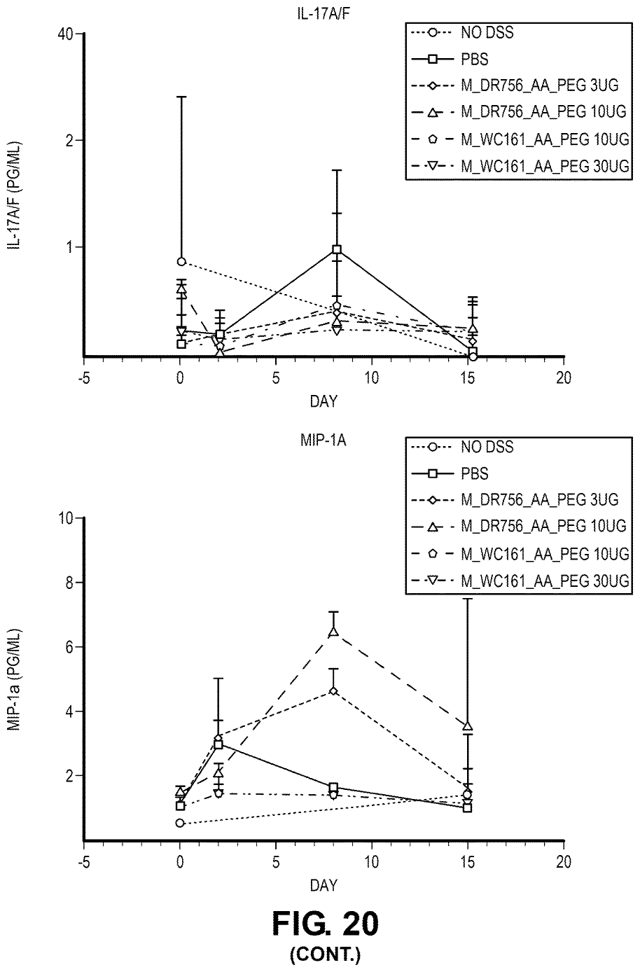

Serum was collected at various timepoints during the study and serum cytokines (I IFNγ, IL10, IL-1B, IL6, TNF-a, IL-4, IL-5, IP10, IL17A/F, MIP1a) were measured. Serum cytokines were assessed using Meso Scale Discovery multiplex kits (Meso Scale Discovery) in substantial accordance with the manufacturer's instructions. The data for the various treatment groups is provided in FIGS. 19 and 20 of the attached drawings and reflects average analyte concentrations (pg/mL)+/− SEM. As illustrated by the data present in FIGS. 19 and 20, treatment with the inverted monomer (m_WC161_AA_PEG) is generally associated with reduced systemic IFNγ and TNFa relative to the wild type molecule (m_DR756_AA_PEG). Treatment with m_DR756_AA_PEG is associated with elevated serum IP-10 and MIP1a. Treatment with the inverted monomer (m_WC161_AA_PEG) is associated with elevated with elevated serum IL-4 and IL-5 (Th2 cytokines).

Following evaluation for colon length, the isolated colons were then placed in formalin for 24 hours at room temperature before processing and embedding into FFPE blocks using standardized procedure on a tissue processor. FFPE blocks were sectioned at 5 m thickness and placed on charged glass slides.

Multiplex immunofluorescence analysis was performed on the harvested tissues for the presence of the following markers: RORγ, CD11b & CD3e. Primary antibodies used for immunohistochemical analysis were: anti-mouse: RORγ (Abcam, catalog ab207802) at a working concentration 1:750; anti-CD11b (Abcam, catalog ab216445) at a working concentration 1:3000; and anti-CD3e (Thermo/Invitrogen, catalog MA1-90582) at a working concentration 1:200. All antibodies were incubated at room temperature for 60 minutes.

Slides were stained with the antibodies on Leica Bond Rx (Leica Biosystems) automated staining system. The automated staining system was programmed to perform the following steps: (a) heat-induced epitope retrieval (HIER) and de-paraffinization was performed for 20 minutes at 97 °C in the presence of BOND-PRIME Epitope Retrieval Solution 2 (ER2) (Leica Biosystems); (b) peroxidase quenching was performed; (c) incubation with primary antibody for 60 minutes; (d) incubation with secondary antibody polymer for 30 minutes; and (e) detection fluorophores for 10 minutes at room temperature. Fluorophores used to visualize the signal included Opal 520, Opal 620, and Opal 690 fluorophores (Akoya Biosciences, Marlborough MA).

For labeling, the staining was performed in a sequential manner by including a second antigen retrieval step between the two procedures to strip out any unbound reagents from the previous markers using BOND-PRIME Epitope Retrieval Solution 1 (ER1) (Leica Biosystems). Appropriate cross-reactivity controls were included for quality check.

Upon completion of fluorescence staining, tissues were counterstained with Akoya spectral DAPI (Akoya Biosciences) and cover slipped using ProLong™ Gold Antifade Mountant (TherrnoFisher Scientifid/Invitrogen, catalog number: P36930) aqueous mounting medium. Whole slide scanning and signal quantification was performed using Akoya Vectra multispectral imaging system (Akoya Biosciences) and Akoya Vectra InForm analysis software suite (Akoya Biosciences). Digital quantification of specific staining signal was performed, and data was collated and aggregated from all images by using Akoya Phenoptr Reports software package (Akoya Biosciences).

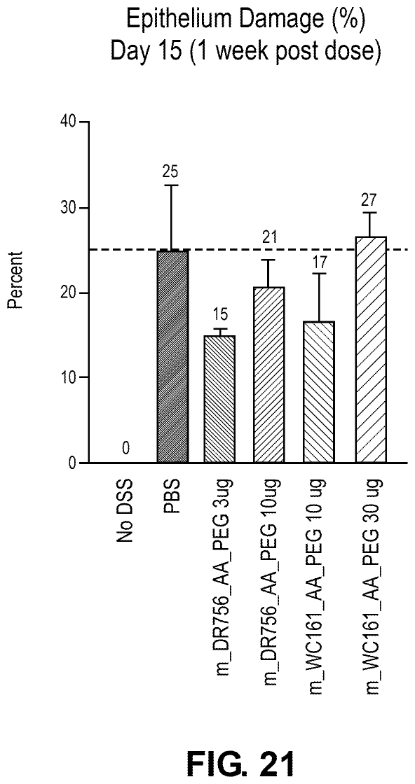

The tissues were evaluated for colon epithelial damage. Whole slide scanned hematoxylin and eosin (H&E) stained mouse colon sections were evaluated with Visiopharm software (Visiopharm A/S, DK 2970-Horsholm, Denmark) for the presence of an intact epithelium layer. The data for the various treatment groups is provided in FIG. 21 of the attached drawings and reflects the average percent colon epithelial damage +/− SEM.

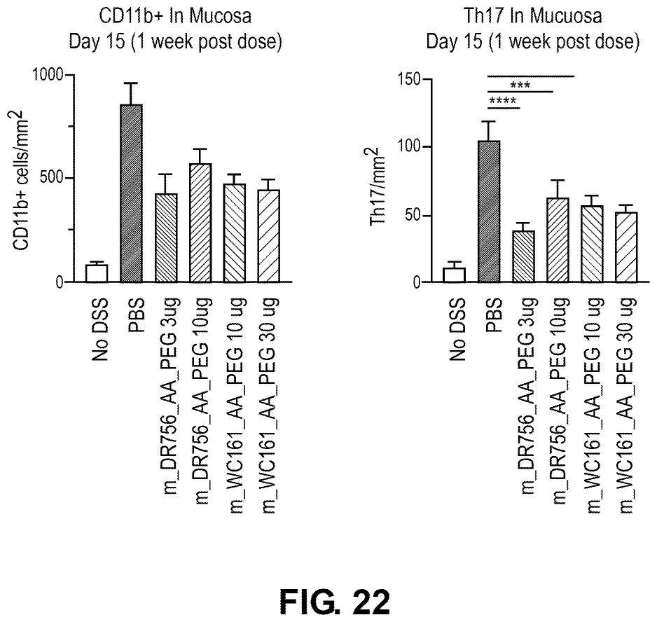

Whole slide scanned multiplex immunofluorescence stained mouse colon sections were evaluated with inForm image analysis software (Akoya Biosciences) for the presence of CD11b and CD4+ RorG T cells. The data for the various treatment groups is provided in FIG. 22 of the attached drawings and reflects the average number of cells/mm² +/− SEM.

In summary the data obtained in the DSS model of ulcerative colitis indicates that the the inverted monomer (m_WC161_AA_PEG): (a) exhibits evidence of protection against DSS-associated outcomes and pathology; (b) exhibits protection against DSS-associated epithelial damage; and (c) exhibits reduced myeloid and Th17 infiltration in the intestinal mucosa.

### Example 15: In Vivo Evaluation of Pharmacokinetic Parameters

Pharmacokinetic parameters of the m_DR756_AA_PEG and m_WC161_AA_PEG test articles were evaluated in an in vivo study in C57Bl/6 mice. Briefly, on Study Day 0 (D0), female C57Bl/6 mice received a single subcutaneous administration of 10 micrograms of m_DR756_AA_PEG, or a single subcutaneous administration of 10, 30, or 90 micrograms m_WC161_AA_PEG. In-life blood samples were collected at various timepoints via submandibular blood collection into serum collection tubes. Terminal blood was harvested via cardiac puncture and collected into serum collection tubes. In-life and terminal serum was isolated by centrifuging clotted blood for 5 minutes at 10,000 RPM. Serum was collected after centrifugation and placed on ice temporarily before freezing for later analysis. Test articles in serum were quantified using a mouse IL-10 MSD U-plex kit (Meso Scale Discovery) in substantial accordance with the manufacturer's instructions. Each individual test article was used as a standard for detection.

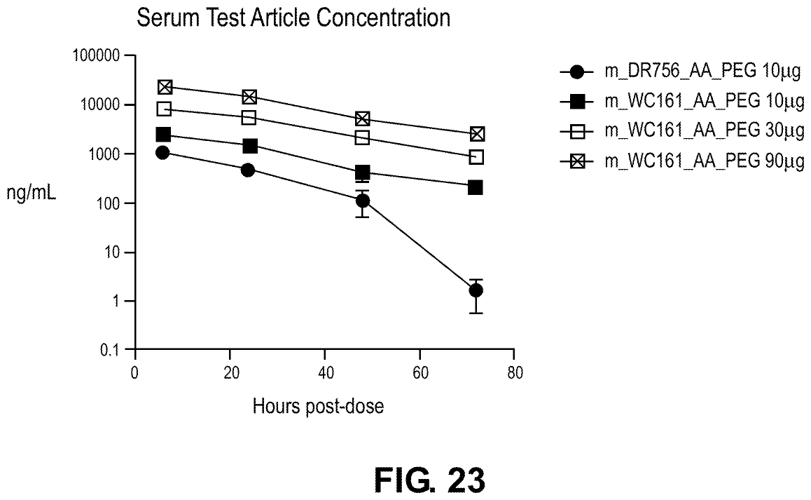

Serum was collected over time and serum concentrations of test articles were quantified over time and the data provided in FIG. 23 of the attached drawings reflecting the average test article concentrations (ng/mL) +/− SEM. As illustrated in FIG. 23, m_WC161_AA_PEG exhibited improved systemic exposure compared to m_DR756_AA_PEG. Furthermore, the inverted monomer (m_WC161_AA_PEG) exhibits high sustained exposure after multiple doses.

### Example 16: Evaluation of Myeloid cell pSTAT3 induction Over Time

The effect of the test articles was evaluated for their ability to induce pSTAT3 in myeloid cells over time as follows. In-life or terminal whole blood from the experiments described in Examples above was collected into EDTA tubes for pSTAT or surface immunophenotyping flow cytometry. 100-200 μL of whole blood that was collected into EDTA tubes was transferred to 96 well deep well plates. BD Phosflow Lyse/Fix Buffer (cat #558049) was added to a 2 ml total volume and incubated at 37 °C for 10 mins. Cells were centrifuged at 600 g for 7 mins, supernatant removed, and washed twice with 1 ml PBS/2% FBS per well. Cell pellets were vortexed to resuspend and 1 ml prechilled BD Perm Buffer III (cat #558050) was added and cells stored at −80 °C. Following conclusion of the in-life sample collection, cells were thawed and washed three times to remove permeabilization buffer. Cells were blocked with 1:25 TruStain FcX (Biolegend #101320) and 1:50 rat serum for 10 mins on ice then incubated with a cocktail of antibodies for 30 mins at 4 °C (see Table 12 below). Cells were washed twice and resuspended in PBS/2% FBS and ran on a Cytek Aurora Spectral cytometer. Data was analyzed using Cell Engine. Cells were gated using lineage markers and the median fluorescent intensity of pSTAT 1 and pSTAT3 was calculated.

TABLE 20
pSTAT staining panel

| Fluoro- | | | | |
|---|---|---|---|---|
| chrome | Antibody | Clone | Source | Catalog# |
| PE | pSTAT-1 (Y701) | REA159 | Miltenyi Biotec | 130-129-183 |
| AF488 | pSTAT-3 (Y705) | C3A7 | Cell Signaling | 4323S |
| | | | Tech | |
| BV786 | CD3 | 17A2 | BD Biosciences | 564010 |
| APC | CD4 | RM4-5 | Biolegend | 100516 |
| BV711 | CD8 | 53-6.7 | BD Biosciences | 563046 |
| PacBlue | B220 | RA3-6B2 | BD Biosciences | 558108 |
| BV750 | cd11b | M1/70 | Biolegend | 101267 |
| | Trustain FcX | | Biolegend | 101320 |

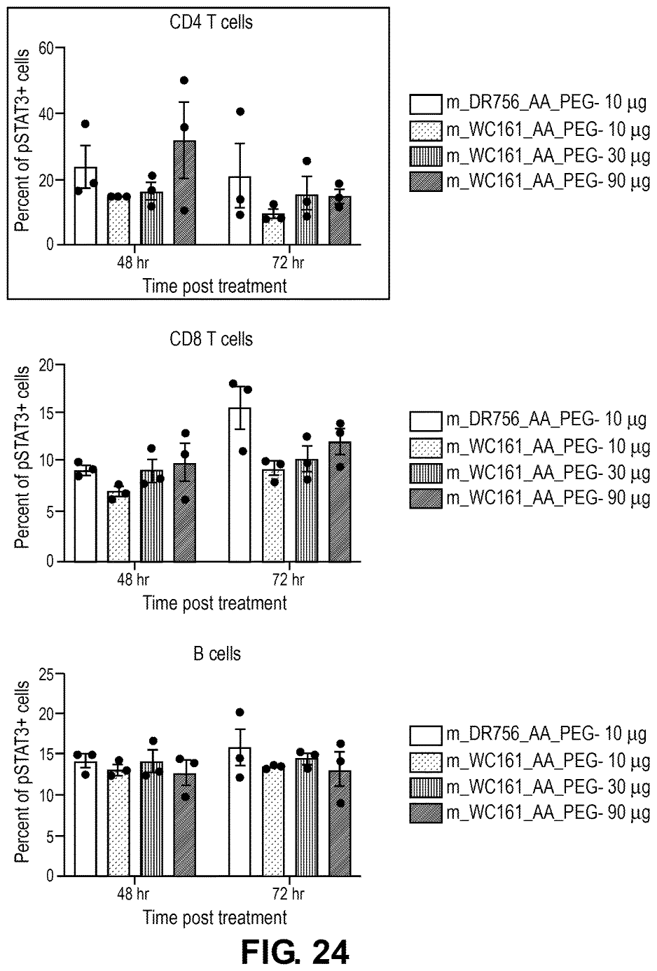

To evaluate myeloid cell pSTAT3 induction over time, peripheral blood was collected over time and pSTAT3 was detected by flow cytometry. The data is provided in FIG. 24 of the attached drawings. As illustrated in FIG. 24, the inverted monomer (m_WC161_AA_PEG) exhibited evidence of pSTAT3 induction in PBMCs at 48 and 72 hours after administration.

### Sequences

The nucleic acid sequences encoding the inverted monomers in Table 6 are provided in Table 5 below.

TABLE 5
Human Inverted Monomer Nucleic Acid Sequence

| SEQ ID | | |
|---|---|---|
| NO | Sequence | Notes |
| 99 | AAGAGTAAAGCTGTCGAGCAGGTTAAGAATGCTTTTAATAAACTGCAA | DR1060 |
| | GAAAAAGGTATCTATAAAGCCATGTCTGAGTTCGATATCTTTATTAAC | |
| | TACATTGAAGCATATATGACGATGAAAATCCGAAACTCCCCCGGGCAG | |
| | GGAACCCAGTCAGAGAACAGCTGTACACATTTTCCTGGCAACCTGCCT | |
| | AATATGCTCCGTGACCTTCGGGACGCCTTTTCGAGAGTGAAGACCTTC | |
| | TTCCAAATGAAGGATCAGTTAGACAATTTACTCCTGAAGGAGTCCCTT | |
| | TTGGAGGACTTCAAGGGGTACCTGGGATGTCAGGCACTCAGCGAGATG | |
| | ATACAGTTCTACCTGGAAGAGGTAATGCCCCAGGCCGAAAACCAAGAT | |
| | CCAGATATTAAGGCGCACGTGAACTCTCTGGGCGAGAATCTAAAAACT | |
| | CTGCGCTTGAGACTCAGGCGGTGCCACAGGTTTCTGCCATGCGAAAAT | |
| | GGTGGATCCCACCATCACCATCACCACCATCAT | |
| 100 | AAGAGCAAAGCTGTAGAGCAGGTTAAAAACGCATTCAATAAGCTGCAA | DR1061 |
| | GAGAAGGGTATTTATAAGGCCATGTCCGAGTTTGACATCTTCATTAAC | |
| | TACATCGAGGCCTACATGACGATGAAGATCAGGAACACTTCTCCCGGA | |
| | CAGGGCACACAGTCTGAAAATTCATGCACCCATTTCCCCGGAAATTTG | |
| | CCAAACATGCTGCGGGACCTGCGAGATGCGTTTAGTCGGGTCAAAACC | |
| | TTTTTTCAGATGAAGGACCAGCTGGATAATTTATTACTCAAGGAGTCC | |
| | CTTCTGGAGGATTTCAAAGGCTATTTGGGGTGCCAAGCACTGTCGGAG | |
| | ATGATACAGTTCTACCTGGAAGAAGTGATGCCACAAGCCGAAAACCAG | |
| | GACCCTGATATTAAAGCTCACGTGAATAGCCTGGGGGAGAATCTAAAA | |
| | ACACTTCGTCTCCGCCTCAGAAGGTGTCACAGATTTCTCCCTTGTGAA | |
| | AACGGTGGATCCCACCATCACCATCACCACCATCAT | |
| 101 | AAGAGCAAGGCAGTCGAACAGGTGAAAAATGCCTTTAATAAACTACAA | DR1062 |
| | GAAAAGGGCATTTATAAAGCTATGTCTGAGTTTGATATCTTTATTAAT | |
| | TACATTGAGGCGTATATGACGATGAAGATCCGCAATCCAGGACAAGGA | |
| | ACCCAGAGTGAAAACTCCTGTACCCACTTCCCTGGTAACTTACCCAAT | |
| | ATGCTGCGGGATCTGCGTGACGCTTTCTCAAGAGTAAAAACTTTTTTC | |
| | CAGATGAAGGACCAGCTTGACAACCTCCTGCTCAAGGAGTCCCTTCTG | |
| | GAGGATTTTAAAGGGTACCTCGGGTGTCAAGCCCTGAGCGAGATGATC | |
| | CAGTTCTACCTGGAGGAAGTGATGCCACAGGCAGAGAACCAGGACCCC | |
| | GATATAAAGGCCCACGTTAACTCTCTGGGCGAGAATCTCAAAACATTG | |
| | AGGTTGAGACTGAGGCGATGCCATCGGTTCTTACCTTGCGAAAACGGT | |
| | GGATCCCACCATCACCATCACCACCATCAT | |
| 102 | ATGTCCAAGGCAGTGGAGCAAGTCAAGAACGCCTTTAATAAATTACAA | SS0166 |
| | GAGAAGGGCATCTACAAAGCGATGTCCGAGTTTGACATCTTCATTAAC | |
| | TACATTGAAGCTTACATGACCATGAAAATAAGGAACAGCCCGGGACAG | |
| | GGCACCCAGAGTGAAAACTCTTGTACGCACTTCCCCGGGAATCTGCCC | |
| | AACATGCTGAGAGATCTGCGGGACGCCTTCTCAAGAGTGAAGACCTTC | |
| | TTCCAAATGAAAGACCAGTTGGACAATCTGCTGCTGAAGGAAAGCTTA | |
| | TTAGAGGATTTCAAAGGCTATCTAGGATGTCAGGCTCTGTCAGAAATG | |
| | ATCCAGTTCTATCTGGAGGAGGTGATGCCACAGGCCGAGAATCAGGAC | |
| | CCAGACATTAAAGCGCACGTCAATTCTCTGGGAGAAAACCTCAAGACT | |
| | CTCCGGTTGCGGCTGAGACGGTGTCATAGATTCTTGCCATGTGAAAAC | |
| 103 | AAGAGTAAGGCAGTGGAACAGGTAAAGAACGCCTTTAACAAACTGCAG | DR1057 |
| | GAGAAAGGCATTTATAAGGCGATGAGCGAATTTGATATCTTTATCAAC | |
| | TATATCGAAGCCTATATGACGATGAAAATAAGGAACGCCGTCCCGAAC | |
| | CAGAGGGATTACGACCCTAATTGCACCCACTTCCCAGGCAACTTACCT | |
| | AATATGCTGCGCGACCTTCGAGACGCTTTTTCAAGAGTTAAAACTTTC | |
| | TTCCAGATGAAAGATCAACTGGATAATCTCCTGTTAAAAGAGTCCCTC | |
| | TTGGAAGACTTCAAGGGGTACTTGGGATGCCAGGCCCTGTCTGAGATG | |
| | ATTCAGTTTTACCTGGAGGAGGTGATGCCCCAGGCAGAAAATCAAGAT | |
| | CCAGACATTAAGGCTCACGTGAATAGCCTGGGTGAGAACCTGAAGACA | |
| | CTCCGGCTCAGACTTCGTCGGTGTCATCGCTTCCTACCCTGTGAGAAT | |
| | GGTGGATCCCACCATCACCATCACCACCATCAT | |
| 104 | AAGAGCAAGGCTGTGGAGCAGGTAAAGAATGCCTTTAATAAGTTGCAG | DR1058 |
| | GAAAAAGGAATCTATAAAGCGATGAGCGAATTTGACATATTCATCAAT | |
| | TACATCGAGGCTTACATGACCATGAAGATTCGGGGCAAAGGTCCACTA | |
| | ACACAACCGTCCGAAGATTGCACCCACTTTCCAGGGAACCTGCCCAAC | |
| | ATGCTGCGAGACCTGCGGGACGCCTTCTCAAGAGTTAAAACGTTTTTC | |
| | CAGATGAAGGATCAACTCGATAATCTCCTGCTCAAAGAGAGTTTACTT | |
| | GAGGATTTTAAAGGCTATCTGGGATGCCAGGCACTGTCTGAAATGATT | |
| | CAGTTCTACCTGGAGGAGGTCATGCCTCAGGCAGAGAATCAGGACCCT | |
| | GACATTAAGGCCCACGTGAACTCCCTTGGGGAGAACTTGAAGACTTTG | |
| | AGGCTTCGTCTGAGAAGGTGTCATCGCTTCCTCCCCTGTGAAAACGGT | |
| | GGATCCCACCATCACCATCACCACCATCAT | |
| 105 | AAATCTAAGGCAGTGGAGCAGGTGAAGAATGCCTTCAATAAATTGCAG | DR1059 |
| | GAGAAAGGAATTTACAAGGCCATGTCCGAGTTTGATATCTTTATCAAC | |
| | TATATAGAAGCATACATGACCATGAAGATCCGTGGGTCAGGAAGTGGC | |
| | TCAGGTTCTGGGAGCGGCAGCTGCACACACTTTCCGGGGAACCTCCCA | |
| | AACATGCTCAGAGATCTCCGCGACGCCTTCTCCCGAGTTAAAACGTTT | |
| | TTCCAAATGAAGGACCAGCTGGACAACCTGCTGCTGAAGGAATCCCTG | |
| | TTGGAGGATTTCAAAGGTTATCTGGGATGTCAGGCGCTAAGTGAGATG | |
| | ATTCAATTCTACCTTGAAGAAGTCATGCCCCAGGCTGAGAATCAGGAC | |
| | CCCGATATTAAGGCTCACGTAAACAGCCTCGGCGAAAATCTTAAAACT | |
| | CTGCGGTTACGGTTAAGGAGATGCCATAGGTTTCTGCCTTGTGAGAAT | |
| | GGTGGATCCCACCATCACCATCACCACCATCAT | |
| 106 | AAGAGTAAAGCTGTCGAGCAGGTTAAGAATGCTTTTAATAAACTGCAA | WC0006 |
| | GAAAAAGGTATCTATAAAGCCATGTCTGAGTTCGATATCTTTATTAAC | |
| | TACATTGAAGCATATATGACGATGAAAATCCGAAACTCCCCCGGGCAG | |
| | GGAACCCAGTCAGAGAACAGCTGTACACATTTTCCTGGCAACCTGCCT | |
| | AATATGCTCCGTAAGCTTCGGGACGCCTTTTCGAGAGTGAAGACCTTC | |
| | TTCCAAATGAAGGATCAGTTAGACAATTTACTCCTGAAGGAGTCCCTT | |
| | TTGGAGGACTTCAAGGGGTACCTGGGATGTCAGGCACTCAGCGAGATG | |
| | ATACAGTTCTACCTGGAAGAGGTAATGCCCCAGGCCGAAAACCAAGAT | |
| | CCAGATATTAAGGCGCACGTGAACTCTCTGGGCGAGAATCTAAAAACT | |
| | CTGCGCTTGAGACTCAGGCGGTGCCACAGGTTTCTGCCATGCGAAAAT | |
| | GGTGGATCCCACCATCACCATCACCACCATCAT | |
| 107 | AAGAGCAAAGCTGTAGAGCAGGTTAAAAACGCATTCAATAAGCTGCAA | WC0007 |
| | GAGAAGGGTATTTATAAGGCCATGTCCGAGTTTGACATCTTCATTAAC | |
| | TACATCGAGGCCTACATGACGATGAAGATCAGGAACACTTCTCCCGGA | |
| | CAGGGCACACAGTCTGAAAATTCATGCACCCATTTCCCCGGAAATTTG | |
| | CCAAACATGCTGCGGAAGCTGCGAGATGCGTTTAGTCGGGTCAAAACC | |
| | TTTTTTCAGATGAAGGACCAGCTGGATAATTTATTACTCAAGGAGTCC | |
| | CTTCTGGAGGATTTCAAAGGCTATTTGGGGTGCCAAGCACTGTCGGAG | |
| | ATGATACAGTTCTACCTGGAAGAAGTGATGCCACAAGCCGAAAACCAG | |
| | GACCCTGATATTAAAGCTCACGTGAATAGCCTGGGGGAGAATCTAAAA | |
| | ACACTTCGTCTCCGCCTCAGAAGGTGTCACAGATTTCTCCCTTGTGAA | |
| | AACGGTGGATCCCACCATCACCATCACCACCATCAT | |

TABLE 6
Human Inverted Monomer Amino Acid Sequences

| SEQ ID | Sequence + GGS linker and HHHHHHHH | |
|---|---|---|
| NO | (SEQ ID NO: 140) chelating peptide | Notes |
| 27 | KSKAVEQVKNAFNKLQEKGIYKAMSEFDIFINYIEAYMTMKIRNSPGQGT | DR1060 |
| | QSENSCTHFPGNLPNMLRDLRDAFSRVKTFFQMKDQLDNLLLKESLLEDF | |
| | KGYLGCQALSEMIQFYLEEVMPQAENQDPDIKAHVNSLGENLKTLRLRLR | |
| | RCHRFLPCENGGSHHHHHHHH | |
| 108 | KSKAVEQVKNAFNKLQEKGIYKAMSEFDIFINYIEAYMTMKIRNTSPGQG | DR1061 |
| | TQSENSCTHFPGNLPNMLRDLRDAFSRVKTFFQMKDQLDNLLLKESLLED | |
| | FKGYLGCQALSEMIQFYLEEVMPQAENQDPDIKAHVNSLGENLKTLRLRL | |
| | RRCHRFLPCENGGSHHHHHHHH | |
| 109 | KSKAVEQVKNAFNKLQEKGIYKAMSEFDIFINYIEAYMTMKIRNPGQGTQ | DR1062 |
| | SENSCTHFPGNLPNMLRDLRDAFSRVKTFFQMKDQLDNLLLKESLLEDFK | |
| | GYLGCQALSEMIQFYLEEVMPQAENQDPDIKAHVNSLGENLKTLRLRLRR | |
| | CHRFLPCENGGSHHHHHHHH | |
| 93 | MSKAVEQVKNAFNKLQEKGIYKAMSEFDIFINYIEAYMTMKIRNSPGQGT | SS0166 |
| | QSENSCTHFPGNLPNMLRDLRDAFSRVKTFFQMKDQLDNLLLKESLLEDF | |
| | KGYLGCQALSEMIQFYLEEVMPQAENQDPDIKAHVNSLGENLKTLRLRLR | |
| | RCHRFLPCEN | |
| 110 | KSKAVEQVKNAFNKLQEKGIYKAMSEFDIFINYIEAYMTMKIRNAVPNQR | DR1057 |
| | DYDPNCTHFPGNLPNMLRDLRDAFSRVKTFFQMKDQLDNLLLKESLLEDF | |
| | KGYLGCQALSEMIQFYLEEVMPQAENQDPDIKAHVNSLGENLKTLRLRLR | |
| | RCHRFLPCENGGSHHHHHHHH | |
| 111 | KSKAVEQVKNAFNKLQEKGIYKAMSEFDIFINYIEAYMTMKIRGKGPLTQ | DR1058 |
| | PSEDCTHFPGNLPNMLRDLRDAFSRVKTFFQMKDQLDNLLLKESLLEDFK | |
| | GYLGCQALSEMIQFYLEEVMPQAENQDPDIKAHVNSLGENLKTLRLRLRR | |
| | CHRFLPCENGGSHHHHHHHH | |
| 112 | KSKAVEQVKNAFNKLQEKGIYKAMSEFDIFINYIEAYMTMKIRGSGSGSG | DR1059 |
| | SGSGSCTHFPGNLPNMLRDLRDAFSRVKTFFQMKDQLDNLLLKESLLEDF | |
| | KGYLGCQALSEMIQFYLEEVMPQAENQDPDIKAHVNSLGENLKTLRLRLR | |
| | RCHRFLPCENGGSHHHHHHHH | |
| 113 | KSKAVEQVKNAFNKLQEKGIYKAMSEFDIFINYIEAYMTMKIRNSPGQGT | WC0006 |
| | QSENSCTHFPGNLPNMLRKLRDAFSRVKTFFQMKDQLDNLLLKESLLEDF | (D25K) |
| | KGYLGCQALSEMIQFYLEEVMPQAENQDPDIKAHVNSLGENLKTLRLRLR | |
| | RCHRFLPCENGGSHHHHHHHH | |
| 114 | KSKAVEQVKNAFNKLQEKGIYKAMSEFDIFINYIEAYMTMKIRNTSPGQG | WC0007 |
| | TQSENSCTHFPGNLPNMLRKLRDAFSRVKTFFQMKDQLDNLLLKESLLED | (D25K) |
| | FKGYLGCQALSEMIQFYLEEVMPQAENQDPDIKAHVNSLGENLKTLRLRL | |
| | RRCHRFLPCENGGSHHHHHHHH | |

TABLE 7
Human Dimerized Inverted Monomer Nucleic Acid Sequences

| SEQ ID | | |
|---|---|---|
| NO | Sequence | Notes |
| 115 | AAAAGCAAGGCCGTGGAGCAAGTGAAGAACGCCTTCAACAAACTGCAA | TP0020 |
| | GAGAAAGGGATCTATAAGGCGATGTCCGAGTTTGACATATTTATCAAC | |
| | TACATCGAGGCATACATGACTATGAAGATCCGTAATAGCCCCGGCCAA | |
| | GGCACACAGTCAGAGAACTCTTGTACCCATTTCCCTGGGAACCTCCCA | |
| | AACATGCTGCGTGATCTGAGAGACGCCTTCAGTAGAGTGAAGACTTTT | |
| | TTTCAGATGAAGGACCAACTCGATAACCTGCTTCTTAAAGAAAGCCTG | |
| | CTGGAGGACTTTAAGGGATACCTTGGCTGCCAAGCCCTAAGCGAAATG | |
| | ATCCAATTTTATTTAGAAGAGGTCATGCCCCAAGCCGAGAACCAAGAC | |
| | CCCGACATTAAGGCCCATGTGAACTCTCTGGGCGAGAACCTGAAAACT | |
| | CTCCGCCTGAGACTAAGAAGATGCCACAGATTTCTGCCCTGCGAGAAC | |
| | AAATCAAAGGCCGTCGAGCAAGTGAAAAACGCGTTTAACAAGCTGCAA | |
| | GAAAAGGGAATATACAAGGCCATGTCAGAGTTCGACATCTTCATAAAT | |
| | TATATCGAAGCATACATGACCATGAAAATCAGAAACAGCCCCGGACAA | |
| | GGCACCCAAAGCGAGAATAGCTGCACCCACTTCCCCGGAAACCTGCCC | |
| | AACATGCTGAGAGACTTGAGAGATGCCTTCTCGAGGGTGAAGACCTTT | |
| | TTTCAAATGAAGGATCAGCTTGATAATCTGTTGCTGAAGGAGTCTCTG | |
| | CTGGAGGATTTCAAGGGCTACCTGGGCTGTCAAGCCCTGAGCGAAATG | |
| | ATTCAGTTTTACCTGGAAGAGGTGATGCCCCAAGCTGAGAATCAAGAC | |
| | CCCGACATTAAAGCACATGTGAACAGCCTGGGGGAGAACTTGAAGACC | |
| | TTACGATTGAGATTAAGAAGGTGTCACAGATTCCTGCCCTGCGAAAAC | |
| | GGCGGCAGCCACCACCACCACCACCACCACCAC | |
| 116 | AAAAGCAAGGCCGTGGAGCAAGTGAAGAACGCCTTCAACAAACTGCAA | TP0021 |
| | GAGAAAGGGATCTATAAGGCGATGTCCGAGTTTGACATATTTATCAAC | |
| | TACATCGAGGCATACATGACTATGAAGATCCGTAATAGCCCCGGCCAA | |
| | GGCACACAGTCAGAGAACTCTTGTACCCATTTCCCTGGGAACCTCCCA | |
| | AACATGCTGCGTGATCTGAGAGACGCCTTCAGTAGAGTGAAGACTTTT | |
| | TTTCAGATGAAGGACCAACTCGATAACCTGCTTCTTAAAGAAAGCCTG | |
| | CTGGAGGACTTTAAGGGATACCTTGGCTGCCAAGCCCTAAGCGAAATG | |
| | ATCCAATTTTATTTAGAAGAGGTCATGCCCCAAGCCGAGAACCAAGAC | |
| | CCCGACATTAAGGCCCATGTGAACTCTCTGGGCGAGAACCTGAAAACT | |
| | CTCCGCCTGAGACTAAGAAGATGCCACAGATTTCTGCCCTGCGAGAAC | |
| | GGCGGTAAATCAAAGGCCGTCGAGCAAGTGAAAAACGCGTTTAACAAG | |
| | CTGCAAGAAAAGGGAATATACAAGGCCATGTCAGAGTTCGACATCTTC | |
| | ATAAATTATATCGAAGCATACATGACCATGAAAATCAGAAACAGCCCC | |
| | GGACAAGGCACCCAAAGCGAGAATAGCTGCACCCACTTCCCCGGAAAC | |
| | CTGCCCAACATGCTGAGAGACTTGAGAGATGCCTTCTCGAGGGTGAAG | |
| | ACCTTTTTTCAAATGAAGGATCAGCTTGATAATCTGTTGCTGAAGGAG | |
| | TCTCTGCTGGAGGATTTCAAGGGCTACCTGGGCTGTCAAGCCCTGAGC | |
| | GAAATGATTCAGTTTTACCTGGAAGAGGTGATGCCCCAAGCTGAGAAT | |
| | CAAGACCCCGACATTAAAGCACATGTGAACAGCCTGGGGGAGAACTTG | |
| | AAGACCTTACGATTGAGATTAAGAAGGTGTCACAGATTCCTGCCCTGC | |
| | GAAAACGGCGGCAGCCACCACCACCACCACCACCACCAC | |
| 117 | AAAAGCAAGGCCGTGGAGCAAGTGAAGAACGCCTTCAACAAACTGCAA | TP0022 |
| | GAGAAAGGCATTTACAAAGCCATGTCCGAGTTTGACATATTTATCAAC | |
| | TACATTGAGGCGTATATGACCATGAAGATCCGTAATACCTCTCCCGGA | |
| | CAAGGCACCCAAAGCGAAAACAGCTGCACACACTTTCCTGGCAATCTC | |
| | CCCAACATGCTACGTGACCTGAGAGACGCCTTTAGCAGAGTGAAAACC | |
| | TTCTTTCAGATGAAGGATCAGCTCGACAACCTTCTTTTAAAAGAGAGC | |
| | CTGCTGGAGGATTTCAAAGGCTATCTGGGCTGCCAAGCCCTGTCTGAG | |
| | ATGATTCAGTTCTACCTGGAAGAGGTGATGCCTCAAGCAGAAAATCAA | |
| | GACCCCGATATCAAGGCACACGTGAATAGCCTGGGGGAAAACTTGAAA | |
| | ACCCTTAGACTGCGTCTGAGACGATGTCACCGCTTCCTGCCCTGCGAG | |
| | AACAAATCCAAGGCTGTGGAGCAAGTGAAGAATGCATTCAACAAGCTT | |
| | CAAGAGAAAGGTATCTACAAGGCCATGAGCGAGTTCGACATCTTCATA | |
| | AATTACATCGAGGCCTACATGACCATGAAAATCAGAAACACAAGCCCC | |
| | GGCCAAGGCACACAGAGCGAAAACAGCTGCACCCACTTCCCCGGCAAC | |
| | CTGCCCAATATGCTGAGGGACCTAAGAGACGCTTTCAGCAGAGTTAAA | |
| | ACTTTTTTTCAGATGAAAGACCAACTAGATAACTTGCTGCTGAAGGAA | |
| | AGCCTGCTGGAAGACTTCAAGGGCTACCTGGGATGCCAAGCCCTGTCA | |
| | GAGATGATTCAATTCTACCTAGAAGAGGTGATGCCCCAAGCCGAAAAC | |
| | CAAGACCCCGATATAAAAGCGCACGTGAACAGCCTGGGCGAGAATCTT | |
| | AAGACCTTAAGGCTGCGACTGAGGCGGTGCCACAGATTCCTGCCCTGT | |
| | GAGAACGGCGGTAGCCACCACCACCACCACCACCACCAC | |
| 118 | AAAAGCAAGGCCGTGGAGCAAGTGAAGAACGCCTTCAACAAACTGCAA | TP0023 |
| | GAGAAAGGCATTTACAAAGCCATGTCCGAGTTTGACATATTTATCAAC | |
| | TACATTGAGGCGTATATGACCATGAAGATCCGTAATACCTCTCCCGGA | |
| | CAAGGCACCCAAAGCGAAAACAGCTGCACACACTTTCCTGGCAATCTC | |
| | CCCAACATGCTACGTGACCTGAGAGACGCCTTTAGCAGAGTGAAAACC | |
| | TTCTTTCAGATGAAGGATCAGCTCGACAACCTTCTTTTAAAAGAGAGC | |
| | CTGCTGGAGGATTTCAAAGGCTATCTGGGCTGCCAAGCCCTGTCTGAG | |
| | ATGATTCAGTTCTACCTGGAAGAGGTGATGCCTCAAGCAGAAAATCAA | |
| | GACCCCGATATCAAGGCACACGTGAATAGCCTGGGGGAAAACTTGAAA | |
| | ACCCTTAGACTGCGTCTGAGACGATGTCACCGCTTCCTGCCCTGCGAG | |
| | AACGGTGGTAAATCCAAGGCTGTGGAGCAAGTGAAGAATGCATTCAAC | |
| | AAGCTTCAAGAGAAAGGTATCTACAAGGCCATGAGCGAGTTCGACATC | |
| | TTCATAAATTACATCGAGGCCTACATGACCATGAAAATCAGAAACACA | |
| | AGCCCCGGCCAAGGCACACAGAGCGAAAACAGCTGCACCCACTTCCCC | |
| | GGCAACCTGCCCAATATGCTGAGGGACCTAAGAGACGCTTTCAGCAGA | |
| | GTTAAAACTTTTTTTCAGATGAAAGACCAACTAGATAACTTGCTGCTG | |
| | AAGGAAAGCCTGCTGGAAGACTTCAAGGGCTACCTGGGATGCCAAGCC | |
| | CTGTCAGAGATGATTCAATTCTACCTAGAAGAGGTGATGCCCCAAGCC | |
| | GAAAACCAAGACCCCGATATAAAAGCGCACGTGAACAGCCTGGGCGAG | |
| | AATCTTAAGACCTTAAGGCTGCGACTGAGGCGGTGCCACAGATTCCTG | |
| | CCCTGTGAGAACGGCGGTAGCCACCACCACCACCACCACCACCAC | |
| 119 | AAAAGCAAGGCCGTGGAGCAAGTGAAGAACGCCTTCAACAAACTGCAA | TP0024 |
| | GAGAAAGGGATCTATAAGGCGATGTCCGAGTTTGACATATTTATCAAC | |
| | TACATCGAGGCATACATGACTATGAAGATCCGTAATAGCCCCGGCCAA | |
| | GGCACACAGTCAGAGAACTCTTGTACCCATTTCCCTGGGAACCTCCCA | |
| | AACATGCTGCGTAAACTGAGAGACGCCTTCAGTAGAGTGAAGACTTTT | |
| | TTTCAGATGAAGGACCAACTCGATAACCTGCTTCTTAAAGAAAGCCTG | |
| | CTGGAGGACTTTAAGGGATACCTTGGCTGCCAAGCCCTAAGCGAAATG | |
| | ATCCAATTTTATTTAGAAGAGGTCATGCCCCAAGCCGAGAACCAAGAC | |
| | CCCGACATTAAGGCCCATGTGAACTCTCTGGGCGAGAACCTGAAAACT | |
| | CTCCGCCTGAGACTAAGAAGATGCCACAGATTTCTGCCCTGCGAGAAC | |
| | AAATCAAAGGCCGTCGAGCAAGTGAAAAACGCGTTTAACAAGCTGCAA | |
| | GAAAAGGGAATATACAAGGCCATGTCAGAGTTCGACATCTTCATAAAT | |
| | TATATCGAAGCATACATGACCATGAAAATCAGAAACAGCCCCGGACAA | |
| | GGCACCCAAAGCGAGAATAGCTGCACCCACTTCCCCGGAAACCTGCCC | |
| | AACATGCTGAGAAAGTTGAGAGATGCCTTCTCGAGGGTGAAGACCTTT | |
| | TTTCAAATGAAGGATCAGCTTGATAATCTGTTGCTGAAGGAGTCTCTG | |
| | CTGGAGGATTTCAAGGGCTACCTGGGCTGTCAAGCCCTGAGCGAAATG | |
| | ATTCAGTTTTACCTGGAAGAGGTGATGCCCCAAGCTGAGAATCAAGAC | |
| | CCCGACATTAAAGCACATGTGAACAGCCTGGGGGAGAACTTGAAGACC | |
| | TTACGATTGAGATTAAGAAGGTGTCACAGATTCCTGCCCTGCGAAAAC | |
| | GGCGGCAGCCACCACCACCACCACCACCACCAC | |
| 120 | AAAAGCAAGGCCGTGGAGCAAGTGAAGAACGCCTTCAACAAACTGCAA | TP0045 |
| | GAGAAAGGGATCTATAAGGCGATGTCCGAGTTTGACATATTTATCAAC | |
| | TACATCGAGGCATACATGACTATGAAGATCCGTAATAGCCCCGGCCAA | |
| | GGCACACAGTCAGAGAACTCTTGTACCCATTTCCCTGGGAACCTCCCA | |
| | AACATGCTGCGTGATCTGAGAGACGCCTTCAGTAGAGTGAAGACTTTT | |
| | TTTCAGATGAAGGACCAACTCGATAACCTGCTTCTTAAAGAAAGCCTG | |
| | CTGGAGGACTTTAAGGGATACCTTGGCTGCCAAGCCCTAAGCGAAATG | |
| | ATCCAATTTTATTTAGAAGAGGTCATGCCCCAAGCCGAGAACCAAGAC | |
| | CCCGACATTAAGGCCCATGTGAACTCTCTGGGCGAGAACCTGAAACTC | |
| | CTCCGCCTGAGACTAAGAAGATGCCACAGATTTCTGCCCTGCGAGAAC | |
| | AAATCAAAGGCCGTCGAGCAAGTGAAAAACGCGTTTAACAAGCTGCAA | |
| | GAAAAGGGAATATACAAGGCCATGTCAGAGTTCGACATCTTCATAAAT | |
| | TATATCGAAGCATACATGACCATGAAAATCAGAAACAGCCCCGGACAA | |
| | GGCACCCAAAGCGAGAATAGCTGCACCCACTTCCCCGGAAACCTGCCC | |
| | AACATGCTGAGAGACTTGAGAGATGCCTTCTCGAGGGTGAAGACCTTT | |
| | TTTCAAATGAAGGATCAGCTTGATAATCTGTTGCTGAAGGAGTCTCTG | |
| | CTGGAGGATTTCAAGGGCTACCTGGGCTGTCAAGCCCTGAGCGAAATG | |
| | ATTCAGTTTTACCTGGAAGAGGTGATGCCCCAAGCTGAGAATCAAGAC | |
| | CCCGACATTAAAGCACATGTGAACAGCCTGGGGGAGAACTTGAAGCTG | |
| | TTACGATTGAGATTAAGAAGGTGTCACAGATTCCTGCCCTGCGAAAAC | |
| | GGCGGCAGCCACCACCACCACCACCACCACCAC | |

TABLE 8
Human Dimerized Inverted Monomer Amino Acid Sequences

| SEQ ID | Sequence + GGS linker and HHHHHHHH | |
|---|---|---|
| NO | (SEQ ID NO: 140) chelating peptide | Notes |
| 121 | KSKAVEQVKNAFNKLQEKGIYKAMSEFDIFINYIEAYMTMKIRNSPGQGT | TP0020 |
| | QSENSCTHFPGNLPNMLRDLRDAFSRVKTFFQMKDQLDNLLLKESLLEDF | |
| | KGYLGCQALSEMIQFYLEEVMPQAENQDPDIKAHVNSLGENLKTLRLRLR | |
| | RCHRFLPCENKSKAVEQVKNAFNKLQEKGIYKAMSEFDIFINYIEAYMTM | |
| | KIRNSPGQGTQSENSCTHFPGNLPNMLRDLRDAFSRVKTFFQMKDQLDNL | |
| | LLKESLLEDFKGYLGCQALSEMIQFYLEEVMPQAENQDPDIKAHVNSLGE | |
| | NLKTLRLRLRRCHRFLPCENGGSHHHHHHHH | |
| 122 | KSKAVEQVKNAFNKLQEKGIYKAMSEFDIFINYIEAYMTMKIRNSPGQGT | TP0021 |
| | QSENSCTHFPGNLPNMLRDLRDAFSRVKTFFQMKDQLDNLLLKESLLEDF | |
| | KGYLGCQALSEMIQFYLEEVMPQAENQDPDIKAHVNSLGENLKTLRLRLR | |
| | RCHRFLPCENGGKSKAVEQVKNAFNKLQEKGIYKAMSEFDIFINYIEAYM | |
| | TMKIRNSPGQGTQSENSCTHFPGNLPNMLRDLRDAFSRVKTFFQMKDQLD | |
| | NLLLKESLLEDFKGYLGCQALSEMIQFYLEEVMPQAENQDPDIKAHVNSL | |
| | GENLKTLRLRLRRCHRFLPCENGGSHHHHHHHH | |
| 123 | KSKAVEQVKNAFNKLQEKGIYKAMSEFDIFINYIEAYMTMKIRNTSPGQG | TP0022 |
| | TQSENSCTHFPGNLPNMLRDLRDAFSRVKTFFQMKDQLDNLLLKESLLED | |
| | FKGYLGCQALSEMIQFYLEEVMPQAENQDPDIKAHVNSLGENLKTLRLRL | |
| | RRCHRFLPCENKSKAVEQVKNAFNKLQEKGIYKAMSEFDIFINYIEAYMT | |
| | MKIRNTSPGQGTQSENSCTHFPGNLPNMLRDLRDAFSRVKTFFQMKDQLD | |
| | NLLLKESLLEDFKGYLGCQALSEMIQFYLEEVMPQAENQDPDIKAHVNSL | |
| | GENLKTLRLRLRRCHRFLPCENGGSHHHHHHHH | |
| 124 | KSKAVEQVKNAFNKLQEKGIYKAMSEFDIFINYIEAYMTMKIRNTSPGQG | TP0023 |
| | TQSENSCTHFPGNLPNMLRDLRDAFSRVKTFFQMKDQLDNLLLKESLLED | |
| | FKGYLGCQALSEMIQFYLEEVMPQAENQDPDIKAHVNSLGENLKTLRLRL | |
| | RRCHRFLPCENGGKSKAVEQVKNAFNKLQEKGIYKAMSEFDIFINYIEAY | |
| | MTMKIRNTSPGQGTQSENSCTHFPGNLPNMLRDLRDAFSRVKTFFQMKD | |
| | QLDNLLLKESLLEDFKGYLGCQALSEMIQFYLEEVMPQAENQDPDIKAHV | |
| | NSLGENLKTLRLRLRRCHRFLPCENGGSHHHHHHHH | |
| 125 | KSKAVEQVKNAFNKLQEKGIYKAMSEFDIFINYIEAYMTMKIRNSPGQGT | TP0024 |
| | QSENSCTHFPGNLPNMLRKLRDAFSRVKTFFQMKDQLDNLLLKESLLEDF | (D25K) |
| | KGYLGCQALSEMIQFYLEEVMPQAENQDPDIKAHVNSLGENLKTLRLRLR | |
| | RCHRFLPCENKSKAVEQVKNAFNKLQEKGIYKAMSEFDIFINYIEAYMTM | |
| | KIRNSPGQGTQSENSCTHFPGNLPNMLRKLRDAFSRVKTFFQMKDQLDNL | |
| | LLKESLLEDFKGYLGCQALSEMIQFYLEEVMPQAENQDPDIKAHVNSLGE | |
| | NLKTLRLRLRRCHRFLPCENGGSHHHHHHHH | |
| 126 | KSKAVEQVKNAFNKLQEKGIYKAMSEFDIFINYIEAYMTMKIRNSPGQGT | TP0045 |
| | QSENSCTHFPGNLPNMLRDLRDAFSRVKTFFQMKDQLDNLLLKESLLEDF | (T100L) |
| | KGYLGCQALSEMIQFYLEEVMPQAENQDPDIKAHVNSLGENLKLLRLRLR | |
| | RCHRFLPCENKSKAVEQVKNAFNKLQEKGIYKAMSEFDIFINYIEAYMTM | |
| | KIRNSPGQGTQSENSCTHFPGNLPNMLRDLRDAFSRVKTFFQMKDQLDNL | |
| | LLKESLLEDFKGYLGCQALSEMIQFYLEEVMPQAENQDPDIKAHVNSLGE | |
| | NLKLLRLRLRRCHRFLPCENGGSHHHHHHHH | |

TABLE 9
Murine Inverted Monomer Nucleic Acid Sequences

| SEQ ID | | |
|---|---|---|
| NO | Sequence | Notes |
| 127 | AACAAGAGCAAGGCCGTGGAGCAAGTGAAGAGCGACTTCAACAAGCT | WC161 |
| | GCAAGACCAAGGCGTGTACAAGGCCATGAACGAGTTCGACATCTTCA | |
| | TCAACGCCATCGAGGCCTACATGATGATCAAGATGAAGAGCAGCAGA | |
| | GGGCAGTACAGCAGAGAGGACAACAACTGCACCCACTTCCCCGTGGG | |
| | GCAGAGCCACATGCTGCTGGAGCTGAGAACCGCCTTCAGCCAAGTGA | |
| | AGACCTTCTTTCAGACCAAGGATCAGCTGGACAACATCCTGCTGACC | |
| | GACAGCCTGATGCAAGACTTCAAGGGCTACCTGGGCTGCCAAGCCCT | |
| | GAGCGAGATGATTCAGTTCTACCTGGTGGAGGTGATGCCCCAAGCCG | |
| | AGAAGCACGGCCCCGAGATCAAGGAGCACCTGAACAGCCTGGGCGAG | |
| | AAGCTGAAGACCCTGAGAATGAGACTGAGAAGATGCCACAGATTCCT | |
| | GCCCTGCGAGAACGGCGGATCCCACCATCACCATCACCACCATCATT | |
| | AGTGAGCGGCCGC | |
| 128 | AACAAGAGCAAGGCCGTGGAGCAAGTGAAGAGCGACTTCAACAAGCT | TP0036 |
| | GCAAGACCAAGGCGTGTACAAGGCCATGAACGAGTTCGACATCTTCA | |
| | TCAACTGCATCGAGGCCTACATGATGATCAAGATGAAGAGCAGCAGA | |
| | GGGCAGTACAGCAGAGAGGACAACAACTGCACCCACTTCCCCGTGGG | |
| | GCAGAGCCACATGCTGCTGGAGCTGAGAACCGCCTTCAGCCAAGTGA | |
| | AGACCTTCTTTCAGACCAAGGATCAGCTGGACAACATCCTGCTGACC | |
| | GACAGCCTGATGCAAGACTTCAAGGGCTACCTGGGCTGCCAAGCCCT | |
| | GAGCGAGATGATTCAGTTCTACCTGGTGGAGGTGATGCCCCAAGCCG | |
| | AGAAGCACGGCCCCGAGATCAAGGAGCACCTGAACAGCCTGGGCGAG | |
| | AAGCTGAAGACCCTGAGAATGAGACTGAGAAGATGCCACAGATTCCT | |
| | GCCCTGCGAGAACGGCGGCAGCCACCACCACCACCACCACCACCACT | |
| | GATAAGCGGCCGC | |
| 129 | AACAAGAGCAAGGCCGTGGAGCAAGTGAAGAGCGACTTCAACAAGCT | TP0041 |
| | GCAAGACCAAGGCGTGTACAAGGCCATGAACGAGTTCGACATCTTCA | |
| | TCAACTGCATCGAGGCCTACATGATGATCAAGATGAAGAGCAGCAGA | |
| | GGGCAGTACAGCAGAGAGGACAACAACTGCACCCACTTCCCCGTGGG | |
| | GCAGAGCCACATGCTGCTGGAGCTGAGAACCGCCTTCAGCCAAGTGA | |
| | AGACCTTCTTTCAGACCAAGGATCAGCTGGACAACATCCTGCTGACC | |
| | GACAGCCTGATGCAAGACTTCAAGGGCTACCTGGGCTGCCAAGCCCT | |
| | GAGCGAGATGATTCAGTTCTACCTGGTGGAGGTGATGCCCCAAGCCG | |
| | AGAAGCACGGCCCCGAGATCAAGGAGCACCTGAACAGCCTGGGCGAG | |
| | AAGCTGAAGCTGCTGAGAATGAGACTGAGAAGATGCCACAGATTCCT | |
| | GCCCTGCGAGAACGGCGGCAGCCACCACCACCACCACCACCACCACT | |
| | GATAAGCGGCCGC | |

TABLE 10
Murine Inverted Monomer Amino Acid Sequences

| SEQ ID | Sequence + GGS linker and HHHHHHHH | |
|---|---|---|
| NO | (SEQ ID NO: 140) chelating peptide | Notes |
| 28 | NKSKAVEQVKSDFNKLQDQGVYKAMNEFDIFINAIEAYMMIKMKSSRGQ | WC161 |
| | YSREDNNCTHFPVGQSHMLLELRTAFSQVKTFFQTKDQLDNILLTDSLM | (Mutation of |
| | QDFKGYLGCQALSEMIQFYLVEVMPQAEKHGPEIKEHLNSLGEKLKTLR | unpaired |
| | MRLRRCHRFLPCENGGSHHHHHHHH | cysteine in |
| | | TP0036) |
| 130 | NKSKAVEQVKSDFNKLQDQGVYKAMNEFDIFINCIEAYMMIKMKSSRGQ | TP0036 |
| | YSREDNNCTHFPVGQSHMLLELRTAFSQVKTFFQTKDQLDNILLTDSLM | |
| | QDFKGYLGCQALSEMIQFYLVEVMPQAEKHGPEIKEHLNSLGEKLKTLR | |
| | MRLRRCHRFLPCENGGSHHHHHHHH | |
| 131 | NKSKAVEQVKSDFNKLQDQGVYKAMNEFDIFINCIEAYMMIKMKSSRGQ | TP0041 |
| | YSREDNNCTHFPVGQSHMLLELRTAFSQVKTFFQTKDQLDNILLTDSLM | |
| | QDFKGYLGCQALSEMIQFYLVEVMPQAEKHGPEIKEHLNSLGEKLKLLR | |
| | MRLRRCHRFLPCENGGSHHHHHHHH | |

Table 10B provides additional murine inverted dimers of the present disclosure. All substitutions referenced in the description are numbered in accordance with the mature mIL10 canonical reference sequence:

(SEQ ID NO: 141)
SRGQYSREDNNCTHFPVGQSHMLLELRTAFSQVKTFFQTKDQLDNILLTD
SLMQDFKGYLGCQALSEMIQFYLVEVMPQAEKHGPEIKEHLNSLGEKLKT
LRMRLRRCHRFLPCENKSKAVEQVKSDFNKLQDQGVYKAMNEFDIFINCI
EAYMMIKMKS

The binding specificity of the murine inverted monomers provided in Table 10A for the immobilized mIL10Ra receptor subunit conjugated to each arm of an immobilized Fc were confirmed by surface plasmon resonance in substantial accordance with the teaching of the Examples and the data provided in Table CA. As discussed in the examples, the affinity of IL10 for the IL10Rb subunit in the absence of IL10 is very low and does not facilitate measurement by SPR. Consequently, the molecules were evaluated by ELISA to confirm binding to both IL10Ra and IL10Rb. The data indicating that some of these inverted monomers exhibited greater binding affinity to the IL10Ra receptor than the WC 131 control molecule described below. Based on the sequence homology of the murine and human molecules, the amino acid substitutions in the murine inverted monomers described in Tables 10 and 10B may be incorporated into a human inverted monomer of formula 1. In some embodiments, the present disclosure provides a human inverted monomer comprising one or more amino acid substitution at a position substituted in a homologous murine inverted monomer of Table 10 or 10B.

TABLE 10A
mIL10Ra SPR Binding Data

| | | k_{ON} | | Affinity | Rmax | Load | |
|---|---|---|---|---|---|---|---|
| Ligand | Analyte | (1/Ms) | k_{OFF} (1/s) | (nM) | (RU) | (RU) | Activity |
| mIL10RaFc | DR2802 | 5.15E+06 | 1.18E−02 | 2.29 | 35.5 | 126.2 | 91% |
| | DR2803 | 2.72E+06 | 4.63E−03 | 1.7 | 32.5 | 125.7 | 84% |
| | DR2804 | 4.13E+06 | 6.57E−03 | 1.59 | 34.5 | 124.8 | 90% |
| | DR2805 | 8.77E+05 | 2.71E−02 | 30.9 | 36.4 | 124.6 | 95% |
| | DR2806 | 3.75E+06 | 1.01E−02 | 2.7 | 34.4 | 122.7 | 91% |
| | DR2807 | 1.32E+05 | 1.91E−02 | 144 | 118.3 | 122.9 | 313% |
| | DR2808 | 8.44E+06 | 1.14E−02 | 1.35 | 33.2 | 121.1 | 89% |
| | DR2809 | 1.28E+06 | 2.21E−02 | 17.3 | 43.1 | 118.5 | 118% |
| | DR2813 | 2.55E+06 | 1.02E−01 | 39.9 | 48.1 | 122 | 128% |
| | DR2815 | 5.75E+06 | 5.73E−03 | 1.0 | 31.6 | 121.2 | 85% |
| | DR2816 | 5.71E+06 | 9.19E−03 | 1.61 | 33.6 | 122.1 | 89% |
| | DR2817 | 9.22E+06 | 1.52E−02 | 1.65 | 29.3 | 122.1 | 78% |
| | DR2818 | 7.43E+06 | 1.74E−02 | 2.34 | 30.4 | 120.1 | 82% |
| | DR2819 | 8.17E+06 | 2.21E−02 | 2.71 | 30.9 | 120.7 | 83% |
| | DR2820 | 4.63E+06 | 1.56E−02 | 3.37 | 32.1 | 120 | 87% |
| | DR2821 | 8.09E+06 | 3.63E−02 | 4.49 | 34.3 | 119.5 | 93% |
| | DR2822 | 3.96E+06 | 2.54E−02 | 6.42 | 33 | 117.8 | 91% |
| | DR2823 | 5.90E+06 | 3.07E−02 | 5.2 | 33.1 | 116.4 | 92% |
| | WC161ctrl | 3.09E+06 | 1.63E−02 | 5.27 | 34.9 | 115.9 | 98% |

TABLE 10B
Murine Inverted Monomer Sequences

| | | | DESCRIPTION/ | |
|---|---|---|---|---|
| SEQ | | ALTERNATE | AMINO ACID | |
| ID | NAME | NAME | SUBSTITUTIONS | AA SEQUENCE |
| 142 | DR2802 | mil10inv_ | G-[mIL10 117-160 | GKSKAVEQVKSIFNKLQDQGV |
| | | HA_29.9_ | D127I/C149A]- | YKAMNEFDIFINAIEAYMMIK |
| | | del SUMO | [mIL10 6-116 | MKSSREDNNCTHFMVAMSHML |
| | | | P16M/G18A/Q19M/ | LELRTAFSQVKTFFQTKDQLD |
| | | | M77L] | NILLTDSLMQDFKGYLGCQAL |
| | | | | SEMIQFYLVEVLPQAEKHGPE |
| | | | | IKEHLNSLGEKLKTLRMRLRR |
| | | | | CHRFLPCEN |
| 143 | DR2803 | mil10inv_ | G-[mIL10 117-160 | GKSKAVEQVKSIFNKLQDQGV |
| | | HA_28.7_ | D127I/C149A]- | YKAMNEFDIFINAIEAYMMIK |
| | | del SUMO | [mIL10 6-116 | MKSSREDNNCTHAPVAFWHML |
| | | | F15A/G18A/Q19F/ | LELRTAFSQVKTFFQTKDQLD |
| | | | S20W/M77L | NILLTDSLMQDFKGYLGCQAL |
| | | | | SEMIQFYLVEVLPQAEKHGPE |
| | | | | IKEHLNSLGEILKTLRMRLRR |
| | | | | CHRFLPCEN |
| 144 | DR2804 | mil10inv_ | G-[mIL10 117-160 | GKSKAVEQVKSIFNKLQDQGV |
| | | HA_26.6_ | D127I/C149A]- | YKAMNEFDIFINAIEAYMMIK |
| | | del SUMO | [mIL10 6-116 | MKSSREDNNCTHAPVGMSHML |
| | | | F15A/G18M/M77L/ | LELRTAFSQVKTFFQTKDQLD |
| | | | N92I/K971] | NILLTDSLMQDFKGYLGCQAL |
| | | | | SEMIQFYLVEVLPQAEKHGPE |
| | | | | IKEHLISLGEILKTLRMRLRR |
| | | | | CHRFLPCEN |
| 145 | DR2805 | | G-[mIL10 117-160 | GKSKAVEQVKSIFNKLQDQGV |
| | | | D127I/F146V/ | YKAMNEFDIVINAIEAYMMIK |
| | | | C149A]-[mIL10 | MKSSREDNNCTHAPVMFSHML |
| | | | 6-116 F15A/G18M/ | LELRTAFSQVKTFFQTKDQLD |
| | | | Q19F/M77L/K97I] | NILLTDSLMQDFKGYLGCQAL |
| | | | | SEMIQFYLVEVLPQAEKHGPE |
| | | | | IKEHLNSLGEILKTLRMRLRR |
| | | | | CHRFLPCEN |
| 146 | DR2806 | mil10inv_ | G-[117-160 | GKSKAVEQVKSIFNKLQDQGV |
| | | HA_22.2_ | D127I/C149A]- | YKAMNEFDIFINAIEAYMMIK |
| | | del SUMO | [mIL10 6-116 | MKSSREDNNCTHFNVGLSHML |
| | | | P16N/Q19L/M77L/ | LELRTAFSQVKTFFQTKDQLD |
| | | | K97I] | NILLTDSLMQDFKGYLGCQAL |
| | | | | SEMIQFYLVEVLPQAEKHGPE |
| | | | | IKEHLNSLGEILKTLRMRLRR |
| | | | | CHRFLPCEN |
| 147 | DR2807 | mil10inv_ | G-[mIL10 117-160 | GKSKAVEQVKSIFNKLQDQGV |
| | | HA_20.0_ | D127I/C149A]- | YKAMNEFDIFINAIEAYMMIK |
| | | del SUMO | [mIL10 6-116 | MKSSREDNNCTHFPVGQSHML |
| | | | wild type] | LELRTAFSQVKTFFQTKDQLD |
| | | | | NILLTDSLMQDFKGYLGCQAL |
| | | | | SEMIQFYLVEVMPQAEKHGPE |
| | | | | IKEHLNSLGEKLKTLRMRLRR |
| | | | | CHRFLPCEN |
| 148 | DR2808 | mil10inv_ | G-[mIL 10 117-160 | GKSKAVEQVKSIFNKLQDQGV |
| | | HA_14.2_ | D127I/C149A]- | YKAMNEFDIFINAIEAYMMIK |
| | | del SUMO | [mIL106-116 F15A/ | MKSSREDNNCTHAPVGQSHML |
| | | | M77L/K97I] | LELRTAFSQVKTFFQTKDQLD |
| | | | | NILLTDSLMQDFKGYLGCQAL |
| | | | | SEMIQFYLVEVLPQAEKHGPE |
| | | | | IKEHLNSLGEILKTLRMRLRR |
| | | | | CHRFLPCEN |
| 149 | DR2809 | mil10inv_ | G-[mIL10117-160 | GKSKAVEQVKSIFNKLQDQGV |
| | | HA_13.2_ | D127I/C149A]- | YKAMNEFDIFINAIEAYMMIK |
| | | del SUMO | [mIL106-116 F15A/ | MKSSREDNNCTHAPVGQSHML |
| | | | M77L] | LELRTAFSQVKTFFQTKDQLD |
| | | | | NILLTDSLMQDFKGYLGCQAL |
| | | | | SEMIQFYLVEVLPQAEKHGPE |
| | | | | IKEHLNSLGEKLKTLRMRLRR |
| | | | | CHRFLPCEN |
| 150 | DR2811 | mil10inv_ | G-[mIL 10 117-160 | GKSKAVEQVKSDFNKLQDQGV |
| | | HA_61.2 | M140N/N148T/ | YKANNEFMIFITAIEAYMMIK |
| | | | C149A]-[mIL10 | MKSSREDNNCTHFWVWMSHML |
| | | | 6-116 P16W/G18W/ | LELRTAASQVKTFFQTKDQLD |
| | | | Q19M/F30A/M77L/ | NILLTDSLMQDFKGYLGCQAL |
| | | | N92I/K97I] | SEMIQFYLVEVLPQAEKHGPE |
| | | | | IKEHLISLGELLKTLRMRLRR |
| | | | | CHRFLPCEN |
| 151 | DR2812 | mil10inv_ | G;[mIL10 007-160 | GKSKAVEQVKSDFNKLQDQGV |
| | | HA_54.5 | N141V/F143I/ | YKAMVEIDIFINAIEAYMMIK |
| | | G- | C149A]-[mIL10 | MKSSREDNNCTHFWVIMLHML |
| | | [117-160 | 6-116P16W/G18I/ | LELRTAMSQVKTFFQTKDQLD |
| | | N141V/ | Q19M/S20L/F30M/ | NILLTDSLMQDFKGYLGCQAL |
| | | F143I/ | M77L/N92I/K97L] | SEMIQFYLVEVLPQAEKHGPE |
| | | C149A] | | IKEHLISLGELLKTLRMRLRR |
| | | | | CHRFLPCEN |
| 152 | DR2813 | mil10inv_ | G-[mIL10117-160 | GKSKAVEQVKSDFNKLQDQGV |
| | | HA_37.7 | C149A]-[mIL10 | YKAMNEFDIFINAIEAYMMIK |
| | | | 6-116 G18A/Q19M/ | MKSSREDNNCTHFWVWMSHML |
| | | | F30A/M77L/N92I/ | LELRTAASQVKTFFQTKDQLD |
| | | | K97I] | NILLTDSLMQDFKGYLGCQAL |
| | | | | SEMIQFYLVEVLPQAEKHGPE |
| | | | | IKEHLISLGEILKTLRMRLRR |
| | | | | CHRFLPCEN |
| 153 | DR2814 | mil10inv_ | G-[mIL10 117-160 | GKSKAVEQVKSDFNKLQDQGV |
| | | HA_43.3 | N141V/C149A]- | YKAMVEFDIFINAIEAYMMIK |
| | | | [mIL106-116 P16M/ | MKSSREDNNCTHFMVALSHML |
| | | | G18A/M77L/N92I/ | LELRTAFSQVKTFFQTKDQLD |
| | | | K97I] | NILLTDSLMQDFKGYLGCQAL |
| | | | | SEMIQFYLVEVLPQAEKHGPE |
| | | | | IKEHLISLGEILKTLRMRLRR |
| | | | | CHRFLPCEN 2814 |
| 154 | DR2815 | mil10inv_ | G-[mIL10 117-160 | GKSKAVEQVKSDFNKLQDQGV |
| | | HA_34.1 | C149A]-[mIL10 | YKAMNEFDIFINAIEAYMMIK |
| | | | 6-116 P16N/ | MKSSREDNNCTHFNVVMSHML |
| | | | G18VQ19M/M77L] | LELRTAFSQVKTFFQTKDQLD |
| | | | | NILLTDSLMQDFKGYLGCQAL |
| | | | | SEMIQFYLVEVLPQAEKHGPE |
| | | | | IKEHLNSLGEKLKTLRMRLRR |
| | | | | CHRFLPCEN |
| 155 | DR2816 | mil10inv_ | G-[mIL10117-160 | GKSKAVEQVKSDFNKLQDQGV |
| | | HA_26.1 | C149A]-[mIL10 | YKAMNEFDIFINAIEAYMMIK |
| | | | 6-116 F15A/M77L/ | MKSSREDNNCTHAPVGQSHML |
| | | | K97I | LELRTAFSQVKTFFQTKDQLD |
| | | | | NILLTDSLMQDFKGYLGCQAL |
| | | | | SEMIQFYLVEVLPQAEKHGPE |
| | | | | IKEHLNSLGEILKTLRMRLRR |
| | | | | CHRFLPCEN |
| 156 | DR2817 | mil10inv_ | G-[mIL10 117-160 | GKSKAVEQVKSDFNKLQDQGV |
| | | HA_24.0 | C149A]-[mIL10 | YKAMNEFDIFINAIEAYMMIK |
| | | | 6-116 F15A/M77L] | MKSSREDNNCTHAPVGQSHML |
| | | | | LELRTAFSQVKTFFQTKDQLD |
| | | | | NILLTDSLMQDFKGYLGCQAL |
| | | | | SEMIQFYLVEVLPQAEKHGPE |
| | | | | IKEHLNSLGEKLKTLRMRLRR |
| | | | | CHRFLPCEN |
| 157 | DR2818 | mil10inv_ | G-[mIL10 117-160 | GKSKCVEQVKSDFNKLQDQGV |
| | | 7loop_5_ | A120C/C149A]- | YKAMNEFDIFINAIEAYMMIK |
| | | 94_disulf | [mIL10 4-116 | MKSQYSREDNNCTHFPVGQSH |
| | | | L52C] | MLLELRTAFSQVKTFFQTKDQ |
| | | | | LDNILLTDSCMQDFKGYLGCQ |
| | | | | ALSEMIQFYLVEVMPQAEKHG |
| | | | | PEIKEHLNSLGEKLKTLRMRL |
| | | | | RRCHRFLPCEN |
| 158 | DR2819 | mil10inv_ | G-[mIL 10 117-160 | GKSKCVEQVKSDFNKLQDQGV |
| | | 7loop_5_ | A120C/C149A]- | YKAMNEFDIFINAIEAYMMIK |
| | | 97_disulf | [mIL10 4-116 | MKSQYSREDNNCTHFPVGQSH |
| | | | D52C] | MLLELRTAFSQVKTFFQTKDQ |
| | | | | LDNILLTDSLMQCFKGYLGCQ |
| | | | | ALSEMIQFYLVEVMPQAEKHG |
| | | | | PEIKEHLNSLGEKLKTLRMRL |
| | | | | RRCHRFLPCEN |
| 159 | DR2820 | mil10inv_ | G-[mIL 10 117-160 | GKCKAVEQVKSDFNKLQDQGV |
| | | 7loop_3_ | S118C/C149A]- | YKAMNEFDIFINAIEAYMMIK |
| | | 97_disulf | [mIL10 4-116 | MKSQYSREDNNCTHFPVGQSH |
| | | | D52C] | MLLELRTAFSQVKTFFQTKDQ |
| | | | | LDNILLTDSLMQCFKGYLGCQ |
| | | | | ALSEMIQFYLVEVMPQAEKHG |
| | | | | PEIKEHLNSLGEKLKTLRMRL |
| | | | | RRCHRFLPCEN 2820 |
| 160 | DR2821 | mil10inv_ | G-[mIL10117-160 | GKSKACEQVKSDFNKLQDQGV |
| | | 7loop_ | V121C/C149A]- | YKAMNEFDIFINAIEAYMMIK |
| | | 6_109_ | [mIL10 4-116 | MKSQYSREDNNCTHFPVGQSH |
| | | disulf | E67C] | MLLELRTAFSQVKTFFQTKDQ |
| | | | | LDNILLTDSLMQDFKGYLGCQ |
| | | | | ALSCMIQFYLVEVMPQAEKHG |
| | | | | PEIKEHLNSLGEKLKTLRMRL |
| | | | | RRCHRFLPCEN |
| 161 | DR2822 | mil10inv_ | G-[mIL10 117-160 | GKSKAVEQVKSDFNKLQDQGV |
| | | 4loop | C149A]-[mIL10 | YKAMNEFDIFINAIEAYMMIK |
| | | | 7-116 wild type] | MKSREDNNCTHFPVGQSHMLL |
| | | | | ELRTAFSQVKTFFQTKDQLDN |
| | | | | ILLTDSLMQDFKGYLGCQALS |
| | | | | EMIQFYLVEVMPQAEKHGPEI |
| | | | | KEHLNSLGEKLKTLRMRLRRC |
| | | | | HRFLPCEN |
| 162 | DR2823 | mil10inv_ | G-[117-160 | GKSKAVEQVKSDFNKLQDQGV |
| | | 5loop | C149A]- | YKAMNEFDIFINAIEAYMMIK |
| | | | [mIL10 6-116 | MKSSREDNNCTHFPVGQSHML |
| | | | wild type] | LELRTAFSQVKTFFQTKDQLD |
| | | | | NILLTDSLMQDFKGYLGCQAL |
| | | | | SEMIQFYLVEVMPQAEKHGPE |
| | | | | IKEHLNSLGEKLKTLRMRLRR |
| | | | | CHRFLPCEN |

TABLE 11
Murine Dimerized Inverted Monomer Nucleic Acid Sequences

| SEQ ID | | |
|---|---|---|
| NO | Sequence | Notes |
| 132 | AACAAAAGCAAGGCCGTTGAGCAAGTGAAATCGGACTTCAACAAACTG | TP0038 |
| | CAAGACCAAGGCGTCTACAAGGCTATGAATGAGTTTGACATATTTATC | |
| | AACTGCATCGAAGCGTACATGATGATAAAGATGAAGAGCAGCCGCGGG | |
| | CAGTACAGCAGAGAAGACAACAATTGTACCCATTTCCCTGTCGGCCAA | |
| | TCCCACATGCTGCTAGAACTGAGAACCGCCTTCAGTCAAGTGAAGACT | |
| | TTCTTTCAGACCAAGGACCAACTCGATAACATCCTTCTTACGGATAGC | |
| | CTTATGCAAGATTTCAAGGGATACCTTGGCTGCCAAGCCCTAAGCGAA | |
| | ATGATCCAATTCTACCTGGTGGAGGTGATGCCGCAAGCTGAGAAACAC | |
| | GGCCCTGAAATTAAGGAGCATCTGAACTCTCTGGGCGAGAAGCTGAAA | |
| | ACTCTGAGAATGAGATTGAGAAGATGTCACAGATTTCTGCCCTGCGAG | |
| | AACGGCGGTGGAAAAAGCAAAGCCGTCGAGCAAGTGAAATCAGACTTC | |
| | AACAAGCTGCAAGACCAAGGCGTGTACAAGGCAATGAACGAGTTCGAC | |
| | ATATTCATCAACTGTATCGAGGCCTACATGATGATCAAGATGAAGAGC | |
| | TCTAGAGGCCAATACAGTAGAGAGGATAACAACTGCACCCACTTCCCC | |
| | GTGGGGCAGAGCCATATGCTGTTGGAGCTAAGAACCGCTTTCAGCCAA | |
| | GTGAAAACTTTTTTTCAGACGAAGGATCAACTCGACAATATCCTGCTG | |
| | ACGGACAGCCTGATGCAAGACTTCAAGGGCTACCTGGGATGCCAAGCC | |
| | CTGTCAGAGATGATTCAATTCTACCTAGTGGAGGTAATGCCCCAAGCC | |
| | GAAAAGCACGGACCCGAAATTAAAGAACACCTGAACAGCCTGGGCGAG | |
| | AAACTGAAGACCTTAAGGATGAGACTGAGGAGATGCCACAGATTCCTG | |
| | CCCTGTGAAAACGGCGGCAGTCACCACCACCACCACCACCACCACTGA | |
| | TAAGCGGCCGC | |
| 133 | AACAAAAGCAAGGCCGTTGAGCAAGTGAAATCGGACTTCAACAAACTG | TP0043 |
| | CAAGACCAAGGCGTCTACAAGGCTATGAATGAGTTTGACATATTTATC | |
| | AACTGCATCGAAGCGTACATGATGATAAAGATGAAGAGCAGCCGCGGG | |
| | CAGTACAGCAGAGAAGACAACAATTGTACCCATTTCCCTGTCGGCCAA | |
| | TCCCACATGCTGCTAGAACTGAGAACCGCCTTCAGTCAAGTGAAGACT | |
| | TTCTTTCAGACCAAGGACCAACTCGATAACATCCTTCTTACGGATAGC | |
| | CTTATGCAAGATTTCAAGGGATACCTTGGCTGCCAAGCCCTAAGCGAA | |
| | ATGATCCAATTCTACCTGGTGGAGGTGATGCCGCAAGCTGAGAAACAC | |
| | GGCCCTGAAATTAAGGAGCATCTGAACTCTCTGGGCGAGAAGCTGAAA | |
| | CTCCTGAGAATGAGATTGAGAAGATGTCACAGATTTCTGCCCTGCGAG | |
| | AACGGCGGTGGAAAAAGCAAAGCCGTCGAGCAAGTGAAATCAGACTTC | |
| | AACAAGCTGCAAGACCAAGGCGTGTACAAGGCAATGAACGAGTTCGAC | |
| | ATATTCATCAACTGTATCGAGGCCTACATGATGATCAAGATGAAGAGC | |
| | TCTAGAGGCCAATACAGTAGAGAGGATAACAACTGCACCCACTTCCCC | |
| | GTGGGGCAGAGCCATATGCTGTTGGAGCTAAGAACCGCTTTCAGCCAA | |
| | GTGAAAACTTTTTTTCAGACGAAGGATCAACTCGACAATATCCTGCTG | |
| | ACGGACAGCCTGATGCAAGACTTCAAGGGCTACCTGGGATGCCAAGCC | |
| | CTGTCAGAGATGATTCAATTCTACCTAGTGGAGGTAATGCCCCAAGCC | |
| | GAAAAGCACGGACCCGAAATTAAAGAACACCTGAACAGCCTGGGCGAG | |
| | AAACTGAAGCTGTTAAGGATGAGACTGAGGAGATGCCACAGATTCCTG | |
| | CCCTGTGAAAACGGCGGCAGTCACCACCACCACCACCACCACCACTGA | |
| | TAAGCGGCCGC | |

TABLE 12
Murine Dimerized Inverted Monomer Amino Acid Sequences

| SEQ ID | Sequence + GGS linker and HHHHHHHH | |
|---|---|---|
| NO | (SEQ ID NO: 140) chelating peptide | Notes |
| 134 | NKSKAVEQVKSDFNKLQDQGVYKAMNEFDIFINCIEAYMMIKMKSSRGQ | TP0038 |
| | YSREDNNCTHFPVGQSHMLLELRTAFSQVKTFFQTKDQLDNILLTDSLM | |
| | QDFKGYLGCQALSEMIQFYLVEVMPQAEKHGPEIKEHLNSLGEKLKTLR | |
| | MRLRRCHRFLPCENGGGKSKAVEQVKSDFNKLQDQGVYKAMNEFDIFIN | |
| | CIEAYMMIKMKSSRGQYSREDNNCTHFPVGQSHMLLELRTAFSQVKTFF | |
| | QTKDQLDNILLTDSLMQDFKGYLGCQALSEMIQFYLVEVMPQAEKHGPE | |
| | IKEHLNSLGEKLKTLRMRLRRCHRFLPCENGGSHHHHHHHH | |
| 135 | NKSKAVEQVKSDFNKLQDQGVYKAMNEFDIFINCIEAYMMIKMKSSRGQ | TP0043 |
| | YSREDNNCTHFPVGQSHMLLELRTAFSQVKTFFQTKDQLDNILLTDSLM | |
| | QDFKGYLGCQALSEMIQFYLVEVMPQAEKHGPEIKEHLNSLGEKLKLLR | |
| | MRLRRCHRFLPCENGGGKSKAVEQVKSDFNKLQDQGVYKAMNEFDIFIN | |
| | CIEAYMMIKMKSSRGQYSREDNNCTHFPVGQSHMLLELRTAFSQVKTFF | |
| | QTKDQLDNILLTDSLMQDFKGYLGCQALSEMIQFYLVEVMPQAEKHGPE | |
| | IKEHLNSLGEKLKLLRMRLRRCHRFLPCENGGSHHHHHHHH | |

What is claimed is:

1. A polypeptide comprising an amino acid sequence of the formula 1:
[A]-Llx-[B]-L2-[C]-L3-[D]-L4y-[E]-L5-[F] (1)
wherein:
x and y are independently selected from 0 (absent) or 1 (present);
[A] is a polypeptide having 0, 1 or 2 amino acid substitutions relative to the amino acid sequence SKAVEQVKNAFNKL (SEQ ID NO:1);
x=0 (no linker, L1 is absent), or x=1 and L1 comprises a polypeptide linker of from 1 to 5 amino acids;
[B] is a polypeptide having 0, 1 or 2 amino acid substitutions relative to the amino acid sequence “EKGIYKAMSEFDIFINYIEAYMTMKIR” (SEQ ID NO:2):
L2 comprises a linker of from 10 to 25 amino acids;
[C] is a polypeptide having 0, 1 or 2 amino acid substitutions relative to the amino acid sequence “NLPNMLRDLRDAFSRVKTFFQMKD” (SEQ ID NO:3);
L3 comprises a linker of from 4 to 11 amino acids;
[D] is a polypeptide having 0, 1 or 2 amino acid substitutions relative to the amino acid sequence “KESLLEDFKG” (SEQ ID NO:4);
y=0 (L4 is absent), or
y=1 and L4 comprises a polypeptide linker of from 1 to 5 amino acids;
[E] is a polypeptide having 0, 1 or 2 amino acid substitutions relative to the amino acid sequence “LGCQALSEMIQFYLEEVMPQAEN” (SEQ ID NO:5);
L5 comprises a linker of from 1 to 7 amino acids; and
[F] is a polypeptide having 0, 1 or 2 amino acid substitutions relative to the amino acid sequence “IKAHVNSLGENLKTLRLRLRRC” (SEQ ID NO:6).

2. The polypeptide of claim 1, wherein L1, L2, L3, L4 and/or L5 comprise a GS-linker.

3. The polypeptide of claim 1 or 2, wherein L1 is the amino acid glutamine (Q).

4. The polypeptide of any one of claims 1 to 3, wherein L2 is a polypeptide having the amino acid sequence NSPGQGTQSENSCTHFPG (SEQ ID NO:7) or NTSPGQGTQSENSCTHFPG (SEQ ID NO:23).

5. The polypeptide of any one of claims 1 to 4, wherein L3 is a polypeptide having the amino acid sequence QLDNLLL (SEQ ID NO:8).

6. The polypeptide of any one of claims 1 to 5, wherein L4 is the amino acid glycine (“G”) or comprises the amino acid sequence GY.

7. The polypeptide of any one of claims 1 to 6, wherein L5 is a polypeptide having the amino acid sequence QDPD (SEQ ID NO:9).

8. The polypeptide of any one of claims 1 to 7, wherein the polypeptide comprises an amino acid sequence having at least 95% sequence identity to SEQ ID NO: 10 or SEQ ID NO:11.

9. The polypeptide of claim 8, wherein the polypeptide comprises the amino acid sequence
(SEQ ID NO: 10)
KSKAVEQVKNAFNKLQEKGIYKAMSEFDIFINYIEAYMTMKIRNSPGQGT
QSENSCTHFPGNLPNMLRDLRDAFSRVKTFFQMKDQLDNLLLKESLLEDF
KGYLGCQALSEMIQFYLEEVMPQAENQDPDIKAHVNSLGENLKTLRLRLR
RCHRFLPCEN.

10. The polypeptide of claim 8, wherein the polypeptide comprises the amino acid sequence
(SEQ ID NO: 11)
KSKAVEQVKNAFNKLQEKGIYKAMSEFDIFINYIEAYMTMKIRNTSPGQG
TQSENSCTHFPGNLPNMLRDLRDAFSRVKTFFQMKDQLDNLLLKESLLED
FKGYLGCQALSEMIQFYLEEVMPQAENQDPDIKAHVNSLGENLKTLRLRL
RRCHRFLPCEN.

11. The polypeptide of any one of claims 1 to 8, wherein the polypeptide comprises an amino acid sequence having at least 90%, 91%, 92%, 93%, 94%, 95%, 96%, 97%, 98%, 99% or 100% sequence identity hIL10 inverted monomer selected from the group consisting of SEQ ID NO: 10, SEQ ID NO: 11, SEQ ID NO:90, SEQ ID NO:91, SEQ ID NO:92, SEQ ID NO:93, SEQ ID NO:94, SEQ ID NO:95, SEQ ID NO:96, SEQ ID NO:97, and SEQ ID NO:98.

12. A polypeptide comprising formula (2):
[Monomer1]-linkerₓ-[Monomer2] (2)
wherein Monomer 1 and Monomer 2 each, independently, comprise a polypeptide selected from a polypeptide of claim 1, and x=0 (linker absent) or 1 (linker present).

13. The polypeptide of claim 12, wherein Monomer 1 and Monomer 2 each, independently, comprise a polypeptide comprising an amino acid sequence having at least 90%, 91%, 92%, 93%, 94%, 95%, 96%, 97%, 98%, 99% or 100% sequence identity to an amino acid sequence selected from the group consisting of SEQ ID NO:10, SEQ ID NO: 11, SEQ ID NO: 90, SEQ ID NO:91, SEQ ID NO:92, SEQ ID NO:93, SEQ ID NO:94, SEQ ID NO:95, SEQ ID NO:96, SEQ ID NO:97, and SEQ ID NO:98.

14. The polypeptide of claim 13, wherein Monomer 1 and Monomer 2 each comprise a polypeptide comprising the amino acid sequence of SEQ ID NO:10.

15. The polypeptide of claim 13, wherein Monomer 1 and Monomer 2 each comprise a polypeptide comprising the amino acid sequence of SEQ ID NO:11.

16. The polypeptide of any one of claims 12 to 15, wherein x is 1 (linker present) and the linker comprises a GS-linker.

17. The polypeptide of any one of claims 12 to 15, wherein x is 0 (linker absent).

18. The polypeptide of claim 12, wherein the polypeptide of formula (2) comprises in the following amino to carboxyl order:
(a) Monomer 1 comprising:
(i) a first polypeptide comprising an amino acid sequence having at least 90%, 91%, 92%, 93%, 94%, 95%, 96%, 97%, 98%, 99% or 100% sequence identity to a polypeptide selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO:14), numbered in accordance with mature wild-type human IL-10 (SEQ ID NO:20);
(ii) a first linkers, wherein x=0 (linker absent) or 1 (linker present); and
(iii) a second polypeptide comprising an amino acid having at least 90%, 91%, 92%, 93%, 94%, 95%, 96%, 97%, 98%, 99% or 100% sequence identity to a polypeptide sequence selected from the group consisting of amino acid residues 1-160 of human IL-10 (SEQ ID NO:20): amino acid residues 2-160 of human IL-10 (SEQ ID NO:36); amino acid residues 3-160 of human IL-10 (SEQ ID NO:37); amino acid residues 4-160 of human IL-10 (SEQ ID NO:38): amino acid residues 5-160 of human IL-10 (SEQ ID NO:39); amino acid residues 6-160 of human IL-10 (SEQ ID NO:40); amino acid residues 7-160 of human IL-10 (SEQ ID NO:41); amino acid residues 8-160 of human IL-10 (SEQ ID NO:42); amino acid residues 9-160 of human IL-10 (SEQ ID NO:43); amino acid residues 10-160 of human IL-10 (SEQ ID NO:44); and amino acid residues 11-160 of human IL-10 (SEQ ID NO:45) numbered in accordance with mature wild-type hIL10 (SEQ ID NO:20);
(b) a second linker_{y}, wherein y=0 (linker absent) or 1 (linker present); and
(c) Monomer 2 comprising a polypeptide having at least 90%, 91%, 92%, 93%, 94%, 95%, 96%, 97%, 98%, 99% or 100% sequence identity to a polypeptide sequence selected from the group consisting of amino acid residues 1-116 of human IL-10 (SEQ ID NO:15); amino acid residues 2-116 of human IL-10 (SEQ ID NO:16); amino acid residues 3-116 of human IL-10 (SEQ ID NO:17), amino acid residues 4-116 of human IL-10 (SEQ ID NO:18), amino acid residues 5-116 of human IL-10 (SEQ ID NO: 19), amino acid residues 6-116 of human IL-10 (SEQ ID NO:29), amino acid residues 7-116 of human IL-10 (SEQ ID NO:30), amino acid residues 8-116 of human IL-10 (SEQ ID NO:31), amino acid residues 9-116 of human IL-10 (SEQ ID NO:32), amino acid residues 10-116 of human IL-10 (SEQ ID NO:33); and amino acid residues 11-116 of human IL-10 (SEQ ID NO:34), numbered in accordance with mature human IL-10 (SEQ ID NO:20).

19. The polypeptide of claim 12, wherein the polypeptide of formula (2) comprises in the following amino to carboxyl order:
(a) Monomer 1 comprising:
(i) a first polypeptide comprising an amino acid sequence selected from the group consisting amino acid residues 117-158 of human IL-10 (SEQ ID NO: 12); amino acid residues 117-159 of human IL-10 (SEQ ID NO: 13); and amino acid residues 117-160 of human IL-10 (SEQ ID NO: 14), numbered in accordance with mature human IL-10 (SEQ ID NO:20),
(ii) a first linkers, wherein x=0 (linker absent) or 1 (linker present);
(iii) a second polypeptide comprising an amino acid sequence selected from the group consisting of amino acid residues 1-116 of human IL-10 (SEQ ID NO:15):
(b) a second linker_{y}, wherein y=0 (linker absent) or 1 (linker present);
(c) Monomer 2 comprising:
(i) a first polypeptide comprising an amino acid sequence selected from the group consisting amino acid residues 117-158 of human IL-10 (SEQ ID NO:12); amino acid residues 117-159 of human IL-10 (SEQ ID NO:13); and amino acid residues 117-160 of human IL-10 (SEQ ID NO: 14), numbered in accordance with mature human IL-10 (SEQ ID NO:20),
(ii) a third linker_{z}, wherein z=0 (linker absent) or 1 (linker present); and
(iii) a second polypeptide comprising an amino acid sequence selected from the group consisting of amino acid residues 1-116 of human IL-10 (SEQ ID NO:15).

20. The polypeptide of claim 12, wherein the polypeptide comprises an amino acid sequence having at least 90%, 91%, 92%, 93%, 94%, 95%, 96%, 97%, 98%, 99% or 100% sequence identity hIL10 inverted monomer selected from the group consisting of SEQ ID NOS:46-51.

21. The polypeptide of any one of claims 1 to 20, wherein the polypeptide is PEGylated.

22. The polypeptide of claim 21, wherein the PEG molecule is linear or branched and has a molecular weight of from about 10 kD to about 80 kD.

23. The polypeptide of claim 22, wherein the PEG molecule is a 40 kD branched PEG molecule comprising two 20 kD arms.

24. The polypeptide of any one of claims 21 to 23, wherein the PEG molecule is covalently attached to the N-terminus of the polypeptide.

25. The polypeptide of any one of claims 1 to 24, wherein the polypeptide exhibits cell type biased activity relative to the wild type IL10 species from which the inverted IL10 monomer was derived.

26. The polypeptide of claim 25 that: (a) exhibits a significant level of at least one anti-inflammatory property of wild type IL10 and (b) exhibits a significantly reduced level of at least one pro-inflammatory property of wild type IL10.

27. The polypeptide of claim 26 wherein the at least one anti-inflammatory property is selected from the group consisting of suppression of expression or secretion of IL1b, TNFa or IL6 in a myeloid cell.

28. The polypeptide of claim 27 wherein the at least one pro-inflammatory property is selected from the group consisting of suppression of expression or secretion of IFNγ, granzyme A or granzyme B in a T cell.

29. The polypeptide of claim 27 wherein the at least one anti-inflammatory property is selected from the group consisting of suppression of expression or secretion of IL1b, TNFa or IL6 in a myeloid cell and the at least one pro-inflammatory property is selected from the group consisting of suppression of expression or secretion of IFNγ, granzyme A or granzyme B in a T cell.

30. The polypeptide of claim 25 wherein the cell type biased activity is the production of phospho-STAT3.

31. The polypeptide of claim 30 wherein the pSTAT3 Eₘₐₓ of the polypeptide is greater than 20%, greater than 30%, greater than 40%, greater than 50%, greater than 60%, or greater than 70% of the pSTAT3 Eₘₐₓ of wild-type hIL10 in myeloid cells.

32. The polypeptide of claim 31 wherein the pSTAT3 Eₘₐₓ in a lymphocyte less than 70%, less than 60%, less than 50%, less than 40%, or less than 30%, of the pSTAT3 Eₘₐₓ of a wild-type hIL10 in the lymphocyte.

33. The polypeptide of claim 30 wherein (a) the pSTAT3 Eₘₐₓ of the polypeptide is greater than 20%, greater than 30%, greater than 40%, greater than 50%, greater than 60%, or greater than 70% of the pSTAT3 Eₘₐₓ of wild-type hIL10 in myeloid cells and (b) the pSTAT3 Eₘₐₓ in a lymphocyte less than 70%, less than 60%, less than 50%, less than 40%, or less than 30%, of the pSTAT3 Eₘₐₓ of a wild-type hIL10 in the lymphocyte.

34. A nucleic acid sequence encoding a polypeptide of any one of claims 1-33.

35. A recombinant vector comprising the nucleic acid sequence of claim 34 operably linked to one or more expression control sequences.

36. A recombinant cell transformed with the recombinant vector of claim 35.

37. A method of making a polypeptide of any one of claims 1 to 33, the method comprising the steps of (a) culturing the host cell of claim 36 under conditions suitable for the expression of the polypeptide and (b) recovering the polypeptide from the host cell culture.

38. The method of claim 37, wherein the host cell is a mammalian host cell.

39. The method of claim 37, wherein the host cell is a bacterial cell.

40. A composition comprising a polypeptide of any one of claims 1 to 33, a nucleic acid sequence of claim 34, or a recombinant vector of claim 35 and one or more pharmaceutically acceptable salts, excipients, and/or diluents.

41. A method of treating a mammalian subject suffering from a disease, disorder or condition, the method comprising administering to said subject a therapeutically effective amount a polypeptide of any one of claims 1 to 33, or a composition of claim 40.

42. The method of claim 41, wherein the disease, disorder or condition is an autoimmune disease, disorder or condition.

43. The method of claim 42, wherein the autoimmune disease, disorder or condition is selected from the group consisting of ulcerative colitis, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, and SEA Syndrome (Seronegativity, Enthesopathy, Arthropathy Syndrome).

44. The method of claim 41, wherein disease disorder or condition is a cancer associated with chronic inflammation.

45. The method of any one of claims 41 to 44, wherein the treating prevents the progression of the disease, disorder or condition.

46. The method of any one of claims 41 to 44, wherein the treating ameliorates one or more symptoms of the disease, disorder or condition.

47. A method of preventing a disease, disorder or condition in a mammalian subject at risk for developing the disease, disorder or condition, the method comprising administering to said subject a prophylactically effective amount of the composition of claim 40 prior to the onset of symptoms of the disease, disorder or condition.

48. The method of claim 47, wherein the disease disorder or condition is a cancer associated with chronic inflammation.
